(19) United States
(12) Patent Application Publication
(10) Pub. No.: US 2023/0130109 A1
(43) Pub. Date: Apr. 27, 2023

(54) MACROCYCLIC INDOLE DERIVATIVES AS INHIBITORS OF MCL-1

(71) Applicant: Janssen Pharmaceutica NV, Beerse (BE)

(72) Inventors: Frederik Jan Rita ROMBOUTS, Wilmington, DE (US); Tristan REUILLON, Somerville, MA (US); Aldo PESCHIULLI, Antwerp (BE); Adriana-Ingrid VELTER, Antwerp (BE); Ann Marleen VOS, Turnhoutseweg 30 (BE)

(21) Appl. No.: 17/904,412

(22) PCT Filed: Feb. 18, 2021
(86) PCT No.: PCT/EP2021/053973

§ 371 Date: Aug. 17, 2022

(30) Foreign Application Priority Data
Feb. 21, 2020 (EP) 20158764.9
Apr. 16, 2020 (EP) 20169887.5
Jul. 9, 2020 (EP) 20184956.9

(51) Int. Cl. C07D 515/22; C07D 498/22; A61P 35/00

(52) U.S. Cl. CPC C07D 515/22; C07D 498/22; A61P 35/00

(57) ABSTRACT

The present invention relates to pharmaceutical agents useful for therapy and/or prophylaxis in a subject, pharmaceutical composition comprising such compounds, and their use as MCL-1 inhibitors, useful for treating diseases such as cancer.

### FIELD OF THE INVENTION

The present invention relates to pharmaceutical agents useful for therapy and/or prophylaxis in a subject, pharmaceutical composition comprising such compounds, and their use as MCL-1 inhibitors, useful for treating or preventing diseases such as cancer.

### BACKGROUND OF THE INVENTION

Cellular apoptosis or programmed cell death is critical to the development and homeostasis of many organs including the hematopoietic system. Apoptosis can be initiated via the extrinsic pathway, which is mediated by death receptors, or by the intrinsic pathway using the B cell lymphoma (BCL-2) family of proteins. Myeloid cell leukemia-1 (MCL-1) is a member of the BCL-2 family of cell survival regulators and is a critical mediator of the intrinsic apoptosis pathway. MCL-1 is one of five principal anti-apoptotic BCL-2 proteins (MCL-1, BCL-2, BCL-XL, BCL-w, and BFL1/A1) responsible for maintaining cell survival. MCL-1 continuously and directly represses the activity of the pro-apoptotic BCL-2 family proteins Bak and Bax and indirectly blocks apoptosis by sequestering BH3 only apoptotic sensitizer proteins such as Bim and Noxa. The activation of Bak/Bax following various types of cellular stress leads to aggregation on the mitochondrial outer membrane and this aggregation facilitates pore formation, loss of mitochondrial outer membrane potential, and subsequent release of cytochrome C into the cytosol. Cytosolic cytochrome C binds Apaf-1 and initiates recruitment of procaspase 9 to form apoptosome structures (Cheng et al. eLife 2016; 5: e17755). The assembly of apoptosomes activates the executioner cysteine proteases 3/7 and these effector caspases then cleave a variety of cytoplasmic and nuclear proteins to induce cell death (Julian et al. Cell Death and Differentiation 2017; 24, 1380-1389).

Avoiding apoptosis is an established hallmark of cancer development and facilitates the survival of tumor cells that would otherwise be eliminated due to oncogenic stresses, growth factor deprivation, or DNA damage (Hanahan and Weinberg. Cell 2011; 1-44). Thus, unsurprisingly, MCL-1 is highly upregulated in many solid and hematologic cancers relative to normal non-transformed tissue counterparts. The overexpression of MCL-1 has been implicated in the pathogenesis of several cancers where it correlated with poor outcome, relapse, and aggressive disease. Additionally, overexpression of MCL-1 has been implicated in the pathogenesis of the following cancers: prostate, lung, pancreatic, breast, ovarian, cervical, melanoma, B-cell chronic lymphocytic leukemia (CLL), acute myeloid leukemia (AML), and acute lymphoblastic leukemia (ALL). The human MCL-1 genetic locus (1q21) is frequently amplified in tumors and quantitatively increases total MCL-1 protein levels (Beroukhim et al. Nature 2010; 463 (7283) 899-905). MCL-1 also mediates resistance to conventional cancer therapeutics and is transcriptionally upregulated in response to inhibition of BCL-2 function (Yecies et al. Blood 2010; 115 (16)3304-3313).

A small molecule BH3 inhibitor of BCL-2 has demonstrated clinical efficacy in patients with chronic lymphocytic leukemia and is FDA approved for patients with CLL or AML (Roberts et al. NEJM 2016; 374:311-322). The clinical success of BCL-2 antagonism led to the development of several MCL-1 BH3 mimetics that show efficacy in preclinical models of both hematologic malignancies and solid tumors (Kotschy et al. Nature 2016; 538 477-486, Merino et al. Sci. Transl. Med; 2017 (9)).

MCL-1 regulates several cellular processes in addition to its canonical role in mediating cell survival including mitochondrial integrity and non-homologous end joining following DNA damage (Chen et al. JCI 2018; 128(1):500-516). The genetic loss of MCL-1 shows a range of phenotypes depending on the developmental timing and tissue deletion. MCL-1 knockout models reveal there are multiple roles for MCL-1 and loss of function impacts a wide range of phenotypes. Global MCL-1-deficient mice display embryonic lethality and studies using conditional genetic deletion have reported mitochondrial dysfunction, impaired activation of autophagy, reductions in B and T lymphocytes, increased B and T cell apoptosis, and the development of heart failure/cardiomyopathy (Wang et al. Genes and Dev 2013; 27 1351-1364, Steimer et al. Blood 2009; (113) 2805-2815).

WO2018178226 discloses MCL-1 inhibitors and methods of use thereof.

WO2017182625 discloses macrocyclic MCL-1 inhibitors for treating cancer.

WO2018178227 discloses the synthesis of MCL-1 inhibitors.

WO2020063792 discloses indole macrocyclic derivatives.

CN110845520 discloses macrocyclic indoles as MCL-1 inhibitors.

WO2020103864 discloses macrocyclic indoles as MCL-1 inhibitors.

There remains a need for MCL-1 inhibitors, useful for the treatment or prevention of cancers such as prostate, lung, pancreatic, breast, ovarian, cervical, melanoma, B-cell chronic lymphocytic leukemia (CLL), acute myeloid leukemia (AML), and acute lymphoblastic leukemia (ALL).

### SUMMARY OF THE INVENTION

The present invention concerns novel compounds of Formula (I):

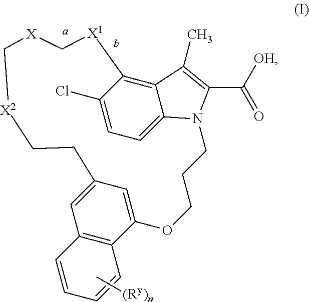

and the tautomers and the stereoisomeric forms thereof, wherein
X¹ represents

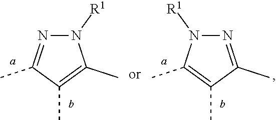

wherein ‘a’ and ‘b’ indicate how variable X¹ is attached to the remainder of the molecule;
R¹ and R² each independently represent hydrogen; methyl; or C₂₋₆alkyl optionally substituted with one or two substituents each independently selected from the group consisting of Het¹, —OR³, and —NR^{4a}R^{4b};
Het¹ represents morpholinyl or tetrahydropyranyl;
R³ represents hydrogen, C₁₋₄alkyl, —C₂₋₄alkyl-O—C₁₋₄alkyl, —C₂₋₄alkyl-OH, or —C₂₋₄alkyl-O—C₂₋₄alkyl-O—C₁₋₄alkyl;
R^{4a} and R^{4b} are each independently selected from the group consisting of hydrogen and C₁₋₄alkyl;
X² represents

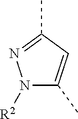

which can be attached to the remainder of the molecule in both directions;
X represents —O—, —S—, —S(═O)—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 0, 1 or 2;
and the pharmaceutically acceptable salts and the solvates thereof.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer.

In a particular embodiment, the invention relates to a compound of Formula (I), a pharmaceutically acceptable salt, or a solvate thereof, for use in the treatment or in the prevention of cancer.

The invention also relates to the use of a compound of Formula (I), a pharmaceutically acceptable salt, or a solvate thereof, in combination with an additional pharmaceutical agent for use in the treatment or prevention of cancer.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer.

Additionally, the invention relates to a method of treating or preventing a cell proliferative disease in a subject which comprises administering to the said subject an effective amount of a compound of Formula (I), a pharmaceutically acceptable salt, or a solvate thereof, as defined herein, or a pharmaceutical composition or combination as defined herein.

### DETAILED DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain fully saturated hydrocarbon radical having from 1 to 4 carbon atoms, such as methyl, ethyl, n-propyl, isopropyl, n-butyl, s-butyl, t-butyl and the like.

The term ‘C₁₋₆alkyl’ as used herein as a group or part of a group represents a straight or branched chain fully saturated hydrocarbon radical having from 1 to 6 carbon atoms, such as methyl, ethyl, n-propyl, isopropyl, n-butyl, s-butyl, t-butyl, n-pentyl, n-hexyl and the like.

The term ‘C₂₋₄alkyl’ as used herein as a group or part of a group represents a straight or branched chain fully saturated hydrocarbon radical having from 2 to 4 carbon atoms, such as ethyl, n-propyl, isopropyl, n-butyl, s-butyl, t-butyl and the like.

The term ‘C₂₋₆alkyl’ as used herein as a group or part of a group represents a straight or branched chain fully saturated hydrocarbon radical having from 2 to 6 carbon atoms, such as ethyl, n-propyl, isopropyl, n-butyl, s-butyl, t-butyl, n-pentyl, n-hexyl and the like.

The term ‘C₃₋₆cycloalkyl’ as used herein as a group or part of a group defines a fully saturated, cyclic hydrocarbon radical having from 3 to 6 carbon atoms, such as cyclopropyl, cyclobutyl, cyclopentyl and cyclohexyl.

It will be clear for the skilled person that S(═O)₂ or SO₂ represents a sulfonyl moiety.

It will be clear for the skilled person that CO or C(═O) represents a carbonyl moiety.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture.

Combinations of substituents and/or variables are permissible only if such combinations result in chemically stable compounds. ‘Stable compound’ is meant to indicate a compound that is sufficiently robust to survive isolation to a useful degree of purity from a reaction mixture.

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

It will be clear for the skilled person that

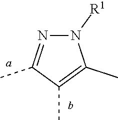

is an alternative representation for

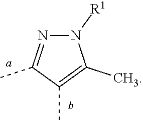

It will be clear for the skilled person that

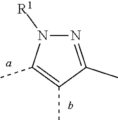

is an alternative representation for

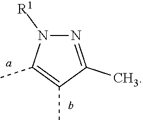

It will be clear that a Compound of Formula (I) includes Compounds of Formula (I-x) and (I-y) (both directions of X² being

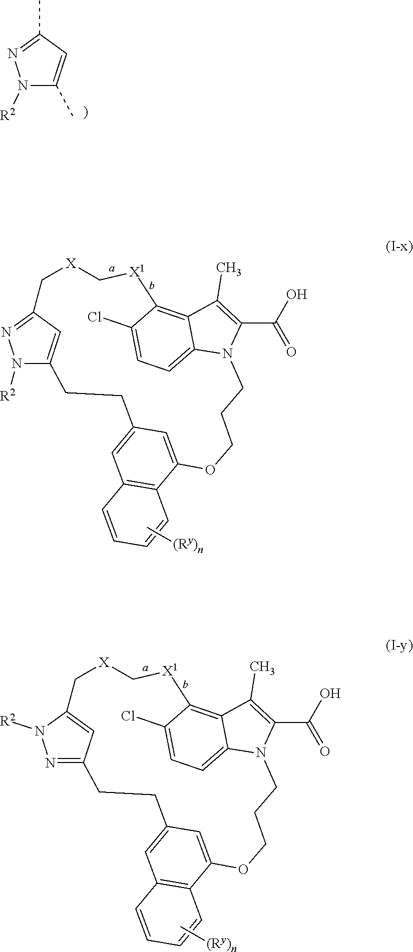

When any variable occurs more than one time in any constituent or in any formula (e.g. Formula (I)), each definition is independent.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, or subject (e.g., human) that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

In particular, the compounds disclosed herein possess axial chirality, by virtue of restricted rotation around a biaryl bond and as such may exist as mixtures of atropisomers. When a compound is a pure atropisomer, the stereochemistry at each chiral center may be specified by either Rₐ or Sₐ. Such designations may also be used for mixtures that are enriched in one atropisomer. Further description of atropisomerism and axial chirality and rules for assignment of configuration can be found in Eliel, E. L. & Wilen, S. H. ‘Stereochemistry of Organic Compounds’ John Wiley and Sons, Inc. 1994. Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light. Optically active (Rₐ)- and (Sₐ)-atropisomers may be prepared using chiral synthons, chiral reagents or chiral catalysts, or resolved using conventional techniques well known in the art, such as chiral HPLC.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer; when a compound of Formula (I) is for instance specified as Rₐ, this means that the compound is substantially free of the Sₐ atropisomer.

Pharmaceutically acceptable salts, in particular pharmaceutically acceptable additions salts, include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I), and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²²I, ¹²³I, ¹²⁵ I, ¹³¹ I, ⁷⁵Br, ⁷⁶Br, ⁷⁷Br and ⁸²Br. Preferably, the isotope is selected from the group of ²H, ³H, ¹¹C and ¹⁸F. More preferably, the isotope is ²H. In particular, deuterated compounds are intended to be included within the scope of the present invention.

Certain isotopically-labeled compounds of the present invention (e.g., those labeled with ³H and ¹⁴C) may be useful for example in substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

The present invention relates in particular to compounds of Formula (I) as defined herein, and the tautomers and the stereoisomeric forms thereof, wherein

X¹ represents

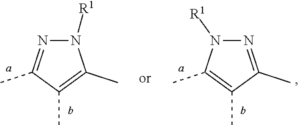

wherein ‘a’ and ‘b’ indicate how variable X¹ is attached to the remainder of the molecule;
R¹ and R² each independently represent hydrogen; methyl; or C₂₋₆alkyl optionally substituted with one or two substituents each independently selected from the group consisting of Het¹, —OR³, and —NR^{4a}R^{4b};
Het¹ represents morpholinyl or tetrahydropyranyl;
R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl;
R^{4a} and R^{4b} are each independently selected from the group consisting of hydrogen and C₁₋₄alkyl;
X² represents

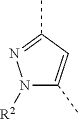

which can be attached to the remainder of the molecule in both directions;
X represents —O—, —S—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 0 or 1;
and the pharmaceutically acceptable salts and the solvates thereof.

The present invention relates in particular to compounds of Formula (I) as defined herein, and the tautomers and the stereoisomeric forms thereof, wherein

X¹ represents

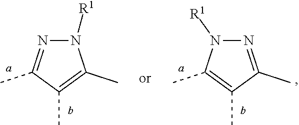

wherein ‘a’ and ‘b’ indicate how variable X¹ is attached to the remainder of the molecule;
R¹ and R² each independently represent hydrogen; methyl; or C₂₋₆alkyl optionally substituted with one or two substituents each independently selected from the group consisting of Het¹, —OR³, and —NR^{4a}R^{4b};
Het¹ represents morpholinyl or tetrahydropyranyl;
R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl;
R^{4a} and R^{4b} are each independently selected from the group consisting of hydrogen and C₁₋₄alkyl;
X² represents

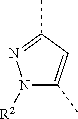

which can be attached to the remainder of the molecule in both directions;
X represents —O—, —S—, —S(═O)₂—, or —N(R)—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 0 or 1;
and the pharmaceutically acceptable salts and the solvates thereof; provided that

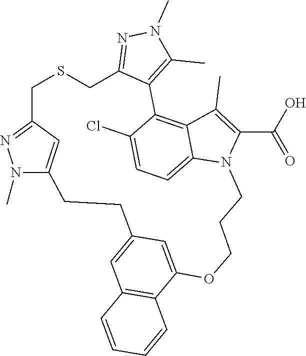 [SMILES: Cc1c(C(=O)O)n2c3ccc(Cl)c(c13)-c1c(nn(C)c1C)CSCc1cc(n(C)n1)CCc1cc(c3ccccc3c1)OCCC2]

and the tautomers and the stereoisomeric forms thereof are excluded

The present invention relates in particular to compounds of Formula (I) as defined herein, and the tautomers and the stereoisomeric forms thereof, wherein

X¹ represents

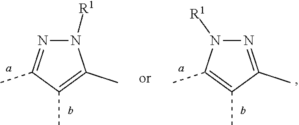

wherein ‘a’ and ‘b’ indicate how variable X¹ is attached to the remainder of the molecule;
R¹ and R² each independently represent hydrogen; methyl; or C₂₋₆alkyl optionally substituted with one substituent selected from the group consisting of Het¹, —OR³, and —NR^{4a}R^{4b}.
Het¹ represents morpholinyl or tetrahydropyranyl;
R³ represents hydrogen, C₁₋₄alkyl, or —C₂₋₄alkyl-O—C₁₋₄alkyl;
R^{4a} and R^{4b} are each independently selected from the group consisting of hydrogen and C₁₋₄alkyl;
X² represents

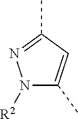

which can be attached to the remainder of the molecule in both directions;
X represents —O—, —S—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 0 or 1;
and the pharmaceutically acceptable salts and the solvates thereof.

The present invention relates in particular to compounds of Formula (I) as defined herein, and the tautomers and the stereoisomeric forms thereof, wherein

X¹ represents

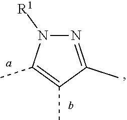

wherein ‘a’ and ‘b’ indicate how variable X¹ is attached to the remainder of the molecule;
R¹ and R² each independently represent hydrogen; methyl; or C₂₋₆alkyl optionally substituted with one substituent selected from the group consisting of Het¹, —OR³, and —NR^{4a}R^{4b}.
Het¹ represents morpholinyl or tetrahydropyranyl;
R³ represents hydrogen, C₁₋₄alkyl, or —C₂₋₄alkyl-O—C₁₋₄alkyl;
R^{4a} and R^{4b} are each independently selected from the group consisting of hydrogen and C₁₋₄alkyl;
X² represents

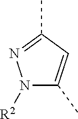

which can be attached to the remainder of the molecule in both directions;
X represents —O—, —S—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 0 or 1;
and the pharmaceutically acceptable salts and the solvates thereof.

The present invention relates in particular to compounds of Formula (I) as defined herein, and the tautomers and the stereoisomeric forms thereof, wherein

X¹ represents

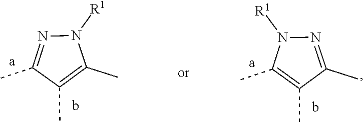

wherein ‘a’ and ‘b’ indicate how variable X¹ is attached to the remainder of the molecule;
R¹ and R² each independently represent hydrogen; methyl; or C₂₋₆alkyl optionally substituted with one substituent selected from the group consisting of Het¹, —OR³, and —NR^{4a}R^{4b}.
Het¹ represents morpholinyl or tetrahydropyranyl;
R³ represents hydrogen, C₁₋₄alkyl, or —C₂₋₄alkyl-O—C₁₋₄alkyl;
R^{4a} and R^{4b} are each independently selected from the group consisting of hydrogen and C₁₋₄alkyl;
X² represents

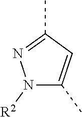

which can be attached to the remainder of the molecule in both directions;
X represents —O—, —S—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 1;
and the pharmaceutically acceptable salts and the solvates thereof.

The present invention relates in particular to compounds of Formula (I) as defined herein, and the tautomers and the stereoisomeric forms thereof, wherein

X¹ represents

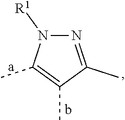

wherein ‘a’ and ‘b’ indicate how variable X¹ is attached to the remainder of the molecule;
R¹ and R² each independently represent hydrogen; methyl; or C₂₋₆alkyl optionally substituted with one or two substituents each independently selected from the group consisting of Het¹, —OR³, and —NR^{4a}R^{4b};
Het¹ represents morpholinyl or tetrahydropyranyl;
R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl;
R^{4a} and R^{4b} are each independently selected from the group consisting of hydrogen and C₁₋₄alkyl;
X² represents

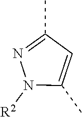

which can be attached to the remainder of the molecule in both directions;
X represents —O—, —S—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 0 or 1;
and the pharmaceutically acceptable salts and the solvates thereof.

The present invention relates in particular to compounds of Formula (I) as defined herein, and the tautomers and the stereoisomeric forms thereof, wherein

X¹ represents

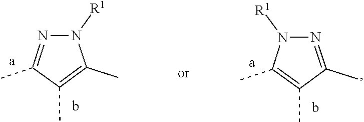

wherein ‘a’ and ‘b’ indicate how variable X¹ is attached to the remainder of the molecule;
R¹ and R² each independently represent hydrogen; methyl; or C₂₋₆alkyl optionally substituted with one or two substituents each independently selected from the group consisting of Het¹, —OR³, and —NR^{4a}R^{4b};
Het¹ represents morpholinyl or tetrahydropyranyl;
R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl;
R^{4a} and R^{4b} are each independently selected from the group consisting of hydrogen and C₁₋₄alkyl;
X² represents

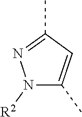

which can be attached to the remainder of the molecule in both directions;
X represents —O—, —S—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 1;
and the pharmaceutically acceptable salts and the solvates thereof.

The present invention relates in particular to compounds of Formula (I) as defined herein, and the tautomers and the stereoisomeric forms thereof, wherein

X¹ represents

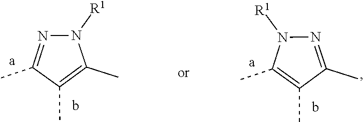

wherein ‘a’ and ‘b’ indicate how variable X¹ is attached to the remainder of the molecule;
R¹ represents C₂₋₆alkyl substituted with two substituents each independently selected from the group consisting of Het¹, —OR³, and —NR^{4a}R^{4b}; wherein R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl;
or
R¹ represents C₂₋₆alkyl substituted with one or two —OR³ substituents; wherein R³ represents —C₂₋₄alkyl-O—C₂₋₄alkyl-O—C₁₋₄alkyl;
R² represents methyl;
Het¹ represents morpholinyl or tetrahydropyranyl;
R^{4a} and R^{4b} are each independently selected from the group consisting of hydrogen and C₁₋₄alkyl;
X² represents

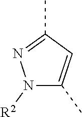

which can be attached to the remainder of the molecule in both directions;
X represents —O—, —S—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 0 or 1;
and the pharmaceutically acceptable salts and the solvates thereof.

The present invention relates in particular to compounds of Formula (I) as defined herein, and the tautomers and the stereoisomeric forms thereof, wherein

X¹ represents

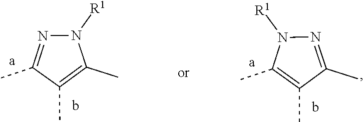

wherein ‘a’ and ‘b’ indicate how variable X¹ is attached to the remainder of the molecule;
R¹ represents C₂₋₆alkyl substituted with two substituents each independently selected from the group consisting of Het¹, —OR³, and —NR^{4a}R^{4b};
R² represents methyl;
Het¹ represents morpholinyl or tetrahydropyranyl;
R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl;
R^{4a} and R^{4b} are each independently selected from the group consisting of hydrogen and C₁₋₄alkyl;
X² represents

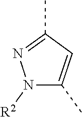

which can be attached to the remainder of the molecule in both directions;
X represents —O—, —S—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 0 or 1;
and the pharmaceutically acceptable salts and the solvates thereof.

The present invention relates in particular to compounds of Formula (I) as defined herein, and the tautomers and the stereoisomeric forms thereof, wherein

X¹ represents

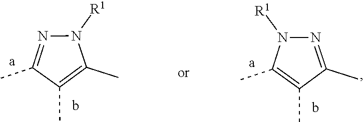

wherein ‘a’ and ‘b’ indicate how variable X¹ is attached to the remainder of the molecule;
R¹ represents C₂₋₆alkyl substituted with one or two —OR³ substituents;
R² represents methyl;
R³ represents —C₂₋₄alkyl-O—C₂₋₄alkyl-O—C₁₋₄alkyl;
X² represents

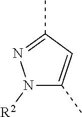

which can be attached to the remainder of the molecule in both directions;
X represents —O—, —S—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 0 or 1;
and the pharmaceutically acceptable salts and the solvates thereof.

The present invention relates in particular to compounds of Formula (I) as defined herein, and the tautomers and the stereoisomeric forms thereof, wherein

X¹ represents

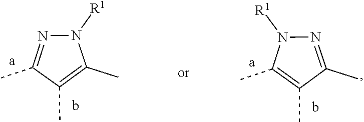

wherein ‘a’ and ‘b’ indicate how variable X¹ is attached to the remainder of the molecule;
R¹ and R² each independently represent methyl; or C₂₋₆alkyl optionally substituted with one or two substituents each independently selected from the group consisting of Het¹, —OR³, and —NR^{4a}R^{4b};
Het¹ represents tetrahydropyranyl;
R³ represents C₁₋₄alkyl, —C₂₋₄alkyl-O—C₁₋₄alkyl, or —C₂₋₄alkyl-O—C₂₋₄alkyl-O—C₁₋₄alkyl;
R^{4a} and R^{4b} represent hydrogen;
X² represents

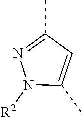

which can be attached to the remainder of the molecule in both directions;
X represents —S—, —S(═O)₂—, or —N(R^{x})—;
R^{x} represents methyl;
R^{y} represents halo;
n represents 0 or 1;
and the pharmaceutically acceptable salts and the solvates thereof.

The present invention relates in particular to compounds of Formula (I) as defined herein, and the tautomers and the stereoisomeric forms thereof, wherein

X¹ represents

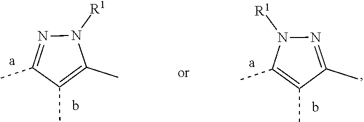

wherein ‘a’ and ‘b’ indicate how variable X¹ is attached to the remainder of the molecule;
R¹ and R² each independently represent hydrogen; methyl; or C₂₋₆alkyl optionally substituted with one or two substituents each independently selected from the group consisting of Het¹, —OR³, and —NR^{4a}R^{4b};
Het¹ represents tetrahydropyranyl;
R³ represents C₁₋₄alkyl, —C₂₋₄alkyl-O—C₁₋₄alkyl, or —C₂₋₄alkyl-O—C₂₋₄alkyl-O—C₁₋₄alkyl;
R^{4a} and R^{4b} represent hydrogen;
X² represents

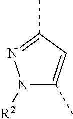

which can be attached to the remainder of the molecule in both directions;
X represents —O—, —S—, —S(═O)₂—, or —N(R^{x})—;
R^{x} represents methyl;
R^{y} represents halo;
n represents 0 or 1;
and the pharmaceutically acceptable salts and the solvates thereof.

The present invention relates in particular to compounds of Formula (I) as defined herein, and the tautomers and the stereoisomeric forms thereof, wherein

X¹ represents

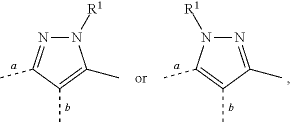

wherein ‘a’ and ‘b’ indicate how variable X¹ is attached to the remainder of the molecule;
R¹ and R² each independently represent hydrogen; methyl; or C₂₋₆alkyl optionally substituted with one or two substituents each independently selected from the group consisting of Het¹, —OR³, and —NR^{4a}R^{4b};
Het¹ represents tetrahydropyranyl;
R³ represents hydrogen, C₁₋₄alkyl, —C₂₋₄alkyl-O—C₁₋₄alkyl, —C₂₋₄alkyl-OH, or —C₂₋₄alkyl-O—C₂₋₄alkyl-O—C₁₋₄alkyl;
R^{4a} and R^{4b} represent hydrogen or C₁₋₄alkyl;
X² represents

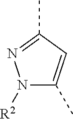

which can be attached to the remainder of the molecule in both directions;
X represents —O—, —S—, —S(═O)—, —S(═O)₂—, or —N(R^{x})—;
R^{x} represents methyl;
R^{y} represents halo;
n represents 0, 1 or 2;
and the pharmaceutically acceptable salts and the solvates thereof.

The present invention relates in particular to compounds of Formula (I) as defined herein, and the tautomers and the stereoisomeric forms thereof, wherein

X¹ represents

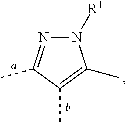

wherein ‘a’ and ‘b’ indicate how variable X¹ is attached to the remainder of the molecule;
R¹ and R² represent methyl;
X² represents

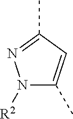

which can be attached to the remainder of the molecule in both directions;
X represents —S—, —S(═O)₂—, or —N(R^{x})—;
R^{x} represents methyl;
R^{y} represents halo;
n represents 0 or 1;
and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein

X represents —N(R^{x})—.

In an embodiment, the present invention relates to those compounds of Formula (I) and the pharmaceutically acceptable salts, and the solvates thereof, or any subgroup thereof as mentioned in any of the other embodiments, wherein

X represents —S—.

In an embodiment, the present invention relates to those compounds of Formula (I) and the pharmaceutically acceptable salts, and the solvates thereof, or any subgroup thereof as mentioned in any of the other embodiments, wherein

X represents —O—.

In an embodiment, the present invention relates to those compounds of Formula (I) and the pharmaceutically acceptable salts, and the solvates thereof, or any subgroup thereof as mentioned in any of the other embodiments, wherein

X represents —N(R^{x})—; and R^{x} represents hydrogen.

In an embodiment, the present invention relates to those compounds of Formula (I) and the pharmaceutically acceptable salts, and the solvates thereof, or any subgroup thereof as mentioned in any of the other embodiments, wherein

X represents —N(R^{x})—; and R^{x} represents methyl.

In an embodiment, the present invention relates to those compounds of Formula (I) and the pharmaceutically acceptable salts, and the solvates thereof, or any subgroup thereof as mentioned in any of the other embodiments, wherein

X represents —O—, —S—, —S(═O)₂—, or —N(R^{x})—

In an embodiment, the present invention relates to those compounds of Formula (I) and the pharmaceutically acceptable salts, and the solvates thereof, or any subgroup thereof as mentioned in any of the other embodiments, wherein R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein

n represents 1; and
R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein

n represents 2; and
R^{y} represents fluoro or chloro.

In an embodiment, the present invention relates to those compounds of Formula (I) and the pharmaceutically acceptable salts, and the solvates thereof, or any subgroup thereof as mentioned in any of the other embodiments, wherein

n represents 2; and
R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein

X¹ represents

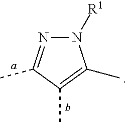

In an embodiment, the present invention relates to those compounds of Formula (I) and the pharmaceutically acceptable salts, and the solvates thereof, or any subgroup thereof as mentioned in any of the other embodiments, wherein

X¹ represents

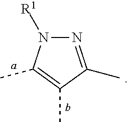

In an embodiment, the present invention relates to those compounds of Formula (I) and the pharmaceutically acceptable salts, and the solvates thereof, or any subgroup thereof as mentioned in any of the other embodiments, wherein n represents 1; and

X¹ represents

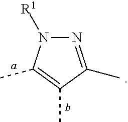

In an embodiment, the present invention relates to those compounds of Formula (I) and the pharmaceutically acceptable salts, and the solvates thereof, or any subgroup thereof as mentioned in any of the other embodiments, wherein n represents 2; and

X¹ represents

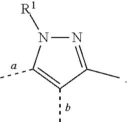

In an embodiment, the present invention relates to those compounds of Formula (I) and the pharmaceutically acceptable salts, and the solvates thereof, or any subgroup thereof as mentioned in any of the other embodiments, wherein R¹ represents hydrogen.

In an embodiment, the present invention relates to those compounds of Formula (I) and the pharmaceutically acceptable salts, and the solvates thereof, or any subgroup thereof as mentioned in any of the other embodiments, wherein R¹ represents C₂₋₆alkyl.

In an embodiment, the present invention relates to those compounds of Formula (I) and the pharmaceutically acceptable salts, and the solvates thereof, or any subgroup thereof as mentioned in any of the other embodiments, wherein R¹ represents methyl.

In an embodiment, the present invention relates to those compounds of Formula (I) and the pharmaceutically acceptable salts, and the solvates thereof, or any subgroup thereof as mentioned in any of the other embodiments, wherein R² represents hydrogen.

In an embodiment, the present invention relates to those compounds of Formula (I) and the pharmaceutically acceptable salts, and the solvates thereof, or any subgroup thereof as mentioned in any of the other embodiments, wherein R² represents C₂₋₆alkyl.

In an embodiment, the present invention relates to those compounds of Formula (I) and the pharmaceutically acceptable salts, and the solvates thereof, or any subgroup thereof as mentioned in any of the other embodiments, wherein R² represents methyl.

In an embodiment, the present invention relates to those compounds of Formula (I) and the pharmaceutically acceptable salts, and the solvates thereof, or any subgroup thereof as mentioned in any of the other embodiments, wherein n represents 0.

In an embodiment, the present invention relates to those compounds of Formula (I) and the pharmaceutically acceptable salts, and the solvates thereof, or any subgroup thereof as mentioned in any of the other embodiments, wherein n represents 1.

In an embodiment, the present invention relates to those compounds of Formula (I) and the pharmaceutically acceptable salts, and the solvates thereof, or any subgroup thereof as mentioned in any of the other embodiments, wherein n represents 2.

In an embodiment, the present invention relates to those compounds of Formula (I) and the pharmaceutically acceptable salts, and the solvates thereof, or any subgroup thereof as mentioned in any of the other embodiments, wherein

R³ represents —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

X¹ represents

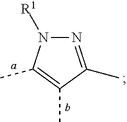

and
R³ represents —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R³ represents —C₂₋₄alkyl-O—C₂₋₄alkyl-O—C₁₋₄alkyl; and n represents 1.

In an embodiment, the present invention relates to those compounds of Formula (I) and the pharmaceutically acceptable salts, and the solvates thereof, or any subgroup thereof as mentioned in any of the other embodiments, wherein

X¹ represents

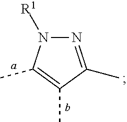

R³ represents —C₂₋₄alkyl-O—C₂₋₄alkyl-O—C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one or two —OR³ substituents;
R² represents methyl; and
R³ represents —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

X¹ represents

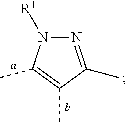

R¹ represents C₂₋₆alkyl substituted with one or two —OR³ substituents;
R² represents methyl; and
R³ represents —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one or two —OR³ substituents;
R² represents methyl;
R³ represents —C₂₋₄alkyl-O—C₂₋₄alkyl-O—C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

X¹ represents

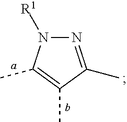

R¹ represents C₂₋₆alkyl substituted with one or two —OR³ substituents;
R² represents methyl;
R³ represents —C₂₋₄alkyl-O—C₂₋₄alkyl-O—C₁₋₄alkyl;
and n represents 1.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with two substituents each independently selected from the group consisting of Het¹, —OR³, and —NR^{4a}R^{4b}; wherein R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl; or
R¹ represents C₂₋₆alkyl substituted with one or two —OR³ substituents; wherein R³ represents —C₂₋₄alkyl-O—C₂₋₄alkyl-O—C₁₋₄alkyl;
R² represents hydrogen; methyl; or C₂₋₆alkyl optionally substituted with one substituent selected from the group consisting of Het¹, —OR³, and —NR^{4a}R^{4b}; wherein R³ represents hydrogen, C₁₋₄alkyl, or —C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with two substituents each independently selected from the group consisting of Het¹, —OR³, and —NR^{4a}R^{4b}; wherein R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl;
or
R¹ represents C₂₋₆alkyl substituted with one or two —OR³ substituents; wherein R³ represents —C₂₋₄alkyl-O—C₂₋₄alkyl-O—C₁₋₄alkyl;
R² represents methyl.

In an embodiment, the present invention relates to those compounds of Formula (I) and the pharmaceutically acceptable salts, and the solvates thereof, or any subgroup thereof as mentioned in any of the other embodiments, wherein

X¹ represents

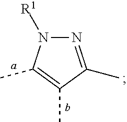

R¹ represents C₂₋₆alkyl substituted with two substituents each independently selected from the group consisting of Het¹, —OR³, and —NR^{4a}R^{4b}; wherein R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl;
or
R¹ represents C₂₋₆alkyl substituted with one or two —OR³ substituents; wherein R³ represents —C₂₋₄alkyl-O—C₂₋₄alkyl-O—C₁₋₄alkyl;
R² represents methyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with two substituents each independently selected from the group consisting of Het¹, —OR³, and —NR^{4a}R^{4b}; wherein R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl;
or
R¹ represents C₂₋₆alkyl substituted with one or two —OR³ substituents; wherein R³ represents —C₂₋₄alkyl-O—C₂₋₄alkyl-O—C₁₋₄alkyl;
R² represents methyl;
and n represents 1.

In an embodiment, the present invention relates to those compounds of Formula (I) and the pharmaceutically acceptable salts, and the solvates thereof, or any subgroup thereof as mentioned in any of the other embodiments, wherein

X¹ represents

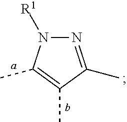

R¹ represents C₂₋₆alkyl substituted with two substituents each independently selected from the group consisting of Het¹, —OR³, and —NR^{4a}R^{4b}; wherein R³ represents hydrogen, C₁₋₄alkyl, —C₂₋₄alkyl-O—C₁₋₄alkyl, or —C₂₋₄alkyl-O—C₂₋₄alkyl-O—C₁₋₄alkyl;
or
R¹ represents C₂₋₆alkyl substituted with one or two —OR³ substituents; wherein R³ represents —C₂₋₄alkyl-O—C₂₋₄alkyl-O—C₁₋₄alkyl;
R² represents methyl;
and n represents 1

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with two —OR³ substituents;
R² represents methyl;
R³ represents C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

X¹ represents

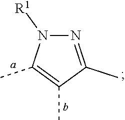

R¹ represents C₂₋₆alkyl substituted with two —OR³ substituents;
R² represents methyl;
R³ represents C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with two —OR³ substituents;
R² represents methyl;
R³ represents C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

X¹ represents

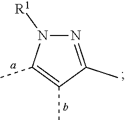

R¹ represents C₂₋₆alkyl substituted with two —OR³ substituents;
R² represents methyl;
R³ represents C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one —OR³ substituent;
R² represents methyl; and
R³ represents —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

X¹ represents

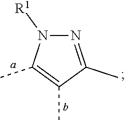

R¹ represents C₂₋₆alkyl substituted with one —OR³ substituent;
R² represents methyl;
R³ represents —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one —OR³ substituent;
R² represents methyl;
R³ represents —C₂₋₄alkyl-O—C₂₋₄alkyl-O—C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

X¹ represents

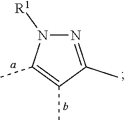

R¹ represents C₂₋₆alkyl substituted with one —OR³ substituent;
R² represents methyl;
R³ represents —C₂₋₄alkyl-O—C₂₋₄alkyl-O—C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one substituent selected from the group consisting of Het¹ or —OR³;
R² represents methyl;
R³ represents —C₂₋₄alkyl-O—C₁₋₄alkyl or —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

X¹ represents

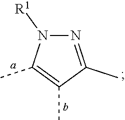

R¹ represents C₂₋₆alkyl substituted with one substituent selected from the group consisting of Het¹ or —OR³;
R² represents methyl;
R³ represents —C₂₋₄alkyl-O—C₁₋₄alkyl or —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one substituent selected from the group consisting of Het¹ or —OR³;
R² represents methyl;
R³ represents —C₂₋₄alkyl-O—C₁₋₄alkyl or —C₂₋₄alkyl-O—C₂₋₄alkyl-O—C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

X¹ represents

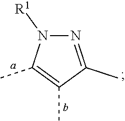

R¹ represents C₂₋₆alkyl substituted with one substituent selected from the group consisting of Het¹ or —OR³;
R² represents methyl;
R³ represents —C₂₋₄alkyl-O—C₁₋₄alkyl or —C₂₋₄alkyl-O—C₂₋₄alkyl-O—C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one —OR³ substituent;
R² represents methyl;
R³ represents —C₂₋₄alkyl-O—C₁₋₄alkyl or —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

X¹ represents

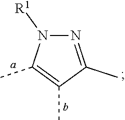

R¹ represents C₂₋₆alkyl substituted with one —OR³ substituent;
R² represents methyl;
R³ represents —C₂₋₄alkyl-O—C₁₋₄alkyl or —C₂₋₄alkyl-O—C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one —OR³ substituent;
R² represents methyl;
R³ represents —C₂₋₄alkyl-O—C₁₋₄alkyl or —C₂₋₄alkyl-O—C₂₋₄alkyl-O—C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

X¹ represents

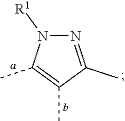

R¹ represents C₂₋₆alkyl substituted with one —OR³ substituent;
R² represents methyl;
R³ represents —C₂₋₄alkyl-O—C₁₋₄alkyl or —C₂₋₄alkyl-O—C₂₋₄alkyl-O—C₁₋₄alkyl; and
n represents 1.

In an embodiment, the present invention relates to those compounds of Formula (I) and the pharmaceutically acceptable salts, and the solvates thereof, or any subgroup thereof as mentioned in any of the other embodiments, wherein

X represents —N(R^{x})—; and R^{y} represents halo.

In an embodiment, the present invention relates to those compounds of Formula (I) and the pharmaceutically acceptable salts, and the solvates thereof, or any subgroup thereof as mentioned in any of the other embodiments, wherein

X represents —N(R^{x})—; and R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein

X represents —S—; and R^{y} represents halo.

In an embodiment, the present invention relates to those compounds of Formula (I) and the pharmaceutically acceptable salts, and the solvates thereof, or any subgroup thereof as mentioned in any of the other embodiments, wherein

X represents —S—; and R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein n is 1 and wherein R^{y} is in position 3 as indicated below:

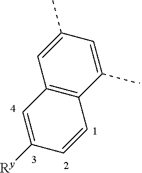

In an embodiment, the present invention relates to those compounds of Formula (I) and the pharmaceutically acceptable salts, and the solvates thereof, or any subgroup thereof as mentioned in any of the other embodiments, wherein n is 1 and wherein R^{y} is in position 3 as indicated below; and wherein R^{y} represents fluoro:

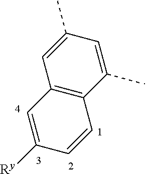

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-x):

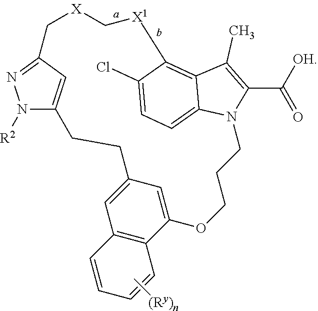

It will be clear that all variables in the structure of Formula (I-x), are defined as defined for the compounds of Formula (I) or any subgroup thereof as mentioned in any of the other embodiments.

The present invention relates in particular to compounds of Formula (I-x) as defined herein, and the tautomers and the stereoisomeric forms thereof, wherein

X¹ represents

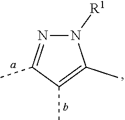

wherein ‘a’ and ‘b’ indicate how variable X¹ is attached to the remainder of the molecule;
R¹ and R² represent methyl;
X represents —S—, —S(═O)₂—, or —N(R^{x})—;
R represents methyl;
R^{y} represents halo;
n represents 0 or 1;
and the pharmaceutically acceptable salts and the solvates thereof.

The present invention relates in particular to compounds of Formula (I-x) as defined herein, and the tautomers and the stereoisomeric forms thereof, wherein

X¹ represents

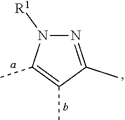

wherein ‘a’ and ‘b’ indicate how variable X¹ is attached to the remainder of the molecule;
R¹ represents C₂₋₆alkyl substituted with one —OR³ substituent;
R² represent methyl;
R³ represents —C₂₋₄alkyl-O—C₁₋₄alkyl;
X represents —S—, —S(═O)₂—, or —N(R^{x})—:
R^{x} represents methyl;
R^{y} represents halo;
n represents 0 or 1;
and the pharmaceutically acceptable salts and the solvates thereof.

The present invention relates in particular to compounds of Formula (I-x) as defined herein, and the tautomers and the stereoisomeric forms thereof, wherein

X¹ represents

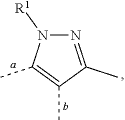

wherein ‘a’ and ‘b’ indicate how variable X¹ is attached to the remainder of the molecule;
R¹ represents C₂₋₆alkyl substituted with one —OR³ substituent;
R² represent methyl;
R³ represents —C₂₋₄alkyl-O—C₁₋₄alkyl;
X represents —S—;
R^{y} represents halo; in particular F;
n represents 1;
and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein the compounds are Rₐ atropisomers.

In an embodiment, the present invention relates to those compounds of Formula (I) and the pharmaceutically acceptable salts, and the solvates thereof, or any subgroup thereof as mentioned in any of the other embodiments, wherein the compounds are Sₐ atropisomers.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-y):

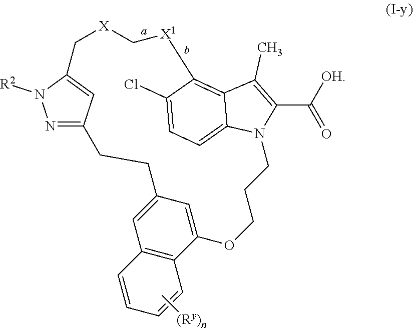

It will be clear that all variables in the structure of Formula (I-y), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, provided that

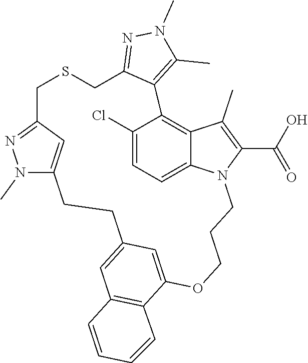 [SMILES: Cc1c(C(=O)O)n2c3ccc(Cl)c(c13)-c1c(nn(C)c1C)CSCc1cc(n(C)n1)CCc1cc(c3ccccc3c1)OCCC2]

and the tautomers and the stereoisomeric forms thereof are excluded. In an embodiment, the scope of the present invention does not include said excluded compound, and the pharmaceutically acceptable salts thereof. In an embodiment, the scope of the present invention does not include said excluded compounds, and the pharmaceutically acceptable salts and the solvates thereof.

In an embodiment, the present invention relates to a subgroup of Formula (I) as defined in the general reaction schemes.

In an embodiment the compound of Formula (I) is selected from the group consisting of any of the exemplified compounds, tautomers and stereoisomeric forms thereof, any pharmaceutically acceptable salts, and the solvates thereof.

All possible combinations of the above indicated embodiments are considered to be embraced within the scope of the invention.

### Methods for the Preparation of Compounds

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or can be prepared by standard synthetic processes commonly used by those skilled in the art of organic chemistry. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

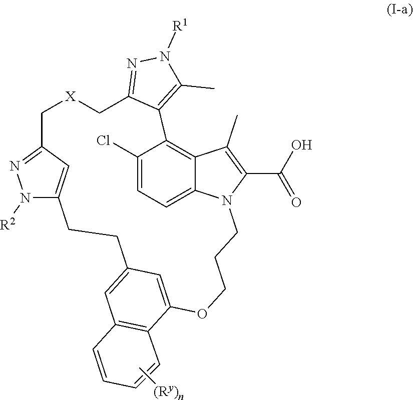

Compounds of Formula (I-a) can be prepared according to Scheme 1,

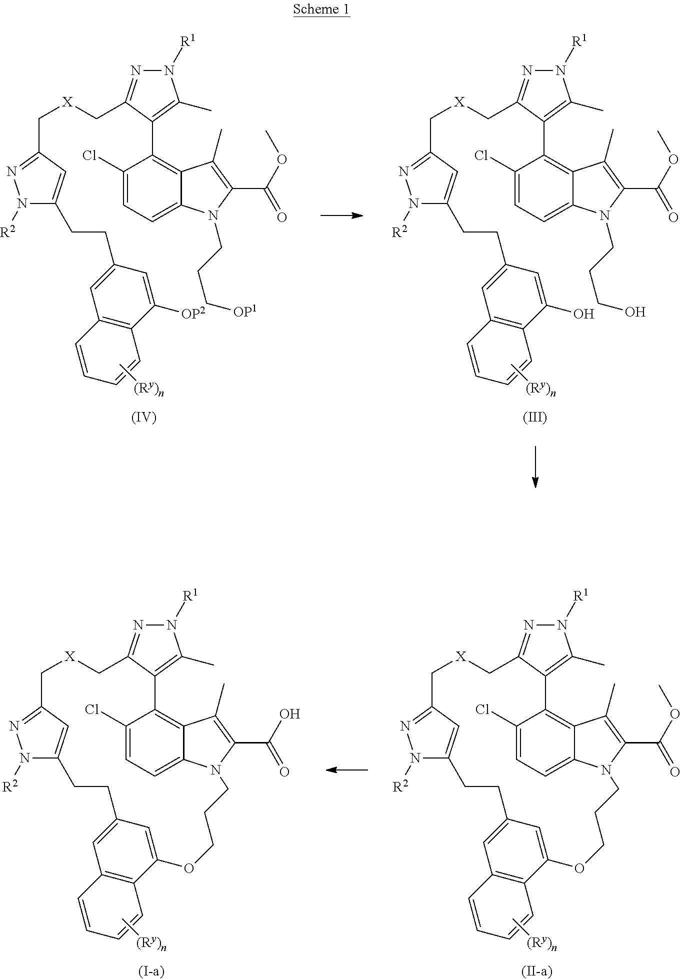

by reacting an intermediate of Formula (II-a) where X, R¹, R², and (R^{y})ₙ are defined as in Formula (I), with a suitable base such as, for example, LiOH or NaOH, in a suitable solvent such as water or a mixture of water and a suitable organic solvent such as dioxane or tetrahydrofuran (THF), or a mixture of methanol (MeOH) and THF, at a suitable temperature such as room temperature or 60° C.
Intermediates of Formula (II-a) can be prepared by reacting an intermediate of Formula (III) where X, R¹, and (R^{y})ₙ are defined as in Formula (I), and R² is a suitable protecting group such as, for example, paramethoxybenzyl (PMB), dimethoxylbenzyl (DMB), or tetrahydropyranyl (THP), or can also be a suitable alkyl substituent such as, for example, methyl, with a suitable reagent, such as, for example, diethyl azodicarboxylate (DEAD) or di-tert-butyl azodicarboxylate (DTBAD), in the presence of a suitable phosphine such as, for example, PPh₃, in a suitable solvent such as, for example, THF, toluene, or a mixture thereof, at a suitable temperature such as, for example, room temperature or 70° C.
Intermediates of Formula (III) can be prepared by reacting an intermediate of Formula (IV) where X, R¹, R², and (R^{y})ₙ are as defined in Formula (III), and P¹ as well as P² are suitable protecting groups, such as, for example, tert-butyldimethylsilyl (TBDMS) or tert-butyldiphenylsilyl (TBDPS), with a suitable deprotecting reagent such as, for example, tetrabutylammonium fluoride (TBAF), in a suitable solvent such as, for example, THF, at a suitable temperature such as, for example, room temperature or 60° C.
Alternatively, when P² in intermediates of Formula (IV) is a PMB group, an additional deprotection step might be necessary, using a suitable deprotection reagent such as, for example, TFA or 2,3-Dichloro-5,6-dicyano-1,4-benzoquinone (DDQ), in a suitable solvent such as, for example, dichloromethane (DCM), at a suitable temperature such as, for example, room temperature.

An intermediate of Formula (II-a) might have a protecting group in the R¹ position such as, for example, tetrahydropyranyl. In such a case, the intermediate of Formula (II) is reacted with a suitable deprotection reagent, such as, for example, pTsOH (p-toluenesulfonic acid) or HCl, in a suitable solvent such as, for example, iPrOH (2-propanol), at a suitable temperature such as, for example, room temperature. In a next step the obtained unprotected intermediate can be reacted with a suitable alkylating agent R¹L (where L is as suitable leaving group) such as, for example, an alkyl halide, in the presence of a suitable base such as, for example, Cs₂CO₃, in a suitable solvent such as, for example, DMF (N,N-dimethylformamide), at a suitable temperature such as, for example, room temperature or 60° C.

It will be clear for someone skilled in the art, that orthogonality of protective groups will have to be considered in this case, for instance when R¹ is a tetrahydropyranyl, P¹ and P² should be preferably TBDMS or TBDPS groups.

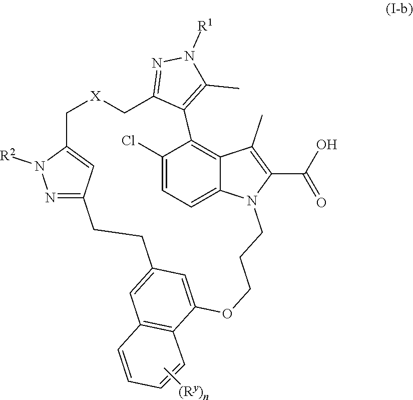

Similarly, compounds of Formula (I-b) can be prepared as described for compounds of Formula (I-a), but starting from the regioisomer of intermediates of Formula (XXI) (where R² is on the other pyrazole nitrogen). It will be clear for a skilled person that in the final synthesis step, an intermediate of Formula (II-b) (where R² is on the other pyrazole nitrogen) is reacted to a compound of Formula (I-b) in that case.

Alternatively, both intermediates of Formula (II-a) and (II-b), where R² is defined as in compounds of Formula (I-a) and (I-b), respectively, can be prepared in two steps.
First, by reacting an intermediate of Formula (II-a) or (II-b), respectively, where R² is then defined as a suitable protecting group such as, for example, THP, with a suitable deprotection reagent such as, for example, HCl, in a suitable solvent such as, for example, dioxane or isopropanol, at a suitable temperature such as, for example, room temperature.

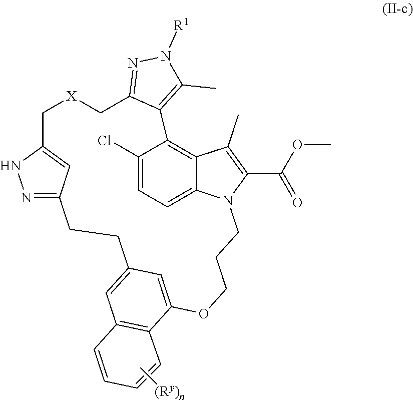

Then, by reacting the obtained intermediate of Formula (II-c) with a suitable alkylating agent R²L, such as, for example, an alkyl halide, in a suitable solvent, such as, for example, DMF, or acetonitrile, in the presence of a suitable base, such as, for example, trietylamine (Et₃N), N,N-Diisopropylethylamine (iPr₂EtN), Cs₂CO₃, or 1,8-Diazabicyclo[5.4.0]undec-7-ene (DBU), at a suitable temperature, such as, for example, room temperature or 60° C., followed by a suitable separation of the isomers (II-a) and (II-b), such as, for example, a chromatographic separation.

Alternatively, compounds of Formula (I) where R¹, R², and (R^{y})ₙ are as defined in Formula (I-a), and X is defined as N(CH₃), can be prepared according to Scheme 2,

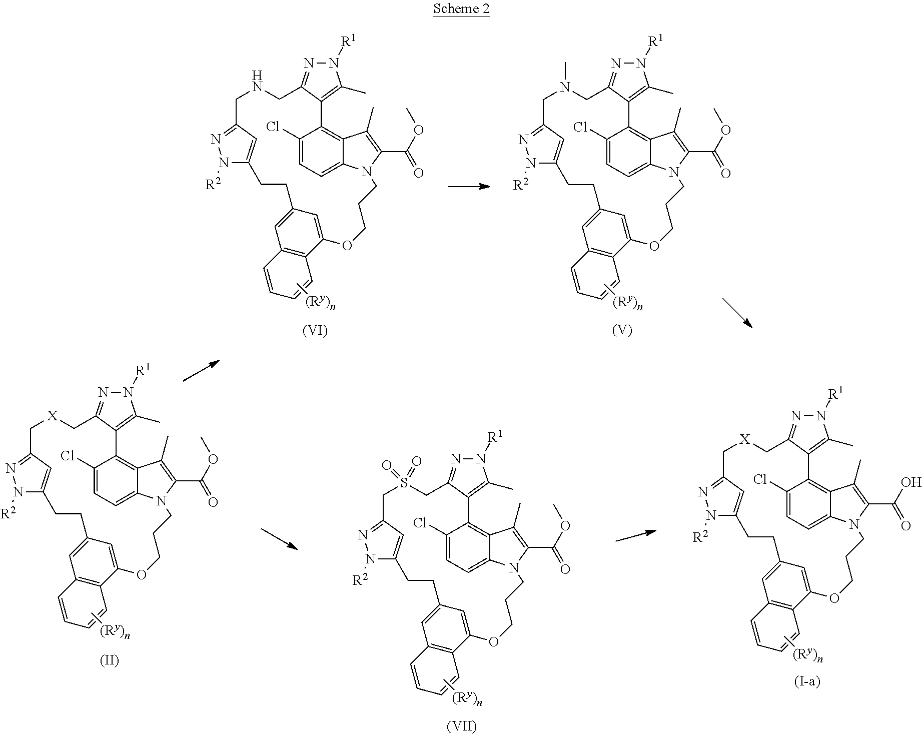

by reacting an intermediate of Formula (V) with a suitable base such as, for example, LiOH or NaOH, in a suitable solvent such as water or a mixture of water and a suitable organic solvent such as dioxane or THF, or a mixture of MeOH and THF, at a suitable temperature such as room temperature or 60° C.
Intermediates of Formula (V) can be prepared by reacting an intermediate of Formula (VI) with a suitable aldehyde such as, for example, formaldehyde, and a suitable reducing agent such as, for example, NaBH(OAc)₃ or NaBH₃CN, in the presence of a suitable acid such as, for example, AcOH, in a suitable solvent such as, for example, CH₂Cl₂, at a suitable temperature such as, for example, room temperature.
Intermediates of Formula (VI) can be be prepared by reacting an intermediate of Formula (II) where X is defined as nitrogen protected by a protecting group such as, for example, 2-nitrophenylsulfonyl, with a suitable deprotecting agent such as, for example, thiophenol, in the presence of a suitable base such as, for example, K₂CO₃, in a suitable solvent such as, for example, acetonitrile, at a suitable temperature such as, for example, room temperature.

Alternatively, compounds of Formula (I) where R¹, R², and (R^{y})ₙ are as defined in Formula (I), and X is defined as S(O)₂, can be prepared according to Scheme 2,
by reacting an intermediate of Formula (VII) with a suitable base such as, for example, LiOH or NaOH, in a suitable solvent such as water or a mixture of water and a suitable organic solvent such as dioxane or THF, or a mixture of MeOH and THF, at a suitable temperature such as room temperature or 60° C.
Intermediates of Formula (VII) can be prepared by reacting an intermediate of Formula (II) where R¹, R², (R^{y})ₙ are as defined in Formula (I), and X is defined as S (sulfur), with a suitable oxidizing agent such as, for example, mCPBA, in a suitable solvent such as, for example, CH₂Cl₂, at a suitable temperature such as, for example 0° C. or room temperature.

When X is defined as S (sulfur), intermediates of Formula (IV) can be prepared according to Scheme 3,

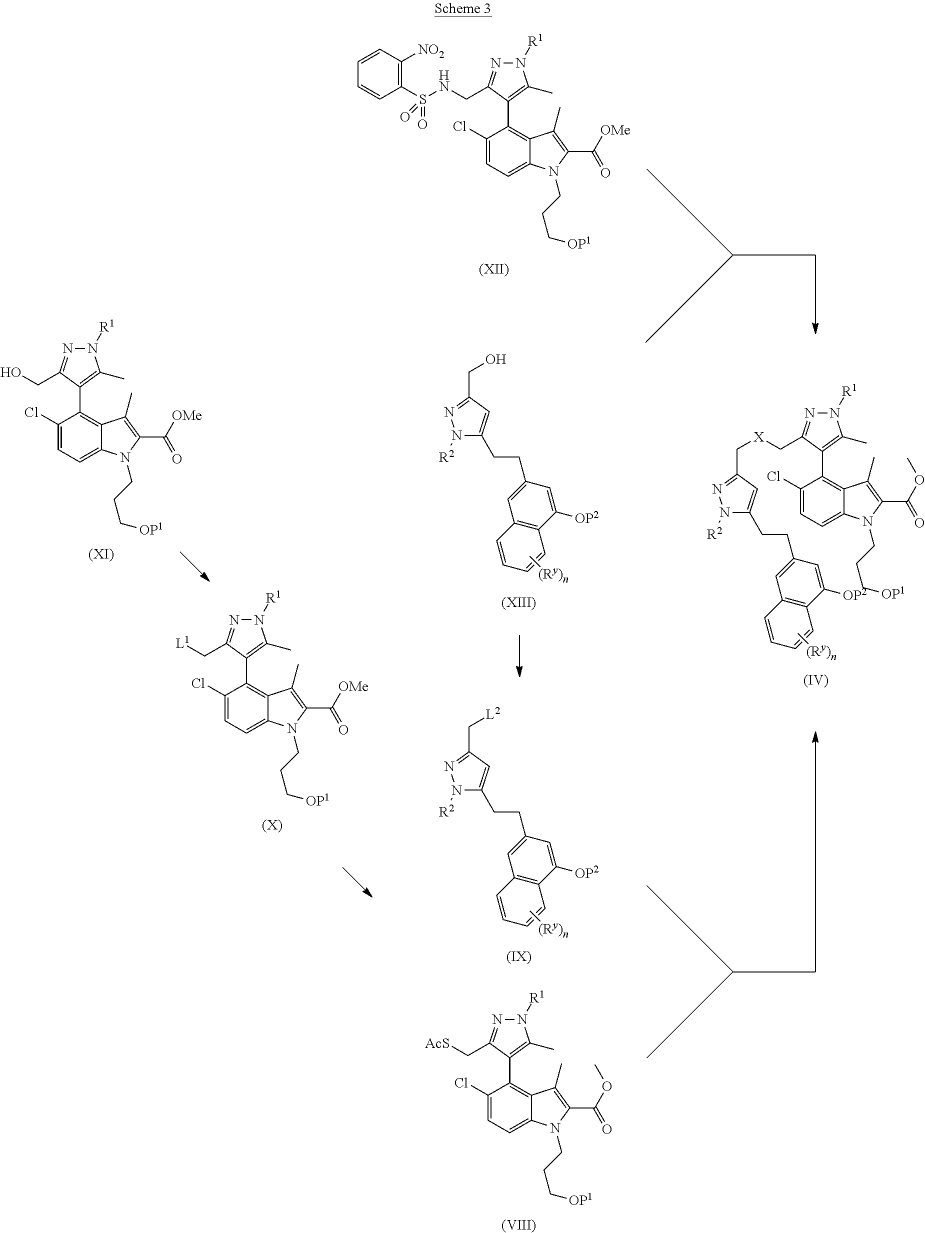

by reacting an intermediate of Formula (VIII), where P¹ is a suitable protecting groups such as, for example, tert-butyldimethylsilyl (TBDMS), with an intermediate of Formula (IX) where L² is a suitable leaving group such as, for example, chloride or mesylate, and P² is a suitable protecting group such as, for example, TBDPS, in the presence of a suitable base such as, for example, K₂CO₃, in a suitable solvent, such as for example, MeOH, at a suitable temperature such as, for example, room temperature.
Intermediates of Formula (VIII) can be prepared by reacting an intermediate of Formula (X), where L¹ is a suitable leaving group such as, for example, iodide or mesylate, with KSAc, in a suitable solvent such as, for example, acetonitrile, at a suitable temperature such as, for example, room temperature.
Intermediates of Formula (IX) can be prepared by reacting an intermediate of Formula (XIII), with a suitable reagent such as for example, mesyl chloride or thionyl chloride, if necessary in the presence of a suitable base such as, for example, triethylamine, in a suitable solvent such as, for example, CH₂Cl₂, at a suitable temperature such as, for example, 0° C. or room temperature.
Intermediates of Formula (X) can be prepared by reacting an intermediate of Formula (XI), with a suitable alkylsulfonyl chloride such as, for example, mesyl chloride, in the presence of a suitable base such as, for example, triethylamine, in a suitable solvent such as, for example, CH₂Cl₂, at a suitable temperature, such as for example, room temperature.
Alternatively, intermediates of Formula (X) can be prepared in two steps, by reacting an intermediate of Formula (XI), with a suitable alkylsulfonyl chloride, such as for example, mesyl chloride, in the presence of a suitable base such as, for example, triethylamine, in a suitable solvent such as, for example, CH₂Cl₂, at a suitable temperature such as, for example, room temperature; followed by reaction with a suitable metal halide such as, for example, potassium iodide, in a suitable solvent such as, for example, acetonitrile, at a suitable temperature, such as for example, room temperature or 60° C.

Alternatively, when X is defined as nitrogen protected by a protecting group such as for example 2-nitrophenylsulfonyl, intermediates of Formula (IV) can be prepared according to Scheme 3,
by reacting an intermediate of Formula (XII) with an intermediate of Formula (XIII) in the presence of a suitable reagent such as, for example, DEAD or DBAD, in the presence of a suitable phosphine such as, for example, triphenylphosphine (PPh₃), in a suitable solvent such as, for example, THF, toluene, or a mixture thereof, at a suitable temperature such as, for example, room temperature or 60° C.
Intermediates of Formula (XII) can be prepared by reacting an intermediate of Formula (XI) with a suitable protected nitrogen such as, for example, 2-nitrophenylsulfonamide, in the presence of a suitable reagent such as, for example, DEAD or DBAD, in the presence of a suitable phosphine such as, for example, PPh₃, in a suitable solvent such as, for example, THF, toluene, or a mixture thereof, at a suitable temperature such as, for example, room temperature or 60° C.

Intermediates of Formula (XI), where P¹ is a suitable protecting group, such as, for example, TBDMS, can be prepared according to Scheme 4,

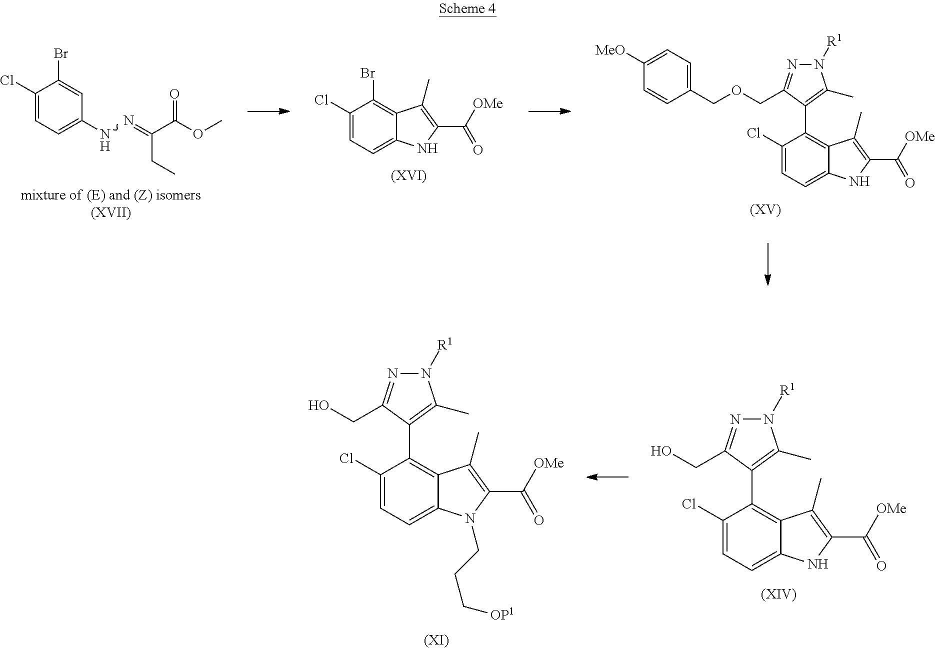

by reacting an intermediate of Formula (XIV), with a suitable O-protected propyl halide or alkylsulfonate such as, for example, (3-bromopropoxy)(tert-butyl)dimethylsilane, in the presence of a suitable base such as, for example, Cs₂CO₃, in a suitable solvent such as, for example, DMF, at a suitable temperature such as, for example, room temperature.
Intermediates of Formula (XIV) can be prepared by reacting an intermediate of Formula (XV), with a suitable deprotecting agent such as, for example, trifluoromethanesulfonic acid, TFA, or DDQ, in a suitable solvent such as, for example, CH₂Cl₂, at a suitable temperature such as, for example, room temperature.
Intermediates of Formula (XV) can be prepared by reacting an intermediate of Formula (XVI), with a suitable substituted pyrazole derivative such as, for example, 3-(((4-methoxybenzyl)oxy)methyl)-1,5-dimethyl-4-(4,4,5,5-tetramethyl-1,3,2-dioxaborolan-2-yl)-1H-pyrazole, in the presence of a suitable catalyst such as, for example, Pd₂(dba)₃, in the presence of a suitable phosphine ligand such as, for example, S-Phos, in the presence of a suitable base such as, for example, sodium bicarbonate, in a suitable solvent such as, for example, dioxane, water, or a mixture thereof, at a suitable temperature such as, for example, 100° C. A skilled person will realize that other suitable substituted pyrazole derivatives, can be for example derivatives wherein the p-methoxybenzyl moiety is replaced by hydrogen or TBDMS.
Intermediates of Formula (XVI) can be prepared by reacting an intermediate of Formula (XVII), with a suitable acid, such as, for example, sulfuric acid, in a suitable solvent, such as, for example, acetic acid, at a suitable temperature, such as, for example, 70° C.
An intermediate of Formula (XVII) can be prepared by reacting (3-bromo-4-chlorophenyl)hydrazine with methyl 2-oxobutanoate, in the presence of a suitable acid, such as, for example, hydrochloric acid, in a suitable solvent, such as, for example, methanol, at a suitable temperature, such as, for example, 65° C.

Intermediates of Formula (XIII), wherein R² and (R^{y})ₙ are defined as in Formula (I), P² is a suitable protecting group, such as, for example, TBDPS, can be prepared according to Scheme 5,

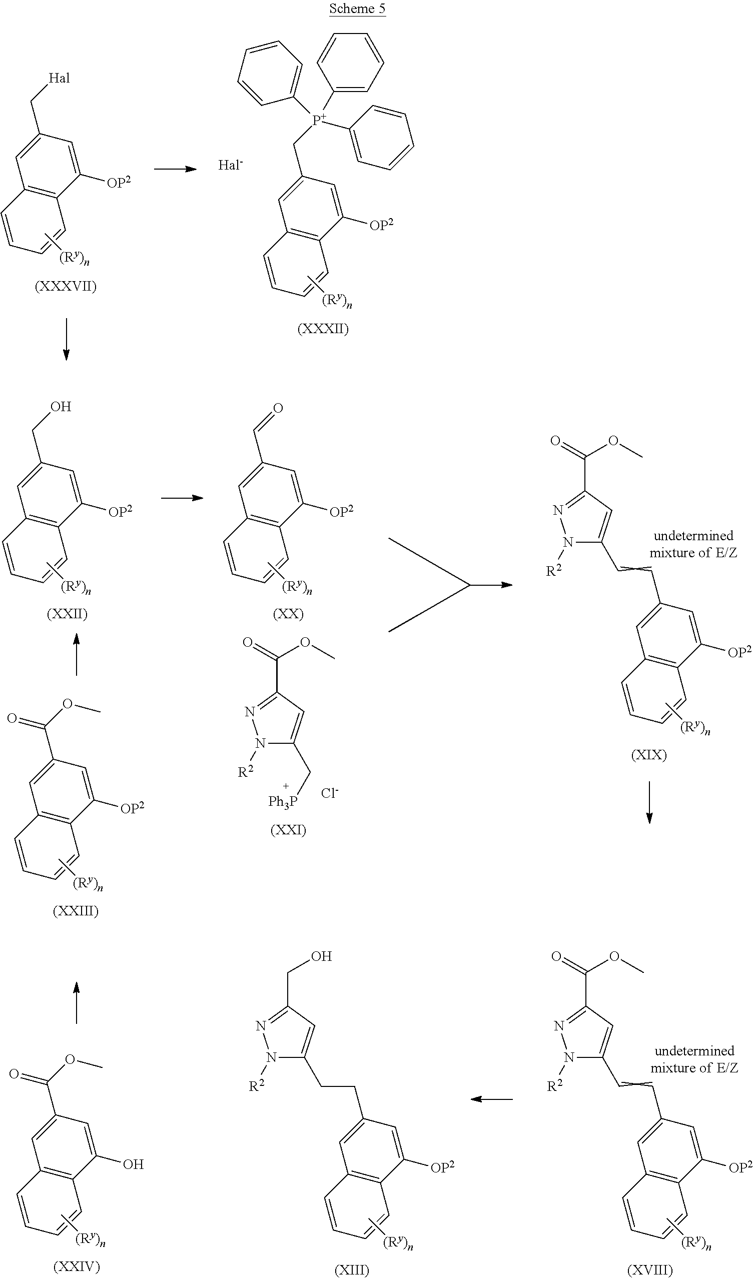

by reacting an intermediate of Formula (XVIII) with a suitable hydrogenating reagent such as, for example, hydrogen gas, in the presence of a suitable catalyst such as, for example, Pd/C, in a suitable solvent such as, for example, MeOH, at a suitable temperature such as, for example, room temperature.
Intermediates of Formula (XVIII) can be prepared by reacting an intermediate of Formula (XIX) with a suitable reducing agent such as, for example, LiAlH₄, in a suitable solvent such as, for example, THF, at a suitable temperature such as, for example, 0° C.
Intermediates of Formula (XIX) can be prepared by reacting an intermediate of Formula (XX) with an intermediate of Formula (XXI), in the presence of a suitable base such as, for example, NaH, in a suitable solvent such as, for example, THF, at a suitable temperature such as, for example, −10° C.
Intermediates of Formula (XX) can be prepared by reacting an intermediate of Formula (XXII) with a suitable oxidizing agent such as, for example, MnO₂, in a suitable solvent such as, for example, acetonitrile, at a suitable temperature such as, for example, 60° C.
Intermediates of Formula (XXII) can be prepared by reacting an intermediate of Formula (XXIII) with a suitable reducing agent such as, for example, LiAlH₄, in a suitable solvent such as, for example, THF, at a suitable temperature such as, for example, 0° C.
Intermediates of Formula (XXIII) can be prepared by reacting an intermediate of Formula (XXIV) with a suitable protecting reagent such as, for example, tert-butyl(chloro)diphenylsilane (TBDPSCl) or 4-methoxybenzyl chloride (PMBCl), in the presence of a suitable base such as, for example, imidazole or NaH, in a suitable solvent such as, for example, DMF, at a suitable temperature such as, for example, room temperature.
Intermediates of Formula (XXI) and Intermediates of Formula (XXIV) are commercially available or can be prepared according to procedures described in literature.

Alternatively, intermediates of Formula (III), wherein R¹, R² and (R^{y})ₙ are defined as in Formula (I), and X is O (oxygen), can be prepared according to Scheme 6,

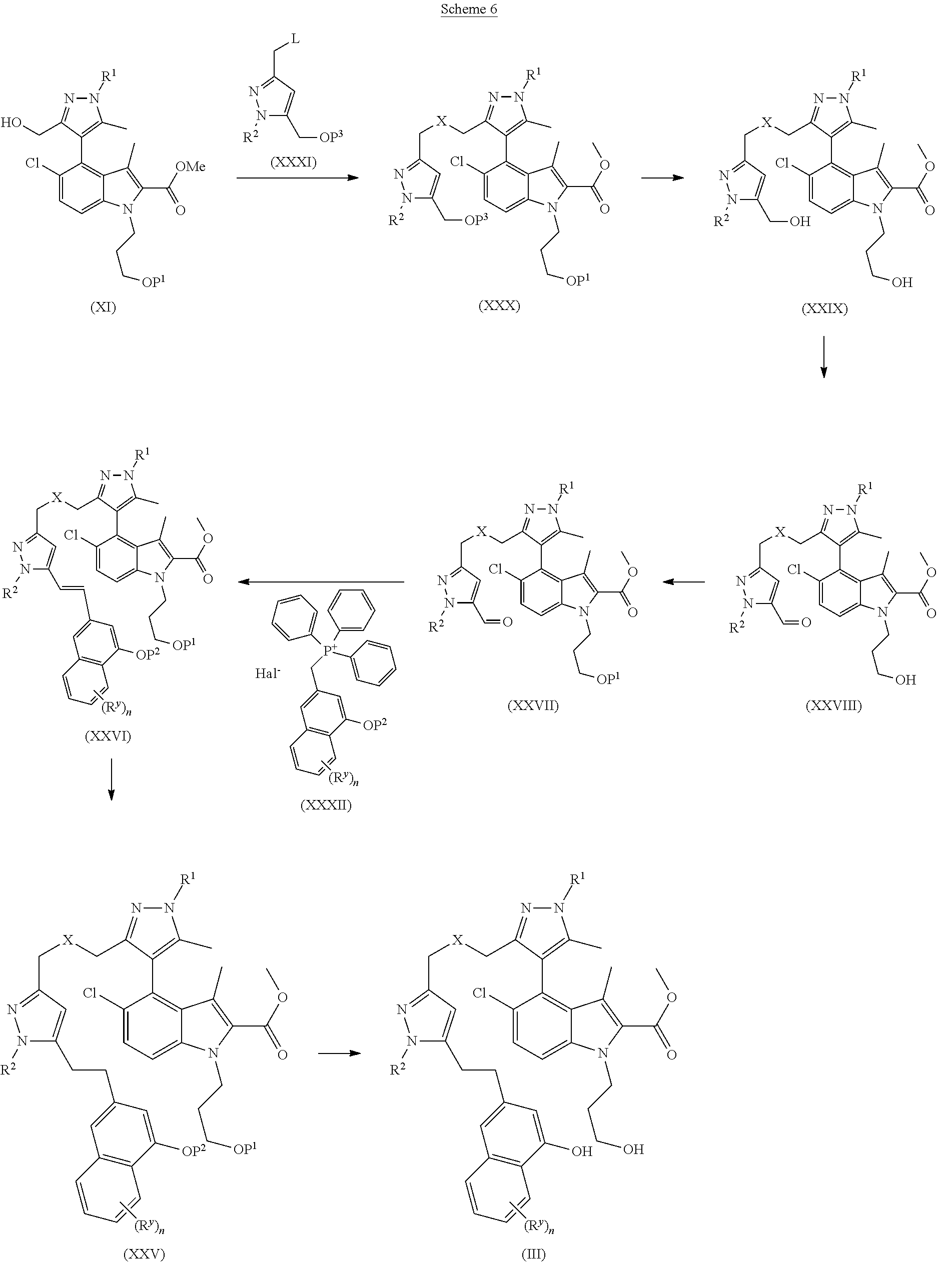

by reacting an intermediate of Formula (XXV) with a suitable deprotection agent such as, for example, p-toluenesulfonic acid (PTSA), in a suitable solvent such as, for example, MeOH, at a suitable temperature such as, for example, room temperature.
Intermediates of Formula (XXV) can be prepared by reacting an intermediate of Formula (XXVI) with a suitable hydrogenating reagent such as, for example, hydrogen gas, in the presence of a suitable catalyst such as Pd/C, in a suitable solvent such as, for example, ethyl acetate (EtOAc), at a suitable temperature such as, for example, room temperature.
Intermediates of Formula (XXVI) can be prepared by reacting an intermediate of Formula (XXVII) with an intermediate of Formula (XXXII), in the presence of a suitable base such as, for example, NaH, in a suitable solvent such as, for example, THF, at a suitable temperature such as, for example, 0° C. or room temperature.
Intermediates of Formula (XXVII) can be prepared by reacting an intermediate of Formula (XXVIII) with a suitable protecting group precursor, such as, for example, tert-butyldimethylsilyl chloride (TBDMSCl), in the presence of a suitable base such as, for example, imidazole, in a suitable solvent such as, for example, DCM, at a suitable temperature such as, for example, room temperature.
Intermediates of Formula (XXVIII) can be prepared by reacting an intermediate of Formula (XXIX) with a suitable oxidizing agent such as, for example, MnO₂, in a suitable solvent such as, for example, DCM, at a suitable temperature such as, for example, room temperature.
Intermediates of Formula (XXIX) can be prepared by reacting an intermediate of Formula (XXX) with with a suitable deprotection agent such as, for example, PTSA, in a suitable in a suitable solvent such as, for example, MeOH, at a suitable temperature such as, for example, 0° C. or room temperature.
Intermediates of Formula (XXX) can be prepared by reacting an intermediate of Formula (XI) with an intermediate of Formula (XXXI), in the presence of a suitable base such as, for example, NaH, in a suitable solvent such as, for example, THF, at a suitable temperature such as, for example, 0° C. or room temperature.
It will be clear for someone skilled in the art, that orthogonality of protective groups will have to be considered, for instance when R¹ is a tetrahydropyranyl, P¹, P² and P³ should be preferably TBDMS or TBDPS groups.

Intermediates of Formula (XXXII) can be prepared according to Scheme 5
by reacting an intermediate of Formula (XXXVII) with a suitable phosphine such as, for example, triphenylphosphine (PPh₃), in a suitable solvent such as, for example, DCM, at a suitable temperature such as, for example, room temperature
Intermediates of Formula (XXXVII) can be prepared by reacting an intermediate of Formula (XXII) with a suitable activating agent such as, for example, thionyl chloride, in a suitable solvent such as, for example, DCM, at a suitable temperature such as, for example, room temperature.

Intermediates of Formula (XXXI), wherein P³ is a suitable protecting group such as, for example, TBDMS, and L is a suitable leaving group such as, for example, I (iodide), can be prepared according to Scheme 7,

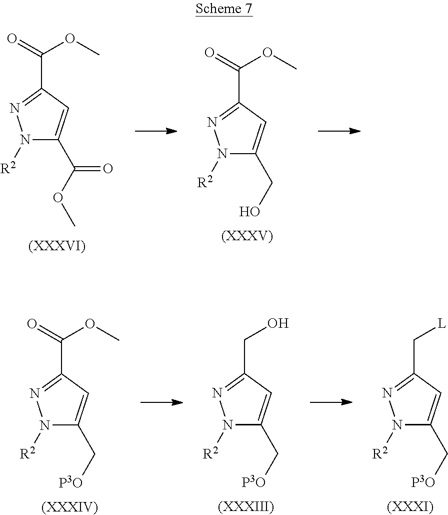

by reacting an intermediate of Formula (XXXIII) with a suitable activating agent such as, for example, mesyl chloride (MsCl), in the presence of a suitable base such as, for example, triethylamine (Et₃N), followed by addition of a suitable leaving group precursor such as, for example, NaI, in a suitable solvent such as, for example, THF, at a suitable temperature such as, for example, room temperature.
Intermediates of Formula (XXXIII) can be prepared by reacting an intermediate of Formula (XXXIV) with a suitable reducing agent such as, for example, LiAlH₄, in a suitable solvent such as, for example, THF, at a suitable temperature such as, for example, 0° C.
Intermediates of Formula (XXXIV) can be prepared by reacting an intermediate of Formula (XXXV) with a suitable protecting group precursor such as, for example, TBDMSCl, in the presence of a suitable base such as, for example, imidazole, in a suitable solvent such as, for example, DCM, at a suitable temperature such as, for example, 0° C. or room temperature.
Intermediates of Formula (XXXV) can be prepared by reacting an intermediate of Formula (XXXVI) with a suitable reducing agent such as, for example, NaBH₄, in a suitable solvent such as, for example, MeOH, 2-methyltetrahydrofuran (2-Me-THF), or a mixture thereof, at a suitable temperature such as, for example, 0° C. or room temperature.
Intermediates of Formula (XXXVI) are commercially available or can be prepared according to reaction protocols known by a skilled person.
It will be clear for a skilled person that, in case R² is a protective group, the protective groups P³ will have to be an orthogonal protective group.

A skilled person will understand that analogous reaction protocols can also be used to prepare Compounds of the invention wherein X¹ represents

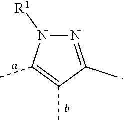

To obtain such compounds, an alternative pyrazole-boronate of Formula (XXXVIII) should be used, which can be prepared according to Scheme 8.

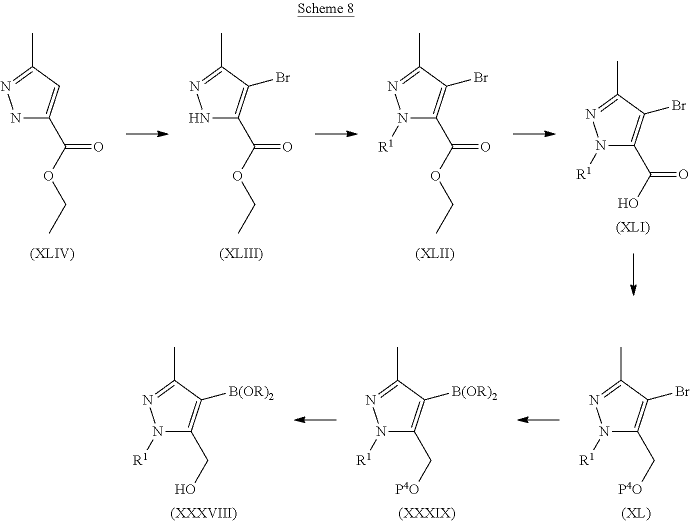

by reacting intermediates of Formula (XXXIX) in which P⁴ is a suitable protective group such as, for example, TBDMS, with a suitable deprotecting reagent such as, for example, tetrabutylammonium fluoride (TBAF), in a suitable solvent such as, for example, THF, at a suitable temperature such as, for example, room temperature or 60° C.
Intermediates of Formula (XXXIX) can be prepared by reacting an intermediate of Formula (XL) with a suitable borylating reagent such as, for example, 2-isopropoxy-4,4,5,5-tetramethyl-1,3,2-dioxaborolane, in the presence of a suitable metalation reagent such as, for example, n-butyllithium, in a suitable solvent such as, for example, THF, at a suitable temperature such as, for example, −78° C.
Intermediates of Formula (XL) can be prepared by reacting an intermediate of Formula (XLI) with a suitable protecting group precursor such as, for example, TBDMSCl, in the presence of a suitable base such as, for example, imidazole, in a suitable solvent such as, for example, DCM, at a suitable temperature such as, for example, 0° C. or room temperature.
Intermediates of Formula (XLI) can be prepared by reacting an intermediate of Formula (XLII) with a suitable reducing agent such as, for example, NaBH₄, in a suitable solvent such as, for example, MeOH or THF, or a mixture thereof, at a suitable temperature such as, for example, 0° C. or room temperature.
Intermediates of Formula (XLII) can be prepared by reacting an intermediate of Formula (XLIII) with a suitable alcohol such as, for example 2-(2-methoxyethoxy)ethanol in the presence of a suitable phosphorane such as, for example, cyanomethylenetributylphosphorane, in a suitable solvent such as, for example THF, at a suitable temperature such as, for example, 0° C. to room temperature.
Intermediates of Formula (XLIII) can be prepared by reacting an intermediate of Formula (XLIV) with a suitable brominating reagent such as, for example, N-bromosuccinimide (NBS), in a suitable solvent such as, for example DCM, at a suitable temperature such as, for example, room temperature.
Intermediates of Formula (XLIV) are commercially available or can be prepared according to procedures described in literature.

Using a similar procedure as described in Scheme 4 for intermediates of Formula (XI), the alternative pyrazole-boronate of Formula (XXXVIII) or its precursor of Formula (XXXIX) can provide intermediates of Formula (XLV) carrying R¹ at the other pyrazole nitrogen position compared to intermediates of Formula (XI).

Alternatively intermediates of Formula (XLV), wherein R¹ is defined as in Formula (I), and P¹ is a suitable protecting group, such as, for example, TBDMS, can be prepared according to Scheme 9,

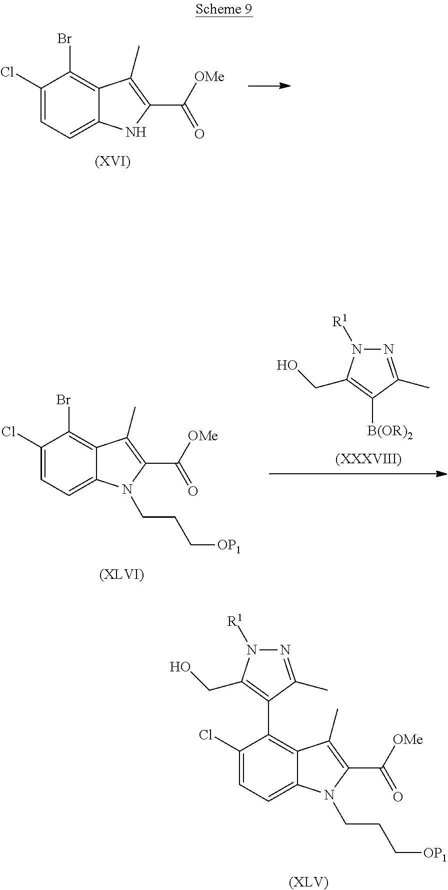

by reacting an intermediate of Formula (XLVI) with an intermediate of Formula (XXXVIII), in the presence of a suitable catalyst such as, for example, dichlorobis[di-tert-butyl(p-dimethylaminophenyl)phosphino]palladium(II) (Pd(amphos)₂Cl₂), in the presence of a suitable base such as, for example, potassium carbonate, in a suitable solvent such as, for example, dioxane, water, or a mixture thereof, at a suitable temperature such as, for example, 65° C.
Intermediates of Formula (XLVI) can be prepared by reacting an intermediate of Formula (XVI) with a suitable O-protected propyl halide or alkylsulfonate such as, for example, (3-bromopropoxy)(tert-butyl)dimethylsilane, in the presence of a suitable base such as, for example, Cs₂CO₃, in a suitable solvent such as, for example, DMF, at a suitable temperature such as, for example, room temperature.
It will be clear to a skilled person that this alternative sequence can also be used to synthesize intermediates of Formula (XI).

It will be clear for a skilled person that Intermediates of Formula (XLV) can be converted to compounds of Formula (I) in a similar way as described for intermediates of Formula (XI).

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl (CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, N.J., 2007.

### Pharmacology of Compounds

It has been found that the compounds of the present invention inhibit one of more MCL-1 activities, such as MCL-1 antiapoptotic activity.

An MCL-1 inhibitor is a compound that blocks one or more MCL-1 functions, such as the ability to bind and repress proapoptotic effectors Bak and Bax or BH3 only sensitizers such as Bim, Noxa or Puma.

The compounds of the present invention can inhibit the MCL-1 pro-survival functions. Therefore, the compounds of the present invention may be useful in treating and/or preventing, in particular treating, diseases that are susceptible to the effects of the immune system such as cancer.

In another embodiment of the present invention, the compounds of the present invention exhibit anti-tumoral properties, for example, through immune modulation.

In an embodiment, the present invention is directed to methods for treating and/or preventing a cancer, wherein the cancer is selected from those described herein, comprising administering to a subject in need thereof (preferably a human), a therapeutically effective amount of a compound of Formula (I), or pharmaceutically acceptable salt, or a solvate thereof.

In an embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of acute lymphoblastic leukemia (ALL), acute myeloid leukemia (AML), B cells acute lymphoblastic leukemia, B-cell chronic lymphocytic leukemia (CLL), bladder cancer, breast cancer, chronic lymphocytic leukemia, chronic myeloid leukemia, colon adenocarcinoma, diffuse large B cell lymphoma, esophageal cancer, follicular lymphoma, gastric cancer, head and neck cancer (including, but not limited to head and neck squamous cell carcinoma), hematopoietic cancer, hepatocellular carcinoma, Hodgkin lymphoma, liver cancer, lung cancer (including but not limited to lung adenocarcinoma), lymphoma, medulloblastoma, melanoma, monoclonal gammopathy of undetermined significance, multiple myeloma, myelodysplastic syndromes, myelofibrosis, myeloproliferative neoplasms, ovarian cancer, ovarian clear cell carcinoma, ovarian serous cystadenoma, pancreatic cancer, polycythemia vera, prostate cancer, rectum adenocarcinoma, renal cell carcinoma, smoldering multiple myeloma, T cell acute lymphoblastic leukemia, T cell lymphoma, and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is preferably selected from the group consisting of acute lymphoblastic leukemia (ALL), acute myeloid leukemia (AML), B cells acute lymphoblastic leukemia, B-cell chronic lymphocytic leukemia (CLL), breast cancer, chronic lymphocytic leukemia, chronic myeloid leukemia, diffuse large B cell lymphoma, follicular lymphoma, hematopoietic cancer, Hodgkin lymphoma, lung cancer (including, but not limited to lung adenocarcinoma) lymphoma, monoclonal gammopathy of undetermined significance, multiple myeloma, myelodysplastic syndromes, myelofibrosis, myeloproliferative neoplasms, smoldering multiple myeloma, T cell acute lymphoblastic leukemia, T cell lymphoma and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of adenocarcinoma, benign monoclonal gammopathy, biliary cancer (including, but not limited to, cholangiocarcinoma), bladder cancer, breast cancer (including, but not limited to, adenocarcinoma of the breast, papillary carcinoma of the breast, mammary cancer, medullary carcinoma of the breast), brain cancer (including, but not limited to, meningioma), glioma (including, but not limited to, astrocytoma, oligodendroglioma; medulloblastoma), bronchus cancer, cervical cancer (including, but not limited to, cervical adenocarcinoma), chordoma, choriocarcinoma, colorectal cancer (including, but not limited to, colon cancer, rectal cancer, colorectal adenocarcinoma), epithelial carcinoma, endothelial sarcoma (including, but not limited to, Kaposi's sarcoma, multiple idiopathic hemorrhagic sarcoma), endometrial cancer (including, but not limited to, uterine cancer, uterine sarcoma), esophageal cancer (including, but not limited to, adenocarcinoma of the esophagus, Barrett's adenocarinoma), Ewing sarcoma, gastric cancer (including, but not limited to, stomach adenocarcinoma), gastrointestinal stromal tumor (GIST), head and neck cancer (including, but not limited to, head and neck squamous cell carcinoma), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, kidney cancer (including, but not limited to, nephroblastoma a.k.a. Wilms' tumor, renal cell carcinoma), liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES), ovarian cancer (including, but not limited to, cystadenocarcinoma, ovarian embryonal carcinoma, ovarian adenocarcinoma), pancreatic cancer (including, but not limited to, pancreatic andenocarcinoma, intraductal papillary mucinous neoplasm (IPMN), Islet cell tumors), prostate cancer (including, but not limited to, prostate adenocarcinoma), skin cancer (including, but not limited to, squamous cell carcinoma (SCC), keratoacanthoma (KA), melanoma, basal cell carcinoma (BCC)) and soft tissue sarcoma (e.g. malignant fibrous histiocytoma (MFH), liposarcoma, malignant peripheral nerve sheath tumor (MPNST), chondrosarcoma, fibrosarcoma, myxosarcoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of benign monoclonal gammopathy, breast cancer (including, but not limited to, adenocarcinoma of the breast, papillary carcinoma of the breast, mammary cancer, medullary carcinoma of the breast), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), and prostate cancer (including, but not limited to, prostate adenocarcinoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of prostate, lung, pancreatic, breast, ovarian, cervical, melanoma, B-cell chronic lymphocytic leukemia (CLL), acute myeloid leukemia (AML), and acute lymphoblastic leukemia (ALL).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is multiple myeloma.

The compounds according to the present invention or pharmaceutical compositions comprising said compounds, may also have therapeutic applications in combination with immune modulatory agents, such as inhibitors of the PD1/PDL1 immune checkpoint axis, for example antibodies (or peptides) that bind to and/or inhibit the activity of PD-1 or the activity of PD-L1 and or CTLA-4 or engineered chimeric antigen receptor T cells (CART) targeting tumor associated antigens.

The compounds according to the present invention or pharmaceutical compositions comprising said compounds, may also be combined with radiotherapy or chemotherapeutic agents (including, but not limited to, anti-cancer agents) or any other pharmaceutical agent which is administered to a subject having cancer for the treatment of said subject's cancer or for the treatment or prevention of side effects associated with the treatment of said subject's cancer.

The compounds according to the present invention or pharmaceutical compositions comprising said compounds, may also be combined with other agents that stimulate or enhance the immune response, such as vaccines.

In an embodiment, the present invention is directed to methods for treating and/or preventing a cancer (wherein the cancer is selected from those described herein) comprising administering to a subject in need thereof (preferably a human), a therapeutically effective amount of co-therapy or combination therapy; wherein the co-therapy or combination therapy comprises a compound of Formula (I) of the present invention and one or more anti-cancer agent(s) selected from the group consisting of (a) immune modulatory agent (such as inhibitors of the PD1/PDL1 immune checkpoint axis, for example antibodies (or peptides) that bind to and/or inhibit the activity of PD-1 or the activity of PD-L1 and or CTLA-4); (b) engineered chimeric antigen receptor T cells (CART) targeting tumor associated antigens; (c) radiotherapy; (d) chemotherapy; and (e) agents that stimulate or enhance the immune response, such as vaccines.

The present invention is directed to compounds of Formula (I) and pharmaceutically acceptable salts, and solvates thereof, for use as a medicament.

The present invention is directed to compounds of Formula (I) and pharmaceutically acceptable salts, and solvates thereof, for use in the inhibition of MCL-1 activity.

As used herein, unless otherwise noted, the term “anti-cancer agents” shall encompass “anti-tumor cell growth agents” and “anti-neoplastic agents”.

The present invention is directed to compounds of Formula (I) and pharmaceutically acceptable salts, and solvates thereof, for use in treating and/or preventing diseases (preferably cancers) mentioned above.

The present invention is directed to compounds of Formula (I) and pharmaceutically acceptable salts, and solvates thereof, for treating and/or preventing diseases (preferably cancers) mentioned above.

The present invention is directed to compounds of Formula (I) and pharmaceutically acceptable salts, and solvates thereof, for treating and/or preventing, in particular for treating, a disease, preferably a cancer, as described herein (for example, multiple myeloma).

The present invention is directed to compounds of Formula (I) and pharmaceutically acceptable salts, and solvates thereof, for use in treating and/or preventing, in particular for treating, a disease, preferably a cancer, as described herein (for example, multiple myeloma).

The present invention is directed to compounds of Formula (I) and pharmaceutically acceptable salts, and solvates thereof, for treating and/or preventing, in particular for treating, MCL-1 mediated diseases or conditions, preferably cancer, more preferably a cancer as herein described (for example, multiple myeloma).

The present invention is directed to compounds of Formula (I) and pharmaceutically acceptable salts, and solvates thereof, for use in treating and/or preventing, in particular for use in treating, MCL-1 mediated diseases or conditions, preferably cancer, more preferably a cancer as herein described (for example, multiple myeloma).

The present invention relates to compounds of Formula (I) and pharmaceutically acceptable salts, and solvates thereof, for the manufacture of a medicament.

The present invention relates to compounds of Formula (I) and pharmaceutically acceptable salts, and solvates thereof, for the manufacture of a medicament for the inhibition of MCL-1.

The present invention relates to compounds of Formula (I) and pharmaceutically acceptable salts, and solvates thereof, for the manufacture of a medicament for treating and/or preventing, in particular for treating, a cancer, preferably a cancer as herein described. More particularly, the cancer is a cancer which responds to inhibition of MCL-1 (for example, multiple myeloma).

The present invention is directed to compounds of Formula (I) and pharmaceutically acceptable salts, and solvates thereof, for the manufacture of a medicament for treating and/or preventing, in particular for treating, any one of the disease conditions mentioned hereinbefore.

The present invention is directed to compounds of Formula (I) and pharmaceutically acceptable salts, and solvates thereof, for the manufacture of a medicament for treating and/or preventing any one of the disease conditions mentioned hereinbefore.

The compounds of Formula (I) and pharmaceutically acceptable salts, and solvates thereof, can be administered to subjects, preferably humans, for treating and/or preventing of any one of the diseases mentioned hereinbefore.

In view of the utility of the compounds of Formula (I) and pharmaceutically acceptable salts, and solvates thereof, there is provided a method of treating subjects, preferably mammals such as humans, suffering from any of the diseases mentioned hereinbefore; or a method of slowing the progression of any of the diseases mentioned hereinbefore in subject, humans; or a method of preventing subjects, preferably mammals such as humans, from suffering from any one of the diseases mentioned hereinbefore.

Said methods comprise the administration, i.e. the systemic or topical administration, preferably oral or intravenous administration, more preferably oral administration, of an effective amount of a compound of Formula (I) or a pharmaceutically acceptable salt, or a solvate thereof, to subjects such as humans.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. In an embodiment, a therapeutically effective daily amount may be from about 0.005 mg/kg to 100 mg/kg.

The amount of a compound according to the present invention, also referred to herein as the active ingredient, which is required to achieve a therapeutic effect may vary on case-by-case basis, for example with the specific compound, the route of administration, the age and condition of the recipient, and the particular disorder or disease being treated. The methods of the present invention may also include administering the active ingredient on a regimen of between one and four intakes per day. In these methods of the present invention, the compounds according to the invention are preferably formulated prior to administration.

The present invention also provides compositions for treating and/or preventing the disorders (preferably a cancer as described herein) referred to herein. Said compositions comprise a therapeutically effective amount of a compound of Formula (I), or a pharmaceutically acceptable salt, or a solvate thereof, and a pharmaceutically acceptable carrier or diluent.

While it is possible for the active ingredient (e.g. a compound of the present invention) to be administered alone, it is preferable to administer it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of the present invention may be prepared by any methods well known in the art of pharmacy, for example, using methods such as those described in, for example, Gennaro et al. Remington's Pharmaceutical Sciences (18^{th} ed., Mack Publishing Company, 1990, see especially Part 8. Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, in an embodiment, the present invention is directed to a product comprising, as a first active ingredient a compound according to the invention and as further, as an additional active ingredient one or more anti-cancer agent(s), as a combined preparation for simultaneous, separate or sequential use in the treatment of patients suffering from cancer.

The one or more other anti-cancer agents and the compound according to the present invention may be administered simultaneously (e.g. in separate or unitary compositions) or sequentially, in either order. In an embodiment, the two or more compounds are administered within a period and/or in an amount and/or a manner that is sufficient to ensure that an advantageous or synergistic effect is achieved. It will be appreciated that the preferred method and order of administration and the respective dosage amounts and regimes for each component of the combination will depend on the particular other anti-cancer agent and the compound of the present invention being administered, their route of administration, the particular condition, in particular tumor, being treated and the particular host being treated.

The following examples further illustrate the present invention.

### EXAMPLES

Several methods for preparing the Compounds of this invention are illustrated in the following examples. Unless otherwise noted, all starting materials were obtained from commercial suppliers and used without further purification, or alternatively can be synthesized by a skilled person by using well-known methods.

| Abbreviation | Meaning |
|---|---|
| ACN | acetonitrile |
| AcOH | acetic acid |
| Celite ® | diatomaceous earth |
| Co | Compound |
| Co. No. | Compound Number |
| DCM | dichloromethane |
| DDQ | 2,3-Dichloro-5,6-dicyano-1,4-benzoquinone |
| Dess-Martin | 3-oxo-1,3-dihydro-1λ⁵,2- |
| periodinane | benziodoxole-1,1,1-triyl triacetate |
| DIBAL | diisobutylaluminium hydride |
| Dicalite ® | diatomaceous earth |
| DIPE | diisopropyl ether |
| DIPEA | N,N-diisopropylethylamine |
| DMAP | 4-dimethylaminopyridine |
| DMF | N,N-dimethylformamide |
| DTBAD | Di-tert-butyl Azodicarboxylate |
| eq. | equivalent(s) |
| Et₃N or TEA | trietylamine |
| Et₃N.(HF)₃ | triethylamine trihydrofluoride |
| EtOAc or AcOEt | ethyl acetate |
| EtOH | ethanol |
| Et₂O | diethyl ether |
| h | hour(s) |
| HPLC | high performance liquid chromatography |
| iPrNH₂ | isopropylamine |
| IPA or iPrOH | isopropanol |
| mCPBA | meta-chloroperoxybenzoic acid |
| KSAc | potassium thioacetate |
| Me | methyl |
| MeI | methyl iodide |
| MeOH | methanol |
| 2-Me-THF | 2-methyltetrahydrofuran |
| MP | melting point |
| MsCl | methanesulfonyl chloride |
| NH₃-MeOH | ammonia solution in methanol |
| nBuLi | n-butyllithium |
| NaBH(OAc)₃ | sodium triacetoxyborohydride |
| NaOAc | sodium acetate |
| Pd/C | palladium on carbon |
| Pd(amphos)₂Cl₂ | dichlorobis[di-tert-butyl(p- |
| | dimethylaminophenyl)phosphino]palladium(II) |
| Pd₂(dba)₃ | tris(dibenzylideneacetone)dipalladium(0) |
| Pd(dtbpf)Cl₂ | 1,1′-Bis(di-t-butylphosphino)ferrocene palladium |
| | dichloride |
| PPh₃ | triphenylphosphine |
| pTsOH | p-toluenesulfonic acid |
| pTsOH•H₂O | p-toluenesulfonic acid monohydrate |
| rac | racemic |
| Rochelle salt | potassium sodium tartrate tetrahydrate |
| RP | reversed phase |
| SFC | supercritical fluid chromatography |
| S-Phos | 2-dicyclohexylphosphino-2′,6′-dimethoxybiphenyl |
| TBAF | tetrabutylammonium fluoride |
| TBDMSCl | tert-butyldimethylsilyl chloride |
| TBDPSCl | tert-butyl(chloro)diphenylsilane |
| THF | tetrahydrofuran |
| TLC | thin layer chromatography |

As understood by a person skilled in the art, Compounds synthesized using the protocols as indicated may contain residual solvent or minor impurities.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

Compounds or intermediates indicated as “Sₐ or Rₐ atropisomer” or “Rₐ or Sₐ atropisomer” are compounds or intermediates which are one atropisomeric form but for which the absolute stereochemistry is undetermined.

### Preparation of Intermediates

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

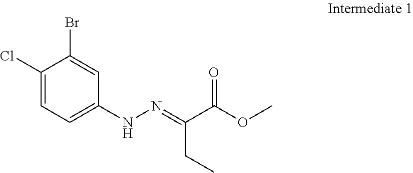 [SMILES: CC/C(=N\Nc1ccc(Cl)c(Br)c1)C(=O)OC]

A solution of (3-bromo-4-chlorophenyl)hydrazine (4.655 g, 18.047 mmol) and methyl 2-oxobutanoate (1.02 eq) in HCl (93 mL, 1.25 M in MeOH) was refluxed for 90 min. The reaction mixture was cooled to room temperature and volatiles were removed under reduced pressure to give 5.768 g of Intermediate 1 as a brown oily residue that solidified within minutes, used without further purification in the next step.

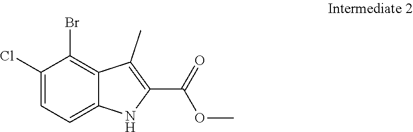 [SMILES: COC(=O)c1[nH]c2ccc(Cl)c(Br)c2c1C]

A suspension of Intermediate 1 (5.768 g, 18 mmol) in acetic acid (37 mL) was heated to 70° C. Sulfuric acid (4.81 mL, 5 eq.) was added dropwise over 10 min (exotherm developed and a precipitate formed). After 15 additional min, the reaction mixture was cooled to room temperature and then to 0° C. by adding ice. The solid precipitate was filtered and washed with water until the filtrate was of neutral pH. The solid was triturated with cold heptane/diisopropylether (8/2, 50 mL) to give an off-white solid. This solid was purified by preparative SFC (Stationary phase: Chiralpak Daicel IG 20×250 mm, Mobile phase: CO₂, EtOH+0.4% iPrNH₂) to give Intermediate 2 (1.745 g, 32%).

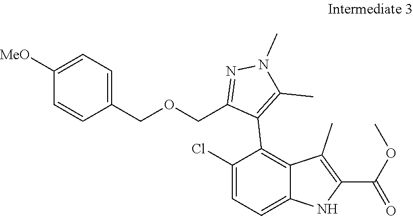 [SMILES: COC(=O)c1[nH]c2ccc(Cl)c(-c3c(COCc4ccc(OC)cc4)nn(C)c3C)c2c1C]

Intermediate 2 (500 mg, 1.65 mmol), 3-(((4-methoxybenzyl)oxy)methyl)-1,5-dimethyl-4-(4,4,5,5-tetramethyl-1,3,2-dioxaborolan-2-yl)-1H-pyrazole [2143010-90-4] (800 mg, 1.3 eq.), Pd₂(dba)₃ (76 mg, 0.05 eq.), and S-Phos (68 mg, 0.1 eq.) were weighed in a pressure tube under nitrogen atmosphere. Dioxane (10.5 mL) and a saturated aqueous NaHCO₃ solution (4.5 mL) were added and the mixture was heated at 100° C. for 2 h. The reaction mixture was cooled to room temperature, diluted with EtOAc (40 mL) and water (40 mL). The organic layer was separated and the aqueous one was extracted with EtOAc (40 mL). The combined organic layer was dried over MgSO₄, filtered and evaporated. The crude mixture was purified by flash chromatography on silica gel (40 g, gradient: from heptane 100% up to heptane/EtOAc 4/6). Intermediate 3 (790 mg, 89%) was obtained as a yellowish oil that solidified on standing. Intermediate 3 was used without further purification in the next reaction step.

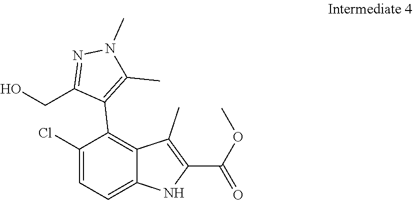 [SMILES: COC(=O)c1[nH]c2ccc(Cl)c(-c3c(CO)nn(C)c3C)c2c1C]

Trifluoromethanesulfonic acid (0.888 mL, 5 eq.) was added to a solution of Intermediate 3 (1080 mg, 2 mmol) in DCM (25 mL). The reaction mixture was stirred at room temperature for 1 h. The reaction mixture was diluted with DCM (100 mL) and treated with saturated aqueous NaHCO₃ (30 mL). The organic layer was separated and the aqueous one was extracted with DCM (50 mL×3). The combined organic layer was dried over MgSO₄, filtered, and evaporated. Intermediate 4 (625 mg, 89%) was obtained as a yellowish solid, used without further purification in the next step.

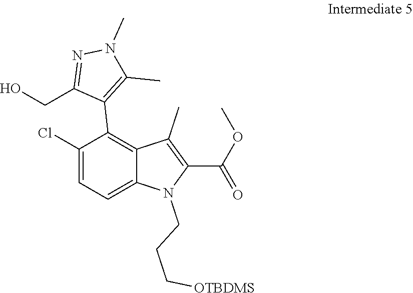 [SMILES: COC(=O)c1c(C)c2c(-c3c(CO)nn(C)c3C)c(Cl)ccc2n1CCCO[Si](C)(C)C(C)(C)C]

Cesium carbonate (732 mg, 1.25 eq.) was added to a solution of Intermediate 4 (625 mg, 1.79 mmol) in DMF (10 mL) under nitrogen atmosphere. (3-Bromopropoxy)(tert-butyl)dimethylsilane (0.458 mL, 1.1 eq.) was added dropwise and the reaction mixture was stirred at room temperature overnight. The reaction mixture was diluted with EtOAc (100 mL) and water (50 mL). The organic layer was separated and washed with brine (2×30 mL). The combined aqueous layers were extracted with EtOAc (50 mL). The combined organic layer was then dried over MgSO₄, filtered and evaporated. The crude mixture was purified by flash chromatography on silica gel (40 g, gradient: from heptane 100% up to EtOAc 100%) to afford Intermediate 5 (360 mg, 38%) as a white solid.

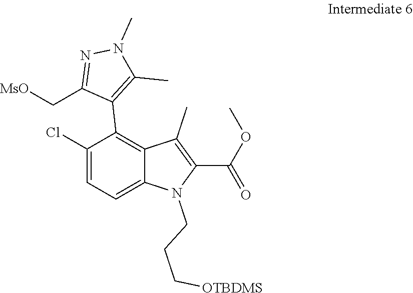 [SMILES: COC(=O)c1c(C)c2c(-c3c(COS(C)(=O)=O)nn(C)c3C)c(Cl)ccc2n1CCCO[Si](C)(C)C(C)(C)C]

Mesyl chloride (0.12 mL, 2.5 eq.) was added dropwise to a solution of Intermediate 5 (320 mg, 0.61 mmol) and triethylamine (0.256 mL, 3 eq.) in DCM (10 mL) stirring at 0° C. under nitrogen. The reaction mixture was then allowed to warm up to room temperature and was stirred at room temperature for 1 h. Additional triethylamine (3 eq.) and mesyl chloride (2.5 eq.) were added and stirring was continued at room temperature for 1 h. The reaction mixture was diluted with DCM (10 mL) and treated with saturated aqueous NaHCO₃ (5 mL). The organic layer was separated and the aqueous one was extracted with DCM (10 mL). The combined organic layer was dried over MgSO₄, filtered, and evaporated to give Intermediate 6 (368 mg, quantitative), used without further purification in the next step.

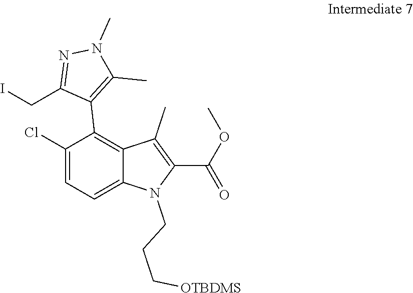 [SMILES: COC(=O)c1c(C)c2c(-c3c(CI)nn(C)c3C)c(Cl)ccc2n1CCCO[Si](C)(C)C(C)(C)C]

Potassium iodide (1.021 g, 10 eq.) was added to a solution of Intermediate 6 (368 mg, 0.61 mmol) in acetonitrile (5 mL). The reaction was stirred at room temperature overnight. The reaction mixture was diluted with EtOAc (50 mL) and filtered over Dicalite®. Water (25 mL) was added to the filtrate and, after some stirring, the organic layer was separated. The aqueous layer was back-extracted with EtOAc (25 mL). The combined organic layer was dried over MgSO₄, filtered, and evaporated to give Intermediate 7, used without further purification in the next step.

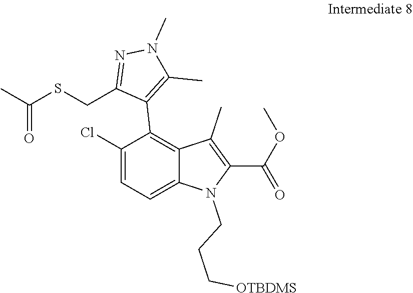 [SMILES: COC(=O)c1c(C)c2c(-c3c(CSC(C)=O)nn(C)c3C)c(Cl)ccc2n1CCCO[Si](C)(C)C(C)(C)C]

KSAc (400 mg, 1.5 eq.) was added to a degassed solution of Intermediate 7 (1.55 g, 2.337 mmol) in ACN (25 mL) at room temperature. The resulting reaction mixture was stirred at room temperature for 16 h. The reaction mixture was filtered through a pad of Celite and concentrated. The crude product was purified by flash column chromatography on silica gel (heptane:EtOAc—1:0 to 6:4) to give Intermediate 8 (1.15 g, yield: 80%) as a yellow oil.

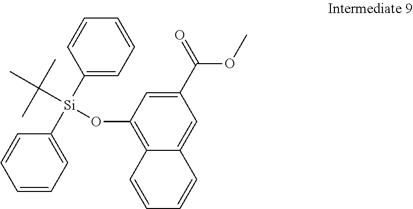 [SMILES: COC(=O)c1cc(O[Si](c2ccccc2)(c2ccccc2)C(C)(C)C)c2ccccc2c1]

TBDPSCl (6.41 mL, 1.25 eq.) was added dropwise to a solution of methyl 4-hydroxy-2-naphthoate ([34205-71-5], 4 g, 19.78 mmol) and imidazole (2.35 g, 1.75 eq.) in DMF (70 mL), cooled to 0° C. Once the addition was complete, the reaction mixture was stirred at room temperature for 14 h. The reaction mixture was diluted with EtOAc (40 mL) and washed subsequently with water, dilute aqueous HCl (0.1 M), saturated aqueous NaHCO₃, and brine (each 30 mL). The organic layer was dried over MgSO₄, filtered, and concentrated. The residue was purified by column chromatography on silica gel (heptane:EtOAc—1:0 to 9:1) to afford Intermediate 9 (8.81 g, yield: 91%) as a yellow oil.

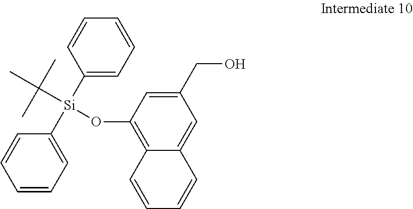 [SMILES: CC(C)(C)[Si](Oc1cc(CO)cc2ccccc12)(c1ccccc1)c1ccccc1]

LiAlH₄ (2 M solution in THF, 9.44 mL, 1.05 eq.) was slowly added to a solution of Intermediate 9 (8.8 g, 17.97 mmol) in THF (70 mL) cooled to 0° C. Once the addition was complete, the reaction mixture was stirred at 0° C. for 30 min. The reaction was quenched by slow addition of EtOAc (20 mL) followed by a saturated solution of Rochelle salt. The heterogeneous mixture was stirred at room temperature for 2 h. The aqueous layer was extracted with EtOAc (2×65 mL), and the combined organic extracts were washed with brine (20 mL), dried over MgSO₄, filtered, and concentrated. The residue was purified by flash column chromatography on silica gel (heptane:EtOAc—1:0 to 3:1) to give Intermediate 10 (5.81 g, yield: 74%) as a white solid.

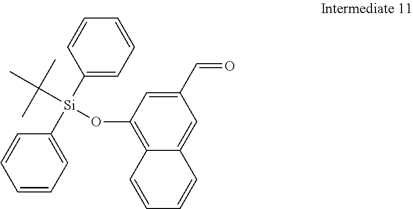 [SMILES: CC(C)(C)[Si](Oc1cc(C=O)cc2ccccc12)(c1ccccc1)c1ccccc1]

MnO₂ (5.81 g, 5 eq.) was added to a solution of Intermediate 10 (5.81 g, 13.38 mmol) in ACN (60 mL) at room temperature. The resulting solution was stirred at 60° C. for 2 h. The reaction mixture was filtered over a pad of Dicalite® and concentrated to give Intermediate 11 (5.47 g, yield: 94%) as a white solid, used without further purification.

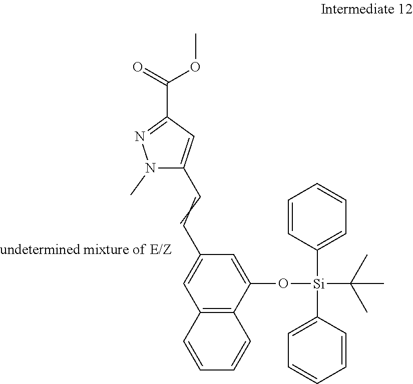 [SMILES: COC(=O)c1cc(C=Cc2cc(O[Si](c3ccccc3)(c3ccccc3)C(C)(C)C)c3ccccc3c2)n(C)n1]

NaH (653 mg, 1.1 eq.) was added to a suspension of intermediate 105 (8.094 g, 1.1 eq.) in THF (90 mL) at 0° C. The resulting solution (solution A) was stirred at 0° C. for 45 min before it was cooled to −25° C. A solution of Intermediate 11 (6.7 g, 15.5 mmol) in THF (16 mL) was added slowly to solution A while maintaining the temperature between −20° C. and −30° C. Once the addition was complete, the reaction mixture was stirred at −10° C. for 1 h. The reaction was quenched by slow addition of saturated aqueous NH₄Cl (10 mL) at −10° C. and was diluted with EtOAc (100 mL). The layers were separated and the aqueous layer was extracted with EtOAc (2×100 mL). The combined organic layer was dried over MgSO₄, filtered, and concentrated. The residue was purified by column chromatography on silica gel (heptane:EtOAc—1:0 to 7:3) to afford Intermediate 12 (6.75 g, yield: 75%) as a white foam.

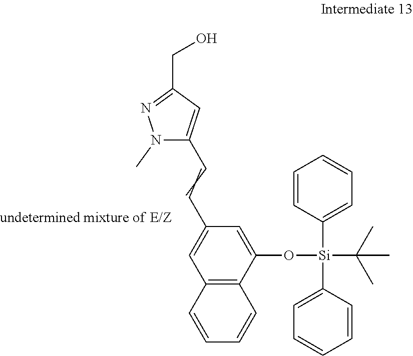 [SMILES: Cn1nc(CO)cc1C=Cc1cc(O[Si](c2ccccc2)(c2ccccc2)C(C)(C)C)c2ccccc2c1]

LiAlH₄ (2 M solution in THF, 6.1 mL, 1.05 eq.) was slowly added to a solution of Intermediate 12 (6.7 g, 11.64 mmol) in THF (45 mL) cooled to 0° C. Once the addition was complete, the reaction mixture was stirred at 0° C. for 30 min. The reaction was quenched by slow addition of EtOAc (20 mL) followed by a saturated solution of Rochelle salt. The heterogeneous mixture was stirred at room temperature for 2 h. The aqueous layer was extracted with EtOAc (2×65 mL), and the combined organic extracts were washed with brine (20 mL), dried over MgSO₄, filtered, and concentrated to afford Intermediate 13 (6.01 g, yield: 94%) as a white foam, used without further purification.

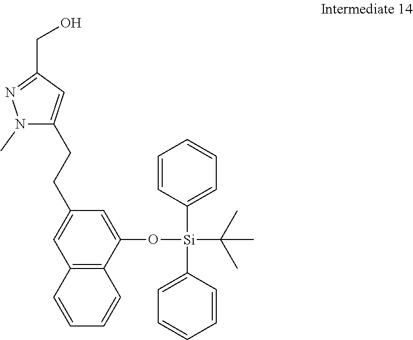 [SMILES: Cn1nc(CO)cc1CCc1cc(O[Si](c2ccccc2)(c2ccccc2)C(C)(C)C)c2ccccc2c1]

Intermediate 13 (5.95 g, 10.89 mmol) was dissolved in MeOH (280 mL). Pd/C (10%, 1159 mg, 0.1 eq.) was added under nitrogen atmosphere. The reaction mixture was then flushed with hydrogen gas and vacuum (3 times), then hydrogen (atmospheric pressure, 244 mL, 1 eq.) was taken up while stirring at room temperature. The reaction mixture was filtered over a pad of Dicalite® and concentrated to give Intermediate 14 (5.9 g, yield: 98%) as a glassy yellow solid, used without further purification.

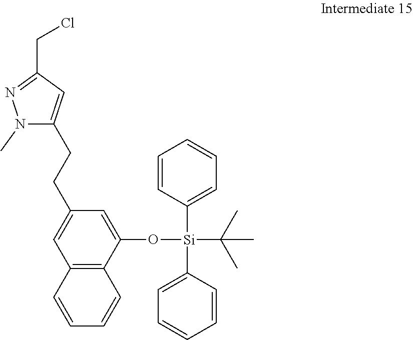 [SMILES: Cn1nc(CCl)cc1CCc1cc(O[Si](c2ccccc2)(c2ccccc2)C(C)(C)C)c2ccccc2c1]

Thionyl chloride (459 μL, 1.15 eq.) was added to a solution of Intermediate 14 (3 g, 5.47 mmol) in DCM (23 mL) cooled to 0° C. Once the addition was complete, the reaction was allowed to warm to room temperature and was stirred for 1 h. The reaction mixture was diluted with DCM (35 mL), washed with saturated aqueous NaHCO₃ (2×50 mL) and brine (50 mL). The organic layer was dried over MgSO₄, filtered, and concentrated. The residue was purified by flash column chromatography on silica gel (heptane:EtOAc—1:0 to 8:2) to give Intermediate 15 (2.65 g, yield: 85%) as a colorless oil that crystallized on standing to a white amorphous solid.

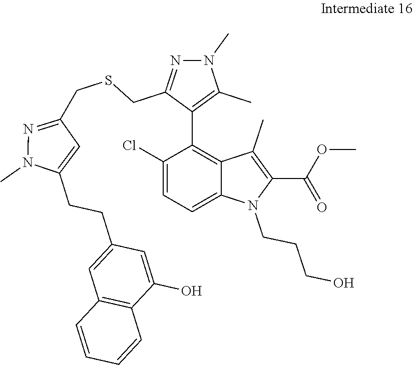

Intermediate 8 (500 mg, 0.821 mmol) and Intermediate 15 (559 mg, 1.2 eq.) were dissolved in MeOH (10 mL). The reaction mixture was degassed and re-filled with nitrogen three times. The reaction mixture was then cooled to 0° C. before addition of K₂CO₃ (227 mg, 2 eq.). After that addition, the reaction mixture was again degassed and re-filled with nitrogen twice. The reaction mixture was allowed to warm to room temperature and was stirred for 3 h. The reaction mixture was concentrated and the residue was partitioned between water (10 mL) and EtOAc (15 mL). The layers were separated and the aqueous layer was extracted with EtOAc (2×20 mL). The combined organic layer was washed with brine (30 mL), dried over MgSO₄, filtered, and concentrated to give a pale yellow foam.

This crude foam was dissolved in MeOH (10 mL) and pTsOH (469 mg, 3 eq.) was added. The resulting reaction mixture was stirred at room temperature for 30 min. The reaction mixture was concentrated. The residue was dissolved in EtOAc (20 mL) and washed with saturated aqueous NaHCO₃ (15 mL). The aqueous layer was extracted with EtOAc (2×20 mL), and the combined organic layer was washed with brine (30 mL), dried over MgSO₄, filtered, and concentrated. The crude product was purified by flash column chromatography on silica gel (heptane:EtOAc—6:4 to 0:1) to give Intermediate 16 (695 mg, yield: 92%) as a yellow oil.

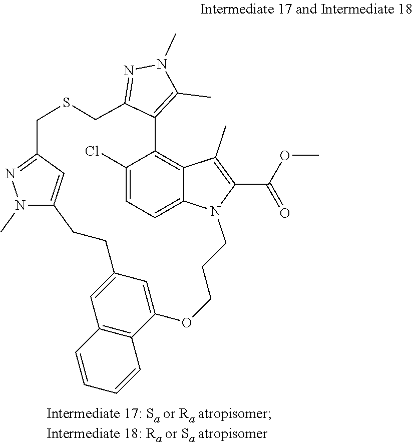

A solution of Intermediate 16 (690 mg, 0.754 mmol) and DTBAD (694 mg, 4 eq.) dissolved in a mixture of toluene (22 mL) and THE (4.5 mL) was added dropwise with a syringe pump (0.1 mL/min) to a solution of PPh₃ (791 mg, 4 eq.) in toluene (22 mL) at 70° C. At the end of the addition, the reaction mixture was concentrated. The residue was purified by flash column chromatography on silica gel (heptane:EtOAc—6:4 to 0:1) to give the racemic mixture (320 mg, yield: 60%) of Intermediate 17 and Intermediate 18 as a white foam.

200 mg of the isolated mixture were purified by preparative SFC (Stationary phase: Chiralpak Daicel ID 20×250 mm, Mobile phase: CO₂, EtOH-iPrOH (50-50)+0.4% iPrNH₂) to give Intermediate 17 (56 mg, yield: 10%) and Intermediate 18 (68 mg, yield: 13%) as colourless oils.

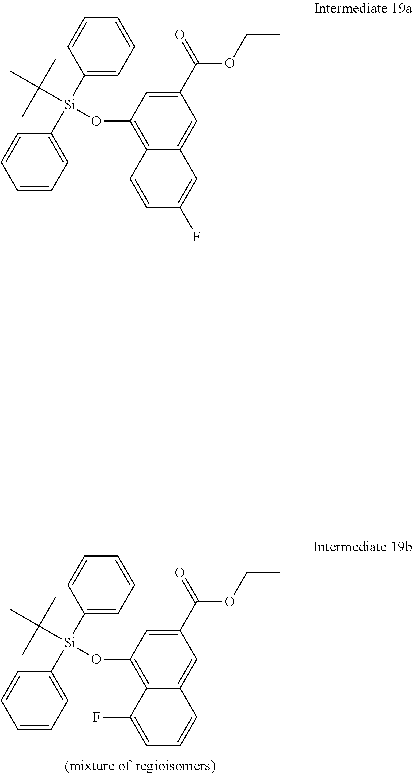 [SMILES: CCOC(=O)c1cc(O[Si](c2ccccc2)(c2ccccc2)C(C)(C)C)c2ccc(F)cc2c1 ; CCOC(=O)c1cc(O[Si](c2ccccc2)(c2ccccc2)C(C)(C)C)c2c(F)cccc2c1]

TBDPSCl (3.726 mL, 3 eq.) was added dropwise to a 5:1 mixture of ethyl 7-fluoro-4-hydroxy-2-naphthoate [1093083-28-3], ethyl 5-fluoro-4-hydroxy-2-naphthoate [1093083-27-2] (2244 mg, 9.58 mmol), and imidazole (1141 mg, 3.5 eq.) in DMF (25 mL), cooled to 0° C. Once the addition was complete, the reaction was stirred at room temperature for 12 h. The reaction mixture was diluted with EtOAc (40 mL) and washed successively with water, dilute aqueous HCl (0.1 M), saturated aqueous NaHCO₃, and brine (each 30 mL). The organic layer was dried over MgSO₄, filtered, and concentrated to afford a pale yellow oil. This oil was purified by column chromatography on silica gel (heptane:EtOAc—1:0 to 9:1) to afford the mixture of Intermediate 19a and Intermediate 19b (2:1 ratio, 5.65 g, still impure, yield considered quantitative) as a yellow oil, used without further purification.

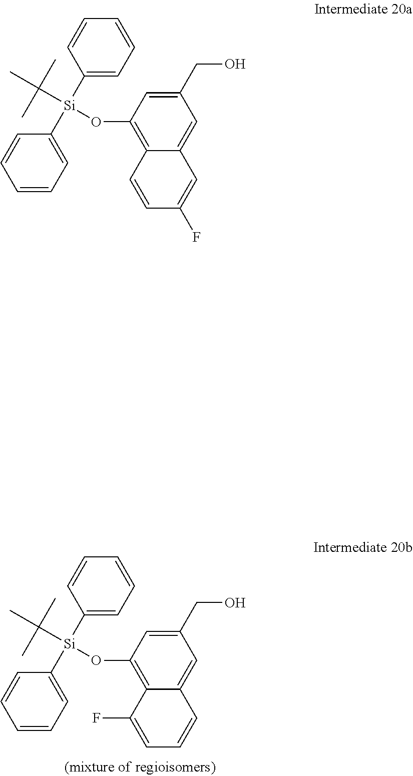 [SMILES: CC(C)(C)[Si](Oc1cc(CO)cc2cc(F)ccc12)(c1ccccc1)c1ccccc1 ; CC(C)(C)[Si](Oc1cc(CO)cc2cccc(F)c12)(c1ccccc1)c1ccccc1]

LiAlH₄ (2 M in THF, 4.888 mL, 1.05 eq.) was added slowly to the mixture of Intermediate 19a and Intermediate 19b (4.4 g, 9.31 mmol) in THF (35 mL), cooled to 0° C. Once the addition was complete, the reaction mixture was stirred at 0° C. for 2 h. The reaction was quenched by slow addition of EtOAc (20 mL) followed by a saturated aqueous solution of Rochelle salt. The heterogeneous mixture was stirred at room temperature for 2 h. The aqueous layer was extracted with EtOAc (2×65 mL), the combined organic extract was washed with brine (20 mL), dried over MgSO₄, filtered, and concentrated to afford an orange oil. The crude product was purified by flash column chromatography on silica gel (heptane:EtOAc—1:0 to 3:1) to give the mixture of Intermediate 20a and Intermediate 20b (4.2 g, yield: 94%) as a white solid.

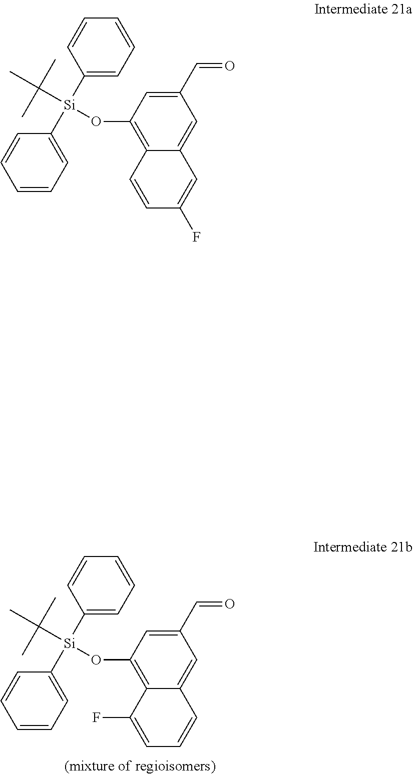 [SMILES: CC(C)(C)[Si](Oc1cc(C=O)cc2cc(F)ccc12)(c1ccccc1)c1ccccc1 ; CC(C)(C)[Si](Oc1cc(C=O)cc2cccc(F)c12)(c1ccccc1)c1ccccc1]

MnO₂ (5.907 g, 5 eq.) was added to the mixture of Intermediate 20a and Intermediate 20b (6.501 g, 13.589 mmol) in ACN (60 mL) at room temperature. The resulting solution was stirred at 60° C. for 2 h. The reaction mixture was filtered over a pad of Dicalite® and concentrated to give the mixture of Intermediate 21a and Intermediate 21b (4.45 g, 80% pure, yield: 61%) as a white solid, used without further purification.

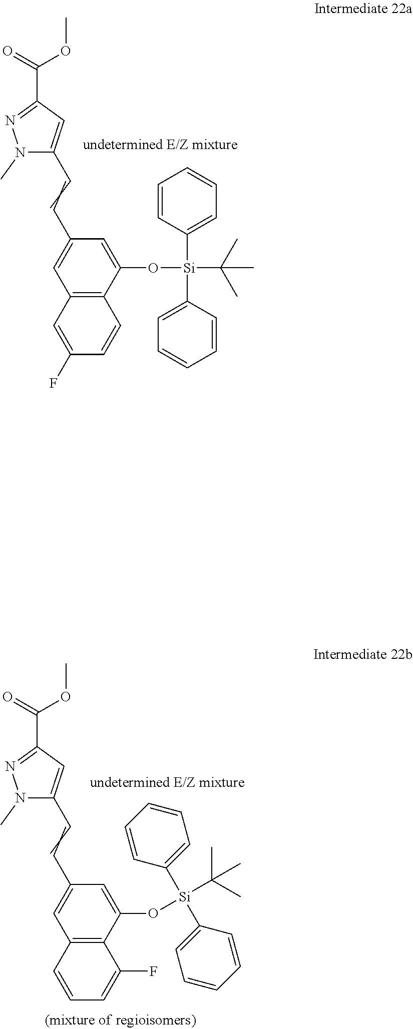

NaH (60% in mineral oil, 354 mg, 1.1 eq.) was added to a suspension of intermediate 105 (4.386 g, 1.1 eq.) in THF (50 mL) at 0° C. The resulting solution was stirred at this temperature for 45 min before being cooled to −25° C. A solution of the mixture of Intermediate 21a and Intermediate 21b (4.5 g, 8.4 mmol) in THF (9 mL) was added slowly to the solution while maintaining the temperature between −20° C. and −30° C. Once the addition was complete, the reaction was stirred at −10° C. for 1.5 h. The reaction was quenched by slow addition of saturated aqueous NH₄Cl (10 mL) at −10° C. The mixture was diluted with EtOAc (50 mL). The layers were separated and the aqueous layer was extracted with EtOAc (2×50 mL). The combined organic layer was dried over MgSO₄, filtered, and concentrated under reduced pressure. The crude product was purified by column chromatography on silica gel (heptane:EtOAc—1:0 to 7:3) to afford the mixture of Intermediate 22a and Intermediate 22b (3.98 g, yield: 79%) as a white foam.

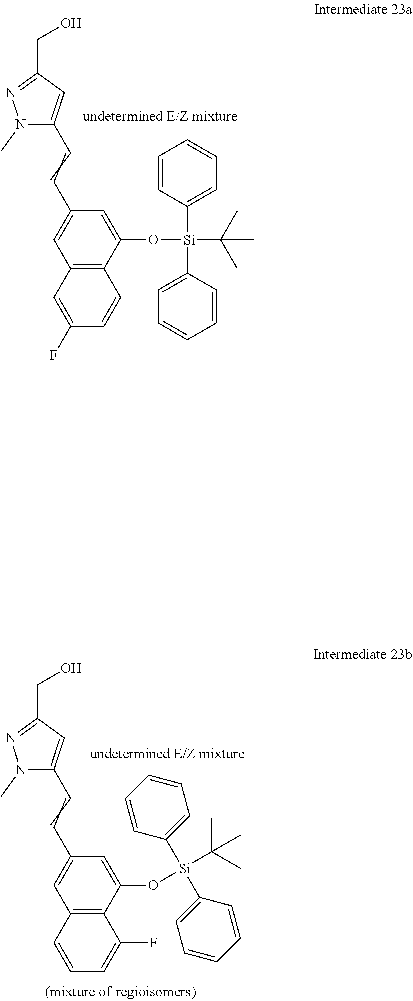

LiAlH₄ (2 M in THF, 4.1 mL, 1.25 eq.) was slowly added to the mixture of Intermediate 22a and Intermediate 22b (3.9 g, 6.561 mmol) in THF (50 mL), cooled to 0° C. Once the addition was complete, the reaction mixture was stirred at 0° C. for 30 min. The reaction was quenched by slow addition of EtOAc (10 mL) followed by a saturated aqueous Rochelle salt solution (50 mL). The aqueous layer was extracted with EtOAc (2×45 mL). The combined organic extract was washed with brine (20 mL), dried over MgSO₄, filtered, and concentrated to afford the mixture of Intermediate 23a and Intermediate 23b (3.51 g, yield: 94%) as a pale yellow foam, used without further purification.

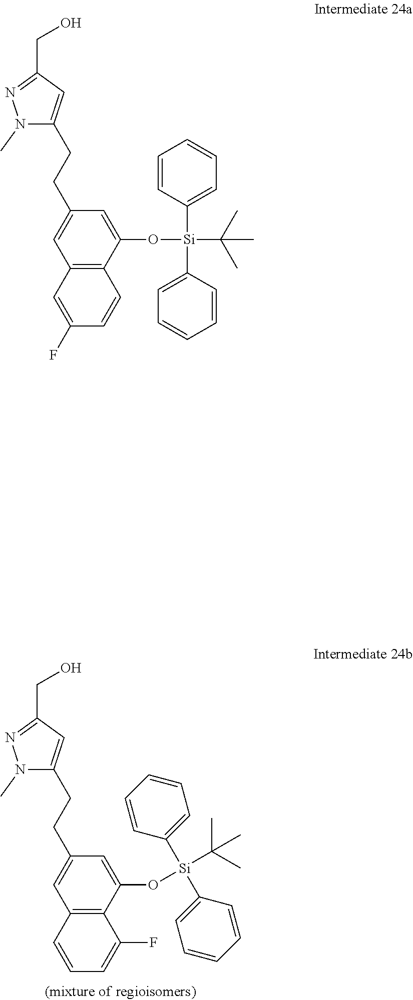

The mixture of Intermediate 23a and Intermediate 23b (3.5 g, 6.195 mmol) was dissolved in MeOH (160 mL). Pd/C (10%, 659 mg, 0.1 eq.) was added under nitrogen atmosphere. The reaction mixture was then flushed with hydrogen gas and vacuum (3 times). The reaction mixture was then stirred at room temperature under an hydrogen atmosphere (1 atm) until 1 equivalent of hydrogen was taken up. The reaction mixture was filtered over a pad of Dicalite® and concentrated to give the mixture of Intermediate 24a and Intermediate 24b (3.16 g, yield: 90%) as an off-white solid, used without further purification.

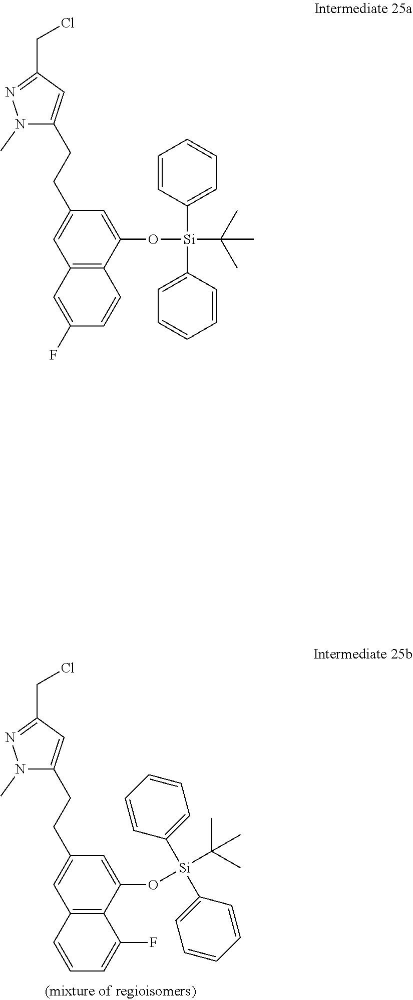

SOCl₂ (0.274 mL, 1.5 eq.) was added to the mixture of Intermediate 24a and Intermediate 24b (1.85 g, 3.262 mmol) in DCM (20 mL), cooled to 0° C. Once the addition was complete, the reaction mixture was allowed to warm to room temperature and was stirred for 1 h. The reaction mixture was diluted with DCM (35 mL), washed with saturated aqueous NaHCO₃ (2×50 mL) and brine (50 mL). The organic layer was dried over MgSO₄, filtered, and concentrated to give an orange oil. This oil was purified by flash column chromatography on silica gel (heptane:EtOAc—1:0 to 8:2) to give the mixture of Intermediate 25a and Intermediate 25b (1.75 g, yield: 91%) as a colorless oil which crystallized on standing to a white solid.

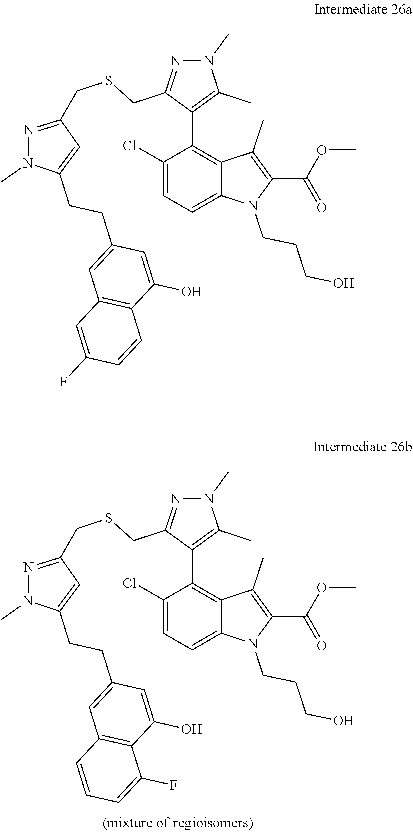

Intermediate 8 (600 mg, 0.986 mmol) and the mixture of Intermediate 25a and Intermediate 25b (659 mg, 1.2 eq.) were dissolved in MeOH (12 mL). The reaction mixture was degassed and re-filled with nitrogen three times. The reaction mixture was then cooled to 0° C. before addition of K₂CO₃ (272 mg, 2 eq.). After that addition, the reaction mixture was again degassed and re-filled with nitrogen twice. The reaction mixture was allowed to warm to room temperature and was stirred for 60 h. The reaction mixture was concentrated and the residue was partitioned between water (10 mL) and EtOAc (15 mL). The layers were separated and the aqueous layer was extracted with EtOAc (2×20 mL). The combined organic layer was washed with brine (30 mL), dried over MgSO₄, filtered, and concentrated.

The crude foam was dissolved in MeOH (10 mL) and pTsOH (469 mg, 3 eq.) was added. The resulting reaction mixture was stirred at room temperature for 30 min. The reaction mixture was concentrated. The residue was dissolved in EtOAc (20 mL) and washed with saturated aqueous NaHCO₃ (15 mL). The aqueous layer was extracted with EtOAc (2×20 mL). The combined organic layer was washed with brine (30 mL), dried over MgSO₄, filtered, and concentrated. The crude product was purified by flash column chromatography on silica gel (heptane:EtOAc—6:4 to 0:1) to give the mixture of Intermediate 26a and Intermediate 26b (620 mg, yield: 85%) as a white foam.

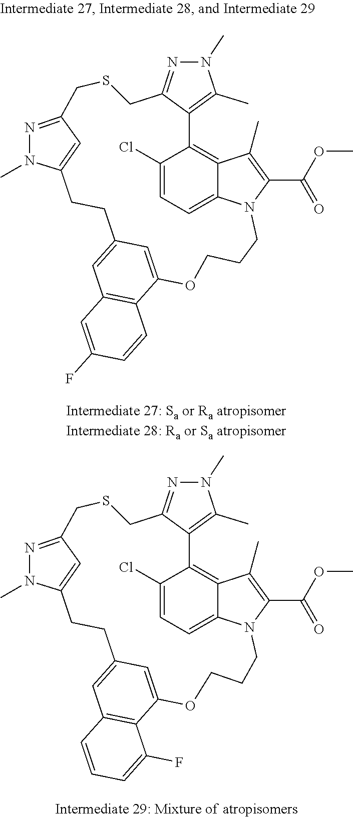

A solution of the mixture of Intermediate 26a and Intermediate 26b (600 mg, 0.809 mmol) and DTBAD (745 mg, 4 eq.) in a mixture of toluene (23 mL) and THE (4.8 mL) was added dropwise with a syringe pump (0.1 mL/min) to a solution of PPh₃ (849 mg, 4 eq.) in toluene (23 mL) at 70° C. After completion of the addition, the reaction mixture was concentrated and the crude product was purified by flash column chromatography on silica gel (heptane:EtOAc—6:4 to 0:1) to give the mixture of Intermediate 27, Intermediate 28, and Intermediate 29 (450 mg, yield: 81%) as a white foam. 225 mg of the isolated product were purified by preparative SFC (Stationary phase: Chiralpak Daicel ID 20×250 mm, Mobile phase: CO₂, EtOH+0.4% iPrNH₂) to give Intermediate 27 (71 mg, yield: 12%) and a mixture of Intermediate 28 and Intermediate 29. This mixture was purified again by preparative SFC (Stationary phase: Chiralpak Daicel AS 20×250 mm, Mobile phase: CO₂, EtOH+0.4% iPrNH₂), to afford Intermediate 28 (61 mg, yield: 10%) and Intermediate 29 (29 mg, yield: 4%) as pale yellow oils that crystallized on standing.

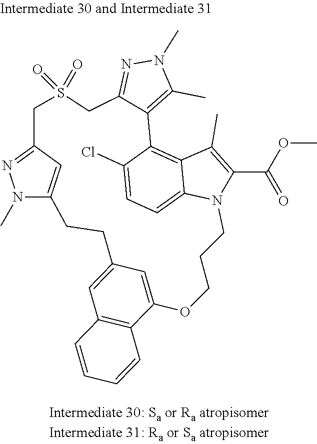

mCPBA (124 mg, 2.1 eq.) was added to the racemic mixture of Intermediate 17 and Intermediate 18 (180 mg, 0.256 mmol) in DCM (10 mL), cooled in an ice bath. After 15 min at 0° C., the reaction mixture was allowed to warm to room temperature and was stirred overnight. The reaction mixture was concentrated. The crude product was purified by flash column chromatography (heptane:EtOAc—3:1 to 2:8) to give the racemic mixture of Intermediate 30 and Intermediate 31 (160 mg, yield: 80%) as a yellow solid. The atropisomers were then separated by preparative SFC (Stationary phase: Chiralpak Diacel AS 20×250 mm, Mobile phase: CO₂, EtOH+0.4% iPrNH₂), affording Intermediate 30 (34 mg, yield: 18%) and Intermediate 31 (27 mg, yield: 14%) as white solids.

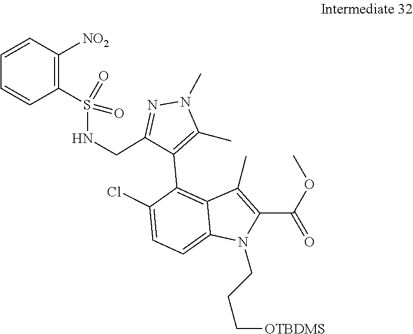 [SMILES: COC(=O)c1c(C)c2c(-c3c(CNS(=O)(=O)c4ccccc4[N+](=O)[O-])nn(C)c3C)c(Cl)ccc2n1CCCO[Si](C)(C)C(C)(C)C]

A solution of di-tert-butyl azodicarboxylate (78 mg, 2 eq.) in DCM (1 mL) was added dropwise to a suspension of Intermediate 5 (88 mg, 0.17 mmol), 2-nitrobenzenesulfonamide (38 mg, 1.1 eq.), and triphenylphospine (89 mg, 2 eq.) in DCM (2.5 mL) stirring at room temperature under nitrogen atmosphere. After 15 min, the reaction mixture was directly loaded onto a silica gel column (12 g) and the product was purified eluting with a gradient from heptane 100% up to heptane/EtOAc 1/1. Intermediate 32 (120 mg, quantitative) was obtained as a yellow solid.

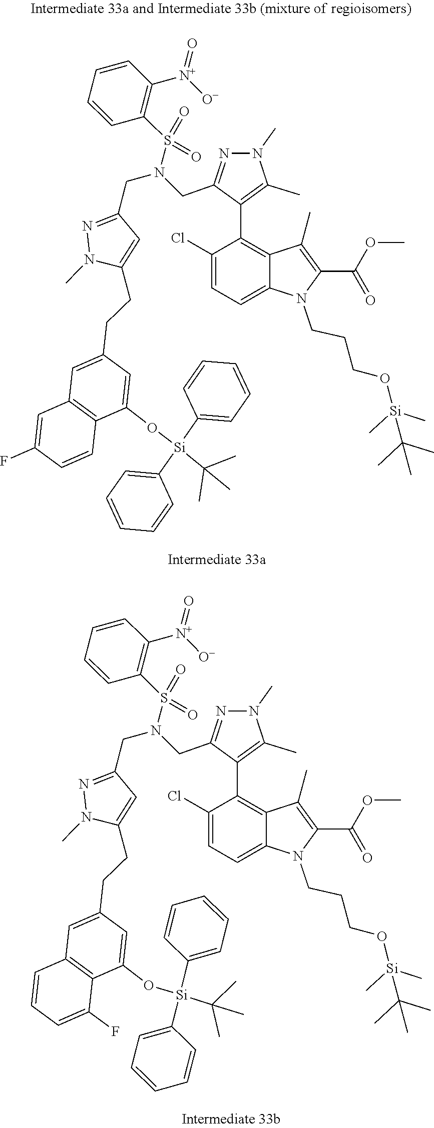 [SMILES: COC(=O)c1c(C)c2c(-c3c(CN(Cc4cc(CCc5cc(O[Si](c6ccccc6)(c6ccccc6)C(C)(C)C)c6ccc(F)cc6c5)n(C)n4)S(=O)(=O)c4ccccc4[N+](=O)[O-])nn(C)c3C)c(Cl)ccc2n1CCCO[Si](C)(C)C(C)(C)C ; COC(=O)c1c(C)c2c(-c3c(CN(Cc4cc(CCc5cc(O[Si](c6ccccc6)(c6ccccc6)C(C)(C)C)c6c(F)cccc6c5)n(C)n4)S(=O)(=O)c4ccccc4[N+](=O)[O-])nn(C)c3C)c(Cl)ccc2n1CCCO[Si](C)(C)C(C)(C)C]

To a suspension of Intermediate 32 (1450 mg, 1.133 mmol), the mixture of Intermediate 24a and Intermediate 24b (642 mg, 1 eq.) and PPh₃ (594, 2 eq.) in DCM (17 mL) was added a solution of DTBAD (521 mg, 2 eq.) in DCM (5 mL). The resulting reaction mixture was stirred at room temperature for 16 h. The reaction mixture was concentrated and the crude product was purified by flash column chromatography on silica gel (heptane:EtOAc—1:0 to 1:1) to give the mixture of Intermediate 33a and Intermediate 33b (1.21 g, yield: 61%) as a yellow foam.

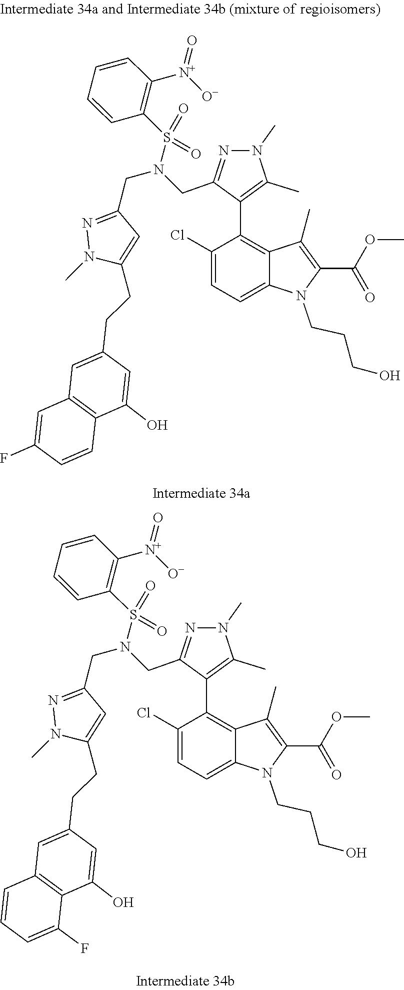 [SMILES: COC(=O)c1c(C)c2c(-c3c(CN(Cc4cc(CCc5cc(O)c6ccc(F)cc6c5)n(C)n4)S(=O)(=O)c4ccccc4[N+](=O)[O-])nn(C)c3C)c(Cl)ccc2n1CCCO ; COC(=O)c1c(C)c2c(-c3c(CN(Cc4cc(CCc5cc(O)c6c(F)cccc6c5)n(C)n4)S(=O)(=O)c4ccccc4[N+](=O)[O-])nn(C)c3C)c(Cl)ccc2n1CCCO]

The mixture of Intermediate 33a and Intermediate 33b (2156 mg, 1.232 mmol) was dissolved in MeOH (15 mL) and pTsOH (782 mg, 6 eq.) was added. The resulting reaction mixture was stirred at room temperature for 30 min. The reaction mixture was concentrated to give a yellow oil. The oil was dissolved in EtOAc (20 mL) and was washed with saturated aqueous NaHCO₃ (15 mL). The aqueous layer was extracted with EtOAc (2×20 mL). The combined organic layer was washed with brine (30 mL), dried over MgSO₄, filtered, and concentrated to give a yellow oil. This yellow oil was dissolved in MeOH (15 mL) and K₂CO₃ (284 mg, 3 eq.) was added. The reaction mixture was stirred at room temperature for 14 h. The reaction mixture was concentrated and the residue was partitioned between DCM (20 mL) and saturated aqueous NH₄Cl (20 mL). The aqueous layer was extracted with DCM (20 mL), the combined organic layer was dried over MgSO₄, filtered, and evaporated. The crude product was purified by flash column chromatography on silica gel (heptane:EtOAc—6:4 to 0:1) to give the mixture of Intermediate 34a and Intermediate 34b (630 mg, yield: 73%) as a yellow foam.

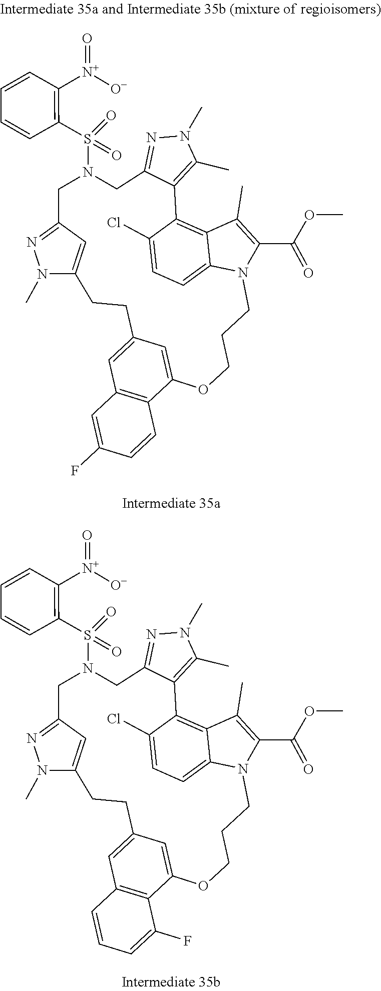 [SMILES: COC(=O)c1c(C)c2c3c(Cl)ccc2n1CCCOc1cc(cc2cc(F)ccc12)CCc1cc(nn1C)CN(S(=O)(=O)c1ccccc1[N+](=O)[O-])Cc1nn(C)c(C)c1-3 ; COC(=O)c1c(C)c2c3c(Cl)ccc2n1CCCOc1cc(cc2cccc(F)c12)CCc1cc(nn1C)CN(S(=O)(=O)c1ccccc1[N+](=O)[O-])Cc1nn(C)c(C)c1-3]

A solution of the mixture of Intermediate 34a and Intermediate 34b (625 mg, 0.502 mmol) and DTBAD (462 mg, 4 eq.) in a mixture of toluene (15 mL) and THF (3 mL) was added dropwise with a syringe pump (0.1 mL/min) to a solution of PPh₃ (526 mg, 4 eq.) in toluene (15 mL) at 70° C. The reaction mixture was concentrated. The residue was purified by flash column chromatography on silica gel (heptane:EtOAc—6:4 to 2:8) to yield a mixture of Intermediate 35a and Intermediate 35b (507 mg, yield: 83%) as a white foam.

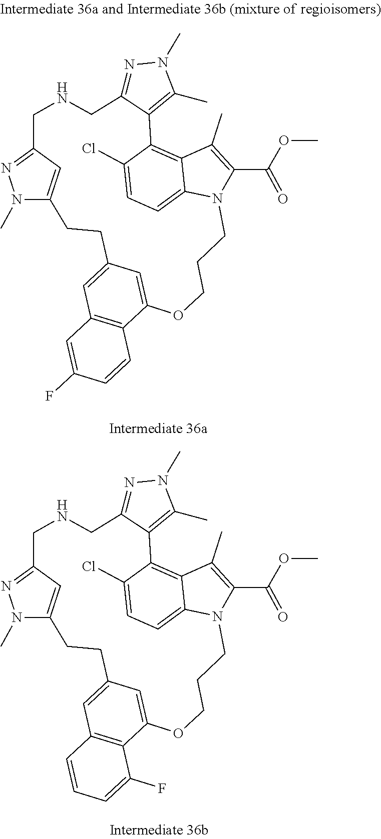

To a suspension of a mixture of Intermediate 35a and Intermediate 35b (500 mg, 0.41 mmol) and K₂CO₃ (566 mg, 10 eq.) in anhydrous ACN (10 mL) was added dropwise thiophenol (0.421 mL, 10 eq.). The reaction mixture was stirred overnight at room temperature. The reaction mixture was filtered over a pad of Dicalite® and the filtrate was evaporated. The crude product was purified by column chromatography on silica gel (DCM:MeOH—1:0 to 9:1) to give a mixture of Intermediate 36a and Intermediate 36b (185 mg, yield: 64%) as a white foam.

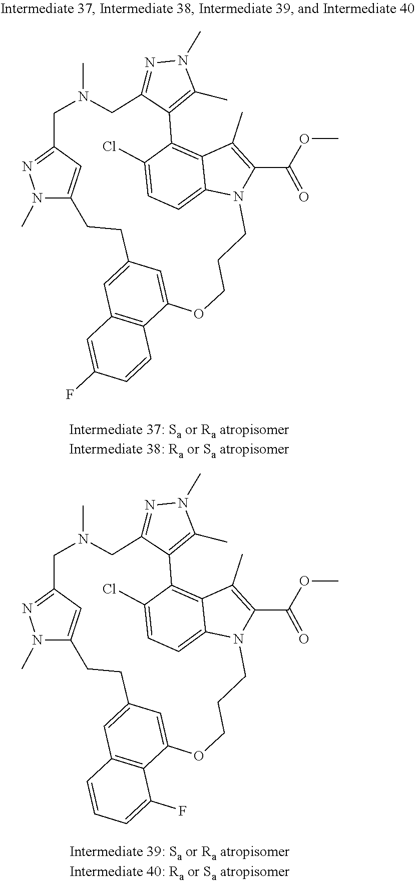

Formaldehyde (37% aqueous solution, 57 μL, 3 eq.) was added to a solution of a mixture of Intermediate 36a and Intermediate 36b (180 mg, 0.256 mmol) and AcOH (44 μL, 3 eq.) in DCM (3 mL) at room temperature. Then, NaBH(OAc)₃ (162 mg, 3 eq.) was added and the reaction mixture was stirred at room temperature for 1 h. The reaction was quenched by addition of saturated aqueous NaHCO₃ (2.5 mL) and was diluted with water (2.5 mL) and DCM (10 mL). The organic layer was separated and the aqueous layer was extracted with DCM (2×10 mL). The combined organic layer was dried over MgSO₄, filtered, and evaporated. The residue was purified by preparative SFC (Stationary phase: Chiralpak Daicel ID 20×250 mm, Mobile phase: CO₂, iPrOH+0.4% iPrNH₂) to give a mixture of Intermediate 37 and Intermediate 38 and a mixture of Intermediate 39 and Intermediate 40. The first mixture was purified by preparative SFC (Stationary phase: Chiralcel Diacel OD 20×250 mm, Mobile phase: CO₂, EtOH+0.4% iPrNH₂) to give Intermediate 37 (40 mg, yield: 22%) and Intermediate 38 (41 mg, yield: 22%). The second mixture was purified by preparative SFC (Stationary phase: Chiralcel Diacel OD 20×250 mm, Mobile phase: CO₂, EtOH+0.4% iPrNH₂) to give Intermediate 39 (14 mg, yield: 7%) and Intermediate 40 (13 mg, yield: 7%).

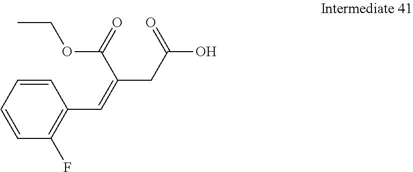 [SMILES: CCOC(=O)/C(=C\c1ccccc1F)CC(=O)O]

Sodium ethoxide (12.918 g, 2 eq.) was slowly added to anhydrous EtOH (175 mL) at room temperature, under nitrogen atmosphere. Once the addition was complete, the reaction mixture was warmed to 50° C. and was stirred for 1 h. A solution of 2-fluorobenzaldehyde (10 mL, 94.912 mmol) and diethyl succinate (16.581 mL, 1.05 eq.) dissolved in EtOH (30 mL) were then added dropwise at 50° C. via syringe pump (0.5 mL/min). Once the addition was complete, the reaction mixture was refluxed for 3 h. The reaction mixture was concentrated under reduced pressure and the residue was partitioned between 1 M aqueous HCl (150 mL) and EtOAc (200 mL). The layers were separated and the aqueous layer was extracted with EtOAc (2×200 mL). The combined organic layer was washed with brine, dried over MgSO₄, filtered, and concentrated to afford Intermediate 41 (26.5 g, yield: 50%) as an orange oil, used without further purification.

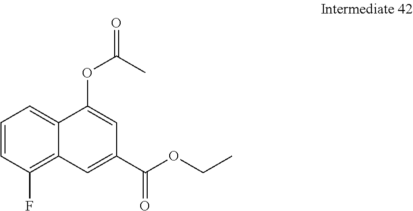 [SMILES: CCOC(=O)c1cc(OC(C)=O)c2cccc(F)c2c1]

Sodium acetate (8.456 g, 1 eq.) was added to Intermediate 41 (26 g, 103.07 mmol) in acetic anhydride (80 mL). The resulting solution was refluxed for 1.5 h. After cooling, the reaction mixture was concentrated under reduced pressure. The residue was partitioned between EtOAc and water (200 mL each). The layers were separated and the aqueous layer was extracted with EtOAc (3×350 mL). The combined organic layer was carefully quenched with saturated aqueous NaHCO₃ and then solid NaHCO₃ until the pH reached 8. The organic layer was washed one more time with saturated aqueous NaHCO₃ (400 mL) and then with brine (400 mL). The organic layer was dried on MgSO₄, filtered, and evaporated. The crude product was purified by flash column chromatography on silica gel (heptane:EtOAc—1:0 to 8:2) to give Intermediate 42 (4.45 g, yield: 12%) as a yellow solid.

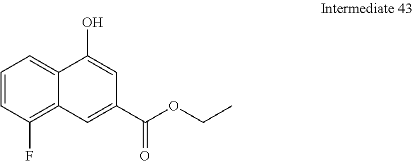 [SMILES: CCOC(=O)c1cc(O)c2cccc(F)c2c1]

K₂CO₃ (2.852 g, 2 eq.) was added to Intermediate 42 (3800 mg, 10.316 mmol) in a mixture of EtOH (40 mL), MeOH (5 mL) and THE (10 mL) and the reaction mixture was stirred for 16 h at room temperature. The reaction mixture was filtered to remove the residual potassium carbonate and concentrated under reduced pressure. The dark brown oil was dissolved in EtOAc (70 mL) and washed with saturated aqueous NH₄Cl (50 mL). The aqueous layer was extracted with EtOAc (2×60 mL). The combined organic layer was washed with brine (100 mL), dried over MgSO₄, filtered, and concentrated under reduced pressure. The crude product was purified by column chromatography on silica gel (heptane:EtOAc—1:0 to 7:3) to give Intermediate 43 (2.42 g, yield: 90%) as an orange solid.

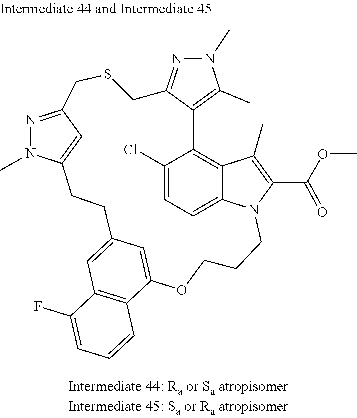

Intermediate 44 and Intermediate 45 were prepared following the same synthetic pathway as for Intermediate 27 and Intermediate 28, respectively, starting initially from Intermediate 43 instead of the mixture of ethyl 7-fluoro-4-hydroxy-2-naphthoate and ethyl 5-fluoro-4-hydroxy-2-naphthoate.

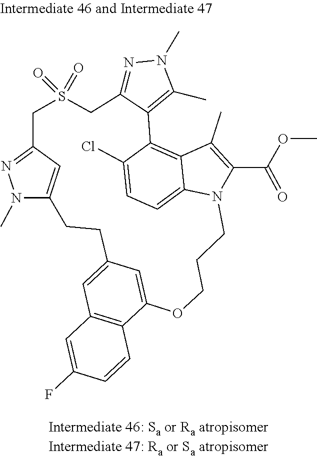

Intermediate 46 and Intermediate 47 were prepared using an analogous procedure as for Intermediate 30 and Intermediate 31, starting from the pure atropisomers Intermediate 27 and Intermediate 28, respectively, instead of the racemic mixture of Intermediate 17 and Intermediate 18.

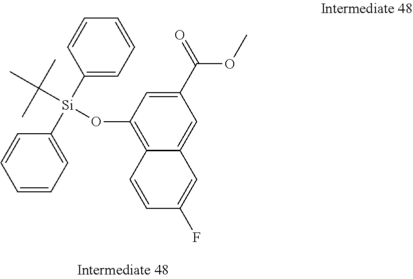 [SMILES: COC(=O)c1cc(O[Si](c2ccccc2)(c2ccccc2)C(C)(C)C)c2ccc(F)cc2c1]

TBDPSCl (14.66 g, 1.5 eq.) was added to a solution of methyl 7-fluoro-4-hydroxy-2-naphthoate (CAS [2092726-85-5]) (8 g, 35.555 mmol) and imidazole (7.26, 3 eq.) in DCM (500 mL), cooled to 0° C. under nitrogen atmosphere. Once the addition was complete, the reaction was stirred at room temperature overnight. The reaction was quenched by addition of water (100 mL). The mixture was extracted with EtOAc (3×200 mL). The combined organic layer was dried over Na₂SO₄, filtered, and concentrated to afford a yellow oil. This oil was purified by flash column chromatography on silica gel (petroleum ether:EtOAc—1:0 to 1:1) to afford Intermediate 48 (14 g, yield: 86%) as a yellow oil.

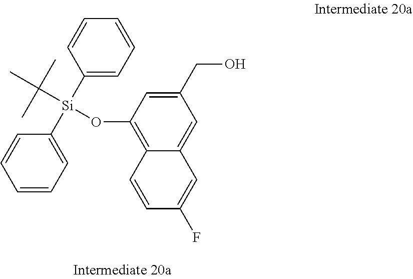 [SMILES: CC(C)(C)[Si](Oc1cc(CO)cc2cc(F)ccc12)(c1ccccc1)c1ccccc1]

LiAlH₄ (1.39 g, 1.2 eq.) was added slowly to a solution of Intermediate 48 (14 g, 30.528 mmol) in THF (200 mL), cooled to 0° C. under nitrogen atmosphere. Once the addition was complete the reaction mixture was stirred at 0° C. for 2 h. The reaction was quenched by slow addition of water (2 mL) followed by a 10% aqueous NaOH solution (2 mL) at 0° C. The heterogeneous mixture was filtered, and the filter cake was washed with DCM (200 mL). The filtrate was evaporated and the residue was purified by flash column chromatography on silica gel (petroleum ether:EtOAc—1:0 to 1:1) to give Intermediate 20a (12 g, yield: 90%) as a yellow solid.

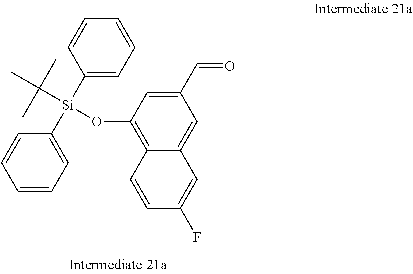 [SMILES: CC(C)(C)[Si](Oc1cc(C=O)cc2cc(F)ccc12)(c1ccccc1)c1ccccc1]

MnO₂ (29.074 g, 12 eq.) was added to a solution of Intermediate 20a (12 g, 27.869 mmol) in DCM (200 mL) at room temperature. The resulting solution was stirred at room temperature overnight. The reaction mixture was filtered and the filtrate was concentrated. The residue was purified by flash column chromatography over silica gel (eluent: petroleum ether/EtOAc, 100/0 to 50/50) to afford Intermediate 21a (12 g, yield: 99%) as a yellow oil.

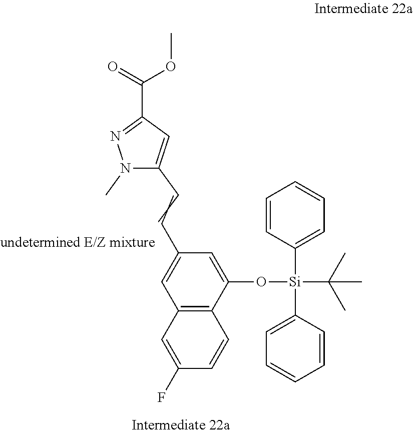 [SMILES: COC(=O)c1cc(C=Cc2cc(O[Si](c3ccccc3)(c3ccccc3)C(C)(C)C)c3ccc(F)cc3c2)n(C)n1]

NaH (60% in mineral oil, 1.448 g, 1.3 eq.) was added to a suspension of intermediate 105 (13.812 g, 1.1 eq.) in THF (200 mL) at 0° C. The resulting solution was stirred at this temperature for 1 h before being cooled to −30° C. Intermediate 21a (12 g, 27.847 mmol) was added slowly to the solution while maintaining the temperature between −20° C. and −30° C. Once the addition was complete, the reaction was stirred at −30° C. for 2 h. The reaction was quenched by slow addition of water (100 mL). The mixture was extracted with DCM (3×300 mL). The combined organic layer was dried over Na₂SO₄, filtered, and concentrated under reduced pressure. The crude product was purified by column chromatography on silica gel (petroleum ether:EtOAc—1:0 to 1:1) to afford Intermediate 22a (13 g, yield: 82%) as a white solid.

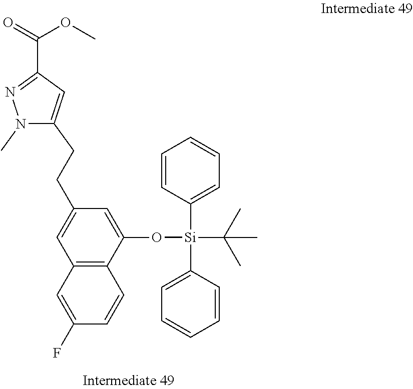 [SMILES: COC(=O)c1cc(CCc2cc(O[Si](c3ccccc3)(c3ccccc3)C(C)(C)C)c3ccc(F)cc3c2)n(C)n1]

A solution of Intermediate 22a (13 g, 23.02 mmol) in MeOH (75 mL) and THE (75 mL) was hydrogenated at 25° C. (15 psi H₂) in the presence of Pd/C (2 g; 10%). The reaction mixture was stirred for 16 h. After uptake of H₂ (1 eq.), the catalyst was filtered off and the filtrate was evaporated to afford Intermediate 49 (13 g, yield: 100%) as a colorless oil.

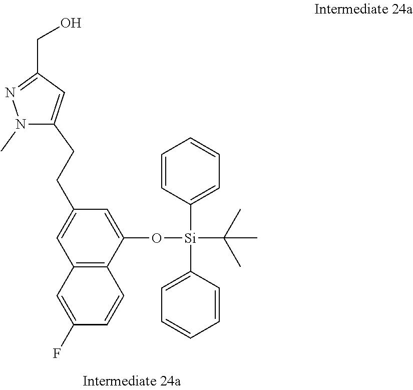 [SMILES: Cn1nc(CO)cc1CCc1cc(O[Si](c2ccccc2)(c2ccccc2)C(C)(C)C)c2ccc(F)cc2c1]

LiAlH₄ (1.045 g, 1.2 eq.) was added portionwise to a solution of Intermediate 49 (13 g, 22.94 mmol) in THF (200 mL) at 0° C., under nitrogen atmosphere. The reaction mixture was stirred at 0° C. for 2 h. Water (1 mL) was then added dropwise, followed by a 10% aqueous NaOH solution (1 mL), at 0° C. The reaction mixture was filtered, the filter cake was washed with DCM (200 mL), and the filtrate was evaporated. The crude product was purified by flash column chromatography over silica gel (eluent: petroleum ether/EtOAc, 100/0 to 0/100) to afford Intermediate 24a (10.4 g, yield: 84%) as a white solid.

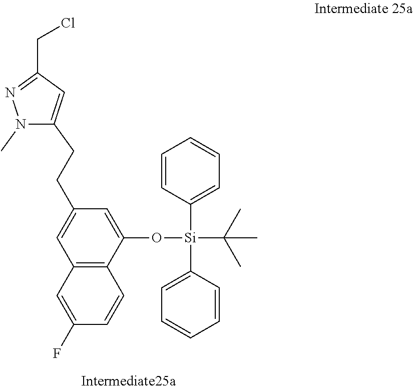 [SMILES: Cn1nc(CCl)cc1CCc1cc(O[Si](c2ccccc2)(c2ccccc2)C(C)(C)C)c2ccc(F)cc2c1]

SOCl₂ (0.78 mL, 1.15 eq.) was added dropwise to a solution of Intermediate 24a (5 g, 9.28 mmol) in anhydrous DCM (57 mL) under nitrogen atmosphere at 0° C. Once the addition was complete, the reaction mixture was allowed to warm to room temperature and was stirred for 1.5 h. The reaction was diluted with DCM, washed with a saturated aqueous NaHCO₃ solution (×2) and brine. The combined aqueous extracts were extracted with DCM (×3). The combined organic extract was dried over MgSO₄, filtered, and concentrated under reduced pressure to give a pale yellow solid. This solid was purified by flash column chromatography (SiO₂, 40 g RediSep, heptane/EtOAc, 100/0 to 0/100) to afford intermediate 25a (4.55 g, yield: 87%) as a white solid.

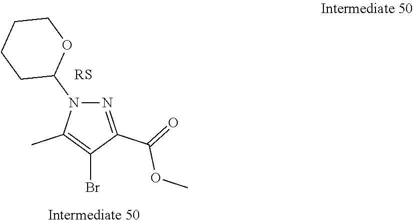 [SMILES: COC(=O)c1nn(C2CCCCO2)c(C)c1Br]

pTsOH (5.4 g, 0.1 eq.) was added to 1H-pyrazole-3-carboxylic acid, 4-bromo-5-methyl-, methyl ester (CAS [1232838-31-1]) (76 g, 315 mmol) and 3,4-dihydro-2H-pyran (53 g, 2 eq.) in DCM (600 mL). The reaction mixture was stirred at room temperature for 2 h. The reaction was quenched by addition of water (300 mL) and the mixture was extracted with DCM (500 mL×2). The combined organic layer was washed with brine (200 mL), dried with Na₂SO₄, and filtered. The filtrate was evaporated and the residue was purified by column chromatography over silica gel (eluent: petroleum ether/EtOAc 100:0 to 80:20) to give Intermediate 50 (130 g crude, 78% pure, quantitative) as a yellow solid.

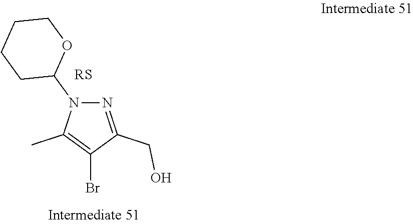

LiAlH₄ (14.4 g, 2 eq.) was added portionwise to THE (1 L) at 0° C. The mixture was stirred at 0° C. for 5 min. Then, Intermediate 50 (64 g, 190 mmol) was added portionwise. The reaction mixture was stirred at 0° C. for 1 h. Water (14 mL) was added dropwise, followed by aqueous NaOH (2 M, 14 mL), and finally MgSO₄ (10 g). The mixture was filtered over a pad of Celite and the filter cake was washed with DCM (1 L×2). The combined organic layer was evaporated to give a yellow oil. This oil was purified by flash column chromatography over silica gel (petroleum ether/EtOAc from 100/0 to 40/60) to give Intermediate 51 (two fractions: 20 g (98% pure, yield: 37%) and 26 g (68% pure, yield: 47%)) as a white solid.

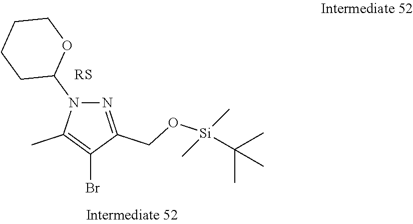 [SMILES: Cc1c(Br)c(CO[Si](C)(C)C(C)(C)C)nn1C1CCCCO1]

DMAP (814 mg, 0.4 eq.) and Et₃N (4.6 mL, 2 eq.) were added to a solution of Intermediate 51 (5 g, 16.66 mmol) in THF (50 mL) at room temperature. TBDMSCl (3.77 g, 1.5 eq.) was added and the reaction mixture was stirred at room temperature for 4 h. The reaction was quenched by addition of saturated aqueous NaHCO₃ (50 mL) and the mixture was extracted with EtOAc (50 mL×2). The combined organic layer was washed with brine (50 mL), dried with Na₂SO₄, filtered, and evaporated. The residue was purified by column chromatography over silica gel (eluent: petroleum ether/EtOAc 100:0 to 20:80) to give Intermediate 52 (6.31 g, yield: 96%) as a colorless oil.

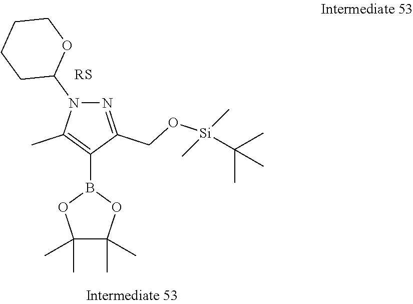 [SMILES: Cc1c(B2OC(C)(C)C(C)(C)O2)c(CO[Si](C)(C)C(C)(C)C)nn1C1CCCCO1]

nBuLi (59 mL, 1.2 eq.) was slowly added to a solution of Intermediate 52 (48 g, 123 mmol) in THF (1 L) at −78° C. under nitrogen atmosphere. The reaction mixture was stirred at −78° C. for 1 h. 2-Isopropoxy-4,4,5,5-tetramethyl-1,3,2-dioxaborolane [61676-62-8] (34.4 g, 1.5 eq.) was slowly added to the reaction mixture. The reaction mixture was stirred at room temperature for 2 h. The reaction mixture was slowly added to saturated aqueous NH₄Cl (200 mL). The resulting mixture was extracted with EtOAc (500 mL×2), and the combined organic layer was washed with brine (100 mL), dried on Na₂SO₄, filtered, and evaporated. The residue was purified by flash column chromatography over silica gel (eluent: petroleum ether/ethyl acetate from 100/0 to 90/10) to give Intermediate 53 (50 g, yield: 69%) as a yellow oil.

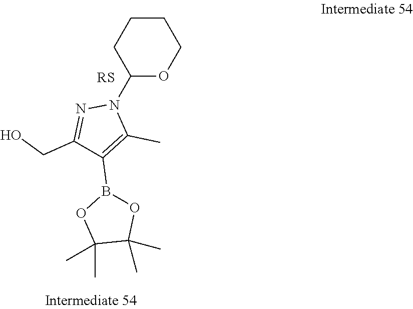 [SMILES: Cc1c(B2OC(C)(C)C(C)(C)O2)c(CO)nn1C1CCCCO1]

TBAF (1 M in THF, 54.99 mL, 1.2 eq.) was added to an ice-cooled solution of Intermediate 53 (20 g, 46 mmol) in anhydrous 2-Me-THF (287 mL) under nitrogen atmosphere. The ice bath was removed and the resulting reaction mixture was stirred at room temperature for 19 h. The reaction mixture was diluted with EtOAc and the layers were separated. The organic layer was washed with an aqueous saturated solution of NaHCO₃ and brine. The combined aqueous layer was extracted with EtOAc (×3) and the combined organic extract was dried over MgSO₄, filtered, and concentrated under reduced pressure. The residue was purified by flash column chromatography (SiO₂, 120 g RediSep column, heptane/EtOAc, gradient 100/0 to 0/100) to afford Intermediate 54 (12 g, yield: 80%) as a colorless oil that solidified to a white solid upon standing.

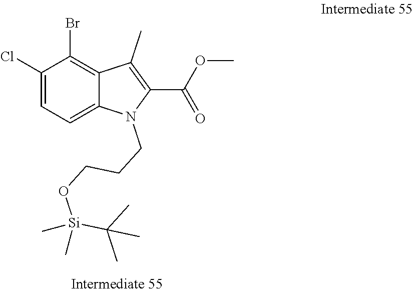 [SMILES: COC(=O)c1c(C)c2c(Br)c(Cl)ccc2n1CCCO[Si](C)(C)C(C)(C)C]

A mixture of Intermediate 2 (37.4 g, 123.6 mmol), (3-bromopropoxy)-tert-butyldimethylsilane (CAS [89031-84-5]) (37.567 g, 1.2 eq.), and K₂CO₃ (51.25 g, 3 eq.) in ACN (300 mL) was stirred at 80° C. overnight. The reaction mixture was cooled to room temperature and was filtered. The filter cake was washed with EtOAc (100 mL). The filtrate was concentrated and the residue was purified by column chromatography over silica gel (eluent: petroleum ether/EtOAc from 100/0 to 10/90) to afford Intermediate 55 (42 g, 71%) as a red gum.

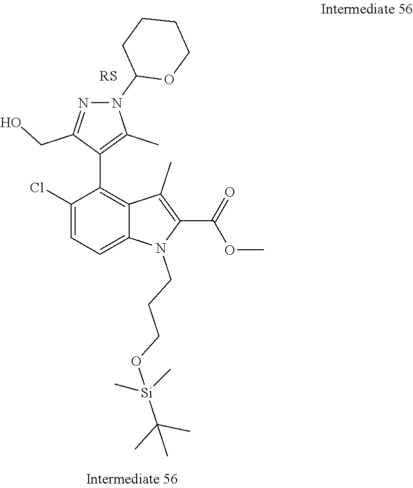 [SMILES: COC(=O)c1c(C)c2c(-c3c(CO)nn(C4CCCCO4)c3C)c(Cl)ccc2n1CCCO[Si](C)(C)C(C)(C)C]

A pressure tube was charged with Intermediate 55 (5 g, 10.17 mmol), Intermediate 54 (4.18 g, 1.19 eq.), Pd(amphos)₂Cl₂ (CAS [887919-35-9]) (364 mg, 0.05 eq.), and K₂CO₃ (2.84 g, 2 eq.) under nitrogen atmosphere. A mixture of 1,4-dioxane (49 mL) and water (12.5 mL), previously nitrogen-purged for 35 min, was added under nitrogen atmosphere to the reaction tube. The tube was sealed and the reaction mixture was heated for 3.5 h at 70° C. After cooling to room temperature, the reaction mixture was diluted with water and EtOAc. The layers were separated and the aqueous layer was extracted with EtOAc (×3). The combined organic layer was dried over MgSO₄, filtered, and concentrated. The residue was purified by flash column chromatography (SiO₂, 120 g RediSep column, heptane/EtOAc, gradient 100/0 to 0/100) to give Intermediate 56 (4.74 g, yield: 76%) as a pale yellow foam.

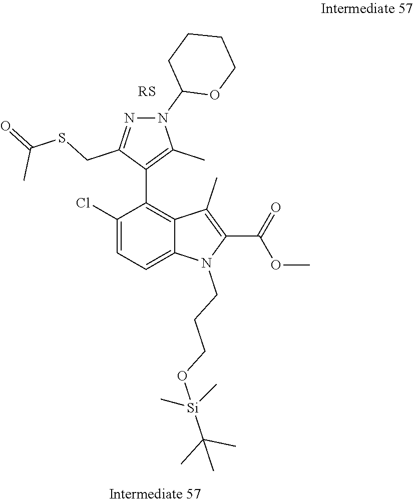 [SMILES: COC(=O)c1c(C)c2c(-c3c(CSC(C)=O)nn(C4CCCCO4)c3C)c(Cl)ccc2n1CCCO[Si](C)(C)C(C)(C)C]

Et₃N (1.21 mL, 1.5 eq.), followed by MsCl (0.56 mL, 1.25 eq.) were added dropwise to a solution of Intermediate 56 (3.57 g, 5.81 mmol) in anhydrous THE (71 mL, degassed 15 by bubbling nitrogen for 15 min) under nitrogen atmosphere at 0° C. The reaction mixture was stirred at 0° C. for 5 min, and then at room temperature for 1 h. The reaction mixture containing the intermediate mesylate was degassed by bubbling nitrogen for 10 min. Then, a nitrogen purged solution of KSAc (6.63 g, 10 eq.) in anhydrous DMF (112 mL, nitrogen-purged for 30 min) was added in one portion to the reaction mixture at room temperature. The resulting mixture was nitrogen-purged for 5 min and then stirred at room temperature for 30 min. The reaction mixture was diluted with EtOAc and water. The aqueous layer was separated and extracted with EtOAc (×3). The combined organic layer was washed with brine (×3), dried over MgSO₄, filtered, and evaporated. The residue was purified by flash column chromatography (SiO₂, 220 g RediSep column, heptane/EtOAc, gradient 100/0 to 0/100) to afford Intermediate 57 (3.9 g, yield: 93%) as an orange oil.

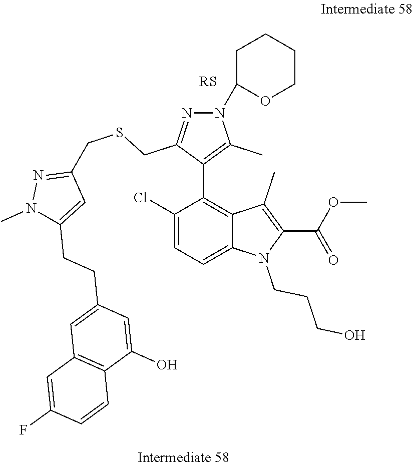

Intermediate 57 (3.9 g, 5.41 mmol), Intermediate 25a (3.69 g, 1.2 eq.), and PPh₃ (142 mg, 0.1 eq.) were charged in a 500 mL round bottom flask. The mixture was degassed and re-filled with nitrogen three times. Dry MeOH (200 mL, degassed by bubbling nitrogen for 20 min) was added. The mixture was degassed and re-filled with nitrogen three times, then degassed by bubbling nitrogen for 15 min. The resulting suspension was cooled to 0° C. before addition of K₂CO₃ (2.24 g, 3 eq.). The reaction mixture was degassed and re-filled again with nitrogen three times, then degassed by bubbling nitrogen for 5 min. The reaction mixture was allowed to warm to room temperature and was stirred for 1.5 h. The reaction mixture was concentrated under reduced pressure. The residue was partitioned between water and EtOAc. The layers were separated and the aqueous layer was extracted with EtOAc (×3). The combined organic layer was washed with brine, dried over MgSO₄, filtered, and evaporated. The residue was dissolved in THF (110 mL) and the solution was cooled to 0° C. TBAF (1M in THF, 32.48 mL, 6 eq.) was added and the reaction mixture was allowed to warm to room temperature and was stirred for 30 min. Additional TBAF (1M in THF, 10.83 mL, 2 eq.) was added and the reaction mixture was stirred at room temperature for 40 min. The reaction was quenched by addition of saturated aqueous NH₄C₁. The layers were separated. The organic layer was washed with brine (×2), the combined aqueous layer was extracted with EtOAc (×3) and DCM and the combined organic extract was dried over MgSO₄, filtered, and concentrated under reduced pressure. The residue was purified by flash column chromatography (SiO₂, 40 g RediSep, heptane/EtOAc, 100/0 to 0/100) to give impure Intermediate 58. This impure product was purified again by flash column chromatography (SiO₂, 120 g RediSep, DCM/MeOH, 100/0 to 90/10) to afford Intermediate 58 (4.13 g, yield: 98%) as a brownish foam.

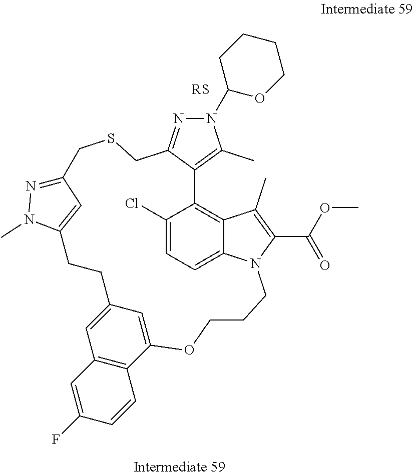

A solution of PPh₃ (1.07 g, 4 eq.) in toluene (31 mL) was degassed and re-filled with nitrogen three times (Solution A). A solution of Intermediate 58 (789 mg, 1.02 mmol) and DTBAD (938 mg, 4 eq.) in a mixture of toluene (31 mL) and THE (6 mL) was degassed and re-filled with nitrogen three times (Solution B). Solution B was added via syringe pump (0.1 ml/min) to Solution A, stirred at 70° C. under nitrogen atmosphere. Once the addition was complete, the reaction mixture was stirred for 15 min at 70° C. The reaction mixture was cooled to room temperature, and the solvents were evaporated. The residue was purified by flash column chromatography (SiO₂, 80 g RediSep, DCM/MeOH, 100/0 to 90/10) to afford Intermediate 59 (2 g, impure, yield considered quantitative) as a yellow oil, used without further purification.

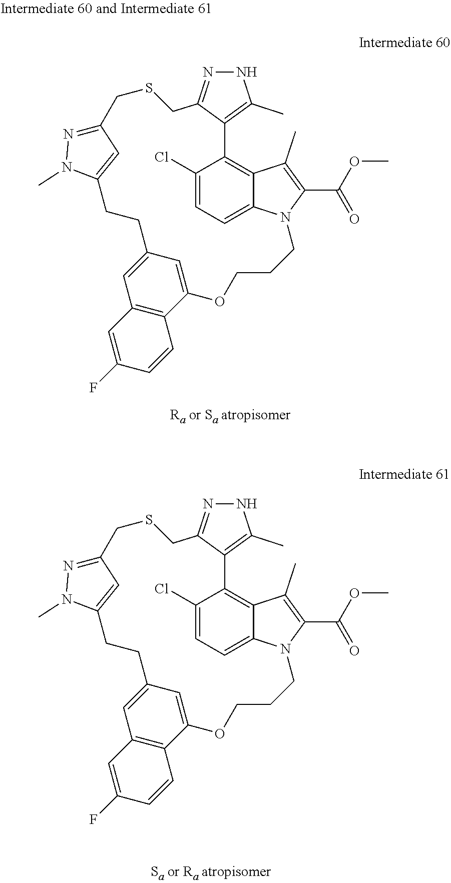

HCl (1.25 M in MeOH, 192 mL, 50 eq.) was added dropwise to a solution of Intermediate 59 (3.62 g, 4.79 mmol) in anhydrous THF (190 mL) at 0° C. The reaction mixture was stirred at room temperature for 3 h. The reaction mixture was concentrated under reduced pressure. The residue was purified by preparative HPLC (Stationary phase: RP XBridge Prep C18 OBD—10 μm, 50×150 mm, Mobile phase: 0.25% NH₄HCO₃ solution in water, CH₃CN) to give the racemic mixture of Intermediate 60 and Intermediate 61. This mixture was separated into its atropisomers by preparative SFC (Stationary phase: Chiralcel Diacel OJ 20×250 mm, Mobile phase: CO₂, EtOH+0.4% iPrNH₂) to afford Intermediate 60 (892 mg, yield: 28%) and Intermediate 61 (932 mg, yield: 29%).

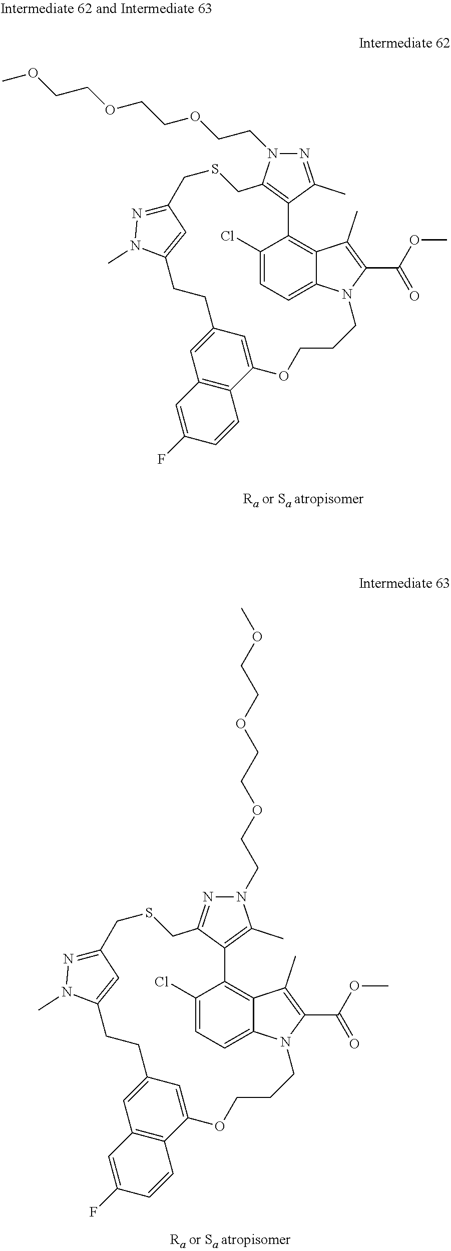

Diethylene glycol 2-bromoethyl methyl ether (CAS [72593-77-2]) (63 mg, 2.5 eq.) was added to a solution of Intermediate 60 (75 mg, 0.11 mmol) and Cs₂CO₃ (182 mg, 5 eq.) in anhydrous DMF (2 mL), stirred at room temperature, under nitrogen atmosphere. The vial was sealed and the reaction mixture was stirred at 60° C. for 4 h. The solvent was evaporated and the residue was diluted with EtOAc and water. The aqueous layer was extracted with EtOAc (3×). The combined organic layer was washed with brine, dried over MgSO₄, filtered, and evaporated to give as a colorless oil. This oil was purified by preparative SFC (Stationary phase: Chiralpak Diacel AD 20×250 mm, Mobile phase: CO₂, iPrOH+0.4% iPrNH₂) to afford Intermediate 62 (21 mg, yield: 23%) and Intermediate 63 (12 mg, yield: 13%).

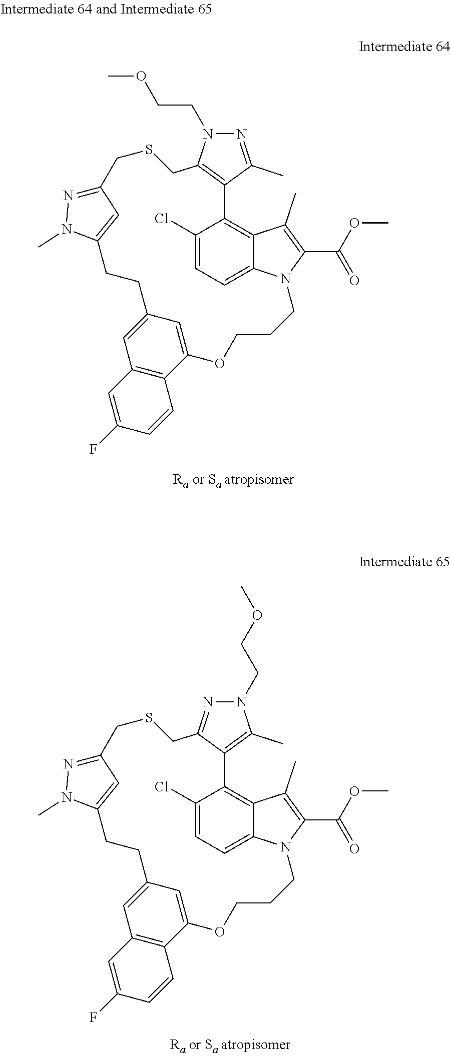

2-Bromoethyl methyl ether (CAS [6482-24-2]) (45 μL, 2.6 eq.) was added to a solution of Intermediate 60 (121 mg, 0.181 mmol) and Cs₂CO₃ (178 mg, 3 eq.) in anhydrous DMF (3 mL) at room temperature under nitrogen atmosphere. The reaction mixture was stirred at room temperature for 6 h. The reaction mixture was diluted with EtOAc and water. The layers were separated. The organic layer was washed with brine (×3), and the combined aqueous extract was extracted with EtOAc (×2) and with DCM (×3). The combined organic layer was dried over MgSO₄, filtered, and evaporated. The residue was purified by preparative SFC (Stationary phase: Chiralpak Daicel IC 20×250 mm, Mobile phase: CO₂, EtOH+0.4% iPrNH₂) to afford Intermediate 64 (54 mg, yield: 41%) and Intermediate 65 (54 mg, yield: 41%). both as white solids.

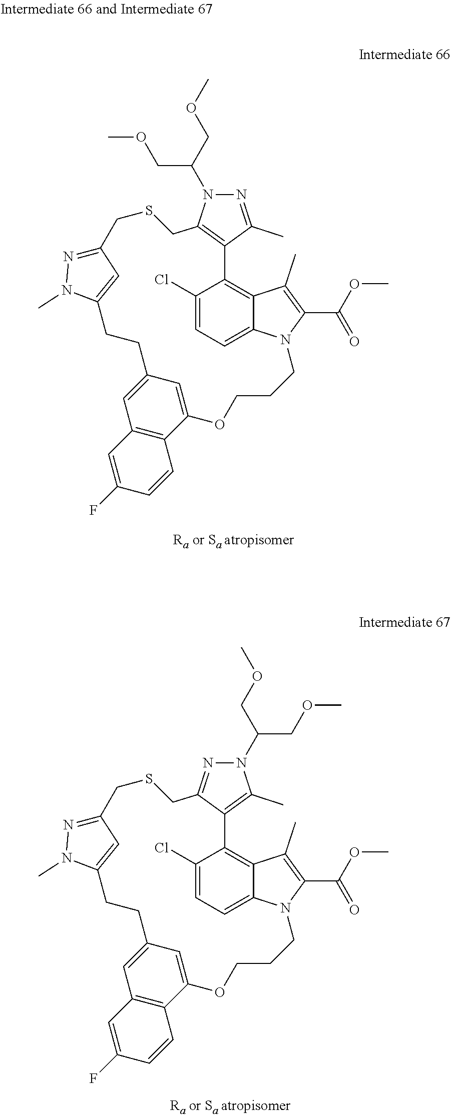

1,3-dimethoxypropan-2-yl methanesulfonate (CAS [215453-88-6]) (142 mg, 5 eq.) and Intermediate 60 (96 mg, 0.144 mmol) were dissolved in anhydrous DMF (2 mL) under nitrogen atmosphere. Cs₂CO₃ (141 mg, 3 eq.) was added at room temperature. The vial was sealed and the reaction mixture was stirred at 70° C. for 16 h. To push the reaction to completion, additional 1,3-dimethoxypropan-2-yl methanesulfonate [215453-88-6](142 mg, 5 eq.) was added under nitrogen atmosphere and the reaction mixture was stirred at 100° C. for 6 h. Again, additional 1,3-dimethoxypropan-2-yl methanesulfonate [215453-88-6] (142 mg, 5 eq.) was added and the reaction mixture was stirred at 100° C. for 3 h. The reaction mixture was cooled to room temperature and was stirred for 17 h. The solvent was evaporated, and the resulting crude mixture was diluted with EtOAc and water. The layers were separated. The organic layer was washed with brine (×3), and the combined aqueous extract was extracted with EtOAc (×3) and DCM. The combined organic layer was dried over MgSO₄, filtered, and evaporated. The residue was purified by flash column chromatography (SiO₂, 40 g RediSep, DCM/MeOH, 100/0 to 90/10) to give a yellow oil. This oil was further purified by preparative HPLC (Stationary phase: RP XBridge Prep C18 OBD—5 μm, 50×250 mm, Mobile phase: 0.25% NH₄HCO₃ solution in water, CH₃CN), followed by preparative SFC (Stationary phase: Chiralpak Diacel AD 20×250 mm, Mobile phase: CO₂, EtOH+0.4% iPrNH₂) to afford Intermediate 66 (5 mg, yield: 5%) and Intermediate 67 (7 mg, yield: 7%), both as white solids.

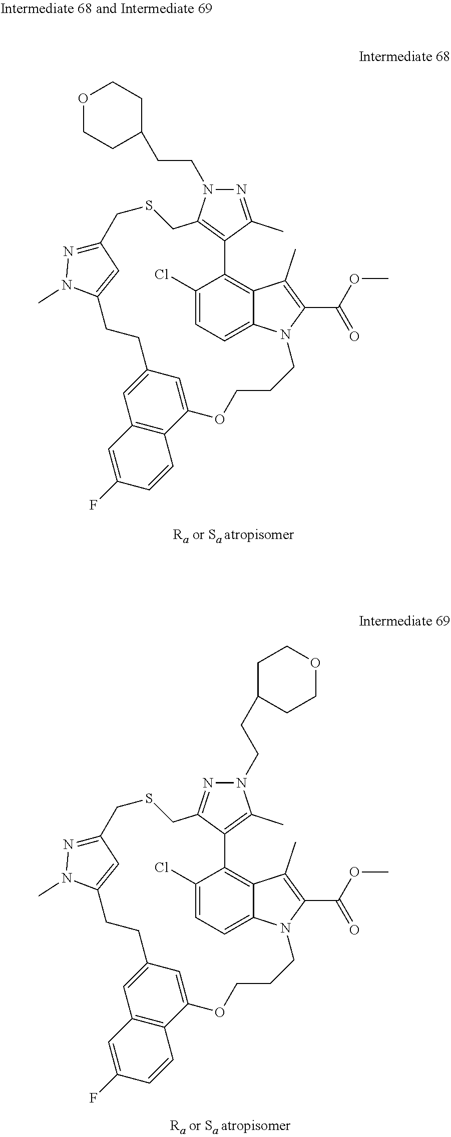

4-(2-Bromoethyl)tetrahydropyran (CAS [4677-20-7]) (62 mg, 2.7 eq.) was added to a solution of Intermediate 60 (80 mg, 0.12 mmol) and Cs₂CO₃ (117 mg, 3 eq.) in anhydrous DMF (2 mL) at room temperature, under nitrogen atmosphere. The reaction mixture was stirred at room temperature for 4.5 h. The solvent was evaporated and the residue was diluted with DCM and water. The layers were separated and the organic layer was washed with brine (×3). The combined aqueous layer was extracted with EtOAc (×2) and with DCM (×3). The combined organic layer was dried over MgSO₄, filtered, and evaporated to give a colorless oil. This oil was purified by preparative SFC (Stationary phase: Chiralpak Diacel AD 20×250 mm, Mobile phase: CO₂, EtOH+0.4% iPrNH₂) to afford Intermediate 68 (39 mg, yield: 42%) and Intermediate 69 (30 mg, yield: 32%), both as white solids.

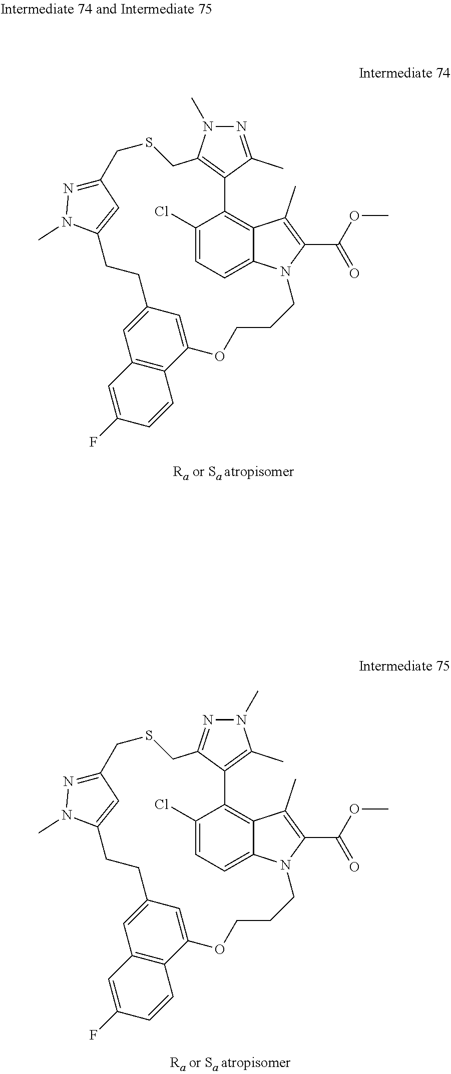

MeI (21 μL, 2.5 eq.) was added to a mixture of Intermediate 60 (90 mg, 0.134 mmol) and Cs₂CO₃ (132 mg, 3 eq.) in anhydrous DMF (2 mL) at room temperature under nitrogen atmosphere. The reaction mixture was stirred at room temperature for 4 h. The solvent was evaporated. The residue was diluted with DCM and water and the layers were separated. The organic layer was washed with brine (×3). The combined aqueous layer was extracted with DCM (×4) and EtOAc. The combined organic layer was dried over MgSO₄, filtered, and evaporated. The residue was purified by flash column chromatography (SiO₂, 24 g RediSep, DCM/MeOH, 100/0 to 90/10) followed by preparative HPLC (Stationary phase: RP XBridge Prep C18 OBD—5 μm, 50×250 mm, Mobile phase: 0.25% NH₄HCO₃ solution in water, CH₃CN), and finally by preparative SFC (Stationary phase: Chiralpak Daicel ID 20×250 mm, Mobile phase: CO₂, EtOH+0.4% iPrNH₂) to give Intermediate 74 (7 mg, yield: 7%) and Intermediate 75 (1 mg, yield: 1%), both as white solids.

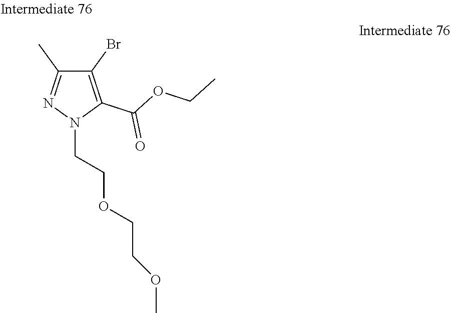 [SMILES: CCOC(=O)c1c(Br)c(C)nn1CCOCCOC]

Cyanomethylenetributylphosphorane (CAS [157141-27-0]) (45.02 mL, 1 eq.) was added dropwise to a solution of 1H-pyrazole-3-carboxylic acid, 4-bromo-5-methyl-, ethyl ester [6076-14-8] (20 g, 85.81 mmol) and 2-(2-methoxyethoxy)ethanol [111-77-3] (14.15 mL, 1.4 eq.) in THF (1.9 L) at room temperature. The reaction mixture was stirred at room temperature overnight. The reaction mixture was poured into water (100 mL) and the mixture was extracted with EtOAc (3×100 mL). The combined organic layer was washed with brine, dried over MgSO₄, filtered, and concentrated in vacuo. The crude product was purified via flash column chromatography on silica gel (heptane/EtOAc, 100/0 to 50/50) to give Intermediate 76 (24 g, yield: 83%).

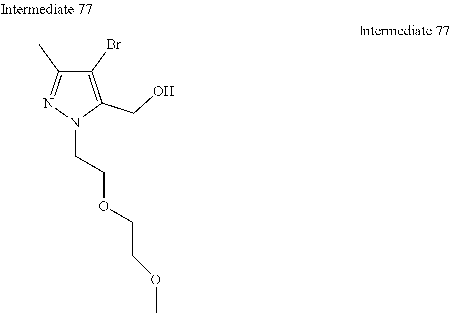 [SMILES: COCCOCCn1nc(C)c(Br)c1CO]

Sodium borohydride (4.26 g, 5 eq.) was added to a solution of Intermediate 76 (7.45 g, 22.23 mmol) in a mixture of THF (130 mL) and MeOH (34 mL) at 0° C. After 5 min, the resulting mixture was allowed to reach room temperature and was stirred for 3 h. The reaction mixture was diluted by very slow addition of acetone (80 mL) and water (80 mL), followed by EtOAc (100 mL). The layers were separated and the aqueous layer was extracted with EtOAc (2×50 mL) followed by a 1:1 mixture of EtOAc/THF (2×50 mL). The combined organic layer was dried over MgSO₄, filtered, and evaporated to afford Intermediate 77 (7.24 g, quantitative) as a tan oil, used without further purification.

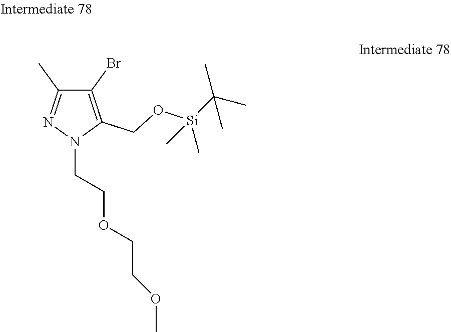 [SMILES: COCCOCCn1nc(C)c(Br)c1CO[Si](C)(C)C(C)(C)C]

TBDMSCl (617 mg, 1.2 eq.) was added portionwise at 0° C. to a stirred and previously degassed (nitrogen) solution of Intermediate 77 (1 g, 3.41 mmol) and imidazole (325 mg, 1.4 eq.) in dry DCM (10 mL). The reaction mixture was stirred at room temperature under nitrogen for 2 h. To push the reaction to completion, additional TBDMSCl (150 mg, 0.3 eq.) was added and the reaction mixture was stirred at room temperature for another 1.5 h. Saturated aqueous NH₄Cl was added and the layers were separated. The organic layer was dried over MgSO₄, filtered, and concentrated in vacuo. The resulting tan oil residue was purified by flash chromatography on silica gel (EtOAc/heptane 0/100 to 30/70) to give Intermediate 78 (1.09 g, yield: 78%) as a light tan clear oil.

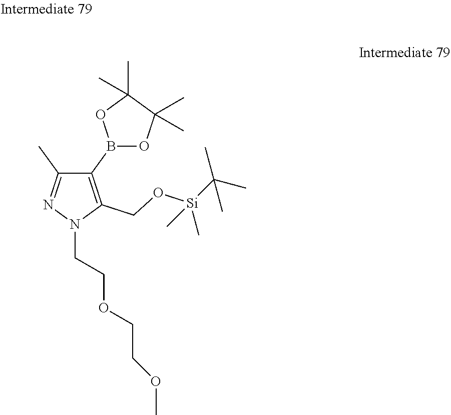 [SMILES: COCCOCCn1nc(C)c(B2OC(C)(C)C(C)(C)O2)c1CO[Si](C)(C)C(C)(C)C]

A solution of Intermediate 78 (1.06 g, 2.60 mmol) in dry THE (11 mL) was cooled to −78° C. under nitrogen atmosphere. nBuLi (2.5 M in hexanes; 1.3 mL, 1.25 eq.) was added dropwise. The reaction mixture was stirred at −78° C. for 1 h. 2-Isopropoxy-4,4,5,5-tetramethyl-1,3,2-dioxaborolane (CAS [61676-62-8]) (0.64 mL, 1.2 eq.) was added dropwise. After the addition, the reaction mixture was allowed to warm to room temperature and was stirred for 1 h. The reaction was quenched by slow addition of EtOAc (25 mL), followed by saturated aqueous NH₄Cl (20 mL). The layers were separated and the aqueous layer was extracted with EtOAc (2×20 mL). The combined organic layer was washed with brine (20 mL), dried over MgSO₄, filtered, and concentrated in vacuo. The residue was purified by flash column chromatography on silica gel (EtOAc/heptane 0/100 to 50/50) to afford Intermediate 79 (934 mg, yield: 79%) as a yellow clear oil.

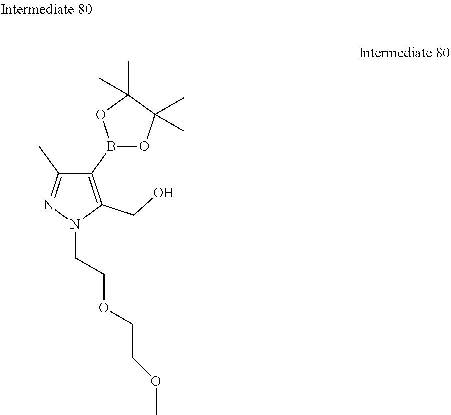

TBAF (1.0 M in THF, 1.2 eq.) was added to a solution of Intermediate 79 (930 mg, 2.05 mmol) in anhydrous 2-Me-THF (12 mL) under nitrogen atmosphere, at 0° C. The ice bath was removed and the resulting mixture was stirred at room temperature for 16 h. The reaction mixture was diluted with EtOAc and saturated aqueous NH₄Cl was added. The layers were separated and the aqueous layer was extracted twice with EtOAc. The combined organic layer was washed with brine, dried over MgSO₄, filtered, and concentrated in vacuo. The residue was purified by flash column chromatography on silica gel (MeOH in DCM 0/100 to 5/95) to afford Intermediate 80 (580 mg, yield: 83%) as a light yellow clear oil.

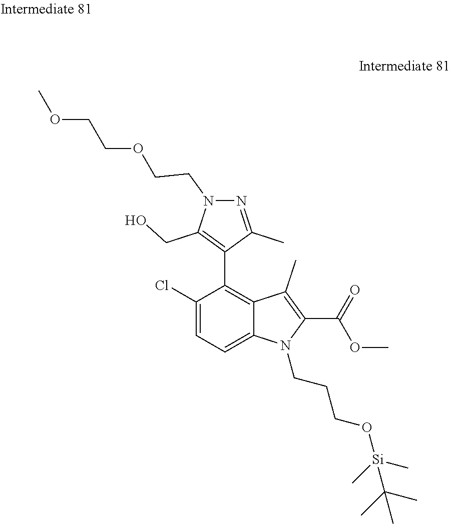 [SMILES: COCCOCCn1nc(C)c(-c2c(Cl)ccc3c2c(C)c(C(=O)OC)n3CCCO[Si](C)(C)C(C)(C)C)c1CO]

Pd(amphos)₂Cl₂ (CAS [887919-35-9]) (51 mg, 0.05 eq.) was added to a stirred and previously nitrogen-degassed mixture of Intermediate 55 (706 mg, 1.44 mmol), Intermediate 80 (580 mg, 1.2 eq.) and K₂CO₃ (400 mg, 2 eq.) in water (2 mL) and 1,4-dioxane (8 mL) in a microwave tube at room temperature and under nitrogen. The reaction mixture was degassed by bubbling nitrogen through. The vial was sealed and the reaction mixture was stirred at 65° C. for 2 h. The reaction mixture was diluted with EtOAc and water and the layers were separated. The aqueous layer was extracted twice with EtOAc. The combined organic layer was dried over MgSO₄, filtered, and evaporated. The residue was purified by flash column chromatography (silica; EtOAc in n-heptane 0/100 to 100/0) to yield Intermediate 81 (700 mg, yield: 80%) as a yellow oil.

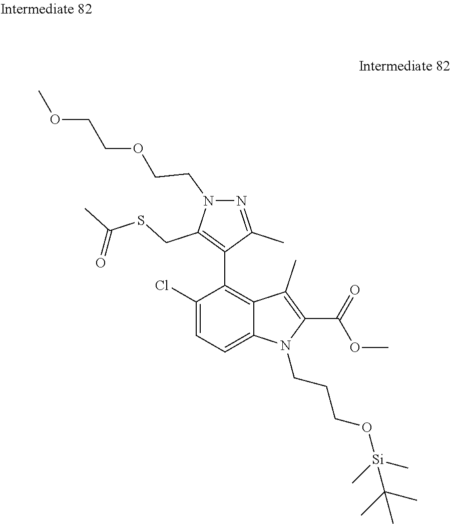 [SMILES: COCCOCCn1nc(C)c(-c2c(Cl)ccc3c2c(C)c(C(=O)OC)n3CCCO[Si](C)(C)C(C)(C)C)c1CSC(C)=O]

MsCl (0.11 mL 1.25 eq.) was added dropwise to a previously nitrogen-degassed solution of Intermediate 81 (700 mg, 1.15 mmol) and Et₃N (0.24 mL, 1.5 eq.) in THF (10 mL), under nitrogen at 0° C. The resulting mixture was allowed to warm up to room temperature and was stirred for 1 h. A previously nitrogen-degassed solution of KSAc (657 mg, 5 eq.) in DMF (20 mL) was added and stirring was continued at room temperature for 2 h. To push the reaction to completion, a nitrogen-degassed solution of KSAc (394 mg, 3 eq.) in DMF (10 mL) was added. The reaction mixture was further stirred for 1 h. the reaction mixture was diluted with EtOAc and water. The layers were separated and the aqueous layer was extracted twice with EtOAc. The combined organic layer was washed with brine, dried over MgSO₄, filtered, and evaporated. The residue was purified by flash column chromatography (silica, 120 g; EtOAc in n-heptane 30/70 to 70/30) to afford Intermediate 82 (287 mg, yield: 37%). Impure fractions were purified again by flash column chromatography (silica, 80 g; EtOAc in n-heptane 0/100 to 70/30) to yield another batch of Intermediate 82 (172 mg, yield: 22%)

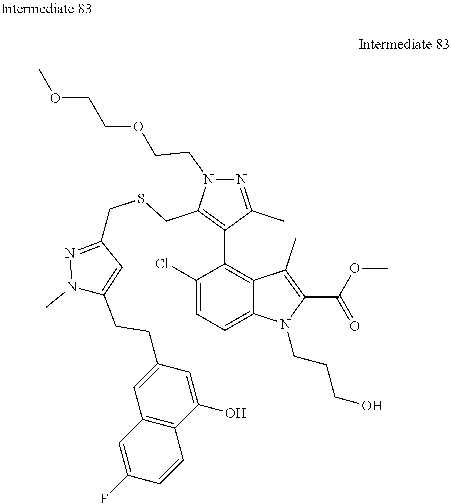

Intermediate 82 (460 mg, 0.69 mmol), Intermediate 25a (466 mg, 1.2 eq.), and PPh₃ (18 mg, 0.1 eq.) were charged in a 100 mL round bottom flask. The mixture was degassed and re-filled with nitrogen three times. Anhydrous MeOH (25 mL; degassed by bubbling nitrogen for 30 min) was added. The suspension was degassed and re-filled with nitrogen three times. The reaction mixture was cooled to 0° C. before addition of K₂CO₃ (286 mg, 3 eq.). The reaction mixture was degassed and re-filled with nitrogen three times. The reaction mixture was allowed to warm to room temperature and was stirred for 1.5 h. The reaction mixture was concentrated under vacuum and the resulting slurry was partitioned between water and EtOAc. The layers were separated and the aqueous layer was extracted twice with EtOAc. The combined organic layer was washed with brine, dried over MgSO₄, filtered, and concentrated under reduced pressure. This residue was dissolved in MeOH (25 mL), and pTsOH.H₂O (394 mg, 3 eq.) was added at room temperature. The solution was stirred for 40 min at room temperature. The solvent was evaporated and the residue was dissolved in EtOAc and water, and saturated aqueous NaHCO₃ was added. The layers were separated and the aqueous layer was extracted twice with EtOAc. The combined organic layer was washed with brine, dried over MgSO₄, filtered, and concentrated under reduced pressure. The residue was purified by flash column chromatography (silica, 120 g; MeOH in DCM 0/100 to 5/95) to afford Intermediate 83 (511 mg, yield: 93%).

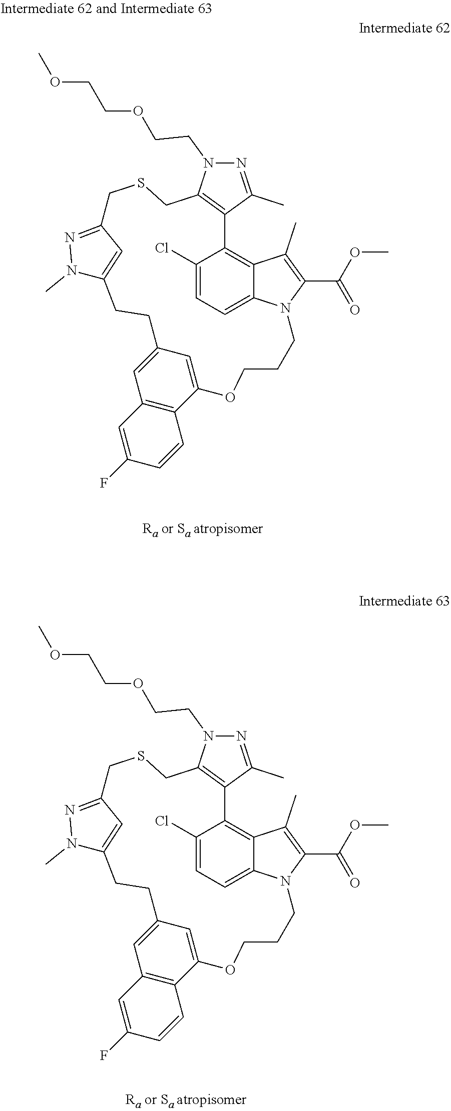

PPh₃ (650 mg, 4 eq.) was dissolved in dry toluene (19 mL, previously vacuum-degassed and re-filled with nitrogen three times) to give Solution A. DTBAD (571 mg, 4 eq.) was added to a solution of Intermediate 83 (491 mg, 0.62 mmol) in dry THF (4 mL, previously vacuum-degassed and re-filled with nitrogen three times) and dry toluene (19 mL, previously vacuum-degassed and re-filled with nitrogen three times) to give Solution B. Solution B was added to solution A via syringe pump (0.1 mL/min) at 70° C. Once the addition was complete, the reaction mixture was stirred for 20 min at 70° C. After cooling to room temperature, the solvents were evaporated and the residue was purified by flash column chromatography (silica, 120 g; EtOAc in n-heptane 0/100 to 20/80, then 100% EtOAc, and finally, MeOH in DCM 5/95) to yield a light yellow solid. This solid was purified by preparative SFC (Stationary phase: Chiralpak Diacel AD 20×250 mm, Mobile phase: CO₂, EtOH+0.4% iPrNH₂) to afford Intermediate 84 (135 mg, yield: 28%) and Intermediate 85 (148 mg, yield: 31%), both as off-white solids.

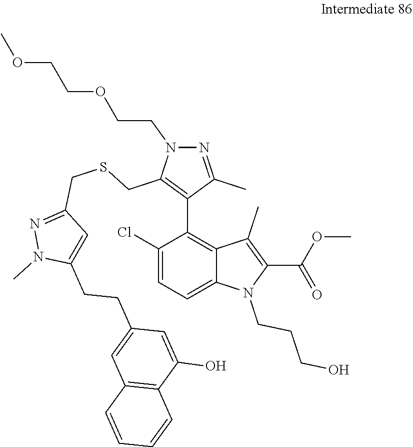

Intermediate 82 (1.96 g, 2.94 mmol), Intermediate 15 (2 g, 1.2 eq.), and PPh₃ (77 mg, 0.1 eq.) were charged in a 500 mL round bottom flask. The mixture was degassed and re-filled with nitrogen three times. Dry MeOH (200 mL, degassed by bubbling nitrogen for 30 min) was added. The suspension was degassed and re-filled with nitrogen three times. The reaction mixture was cooled to 0° C. before addition of K₂CO₃ (1.22 g, 3 eq.). After this addition, the reaction mixture was degassed and re-filled with nitrogen three times. The reaction mixture was allowed to warm to room temperature and was stirred for 1.5 h, then heated up to 40° C. and stirred for 1.5 h. The reaction mixture was concentrated under reduced pressure and the resulting slurry was partitioned between water and EtOAc. The layers were separated and the aqueous layer was extracted twice with EtOAc. The combined organic layer was washed with brine, dried over MgSO₄, filtered, and concentrated under reduced pressure. The residue was dissolved in MeOH (200 mL) and pTsOH.H₂O (1.68 g, 3 eq.) was added at room temperature. The reaction mixture was stirred at room temperature for 30 min. The solvent was evaporated and the residue was partitioned between EtOAc and water. Saturated aqueous NaHCO₃ was added. The aqueous layer was extracted twice with EtOAc. The combined organic layer was washed with brine, dried over MgSO₄, filtered, and concentrated under reduced pressure. The residue was purified by flash column chromatography (silica, 220 g; EtOAc in n-heptane 0/100 to 100/0 followed by MeOH in DCM 0/100 to 5/95) to yield Intermediate 86 (1.89 g, yield: 76%) as a tan clear oil.

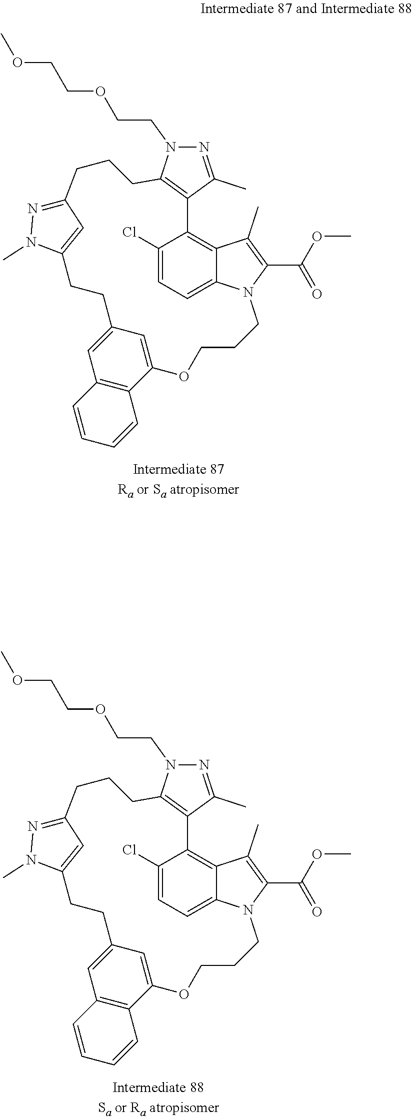

Intermediate 87 and Intermediate 88 were prepared according to an analogous procedure as for Intermediate 84 and Intermediate 85, respectively, starting from Intermediate 86 instead of Intermediate 83.

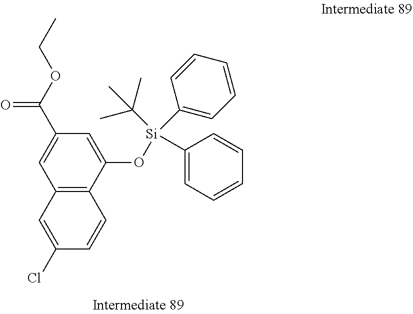 [SMILES: CCOC(=O)c1cc(O[Si](c2ccccc2)(c2ccccc2)C(C)(C)C)c2ccc(Cl)cc2c1]

TBDPSCl (4.93 g, 1.5 eq) was added dropwise to a solution of ethyl 7-chloro-4-hydroxy-2-naphthoate (CAS [2122548-70-1]) (3 g, 11.97 mmol) and imidazole (1.22 g, 1.5 eq) in dry DMF (60 mL) at 0° C. The resulting mixture was stirred overnight at room temperature under nitrogen atmosphere. The reaction mixture was diluted with EtOAc (100 mL) and washed with water. The organic layer was dried with Na₂SO₄, filtered, and concentrated under reduced pressure. The residue was purified by silica gel column chromatography to give Intermediate 89 (5.8 g, yield: 90% yield) as a yellow oil.

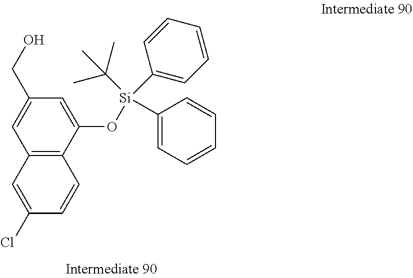 [SMILES: CC(C)(C)[Si](Oc1cc(CO)cc2cc(Cl)ccc12)(c1ccccc1)c1ccccc1]

DIBAL (1 M in hexane, 5.11 mL, 2.5 eq) was added dropwise to a solution of Intermediate 89 (1 g, 2.045 mmol) in dry toluene (40 mL) at −78° C. The reaction mixture was stirred at −78° C. for 10 min under nitrogen atmosphere, then warmed to 0° C. and kept at this temperature for 1 h. The reaction was quenched by addition of saturated aqueous NH₄Cl and the reaction mixture was extracted with EtOAc. The organic layer was dried over Na₂SO₄, filtered, and evaporated. The residue was purified by silica gel chromatography (EtOAc/petroleum ether 0/100 to 15/85) to afford Intermediate 90 (463 mg, yield: 51%) as a pale yellow solid.

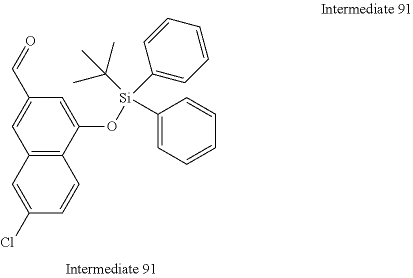 [SMILES: CC(C)(C)[Si](Oc1cc(C=O)cc2cc(Cl)ccc12)(c1ccccc1)c1ccccc1]

Dess-Martin periodinane (440 mg, 1 eq) was added to a solution of Intermediate 90 (463 mg, 1.037 mmol) in DCM (40 mL) at room temperature. The reaction mixture was stirred at room temperature for 2 h. The reaction was quenched by addition of saturated aqueous Na₂SO₃, and the mixture was extracted with EtOAc. The organic layer was dried over Na₂SO₄, filtered, and evaporated. The residue was purified by silica gel chromatography (EtOAc/petroleum ether 0/100 to 10/90) to afford Intermediate 91 (439 mg, yield: 95%) as a pale yellow solid.

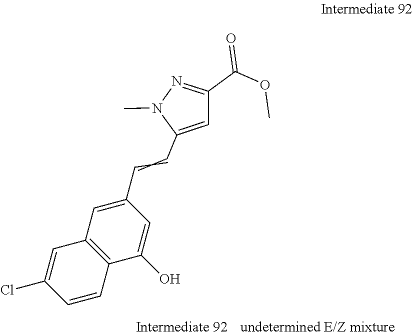 [SMILES: COC(=O)c1cc(C=Cc2cc(O)c3ccc(Cl)cc3c2)n(C)n1]

NaH (60% in mineral oil, 20 mg, 1.1 eq) was added to a suspension of intermediate 105 (223 mg, 1.1 eq) in dry THF (5 mL) at 0° C., under nitrogen atmosphere. After stirring at 0° C. for 40 min, the reaction mixture was cooled to −20° C. and Intermediate 91 (200 mg, 0.449 mmol) in THF (1 mL) was added slowly at −20° C. After the addition, the reaction mixture was stirred at −10° C. for 2 h. Water was added to quench the reaction at 0° C. The resulting mixture was extracted with EtOAc. The separated organic layer was dried over Na₂SO₄, filtered, and evaporated. The residue was purified by silica gel column chromatography (petroleum ether/EtOAc 100/0 to 30/70) to afford Intermediate 92 (150 mg, yield: 97%) as a white solid.

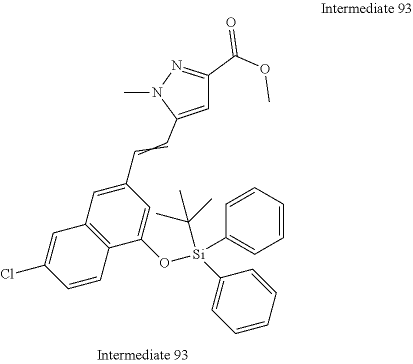 [SMILES: COC(=O)c1cc(C=Cc2cc(O[Si](c3ccccc3)(c3ccccc3)C(C)(C)C)c3ccc(Cl)cc3c2)n(C)n1]

TBDPSCl (307 mg, 1.5 eq) was added dropwise to a mixture of Intermediate 92 (255 mg, 0.744 mmol) and imidazole (76 mg, 1.5 eq) in dry DMF (10 mL) at 0° C. The reaction mixture was stirred overnight at room temperature under nitrogen atmosphere. The reaction mixture was diluted with EtOAc (30 mL) and washed with water. The organic layer was dried with Na₂SO₄, filtered, and concentrated under reduced pressure. The residue was purified by silica gel column chromatography to give Intermediate 93 (400 mg, yield: 92%) as a white solid.

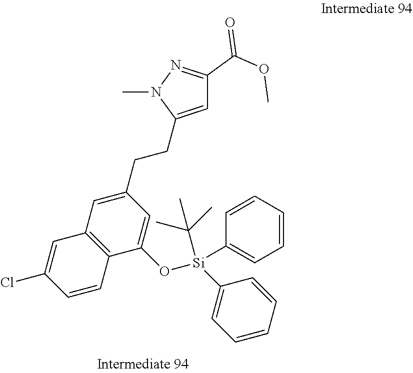 [SMILES: COC(=O)c1cc(CCc2cc(O[Si](c3ccccc3)(c3ccccc3)C(C)(C)C)c3ccc(Cl)cc3c2)n(C)n1]

Pd/C (10% 37 mg, 0.8 eq.) was added to a solution of Intermediate 93 (250 mg, 0.43 mmol) in dry EtOAc (5 mL). The reaction mixture was degassed, filled with H₂ three times, and stirred under an atmosphere of H₂ at room temperature for 16 h. The reaction mixture was filtered through a Celite pad and the solid cake was washed with EtOAc. The filtrate was evaporated and the residue was purified by silica gel column chromatography to afford Intermediate 94 (245 mg, yield: 97%) as a colorless oil.

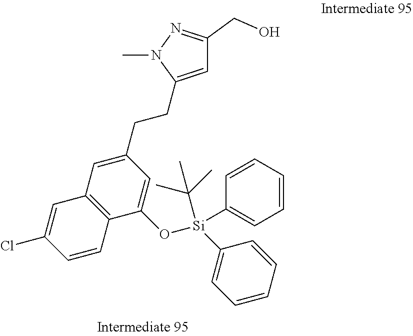 [SMILES: Cn1nc(CO)cc1CCc1cc(O[Si](c2ccccc2)(c2ccccc2)C(C)(C)C)c2ccc(Cl)cc2c1]

DIBAL (1.5 M in toluene, 1.05 mL, 3.5 eq) was added dropwise to a solution of Intermediate 94 (263 mg, 0.451 mmol) in dry toluene (5 mL) at −78° C. The reaction mixture was stirred at −78° C. for 10 min then warmed to 0° C. and kept at this temperature for 1 h. The reaction was quenched by addition of saturated aqueous NH₄Cl and the mixture was extracted with EtOAc. The organic layer was dried over Na₂SO₄, filtered, and evaporated. The residue was purified by silica gel chromatography (DCM/MeOH 100/0 to 90/10) to afford Intermediate 95 (214 mg, yield: 85% yield) as a white solid.

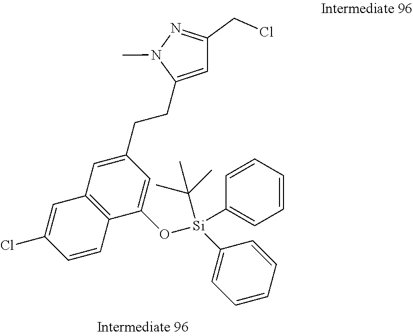 [SMILES: Cn1nc(CCl)cc1CCc1cc(O[Si](c2ccccc2)(c2ccccc2)C(C)(C)C)c2ccc(Cl)cc2c1]

Thionyl chloride (32 μL, 1.15 eq) was added dropwise to a solution of Intermediate 95 (214 mg, 0.385 mmol) in dry DCM (5 mL) at 0° C. The reaction mixture was stirred at 0° C. under nitrogen atmosphere for 10 min then warmed to room temperature and kept at this temperature for 1 h. The reaction was quenched by addition of saturated aqueous NH₄Cl and the mixture was extracted with EtOAc. The organic layer was dried over Na₂SO₄, filtered, and evaporated to afford Intermediate 96 (223 mg, considered quantitative), used without purification.

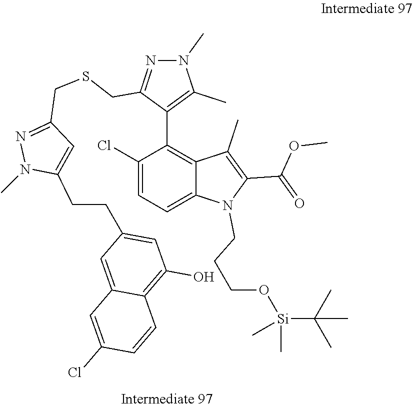 [SMILES: COC(=O)c1c(C)c2c(-c3c(CSCc4cc(CCc5cc(O)c6ccc(Cl)cc6c5)n(C)n4)nn(C)c3C)c(Cl)ccc2n1CCCO[Si](C)(C)C(C)(C)C]

Intermediate 8 (1.243 g, 2.15 mmol) and Intermediate 96 (1.418 g, 1.15 eq) were dissolved in MeOH (15 mL). The reaction mixture was degassed and re-filled with nitrogen five times. K₂CO₃ (594 mg, 2 eq) was then added and the reaction mixture was stirred at room temperature overnight. The solvent was evaporated and the residue was partitioned between water and EtOAc. The layers were separated and the organic layer was washed with brine, dried over Na₂SO₄, filtered, and evaporated. The residue was purified by silica gel chromatography (hexane/EtOAc 100/0 to 20/80) to afford Intermediate 97 (1.294 g, yield: 71%) as an off-white foamy solid.

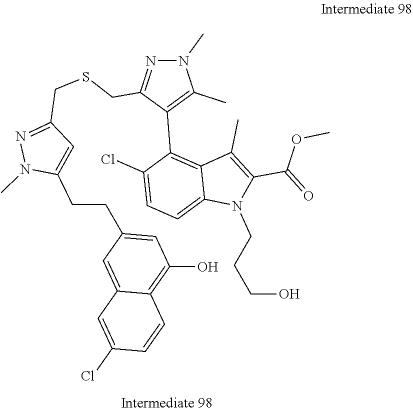

pTsOH.H₂O (324 mg, 1.1 eq) was added to a solution of Intermediate 97 (1.294 g, 1.549 mmol) in MeOH (30 mL). The reaction mixture was stirred at room temperature for 1.5 h. The solvent was evaporated and the residue was partitioned between water and EtOAc. The layers were separated and the organic layer was washed with brine, dried over Na₂SO₄, filtered, and evaporated. The residue was purified by silica gel chromatography (DCM/MeOH 100/0 to 95/5) to afford Intermediate 98 (930 mg, yield: 83%) as a pale yellow foamy solid.

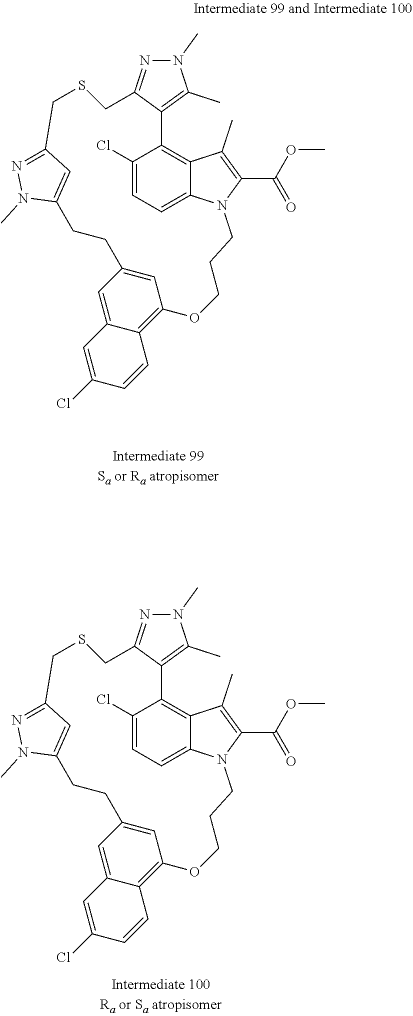

A solution of Intermediate 98 (1.506 g, 2.165 mmol) and DTBAD (1.994 g, 4 eq.) in toluene (55 mL) and THF (8 mL) was added dropwise over 60 min, at 70° C. under nitrogen to a solution of PPh₃ (2.271 g, 4 eq) in toluene (55 mL). After the addition, the reaction mixture was further stirred at the same temperature for 1 h. The solvents were evaporated and the residue was partitioned between water and DCM. The layers were separated and the aqueous layer was extracted with DCM (50 mL×3). The combined organic layer was washed with brine, dried over Na₂SO₄, filtered, and evaporated. The residue was purified by silica gel chromatography (hexane/EtOAc 100/0 to 20/80) to give the racemic mixture of Intermediate 99 and Intermediate 100. This racemic mixture was separated by preparative chiral-HPLC (Column: CHIRAL ART Cellulose-SB, 30*250 mm, 5 um; Mobile Phase A: CO₂, Mobile Phase B: IPA (0.5% 2 M NH3-MeOH); Flow rate: 50 mL/min; Gradient: 40% B) to afford Intermediate 99 (490 mg, yield: 32%) and Intermediate 100 (420 mg, yield: 27%), both as a pale yellow foamy solids.

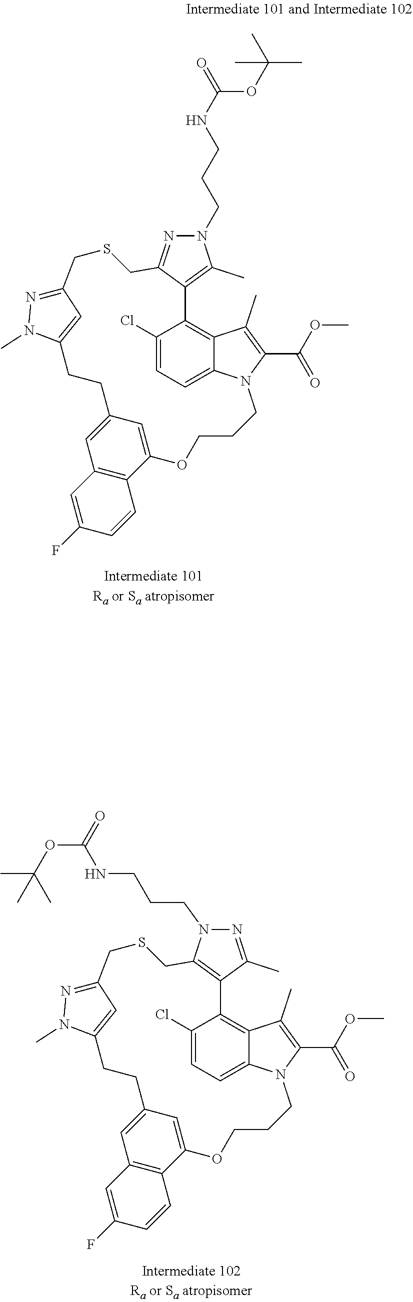

3-(Boc-amino)propyl bromide (CAS [83948-53-2]) (191 mg, 3 eq.) was added to a stirred mixture of Intermediate 60 (180 mg, 0.268 mmol) and Cs₂CO₃ (264 mg, 3 eq.) in anhydrous DMF (4 mL) at room temperature, under nitrogen atmosphere. The reaction mixture was stirred at room temperature under nitrogen atmosphere for 18 h. The solvent was removed under reduced pressure. The residue was diluted with DCM and brine. The layers were separated and the organic layer was washed with brine (×3). The combined aqueous layer was extracted with DCM (×4). The combined organic layer was dried over MgSO₄, filtered, and evaporated to give a colorless oil. This oil was further purified by preparative SFC (Stationary phase: Chiralpak Daicel ID 20×250 mm, Mobile phase: CO₂, iPrOH+0.4% iPrNH₂) to afford Intermediate 101 (90 mg, yield: 40%) and Intermediate 102 (93 mg, yield: 42%) both as pale yellow oils.

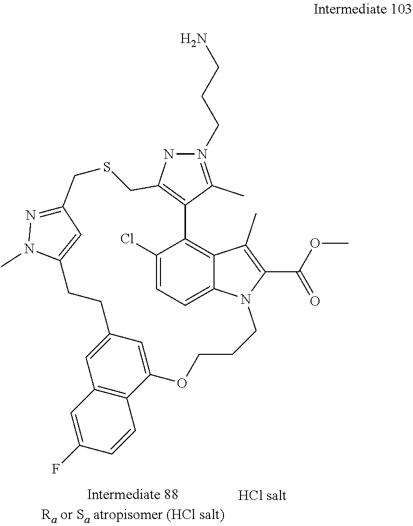

HCl (6 M in iPrOH, 1.81 mL, 100 eq.) was added to a solution of Intermediate 101 (90 mg, 0.109 mmol) in MeOH (2 mL) at room temperature under nitrogen atmosphere. The reaction mixture was stirred at room temperature for 5 h. The solvent was evaporated to give Intermediate 103 (96 mg, considered quantitative) as a pale yellow solid, used without further purification.

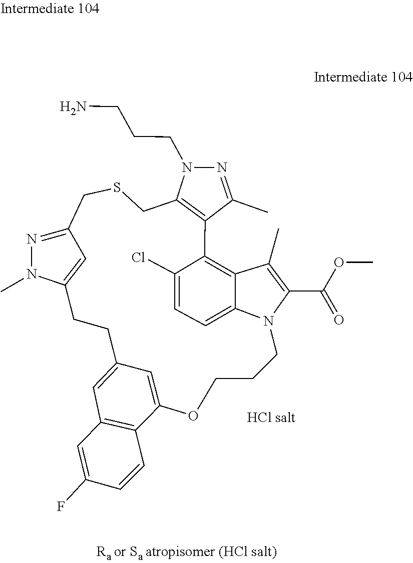

Intermediate 104 was prepared according to an analogous procedure as for Intermediate 103, starting from Intermediate 102 instead of Intermediate 101.

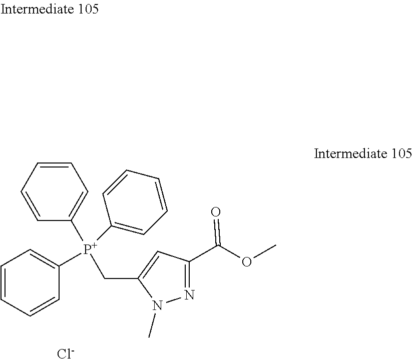 [SMILES: COC(=O)c1cc(C[P+](c2ccccc2)(c2ccccc2)c2ccccc2)n(C)n1 ; [Cl-]]

A solution of 5-(chloromethyl)-1-methyl-1H-pyrazole-3-carboxylic acid, methyl ester (CAS [2245938-86-5]) (24 g, 0.127 mol) and PPh₃ (37 g, 1 eq.) in ACN (250 mL) was stirred under reflux for 16 h. The white suspension was concentrated in vacuo and triturated with EtOAc (100 mL). The resulting solid was collected by filtration and dried to afford Intermediate 105 (54.8 g, yield: 96%) as a white solid.

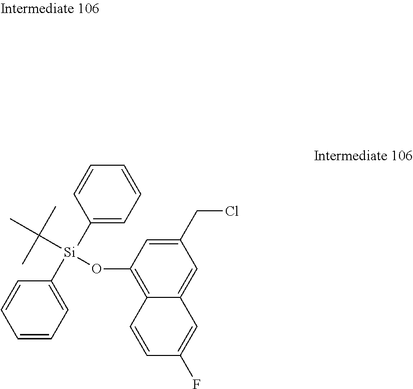 [SMILES: CC(C)(C)[Si](Oc1cc(CCl)cc2cc(F)ccc12)(c1ccccc1)c1ccccc1]

Thionyl chloride (13 g, 1.5 eq.) was added to a solution of Intermediate 20a (31 g, 72 mmol) in DCM (300 mL) at room temperature and the reaction mixture was stirred at room temperature for 3 h. The reaction mixture was concentrated under reduced pressure to give Intermediate 106 (32 g, yield: 99%) as a yellow oil, used without further purification.

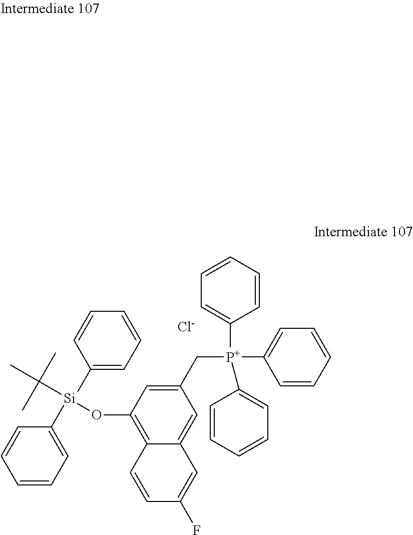 [SMILES: CC(C)(C)[Si](Oc1cc(C[P+](c2ccccc2)(c2ccccc2)c2ccccc2)cc2cc(F)ccc12)(c1ccccc1)c1ccccc1 ; [Cl-]]

PPh₃ (37.68 g, 2 eq.) was added to a solution of Intermediate 106 (32 g, 71.26 mmol) in DCM (300 mL) at room temperature. The solvent was evaporated and the residue was stirred at 140° C. for 16 h (neat reaction). The resulting residue was triturated with EtOAc (150 mL) and filtered to give Intermediate 107 (27 g, yield: 46%) as a white solid.

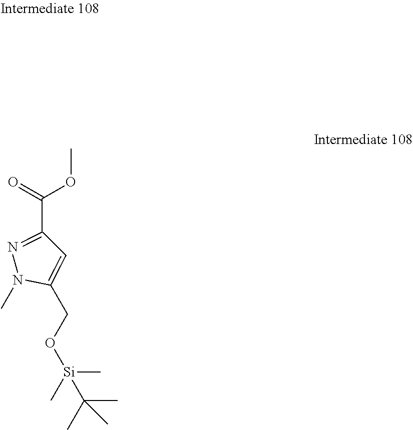 [SMILES: COC(=O)c1cc(CO[Si](C)(C)C(C)(C)C)n(C)n1]

TBDMSCl (77 g, 1.1 eq.), followed by imidazole (35 g, 1.1 eq.) were added to a solution of methyl 5-hydroxymethyl-1-methyl-1 h-pyrazole-3-carboxylate (CAS [1208081-63-3], 79 g, 464 mmol) in DCM (800 mL) and the resulting solution was stirred at room temperature for 16 h. The solvent was evaporated and the residue was purified by silica gel column chromatography (EtOAc/petroleum ether, 3/1) to give Intermediate 108 (126 g, yield: 78%) as a light yellow oil.

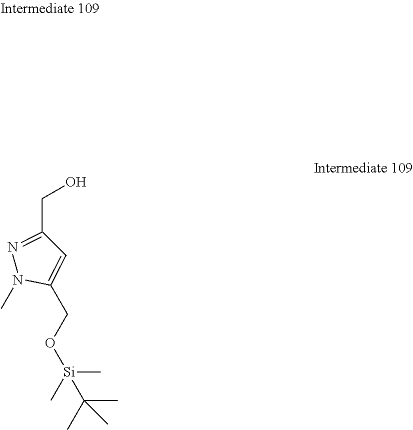 [SMILES: Cn1nc(CO)cc1CO[Si](C)(C)C(C)(C)C]

DIBAL (1 M in hexane, 1.33 L, 3 eq.) was added dropwise at 0° C. to a solution of Intermediate 108 (126 g, 443 mmol) in THF (1 L). The reaction mixture was stirred for 2 h at 0° C., then allowed to warm to room temperature. The reaction mixture was carefully poured into a Rochelle salt solution (1.5 L). EtOAc (1.5 L) was added and the resulting biphasic mixture was stirred for 1.5 h. The aqueous layer was separated and then extracted with EtOAc (2×1.5 L). The combined organic layer was dried over MgSO₄, filtered, and evaporated to give Intermediate 109 (108 g, yield: 87%) as a white solid, used without further purification.

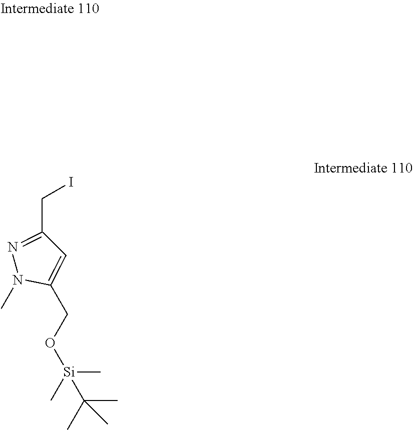 [SMILES: Cn1nc(CI)cc1CO[Si](C)(C)C(C)(C)C]

Intermediate 109 (81 g, 315.8 mmol), followed by methanesulfonic anhydride (71.5 g, 1.4 eq.), were added to a solution of DIPEA (61.2 g, 1.5 eq.) in THF (900 mL) at 0° C. 20 The resulting mixture was stirred at 0° C. for 5 min, then at room temperature for 30 min. NaI (213 g, 4.5 eq.) was then added to the reaction mixture and it was stirred at 50° C. for 2 h. After cooling, the solvent was evaporated. The residue was partitioned between EtOAc and water. The organic layer was separated and the aqueous layer was extracted with EtOAc. The combined organic layer was washed with brine, dried over Na₂SO₄, filtered, and evaporated. The residue was purified by flash column chromatography on silica gel (petroleum ether/EtOAc 4/1) to afford Intermediate 110 (30 g, yield: 26%) as a yellow oil.

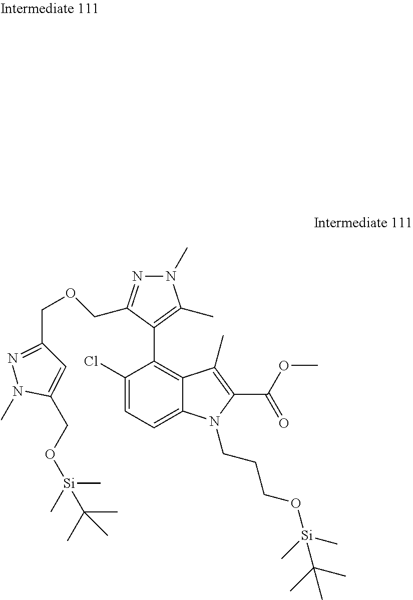 [SMILES: COC(=O)c1c(C)c2c(-c3c(COCc4cc(CO[Si](C)(C)C(C)(C)C)n(C)n4)nn(C)c3C)c(Cl)ccc2n1CCCO[Si](C)(C)C(C)(C)C]

NaH (60% in mineral oil, 415 mg, 1.2 eq.) was added at 0° C. to a solution of Intermediate 5 (4.5 g, 8.65 mmol) in anhydrous THE (90 mL) under nitrogen atmosphere. The reaction mixture was stirred at 0° C. for 30 min before addition of a solution of Intermediate 110 (3.80 g, 1.2 eq.) in THF (10 mL). After stirring at 0° C. for 10 min, the mixture was warmed to room temperature and stirred for 4 h. The reaction was quenched by addition of a solution of saturated aqueous NH₄Cl and EtOAc was added. The organic layer was separated, dried over Na₂SO₄, filtered, and concentrated under reduced pressure. The residue was purified by silica gel chromatography (hexane/EtOAc 100/0 to 20/80) to afford Intermediate 111 (5 g, yield: 76%) as yellow oil.

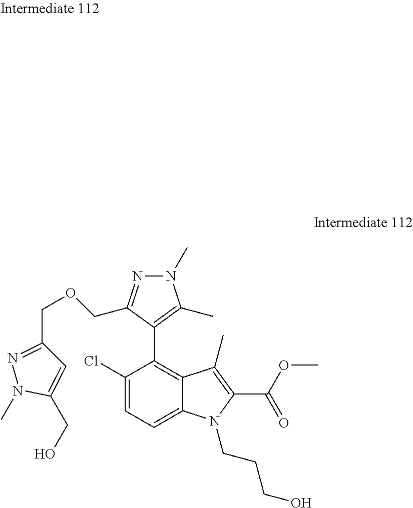

pTsOH.H₂O (2.89 g, 2.4 eq.) was added at 0° C. to a solution of Intermediate 111 (4.8 g, 6.33 mmol) in MeOH (100 mL) under nitrogen atmosphere. The reaction mixture was stirred at 0° C. for 10 min. Then the reaction mixture was warmed to room temperature and stirred for 3 h before being quenched with water (50 mL). The volatiles were removed under reduced pressure and the aqueous residue was extracted with DCM (3×50 mL). The combined organic layer was dried over Na₂SO₄, filtered, and evaporated. The residue was purified by column chromatography on silica gel (MeOH/DCM 0/100 to 10/90) to afford Intermediate 112 (3.2 g, yield: 94%) as a white solid.

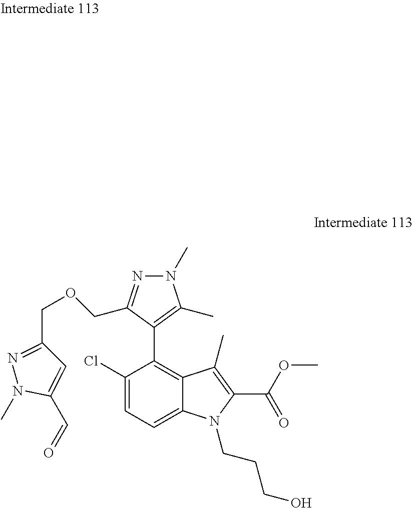

Activated MnO₂ (7.8 g, 15 eq.) was added at 0° C. to a solution of Intermediate 112 (3.2 g, 6.04 mmol) in DCM (100 mL) under nitrogen atmosphere. The reaction mixture was stirred at room temperature overnight. It was then filtered and the filter pad was washed with DCM (200 mL). The combined filtrate was concentrated in vacuo to afford Intermediate 113 (3.3 g, yield: 90%) as a yellow oil, used without further purification.

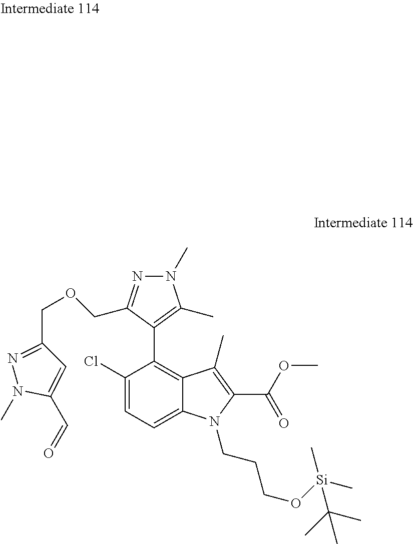 [SMILES: COC(=O)c1c(C)c2c(-c3c(COCc4cc(C=O)n(C)n4)nn(C)c3C)c(Cl)ccc2n1CCCO[Si](C)(C)C(C)(C)C]

TBDMSCl (548 mg, 1.2 eq.), followed by imidazole (248 mg, 1.2 eq.) were added to a solution of Intermediate 113 (1.6 g, 3.03 mmol) in DCM (15 mL) and the reaction mixture was stirred at room temperature for 4 h under nitrogen atmosphere. The reaction mixture was filtered through a Celite pad, and the filtrate was concentrated under reduced pressure. The residue was combined with a residue coming from the same reaction performed with another batch of Intermediate 113. The combined residue was purified by flash column chromatography on silica gel (petroleum ether/EtOAc 2/1) to afford Intermediate 114 (2.7 g) as a yellow oil.

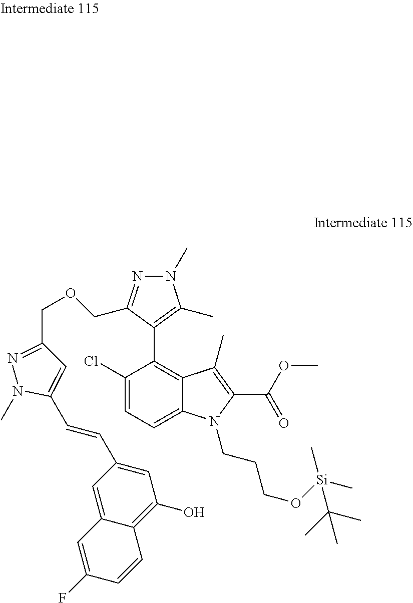 [SMILES: COC(=O)c1c(C)c2c(-c3c(COCc4cc(/C=C/c5cc(O)c6ccc(F)cc6c5)n(C)n4)nn(C)c3C)c(Cl)ccc2n1CCCO[Si](C)(C)C(C)(C)C]

NaH (60% in mineral oil, 146 mg, 1.5 eq.) was added to a solution of Intermediate 114 (2.6 g, 4.048 mmol) and Intermediate 107 (3.2 g, 4.45 mmol) in THF (30 mL) cooled to 0° C. under nitrogen atmosphere. The reaction mixture was stirred at room temperature for 48 h, then it was quenched with aqueous NH₄Cl (100 mL) and extracted with Et₂O (3×100 mL). The organic layer was dried over Na₂SO₄, filtered, and evaporated. The residue was purified by flash column chromatography on silica gel (petroleum ether/EtOAc, 1/1) to afford Intermediate 115 (2 g, yield: 62%) as a yellow oil.

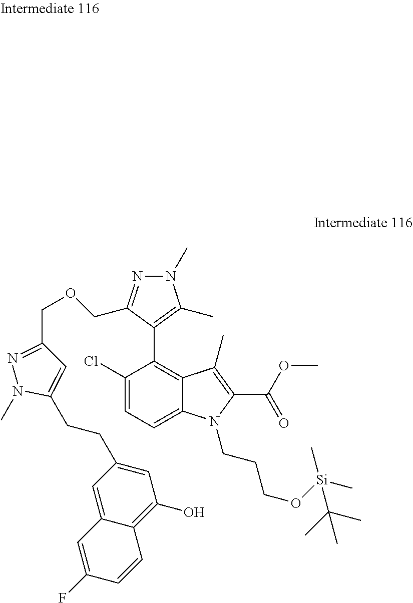 [SMILES: COC(=O)c1c(C)c2c(-c3c(COCc4cc(CCc5cc(O)c6ccc(F)cc6c5)n(C)n4)nn(C)c3C)c(Cl)ccc2n1CCCO[Si](C)(C)C(C)(C)C]

Pd/C (2 g, 1 eq.) was added to a solution of Intermediate 115 (2 g, 2.5 mmol) in EtOAc 15 (100 mL). The reaction mixture was stirred at 35° C. for 16 h under an hydrogen atmosphere, then it was filtered through a Celite pad. The filtrate was concentrated under reduced pressure to afford Intermediate 116 (1.9 g, yield: 95%) as a yellow oil, used without further purification.

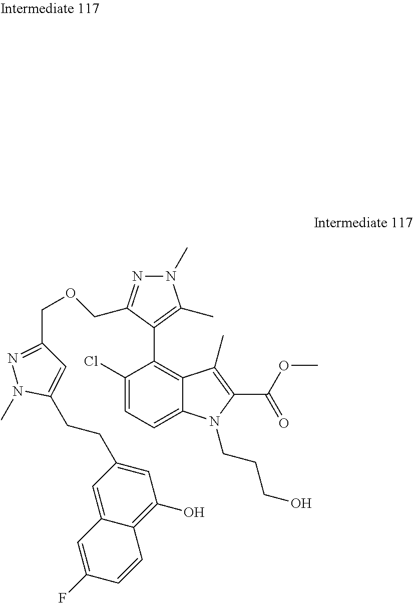

pTsOH.H₂O (1 g, 1.1 eq.) was added to a solution of Intermediate 116 (4.9 g, 4.71 mmol) in MeOH (100 mL) at room temperature. The reaction mixture was stirred at room temperature for 1 h. The reaction was quenched by addition of water. The mixture was extracted with Et₂O. The organic layer was washed with brine (100 mL) followed by aqueous NaHCO₃ (100 mL). The organic layer was dried over Na₂SO₄ and concentrated under reduced pressure. The residue was purified by flash column chromatography on silica gel (DCM/MeOH 20/1) to afford Intermediate 117 (1.2 g, yield: 37%) as a yellow oil.

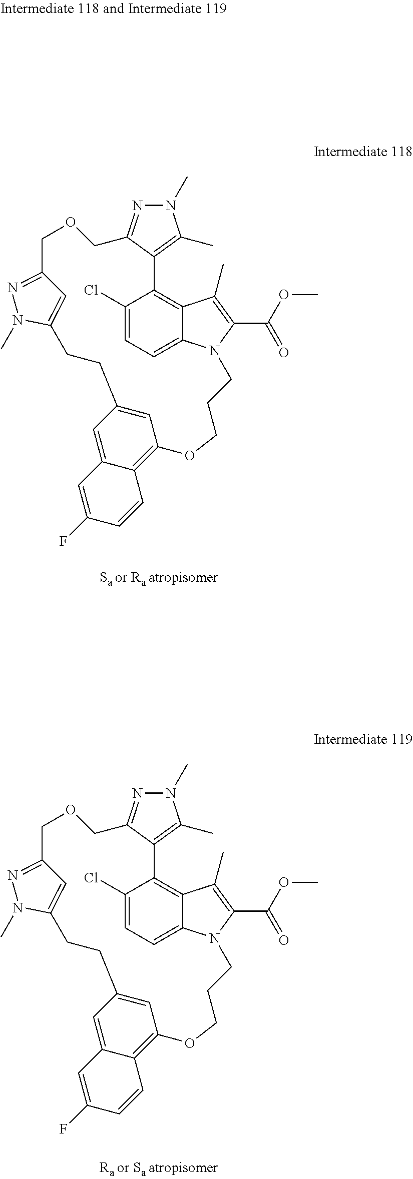

DTBAD (502 mg, 1.5 eq.) was added to a solution of Intermediate 117 (1 g, 1.453 mmol) in THF (2 mL) and toluene (15 mL). The resulting mixture was filled with nitrogen, stirred for 15 min at room temperature, and then added dropwise to a solution of PPh₃ (572 mg, 1.5 eq.) in toluene (5 mL) at 70° C. under nitrogen atmosphere. The reaction mixture was stirred for 15 min at 70° C. under nitrogen atmosphere. After cooling, the reaction mixture was concentrated under reduced pressure. The residue was purified by reverse phase chromatography (Column: C18 spherical, 20-35 μm, 100A, 330 g; Mobile Phase A: ACN, Mobile Phase B: H₂O (0.05% 0.5 M NH₄HCO₃—H₂O); Gradient: A/B 40/60 to 100/0) to afford the racemic mixture of Intermediate 118 and Intermediate 119. The atropisomers were separated by preparative chiral SFC (Column: CHIRAL ART Cellulose-SB, 3×25 cm, 5 um; Mobile Phase A: CO₂, Mobile Phase B: MeOH (0.5% 2 M NH₃ in MeOH) to afford Intermediate 118 (90 mg, yield: 9%) and Intermediate 119 (110 mg, yield: 11%), both as white solids.

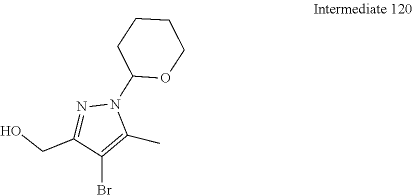

Lithium borohydride (32.2 g, 4 eq.) was added slowly to a solution of 1H-pyrazole-3-carboxylic acid, 4-bromo-5-methyl-1-(tetrahydro-2H-pyran-2-yl)-, ethyl ester (CAS [2246368-58-9]) (130 g, 369.7 mmol) in 2-Me-THF (1 L) at 0° C. The reaction mixture was allowed to warm to room temperature and was left stirring at room temperature overnight. The reaction was quenched by addition of water (800 mL). The mixture was extracted with EtOAc (800 mL×2). The combined organic layer was washed with brine (500 mL), dried with Na₂SO₄, filtered, and evaporated to afford Intermediate 120 (105 g, yield: 94%) as a white solid.

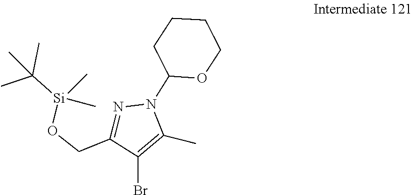 [SMILES: Cc1c(Br)c(CO[Si](C)(C)C(C)(C)C)nn1C1CCCCO1]

DMAP (16.28 g, 0.4 eq.) and Et₃N (92.38 mL, 2 eq.) were added to a solution of Intermediate 120 (100 g, 333.2 mmol) in THF (1 L). TBDMSCl (75.3 g, 1.5 eq.) was added at room temperature and the reaction mixture was stirred for 16 h. The reaction was quenched by addition of saturated aqueous NaHCO₃ (800 mL) and the mixture was extracted with EtOAc (1 L×2). The combined organic layer was washed with brine (800 mL), dried with Na₂SO₄, filtered, and evaporated. The residue was purified by column chromatography over silica gel (petroleum ether/EtOAc 100/0 to 30/70) to afford Intermediate 121 (130 g, yield: 94%) as a colorless oil.

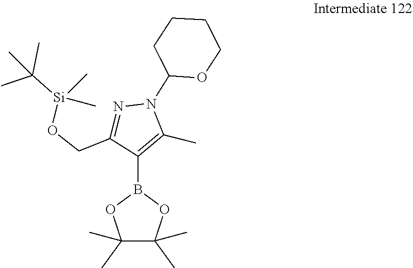 [SMILES: Cc1c(B2OC(C)(C)C(C)(C)O2)c(CO[Si](C)(C)C(C)(C)C)nn1C1CCCCO1]

nBuLi (104.55 mL, 1 eq.) was slowly added to a solution of Intermediate 121 (108 g, 261.4 mmol) in THF (1 L) at −78° C., under nitrogen atmosphere, and the reaction mixture was stirred at −78° C. for 1 h. Then, 2-isopropoxy-4,4,5,5-tetramethyl-1,3,2-dioxaborolane (97.2 g, 2 eq.) was added slowly and the reaction mixture was stirred at room temperature for 2 h. Saturated aqueous NH₄Cl (800 mL) was added slowly to quench the reaction. The mixture was extracted with EtOAc (1 L×2). The combined organic layer was washed with brine (800 mL), dried with Na₂SO₄, filtered, and evaporated to afford Intermediate 122 (140 g, assumed quantitative) as a yellow oil.

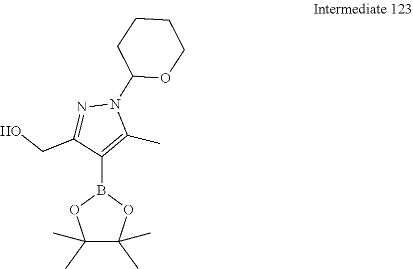 [SMILES: Cc1c(B2OC(C)(C)C(C)(C)O2)c(CO)nn1C1CCCCO1]

TBAF (1 M in THF, 192.4 mL, 1.2 eq.) was added dropwise to a solution of Intermediate 122 (70 g, 160 mmol) in DCM (700 mL) at room temperature under nitrogen atmosphere. The reaction mixture was stirred overnight at room temperature. The reaction mixture was added to a stirring solution of saturated aqueous NaHCO₃ (500 mL) and this mixture was extracted with EtOAc (700 mL×2). The combined organic layer was washed with brine (500 mL), dried with Na₂SO₄, filtered, and evaporated. The residue was purified by column chromatography over silica gel (petroleum ether/EtOAc 100/0 to 50/50) to afford Intermediate 123 (35 g, yield: 62%) as a white solid.

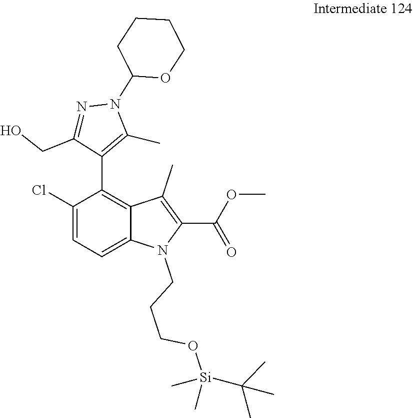 [SMILES: COC(=O)c1c(C)c2c(-c3c(CO)nn(C4CCCCO4)c3C)c(Cl)ccc2n1CCCO[Si](C)(C)C(C)(C)C]

K₂CO₃ (6.9 g, 2 eq.) was added to a solution of Intermediate 55 (12 g, 24.9 mmol) and Intermediate 123 (9.6 g, 1.2 eq.) in water (40 mL) and dioxane (200 mL). Pd(amphos)₂Cl₂ (CAS [887919-35-9]) (0.8 g, 0.05 eq.) was added under nitrogen atmosphere and the reaction mixture was stirred at 60° C. for 2 h. Water (40 mL) was added to the mixture and it was extracted with EtOAc (60 mL×2). The combined organic layer was washed with brine, dried with Na₂SO₄, filtered, and evaporated. The residue was purified by flash column chromatography over silica gel (petroleum ether/EtOAc 100/0 to 60/40) to afford Intermediate 124 (15 g, yield: 99%) as a yellow solid.

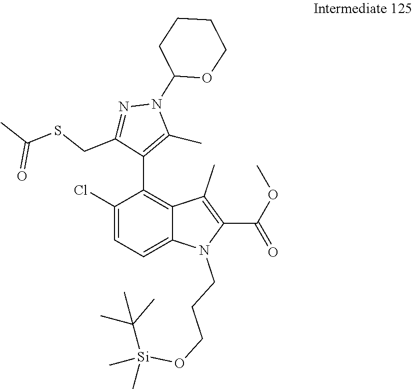 [SMILES: COC(=O)c1c(C)c2c(-c3c(CSC(C)=O)nn(C4CCCCO4)c3C)c(Cl)ccc2n1CCCO[Si](C)(C)C(C)(C)C]

Et₃N (5.1 mL, 1.5 eq.) followed by MsCl (2.4 mL, 1.25 eq.) were added dropwise to a solution of Intermediate 124 (14.5 g, 24.567 mmol) in dry THE (180 mL) (degassed by bubbling nitrogen for 15 min) at 0° C. under nitrogen atmosphere. The reaction mixture was stirred for 10 min at room temperature. Then, a degassed solution (degassed by bubbling nitrogen for 30 min) of potassium thioacetate (28.1 g, 10 eq.) in DMF (400 mL) (previously degassed by bubbling nitrogen for 30 min) was added at room temperature. The resulting mixture was degassed by bubbling nitrogen for 5 min and was then stirred at room temperature for 30 min. The reaction mixture was diluted with EtOAc (500 mL) and water (300 mL). The layers were separated and the aqueous layer was extracted with EtOAc (2×500 mL). The combined organic layer was washed with brine (3×300 mL), dried over Na₂SO₄, filtered, and concentrated. The residue was purified by silica gel chromatography (EtOAc/petroleum ether 0/100 to 30/70) to afford Intermediate 125 (15.3 g, yield: 96%) as a brown oil.

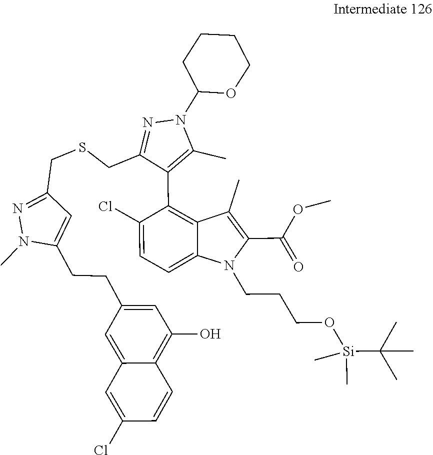 [SMILES: COC(=O)c1c(C)c2c(-c3c(CSCc4cc(CCc5cc(O)c6ccc(Cl)cc6c5)n(C)n4)nn(C4CCCCO4)c3C)c(Cl)ccc2n1CCCO[Si](C)(C)C(C)(C)C]

Intermediate 96 (8.005 g, 1.2 eq.) was added to a solution of Intermediate 125 (7.54 g, 11.63 mmol) in MeOH (100 mL). The reaction mixture was degassed and re-filled with nitrogen five times. Then, K₂CO₃ (3.215 g, 2 eq.) was added. The resulting mixture was stirred at room temperature overnight. The reaction mixture was concentrated under reduced pressure. The residue was diluted with EtOAc (300 mL) and water (300 mL).

The layers were separated and the aqueous layer was extracted with EtOAc (2×300 mL). The combined organic layer was washed with brine (3×300 mL), dried over Na₂SO₄, filtered, and concentrated. The residue was purified by silica gel chromatography (EtOAc/petroleum ether 25/75 to 50/50) to afford Intermediate 126 (7 g, yield: 66%) as a yellow solid.

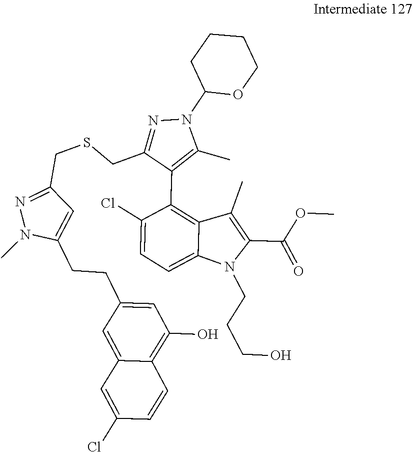

Et₃N.(HF)₃ (1.857 g, 1.5 eq.) was added to a solution of Intermediate 126 (6.95 g, 7.679 mmol) in THF (70 mL) at room temperature under nitrogen atmosphere. The reaction mixture was stirred at room temperature for 18 h. The reaction mixture was diluted with EtOAc (200 mL) and water (200 mL). The layers were separated and the aqueous layer was extracted with EtOAc (2×200 mL). The combined organic layer was washed with brine (2×100 mL), dried over Na₂SO₄, filtered, and concentrated to afford Intermediate 127 (6 g, yield: 99%) as a light yellow solid, used without further purification.

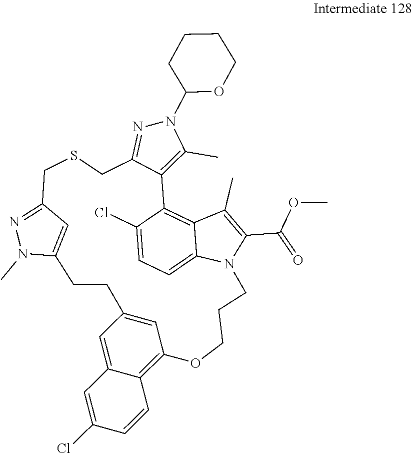

DTBAD (6.988 g, 4 eq.) was added to a solution of Intermediate 127 (6 g, 7.587 mmol) in THF (40 mL) and toluene (80 mL) (both degassed and re-filled with nitrogen five times). The reaction mixture was stirred for 15 min at room temperature. Then, this solution was added dropwise to a solution of PPh₃ (7.960 mg, 4 eq.) in toluene (80 mL) at 70° C. under nitrogen atmosphere. The reaction mixture was stirred for 10 min at 70° C. under nitrogen atmosphere. After cooling, the reaction mixture was diluted with water (150 mL) and EtOAc (3×200 mL). The layers were separated and the organic layer was washed with brine (3×200 mL), dried over Na₂SO₄, filtered, and concentrated. The residue was purified by silica gel chromatography (EtOAc/petroleum ether 25/75 to 70/30) to afford Intermediate 128 (5 g, yield: 85%) as a yellow solid.

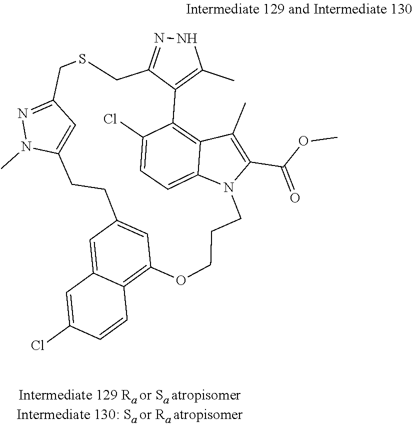

Intermediate 128 (5 g, 6.470 mmol) was dissolved in a 4 M solution of HCl in 1,4-dioxane (30 mL). The reaction mixture was stirred at room temperature for 2 h. The reaction mixture was then concentrated under reduced pressure. The residue was purified by reverse phase chromatography (ACN/H₂O—5 mmol NH₄HCO₃, 50/50 to 90/10) to afford the racemic mixture of Intermediate 129 and Intermediate 130 as a light yellow solid. This solid was separated into its atropisomers by preparative chiral SFC (Column: CHIRAL ART Cellulose-SB, 3×25 cm, 5 um; Mobile Phase A: CO₂, Mobile Phase B: IPA (0.5% 2 M NH₃-MeOH); A/B 50/50) to afford Intermediate 129 (800 mg, yield: 18%) and Intermediate 130 (800 mg, yield: 18%)

Intermediate 129: OR: [a]=+18.6° (589 nm, 28.7° C., 5.0 mg in 10 mL MeOH).

Intermediate 130: OR: [a]=−23.9° (589 nm, 28.7° C., 5.0 mg in 10 mL MeOH).

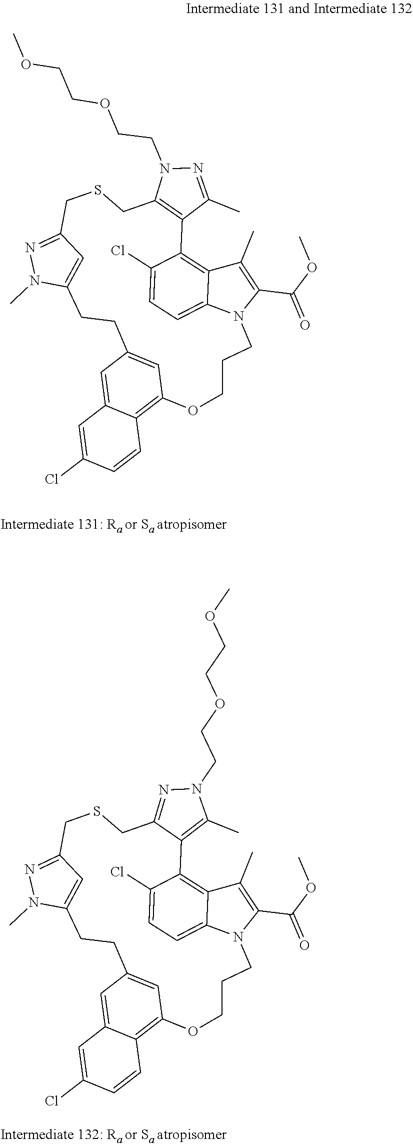 [SMILES: COCCOCCn1nc(C)c2c1CSCc1cc(n(C)n1)CCc1cc(c3ccc(Cl)cc3c1)OCCCn1c(C(=O)OC)c(C)c3c-2c(Cl)ccc31 ; COCCOCCn1nc2c(c1C)-c1c(Cl)ccc3c1c(C)c(C(=O)OC)n3CCCOc1cc(cc3cc(Cl)ccc13)CCc1cc(nn1C)CSC2]

Intermediate 131: Rₐ or Sₐ atropisomer Intermediate 132: Rₐ or Sₐ atropisomer Cs2CO₃ (397 mg, 3 eq.) was added to a solution of Intermediate 129 (280 mg, 0.407 mmol) in DMF (5 mL) under nitrogen atmosphere. 1-Bromo-2-(2-methoxyethoxy)ethane (223 mg, 3 eq.) was added and the resulting mixture was stirred at room temperature under nitrogen for 16 h. The reaction mixture was diluted with H₂O (20 mL) and EtOAc (20 mL). The layers were separated and the aqueous layer was extracted again with EtOAc (2×20 mL). The combined organic layer was washed with brine (3×20 mL), dried over Na₂SO₄, filtered, and concentrated to afford the mixture of Intermediate 131 and Intermediate 132 (300 mg) as a light yellow solid, used without further purification.

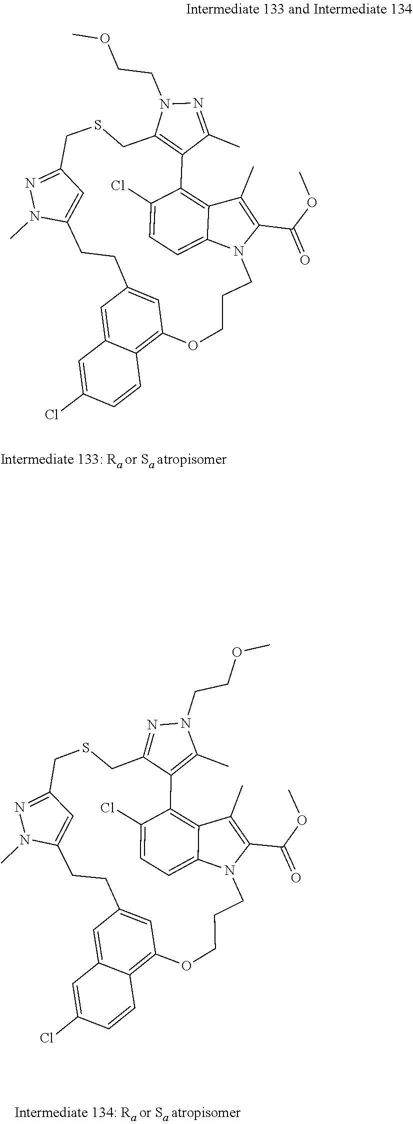 [SMILES: COCCn1nc(C)c2c1CSCc1cc(n(C)n1)CCc1cc(c3ccc(Cl)cc3c1)OCCCn1c(C(=O)OC)c(C)c3c-2c(Cl)ccc31 ; COCCn1nc2c(c1C)-c1c(Cl)ccc3c1c(C)c(C(=O)OC)n3CCCOc1cc(cc3cc(Cl)ccc13)CCc1cc(nn1C)CSC2]

The mixture of Intermediate 133 and Intermediate 134 was prepared according to the same procedure as for the mixture of Intermediate 131 and Intermediate 132, using 1-bromo-2-(2-methoxy)ethane instead of 1-bromo-2-(2-methoxyethoxy)ethane.

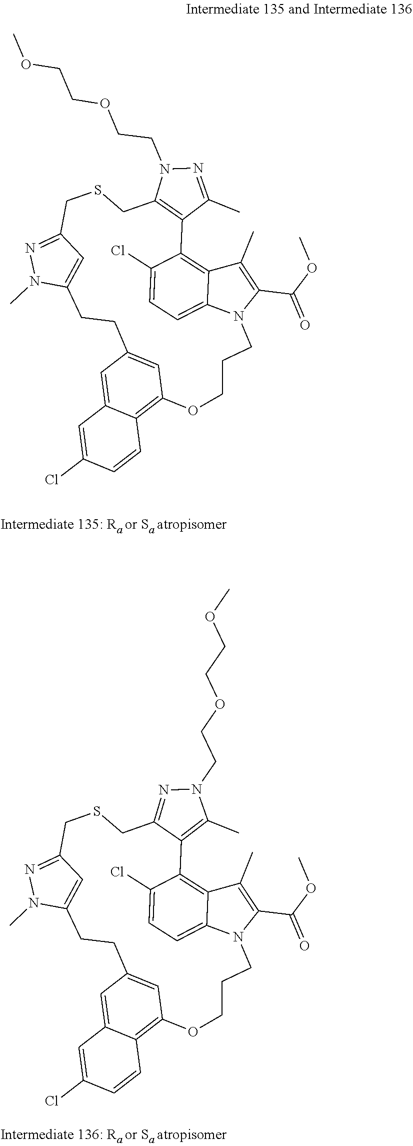 [SMILES: COCCOCCn1nc(C)c2c1CSCc1cc(n(C)n1)CCc1cc(c3ccc(Cl)cc3c1)OCCCn1c(C(=O)OC)c(C)c3c-2c(Cl)ccc31 ; COCCOCCn1nc2c(c1C)-c1c(Cl)ccc3c1c(C)c(C(=O)OC)n3CCCOc1cc(cc3cc(Cl)ccc13)CCc1cc(nn1C)CSC2]

The mixture of Intermediate 135 and Intermediate 136 was prepared according to the same procedure as for the mixture of Intermediate 131 and Intermediate 132, starting from Intermediate 130 instead of Intermediate 129.

The mixture of Intermediate 135 and Intermediate 136 was then separated by preparative chiral HPLC (Column: CHIRAL ART Cellulose-SC, 2×25 cm, 5 um; Mobile Phase A: hexane:DCM 3:1 (0.5% 2 M NH₃-MeOH), Mobile Phase B: EtOH; 95% A/5% B) to afford pure Intermediate 135 and Intermediate 136.

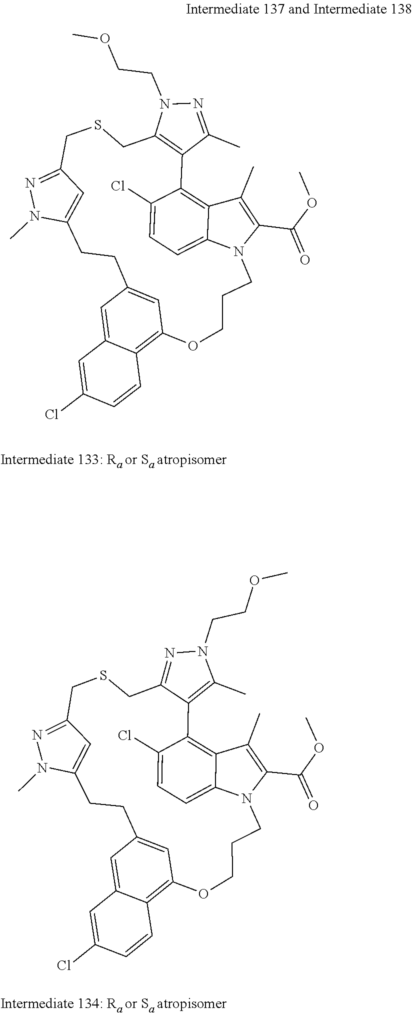 [SMILES: COCCn1nc(C)c2c1CSCc1cc(n(C)n1)CCc1cc(c3ccc(Cl)cc3c1)OCCCn1c(C(=O)OC)c(C)c3c-2c(Cl)ccc31 ; COCCn1nc2c(c1C)-c1c(Cl)ccc3c1c(C)c(C(=O)OC)n3CCCOc1cc(cc3cc(Cl)ccc13)CCc1cc(nn1C)CSC2]

The mixture of Intermediate 137 and Intermediate 138 was prepared according to the same procedure as for the mixture of Intermediate 133 and Intermediate 134, starting from Intermediate 130 instead of Intermediate 129.

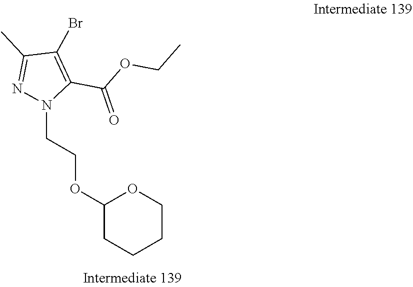 [SMILES: CCOC(=O)c1c(Br)c(C)nn1CCOC1CCCCO1]

Cyanomethylenetributylphosphorane (CAS [157141-27-0], 37.15 mL, 141.6 mmol, 1.5 eq.) was added dropwise to a solution of 1H-pyrazole-3-carboxylic acid, 4-bromo-5-methyl-, ethyl ester (CAS [6076-14-8], 22 g, 94.4 mmol) and 2-(tetrahydro-2H-pyran-2-yloxy)ethanol (CAS [2162-31-4], 15.7 mL, 113.3 mmol, 1.2 eq.) in THF (100 mL) at 0° C. and the mixture was stirred overnight at room temperature. The solvent was evaporated and the residue was taken up in EtOAc/water. The organic layer was separated, dried over MgSO₄, filtered, and evaporated. The residue was purified by column chromatography on silica gel (heptane/EtOAc, 100/0 to 80/20) to afford Intermediate 139 (17.2 g, yield: 50%).

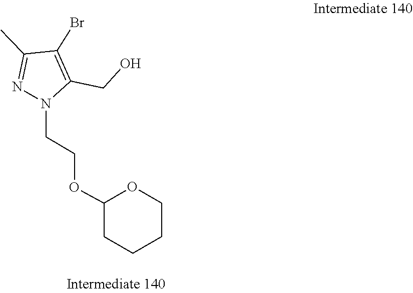 [SMILES: Cc1nn(CCOC2CCCCO2)c(CO)c1Br]

NaBH₄ (226 mg, 5.979 mmol, 2 eq.) was added to a solution of Intermediate 139 (1.08 g, 2.99 mmol) in THF (18 mL) and MeOH (4 mL) at 0° C. The reaction mixture was then stirred at room temperature for 24 h. To push the reaction to completion, more NaBH₄ (679 mg, 17.94 mmol, 6 eq.) was added and the reaction mixture was stirred at room temperature overnight. The reaction mixture was cooled to 0° C., treated with NH₄Cl and AcOEt, stirred for 15 min at room temperature, and extracted with more AcOEt. The combined organic layer was dried on MgSO₄, filtered, and evaporated to give Intermediate 140 (917 mg, yield: 96%), used without further purification.

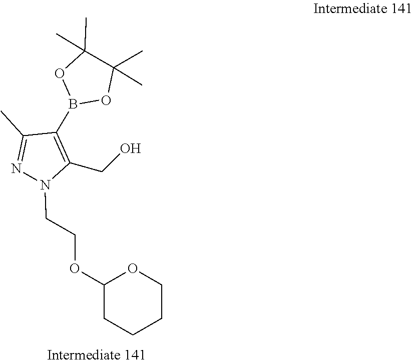 [SMILES: Cc1nn(CCOC2CCCCO2)c(CO)c1B1OC(C)(C)C(C)(C)O1]

Et₃N (7.708 mL, 55.451 mmol, 3 eq.) followed by pinacolborane (CAS [25015-63-8], 5.9 mL, 39.441 mmol, 2.1 eq.) were added dropwise to a nitrogen-degassed solution of Intermediate 140 (5.9 g, 18.484 mmol), bis(acetonitrile)dichloropalladium (II) (CAS [14592-56-4], 240 mg, 0.924 mmol, 0.05 eq.), and 2-dicyclohexylphosphino-2′,6′-dimethoxybiphenyl (CAS [657408-07-6], 1.518 g, 3.697 mmol, 0.2 eq.) in 1,4-dioxane (65 mL). The reaction mixture was stirred at 80° C. for 1 h. The mixture was diluted with of water (20 mL) and was extracted with EtOAc (3×). The combined organic layer was washed with water and brine, dried (MgSO₄), filtered, and evaporated. The residue was purified by column chromatography on silica gel (heptane/EtOAc, 100/0 to 50/50) to afford Intermediate 141 (4.7 g, yield: 69%).

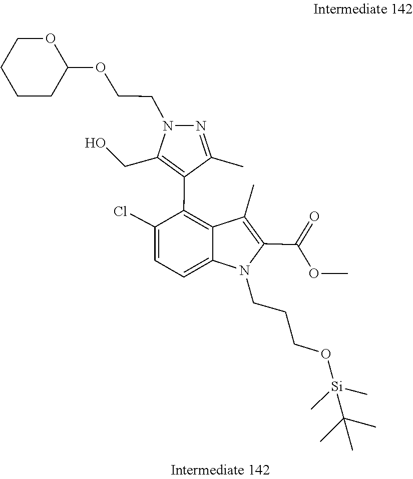 [SMILES: COC(=O)c1c(C)c2c(-c3c(C)nn(CCOC4CCCCO4)c3CO)c(Cl)ccc2n1CCCO[Si](C)(C)C(C)(C)C]

A 20 mL vial was charged with a solution of Intermediate 55 (1.23 g, 2.5 mmol) and Intermediate 141 (1.1 g, 3 mmol, 1.2 eq.) in 1,4-dioxane (15 mL) and this was purged with nitrogen for 15 min. Bis(di-tert-butyl(4-dimethylaminophenyl)phosphine)dichloropalladium(II) (CAS [887919-35-9], 88 mg, 0.12 mmol, 0.05 eq.) and a solution of K₂CO₃ (0.69 g, 5 mmol, 2 eq.) in water (3 mL) were added. The vial was capped and heated at 65° C. for 2 h. The reaction mixture was diluted with water and EtOAc. The layers were separated and the aqueous layer was extracted with EtOAc. The combined organic layer was dried with MgSO₄ and a little Norit, filtered, and concentrated in vacuo. The residue was purified by flash column chromatographyy (40 g Redisep Flash column eluting with heptane/EtOAc 100/0 to 50/50) to afford Intermediate 142 (1.13 g, yield: 71%) as a colorless oil.

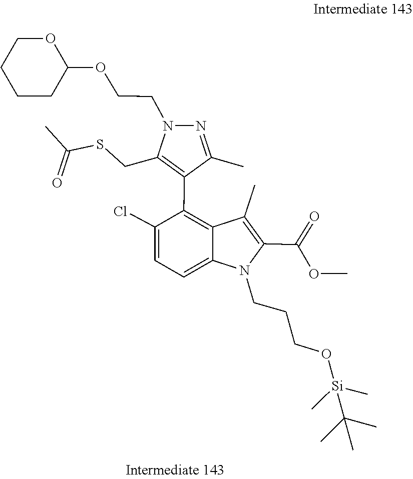 [SMILES: COC(=O)c1c(C)c2c(-c3c(C)nn(CCOC4CCCCO4)c3CSC(C)=O)c(Cl)ccc2n1CCCO[Si](C)(C)C(C)(C)C]

Methanesulfonyl chloride (175 μL, 2.24 mmol, 1.25 eq.) was added dropwise to an ice-cooled solution of Intermediate 142 (1.13 g, 1.78 mmol) and Et₃N (375 μL, 2.71 mmol, 1.5 eq.) in dry THF (15 mL). The ice bath was removed and stirring was continued for 30 min. A solution of potassium thioacetate (2.03 g, 17.82 mmol, 10 eq.) in dry DMF (30 mL) was added and the mixture was diluted with THF (15 mL). After 30 min at room temperature, the orange viscous solution was partitioned between saturated aqueous NaHCO₃ and EtOAc, and the layers were separated. The organic layer was washed with brine, dried on MgSO₄, filtered, and concentrated in vacuo. The residue was purified by flash column chromatography (40 g Redisep column eluting with heptane/EtOAc 100/0 to 50/50) to afford Intermediate 143 (1.22 g, yield: 100%) as a tan oil.

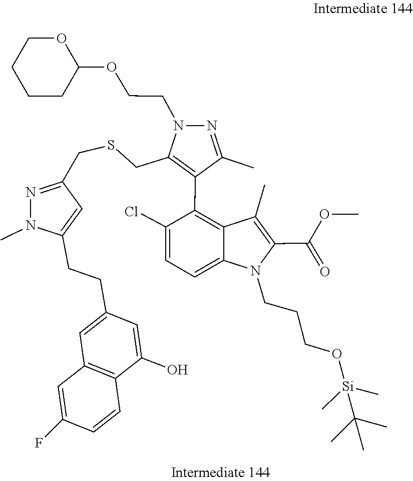 [SMILES: COC(=O)c1c(C)c2c(-c3c(C)nn(CCOC4CCCCO4)c3CSCc3cc(CCc4cc(O)c5ccc(F)cc5c4)n(C)n3)c(Cl)ccc2n1CCCO[Si](C)(C)C(C)(C)C]

A solution of Intermediate 143 (1.23 g, 1.78 mmol), Intermediate 25a (1.19 g, 2.13 mmol, 1.2 eq.), and triphenylphosphine (49 mg, 0.19 mmol, 0.1 eq.) in MeOH (110 mL) was degassed and re-filled with nitrogen three times. The suspension was cooled to 0° C. before addition of K₂CO₃ (0.75 g, 5.43 mmol, 3 eq.). The reaction mixture was degassed with nitrogen again and was stirred at room temperature for 3.5 h. The reaction mixture was concentrated under reduced pressure and the resulting slurry was partitioned between water and EtOAc. The layers were separated and aqueous layer was extracted with EtOAc (3×). The combined organic layer was washed with brine, dried over MgSO₄, filtered, and concentrated under reduced pressure to afford Intermediate 144 (833 mg, yield: 50%), used without further purification.

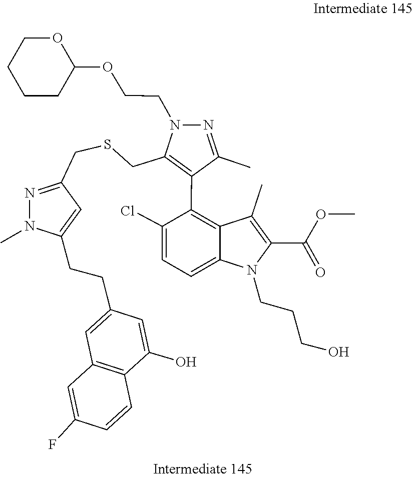

TBAF (1M in THF, 1.33 mL, 1.33 mmol, 1.5 eq.) was added to a solution of Intermediate 144 (0.83 g, 0.88 mmol) in THF (20 mL) at 0° C. The reaction mixture was stirred at room temperature for 4.5 h. After cooling to 0° C., the reaction mixture was treated with saturated aqueous NH₄Cl and was stirred for 15 min. The mixture was extracted with EtOAc (3×). The combined organic layer was washed with brine, dried (MgSO₄), filtered, and evaporated. The residue was purified by flash column chromatography on silica gel (DCM/MeOH, 100/0 to 95/5) to afford Intermediate 145 (410 mg, yield: 57%) as a off-white foam.

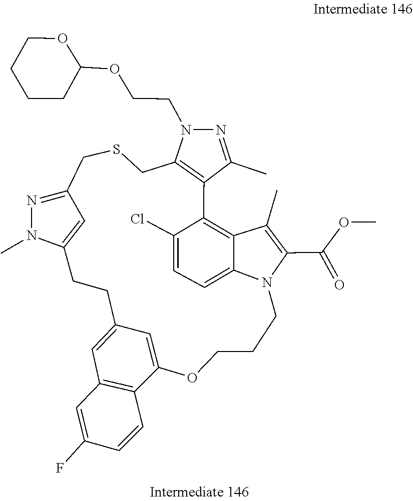

A solution of Intermediate 145 (3.89 g, 0.0048 mol) and di-tert-butyl azodicarboxylate (4.5 g, 0.02 mol, 4.1 eq.) in previously nitrogen-degassed THF/toluene (10 mL/50 mL) was added dropwise via a syringe pump (0.3 mL/min) to a previously thoroughly nitrogen-degassed solution of triphenylphosphine (5.1 g, 0.019 mol, 4.1 eq.) in toluene (600 mL), stirring at 70° C. When the addition was complete, the solution was cooled to room temperature and concentrated in vacuo. The residue was purified by flash column chromatography (220 g Redisep flash column, DCM/MeOH 100/0 to 98/2) to afford Intermediate 146 (1.56 g, yield: 41%) as a tan oil.

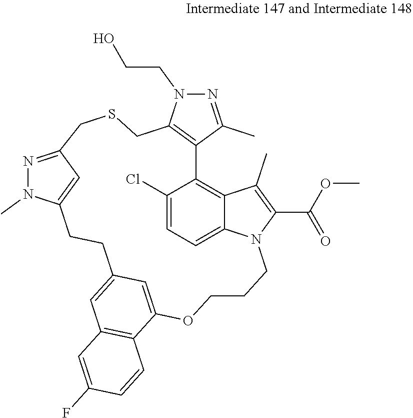

p-Toluenesulfonic acid monohydrate (0.56 g, 2.92 mmol, 1.5 eq.) was added to a solution of Intermediate 146 (1.56 g, 1.95 mmol) in MeOH (50 mL) and the reaction mixture was stirred at room temperature for 16 h. The solvent was evaporated and the residual oil was partitioned between DCM and saturated aqueous NaHCO₃. The layers were separated and the organic layer was dried over MgSO₄, filtered, and concentrated in vacuo. The residue was purified by preparative SFC (Stationary phase: Chiralpak Daicel IG 20×250 mm, Mobile phase: CO₂, EtOH+0.4% iPrNH₂) to afford Intermediate 147 (502 mg, yield: 36%) and Intermediate 148 (476 mg, yield: 34%) both as white solids.

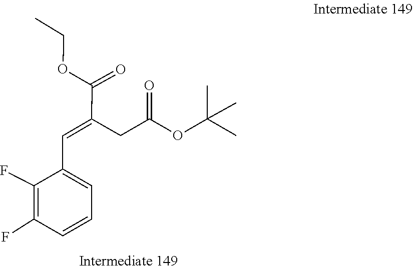 [SMILES: CCOC(=O)/C(=C/c1cccc(F)c1F)CC(=O)OC(C)(C)C]

NaH (60% in mineral oil, 61.9 g, 1548.2 mmol, 1.1 eq.) was added to a solution of 4-(tert-butyl) 1-ethyl 2-(diethoxyphosphoryl)succinate (CAS [77924-28-8], 523.8 g, 1548.2 mmol, 1.1 eq.) in THF (3500 mL) at 0° C. The resulting solution was stirred at 0° C. for 1 h. Then, 2,3-difluorobenzaldehyde (200 g, 1407.4 mmol), dissolved in TF (1500 mL), was added to the solution and the reaction mixture was stirred at room temperature for 3 h. The reaction was quenched by addition of cold water (2000 mL).

The resulting mixture was extracted with EtOAc (3×3000 mL). The combined organic layer was dried over Na₂SO₄, filtered, and concentrated to afford Intermediate 149 (538 g, assumed quantitative) as a yellow oil, used without further purification.

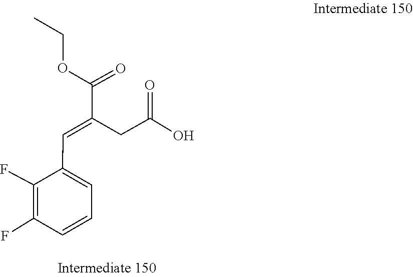 [SMILES: CCOC(=O)/C(=C/c1cccc(F)c1F)CC(=O)O]

Intermediate 149 (538 g, 1648.6 mmol) was dissolved in TFA (2000 mL) and the reaction mixture was stirred at room temperature for 1 h. The reaction mixture was concentrated under reduced pressure. Toluene was added and evaporated under reduced pressure to afford Intermediate 150 (533 g, assumed quantitative) as a yellow solid, used without further purification.

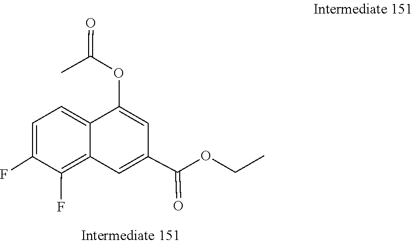 [SMILES: CCOC(=O)c1cc(OC(C)=O)c2ccc(F)c(F)c2c1]

NaOAc (161.8 g, 1972.4 mmol, 1 eq.) was added to a solution of Intermediate 150 (533 g, 1972.4 mmol) in acetic anhydride (3600 mL). The resulting solution was stirred at 130° C. for 1 h. After cooling down to room temperature, the reaction mixture was concentrated under reduced pressure. The residue was diluted with water (1000 mL) and extracted with EtOAc (3×3000 mL). The combined organic layer was dried over Na₂SO₄, filtered, and concentrated. The residue was purified by silica gel chromatography (EtOAc/petroleum ether 0/100 to 30/70) to afford Intermediate 151 (190 g, yield: 33%) as a yellow solid.

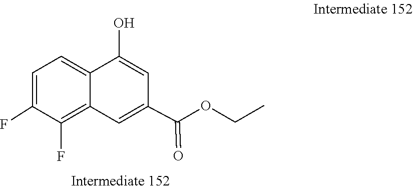 [SMILES: CCOC(=O)c1cc(O)c2ccc(F)c(F)c2c1]

K₂CO₃ (75.86 g, 548.85 mmol, 1.7 eq.) was added to a solution of Intermediate 151 (95 g, 322.85 mmol) in EtOH (1500 mL). The resulting solution was stirred at room temperature for 1 h. The solution was filtered and concentrated under reduced pressure.

Aqueous HCl (0.5 M, 500 mL) was added to the residue and the mixture was extracted with EtOAc (3×2000 mL). The combined organic layer was dried over Na₂SO₄, filtered, and concentrated to afford Intermediate 152 (70.4 g, yield: 86%) as a yellow solid, used without further purification.

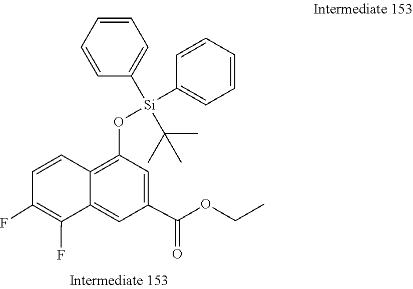 [SMILES: CCOC(=O)c1cc(O[Si](c2ccccc2)(c2ccccc2)C(C)(C)C)c2ccc(F)c(F)c2c1]

Tert-butylchlorodiphenylsilane (92.066 g, 334.955 mmol, 1.2 eq.) and DMAP (6.820 g, 55.826 mmol, 0.2 eq.) were added to a solution of Intermediate 152 (70.4 g, 279.129 mmol) in THF (1500 mL) under nitrogen atmosphere. Imidazole (28.471 g, 418.694 mmol, 1.5 eq.) was then added. The resulting solution was stirred at 50° C. for 16 h.

After cooling down to room temperature, the reaction was quenched with water (500 mL). The resulting mixture was extracted with EtOAc (3×1000 mL). The combined organic layer was combined, dried over Na₂SO₄, filtered, and concentrated. The residue was purified by silica gel chromatography (EtOAc/petroleum ether 0/100 to 20/80) to afford Intermediate 153 (114 g, yield: 83%) as a yellow solid.

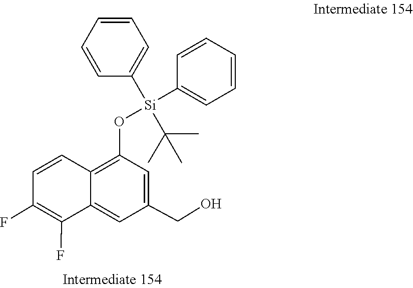 [SMILES: CC(C)(C)[Si](Oc1cc(CO)cc2c(F)c(F)ccc12)(c1ccccc1)c1ccccc1]

LiAlH₄ (10.596 g, 278.835 mmol, 1.2 eq.) dissolved in THF (200 mL) was added to a solution of Intermediate 153 (114 g, 232.362 mmol) in THF (1500 mL) at 0° C. The resulting solution was stirred at room temperature for 1 h. The reaction was quenched by addition of sodium sulfate decahydrate. The resulting mixture was filtered and the filter cake was washed with EtOAc (3×1000 mL). The combined organic layer was concentrated to afford Intermediate 154 (94.6 g, yield: 91%) as a white solid, used without further purification.

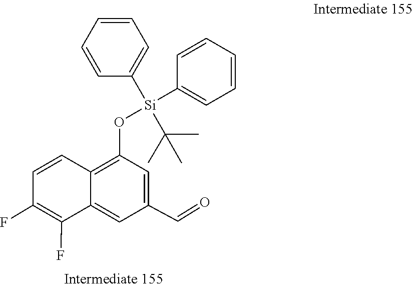 [SMILES: CC(C)(C)[Si](Oc1cc(C=O)cc2c(F)c(F)ccc12)(c1ccccc1)c1ccccc1]

Dess-Martin periodinane (CAS [87413-09-0], 267.773 g, 631.331 mmol, 3 eq.) was added to a solution of Intermediate 154 (94.4 g, 210.444 mmol) in DCM (1500 mL). The resulting mixture was stirred at room temperature for 1 h. The reaction was quenched by addition of saturated aqueous sodium thiosulfate (1000 mL). The resulting mixture was extracted with DCM (3×2000 mL). The combined organic layer was dried over Na₂SO₄, filtered, and concentrated. The residue was purified by silica gel chromatography (petroleum ether/EtOAc 100/0 to 50/50) to afford Intermediate 155 (70 g, yield: 74%) as a white solid.

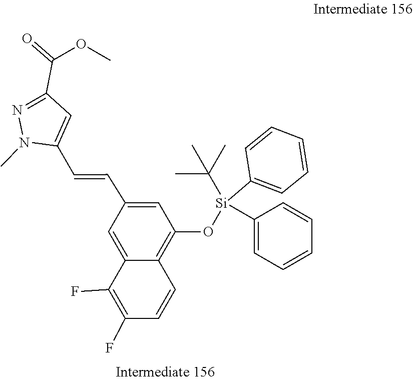 [SMILES: COC(=O)c1cc(/C=C/c2cc(O[Si](c3ccccc3)(c3ccccc3)C(C)(C)C)c3ccc(F)c(F)c3c2)n(C)n1]

Intermediate 105 (61.794 g, 137.047 mmol, 1.2 eq.) was added to a mixture of Intermediate 155 (51 g, 114.206 mmol) in THF (2 L). NaH (60% in mineral oil, 6.8 g, 171.309 mmol, 1.5 eq.) was added to the reaction mixture at 0° C. and the mixture was stirred at room temperature for 40 min. The reaction was quenched by addition of saturated aqueous NH₄Cl (2 L). The mixture was extracted with EtOAc (3×1 L). The combined organic layer was dried over Na₂SO₄, filtered, and concentrated under reduced pressure. The residue was purified by column chromatography on silica gel (petroleum ether/EtOAc 8/1) to afford Intermediate 156 (59 g, yield: 88%) as a white solid.

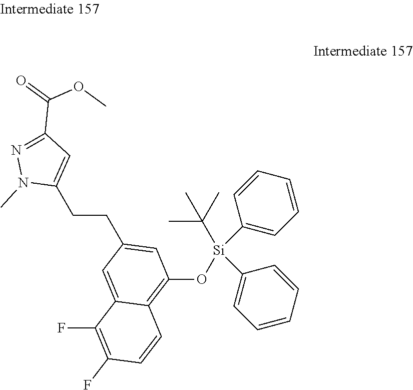 [SMILES: COC(=O)c1cc(CCc2cc(O[Si](c3ccccc3)(c3ccccc3)C(C)(C)C)c3ccc(F)c(F)c3c2)n(C)n1]

Pd/C (10%, 10 g, 0.17 eq.) was added to a solution of Intermediate 156 (58 g, 99.535 mmol) in EtOAc (1 L) and THF (200 mL). The mixture was stirred at 40° C. for 16 h under hydrogen atmosphere. The reaction mixture was filtered through a celite pad and the filtrate was concentrated under reduced pressure. The residue was purified by column chromatography (petroleum ether/EtOAc 5/1) to afford Intermediate 157 (38 g, yield: 65%) as a colorless oil, used without further purification.

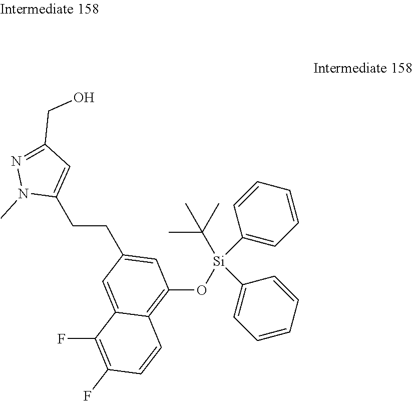 [SMILES: Cn1nc(CO)cc1CCc1cc(O[Si](c2ccccc2)(c2ccccc2)C(C)(C)C)c2ccc(F)c(F)c2c1]

LiAlH₄ (2.885 g, 75.933 mmol, 1.2 eq.) dissolved in THF (20 mL) was added to a solution of Intermediate 157 (37 g, 63.277 mmol) in THF (240 mL) at 0° C. The resulting solution was stirred at room temperature for 1 h. The reaction was quenched by addition of sodium sulfate decahydrate. The resulting mixture was filtered and the filter cake was washed with EtOAc (3×200 mL). The combined organic layer was concentrated and the residue was triturated with petroleum ether and diethyl ether to afford Intermediate 158 as a white solid (15.5 g, yield: 41%) used without further purification.

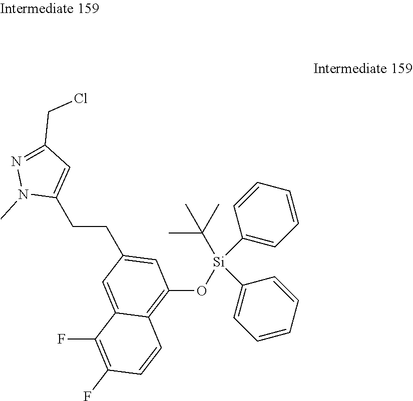 [SMILES: Cn1nc(CCl)cc1CCc1cc(O[Si](c2ccccc2)(c2ccccc2)C(C)(C)C)c2ccc(F)c(F)c2c1]

A solution of Intermediate 158 (1.0 g, 1.696 mmol) in dry DCM (15 mL) was cooled to 0° C. under nitrogen atmosphere. SOCl₂ (0.141 mL, 1.950 mmol, 1.15 eq.) was added dropwise and the reaction mixture was stirred at room temperature for 1 h. The reaction mixture was diluted with DCM (35 mL) and saturated aqueous NaHCO₃ (15 mL). The layers were separated and the organic one was washed with saturated aqueous NaHCO₃ (15 mL) and brine (15 mL). The organic layer was dried over MgSO₄, filtered, and concentrated under reduced pressure to give Intermediate 159 (1030 mg, yield: 98%) as a colorless paste, used without further purification.

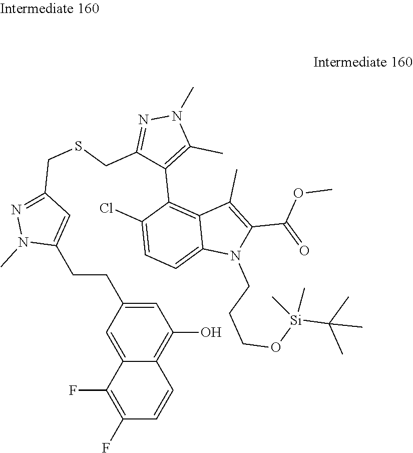 [SMILES: COC(=O)c1c(C)c2c(-c3c(CSCc4cc(CCc5cc(O)c6ccc(F)c(F)c6c5)n(C)n4)nn(C)c3C)c(Cl)ccc2n1CCCO[Si](C)(C)C(C)(C)C]

K₂CO₃ (620 mg, 4.496 mmol, 2 eq.) was added to a solution of Intermediate 8 (1.3 g, 2.248 mmol) and Intermediate 159 (1.4 g, 2.473 mmol, 1.1 equiv) in MeOH (30 mL) under nitrogen atmosphere. The reaction mixture was stirred at room temperature for 16 h. The reaction was quenched by adding water (50 mL). The resulting mixture was extracted with EtOAc (3×50 mL). The combined organic layer was dried over Na₂SO₄, filtered, and concentrated. The residue was purified by silica gel chromatography (petroleum ether/EtOAc 100/0 to 20/80) to afford Intermediate 160 as a yellow oil (1.7 g, yield: 90%).

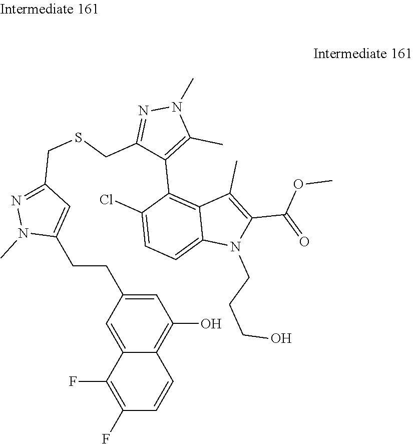

pTsOH.H₂O (375 mg, 1.972 mmol, 1.1 eq.) was added to a solution of Intermediate 160 (1.5 g, 1.793 mmol) in MeOH (30 mL). The reaction mixture was stirred at room temperature for 1.5 h. The solvent was evaporated and the residue was diluted with water and DCM. The layers were separated and the aqueous layer was extracted with DCM (40 mL×3). The combined organic layer was washed with aqueous NaHCO₃ (30 mL), brine (30 mL), dried over Na₂SO₄, filtered, and evaporated to afford Intermediate 161 (1.2 g, yield: 93%) as a white solid.

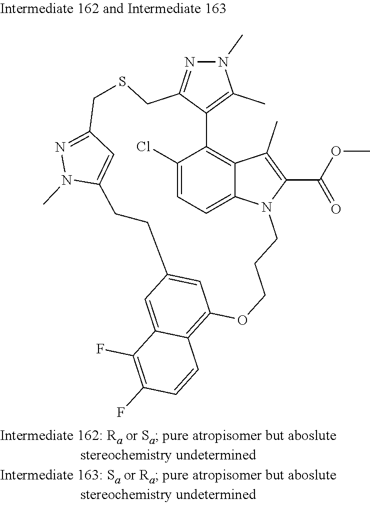

Intermediate 161 (1.05 g, 1.454 mmol) and DTBAD (502 mg, 2.181 mmol, 1.5 eq.) in toluene (10 mL) and THF (1 mL) was added dropwise over 10 min to a solution of triphenylphosphine (571 mg, 2.181 mmol, 1.5 eq.) in toluene (10 mL) at 70° C. under nitrogen atmosphere. After the addition was complete, the reaction mixture was further stirred at the same temperature for 10 min. The solvents were evaporated and the residue was extracted with DCM (10 mL×3). The combined organic layer was washed with brine (10 mL), dried over Na₂SO₄, filtered, and evaporated. The residue was purified by reverse phase flash chromatography (40-100% 0.05% NH₄HCO₃H₂O/CH₃CN) followed by preparative SFC (CHIRALPAK IG, 3*25 cm, 5 um; Mobile Phase A: CO₂, Mobile Phase B: IPA:ACN=1:1 (0.1% 2 M NH₃-MeOH); Gradient: 50% B) to afford Intermediate 162 (300 mg, yield: 29%) and Intermediate 163 (300 mg, yield: 29%), both as pale yellow foamy solids.

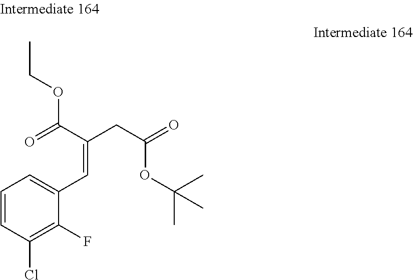 [SMILES: CCOC(=O)/C(=C\c1cccc(Cl)c1F)CC(=O)OC(C)(C)C]

A suspension of sodium hydride (27.7 g, 693.76 mmol, 1 eq.) in TF was added dropwise to a stirred solution of 4-(tert-butyl) 1-ethyl 2-(diethoxyphosphoryl)succinate (CAS [77924-28-8], 258.2 g, 763.13 mmol. 1.1 eq) in THF (1.5 L) at 0° C. The reaction mixture was stirred for 1 h at room temperature before 3-chloro-2-fluorobenzaldehyde (110 g, 693.8 mmol) was added at room temperature. The reaction was further stirred at room temperature for 3 h. The reaction was quenched by adding ice/water (500 mL) and the mixture was extracted by EtOAc (300 mL×3). The organic layer was dried over Na₂SO₄, filtered, and concentrated under reduced pressure to afford Intermediate 164 (237 g, assumed quantitative), used without further purification.

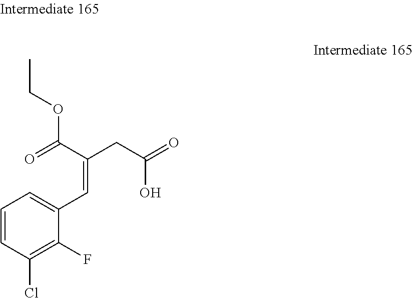 [SMILES: CCOC(=O)/C(=C\c1cccc(Cl)c1F)CC(=O)O]

A solution of Intermediate 164 (543 g, 1584 mmol) in TFA (1.5 L) was stirred at 25° C. for 1 h. The mixture was concentrated under reduced pressure to afford Intermediate 165 (454 g, assumed quantitative), used without further purification.

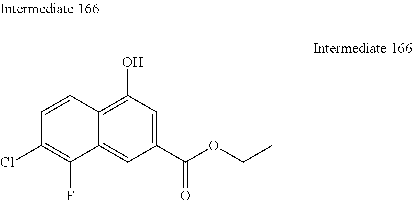 [SMILES: CCOC(=O)c1cc(O)c2ccc(Cl)c(F)c2c1]

Sodium acetate (0.486 g, 5.93 mmol, 1.7 eq.) was added to a solution of Intermediate 165 (1 g, 3.49 mmol) in TFA (10 mL) and the reaction mixture was stirred at 130° C. for 2 h. The mixture was concentrated under reduced pressure. The residue was dissolved in EtOH (10 mL) and K₂CO₃ (0.756 g, 5.471 mmol, 1.7 eq.) was added. The reaction mixture was stirred at room temperature for 2 h. The solvent was evaporated to give Intermediate 166, used in the next step without further purification.

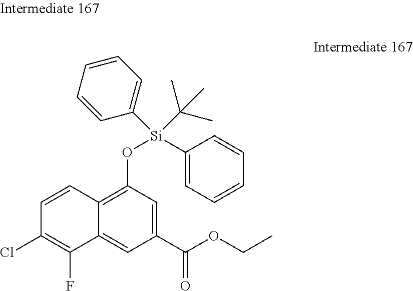 [SMILES: CCOC(=O)c1cc(O[Si](c2ccccc2)(c2ccccc2)C(C)(C)C)c2ccc(Cl)c(F)c2c1]

Imidazole (24.7 g, 362.9 mmol, 1.5 eq.), tert-butylchlorodiphenylsilane (79.8 g, 290.3 mmol, 1.2 eq.) and DMAP (5.9 g, 48.4 mmol, 0.2 eq.) were added to a solution of Intermediate 166 (65 g, 241.9 mmol) in THF (1 L). The reaction mixture was stirred at room temperature overnight. The reaction was quenched by addition of water (1 L). The resulting mixture was extracted with EtOAc (3×500 mL). The organic layer was washed with brine (1 L), dried over Na₂SO₄, filtered through a celite pad, and concentrated under reduced pressure. The residue was purified by flash chromatography on silica gel (petroleum ether/EtOAc=4/1) to afford Intermediate 167 (105 g, yield: 85% yield) as a yellow oil.

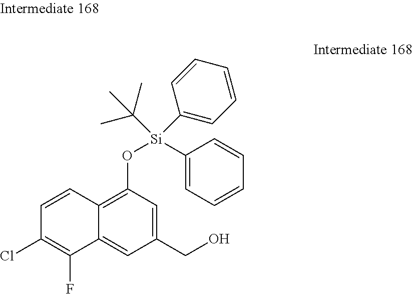 [SMILES: CC(C)(C)[Si](Oc1cc(CO)cc2c(F)c(Cl)ccc12)(c1ccccc1)c1ccccc1]

LiAlH₄ (9.43 g, 248.5 mmol, 1.2 eq.) was added portionwise to a solution of Intermediate 167 (105 g, 207.1 mmol) in THF (1 L) at 0° C. The reaction mixture was stirred at room temperature for 1 h. The reaction was quenched by addition of sodium sulfate decahydrate (10 g). The resulting mixture was filtered and the filtrate was combined and concentrated. The crude product was triturated with petroleum ether and diethyl ether to afford Intermediate 168 (95 g, yield; 93%) as a white solid.

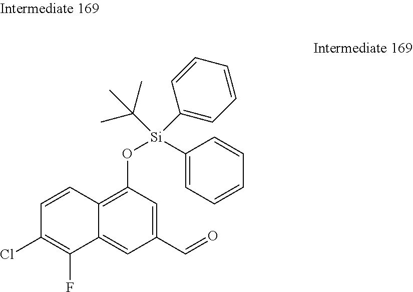 [SMILES: CC(C)(C)[Si](Oc1cc(C=O)cc2c(F)c(Cl)ccc12)(c1ccccc1)c1ccccc1]

Dess-Martin periodinane (CAS [87413-09-0], 150.5 g, 354.8 mmol, 3 eq.) was added to a mixture of Intermediate 168 (55 g, 118.3 mmol) in DCM (1 L). The reaction mixture was stirred at room temperature for 1 h. The resulting mixture was filtered through a celite pad. The filtrate was diluted with water (1 L) and was extracted with DCM (500 mL×3). The combined organic layer was washed with brine (2 L), dried over MgSO₄, filtered through a celite pad, and concentrated under reduced pressure. The crude product was triturated with petroleum ether (100 mL) and diethyl ether (100 mL) to afford Intermediate 169 (45 g, yield: 82%) as a white solid.

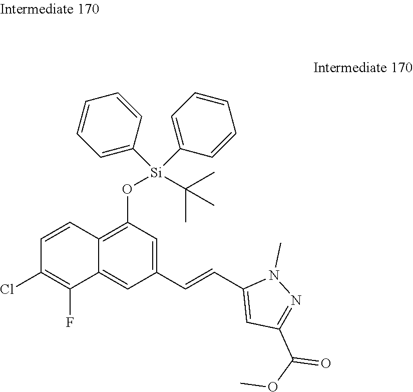 [SMILES: COC(=O)c1cc(/C=C/c2cc(O[Si](c3ccccc3)(c3ccccc3)C(C)(C)C)c3ccc(Cl)c(F)c3c2)n(C)n1]

Sodium hydride (60% in mineral oil, 7.1 g, 178.2 mmol, 1.5 eq.) was added to a solution of Intermediate 169 (55 g, 118.8 mmol) and Intermediate 105 (53.5 g, 118.8 mmol, 1.5 eq.) in THF (600 mL) at 0° C. and the resulting solution was stirred at room temperature for 1 h. The reaction was quenched by adding saturated aqueous NH₄Cl (100 mL) and the resulting mixture was extracted with EtOAc (3×500 mL). The organic layer was washed with brine (1 L), dried over Na₂SO₄, filtered through a celite pad, and concentrated under reduced pressure. The residue was purified by flash chromatography on silica gel (petroleum ether/EtOAc 4/1) to afford Intermediate 170 (38 g, yield: 53%) as a white solid.

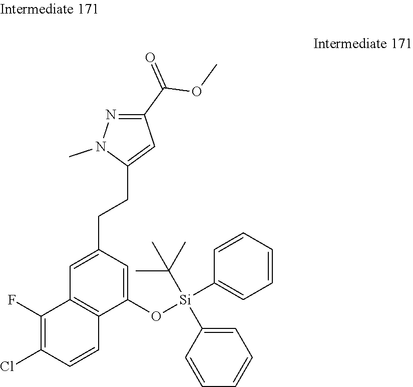 [SMILES: COC(=O)c1cc(CCc2cc(O[Si](c3ccccc3)(c3ccccc3)C(C)(C)C)c3ccc(Cl)c(F)c3c2)n(C)n1]

Pd/C (10%, 15 g, 140.9 mmol, 0.225 eq.) was added to a solution of Intermediate 170 (37.5 g, 62.6 mmol) in EtOAc (500 mL) under nitrogen atmosphere and the resulting solution was stirred under hydrogen atmosphere at room temperature for 16 h. The reaction mixture was filtered through a celite pad and the filtrate was concentrated under reduced pressure. The residue was purified by flash chromatography on silica gel (petroleum ether/EtOAc 4/1) to afford Intermediate 171 (25 g, yield: 66% yield) as a white solid

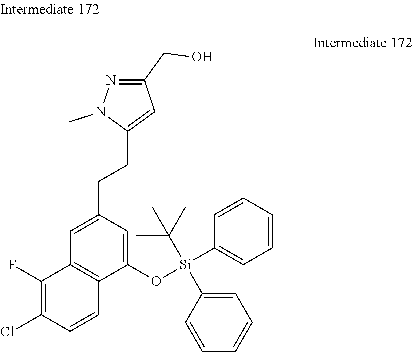 [SMILES: Cn1nc(CO)cc1CCc1cc(O[Si](c2ccccc2)(c2ccccc2)C(C)(C)C)c2ccc(Cl)c(F)c2c1]

Diisobutylaluminium hydride (1 M in toluene, 83.2 mL, 124.7 mmol, 3 eq.) was added dropwise to a mixture of Intermediate 171 (25 g, 41.6 mmol) in DCM (500 mL) under nitrogen atmosphere at −78° C. The reaction mixture was stirred at room temperature for 1 h. The reaction was quenched by adding saturated aqueous potassium sodium tartrate (200 mL). The resulting mixture was filtered and the filtrate was extracted with DCM (3×200 mL). The combined organic layer was evaporated and the crude product 10 was triturated with petroleum ether (100 mL) and diethyl ether (100 mL) to afford Intermediate 172 (19 g, yield: 78%) as a white solid.

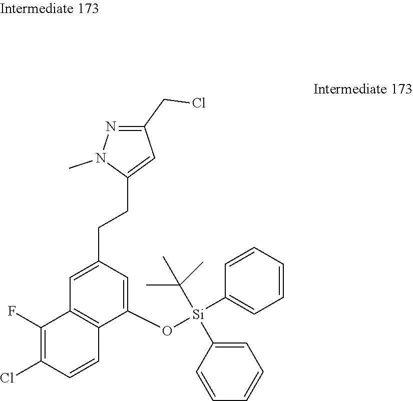 [SMILES: Cn1nc(CCl)cc1CCc1cc(O[Si](c2ccccc2)(c2ccccc2)C(C)(C)C)c2ccc(Cl)c(F)c2c1]

SOCl₂ (1.08 g, 9.07 mmol, 1.3 eq.) was added to a solution of Intermediate 172 (4 g, 6.98 mmol, 1 eq.) in DCM (100 mL) at 0° C. The reaction mixture was stirred at room temperature for 1 h. The reaction was quenched by adding saturated aqueous NaHCO₃ (100 mL). The mixture was extracted with DCM (100 mL×3). The combined organic layer was washed with brine (100 mL), dried over Na₂SO₄, filtered, and concentrated under reduced pressure to afford Intermediate 173 (4.1 g, yield: 99%) as a white solid.

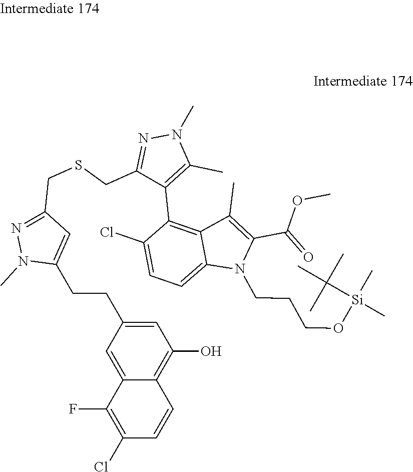

Intermediate 173 (2.25 g, 3.80 mmol, 1.1 eq.) was added to a solution of Intermediate 8 (2 g, 3.46 mmol) in MeOH (50 mL) at room temperature. The reaction mixture was stirred at room temperature for 10 min under nitrogen atmosphere. K₂CO₃ (0.96 g, 6.92 mmol, 2 eq.) was added and the mixture was stirred at room temperature for 16 h under nitrogen atmosphere. Water (30 mL) was added and the mixture was extracted with EtOAc (30 mL×3). The organic layer was washed with brine (30 mL), dried over Na₂SO₄, and concentrated under reduced pressure. The residue was purified by flash chromatography (petroleum ether/EtOAc 1/2) to afford Intermediate 174 (2.2 g, yield: 51%) as a red solid.

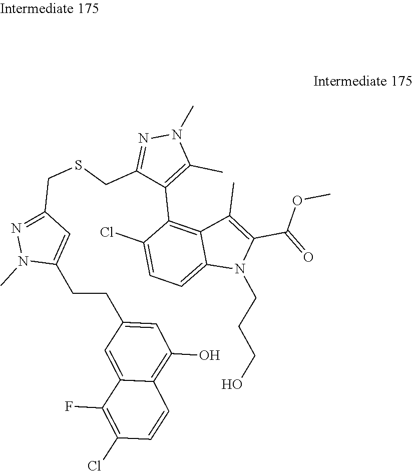

p-Toluenesulfonic acid (806 mg, 2.95 mmol, 1.2 eq.) was added to a solution of Intermediate 174 (2.1 g, 2.46 mmol) in MeOH (30 mL) and the reaction mixture was stirred at room temperature for 1 h. Water (30 mL) was added and the mixture was extracted with EtOAc (30 mL×3). The organic layer was washed with saturated aqueous NaHCO₃ (30 mL×2) and brine (50 mL). The organic layer was concentrated under reduced pressure to afford Intermediate 175 (1.6 g, yield: 73%) used without further purification.

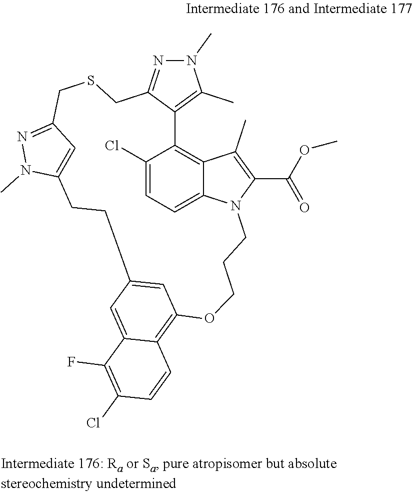

Intermediate 177: Sₐ or Rₐ, pure atropisomer but absolute stereochemistry undetermined A solution of Intermediate 175 (1.5 g, 2.03 mmol, 1 eq.) and di-tert-Butyl azodicarboxylate (0.9 g, 4.06 mmol, 2 eq.) in toluene (30 mL) and THF (5 mL) was added dropwise over 10 min to a solution of triphenylphosphine (1.1 g, 4.06 mmol, 2 eq.) in toluene (30 mL) at 70° C. under nitrogen atmosphere. After the addition was complete, the reaction mixture was further stirred at the same temperature for 10 min. The mixture was concentrated under reduced pressure and the residue was purified by reverse-phase flash chromatography (50-99% ACN/Water-5 mmol NH₄HCO₃) followed by preparative chiral HPLC (Column: CHIRAL ART Amylose-SA S, 3*25 cm, 5 μm; Mobile Phase A: CO₂, Mobile Phase B: IPA:ACN=1:1 (0.1% 2 M NH₃-MeOH); Gradient: 50% B) to afford Intermediate 176 (250 mg, yield: 17%) and Intermediate 177 (350 mg, yield: 24%), both as a white solids.

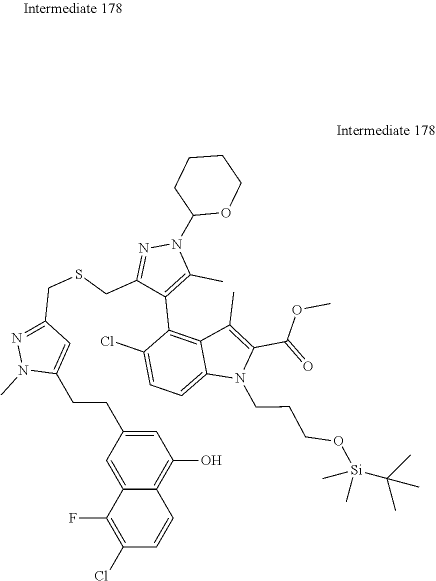 [SMILES: COC(=O)c1c(C)c2c(-c3c(CSCc4cc(CCc5cc(O)c6ccc(Cl)c(F)c6c5)n(C)n4)nn(C4CCCCO4)c3C)c(Cl)ccc2n1CCCO[Si](C)(C)C(C)(C)C]

Intermediate 173 (3.7 g, 6.28 mmol, 1.1 eq.) was added to a solution of Intermediate 125 (3.7 g, 5.71 mmol) in MeOH (100 mL) under nitrogen atmosphere. K₂CO₃ (1.57 g, 11.41 mmol, 2 eq.) was added and the reaction mixture was stirred at room temperature for 16 h under nitrogen atmosphere. The reaction was quenched by adding water (100 mL). The resulting mixture was extracted with EtOAc (3×100 mL). The combined organic layer was dried over Na₂SO₄, filtered, and evaporated. The residue was purified by silica gel chromatography (petroleum ether/EtOAc 100/0 to 80/20) to afford Intermediate 178 (4.2 g, yield: 80%) as a yellow oil.

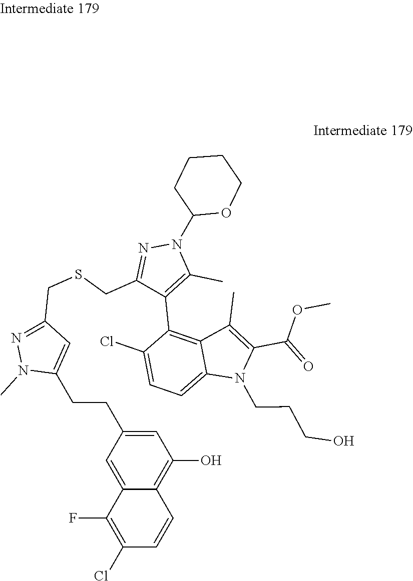 [SMILES: COC(=O)c1c(C)c2c(-c3c(CSCc4cc(CCc5cc(O)c6ccc(Cl)c(F)c6c5)n(C)n4)nn(C4CCCCO4)c3C)c(Cl)ccc2n1CCCO]

Triethylamine trihydrofluoride (1.1 g, 6.82 mmol, 1.5 eq.) was added to a solution of Intermediate 178 (4.2 g, 4.55 mmol) in THF (100 mL) and the reaction mixture was stirred at room temperature for 16 h. The reaction was quenched by adding water (100 mL). The resulting mixture was extracted with EtOAc (3×50 mL). The combined organic layer was washed with brine, dried over Na₂SO₄, filtered, and concentrated to afford Intermediate 179 (3.6 g, yield: 98%) as a yellow solid, used without further purification.

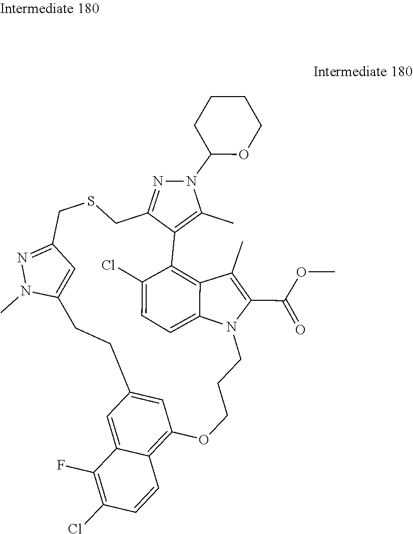

A solution of Intermediate 179 (3.6 g, 4.45 mmol) and DTBAD (3.0 g, 13.35 mmol, 3 eq.) in toluene (50 mL) and THE (5 mL) was added dropwise over 5 min to a solution of PPh₃ (3.5 g, 13.31 mmol, 3 eq.) in toluene (50 mL), stirring at 70° C. under nitrogen atmosphere. After the addition, the reaction mixture was further stirred at the same temperature for 20 min. The solvents were evaporated and the residue was partitioned between water and DCM. The layers were separated and the aqueous layer was extracted with DCM (50 mL×3). The organic layer was washed with brine (50 mL), dried over Na₂SO₄, filtered, and evaporated. The residue was purified by reverse phase flash chromatography (40-100% 0.05% NH₄HCO₃H₂O/CH₃CN) to afford Intermediate 180 (1.9 g, yield: 54%) as a yellow solid.

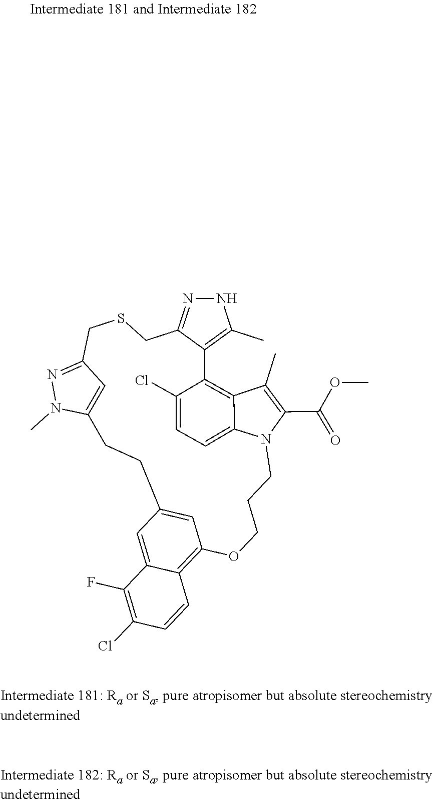

A solution of Intermediate 180 (1.9 g, 2.40 mmol) in HCl (4 M in dioxane, 30 mL) was stirred at room temperature for 16 h. The solid that appeared was collected by filtration. The residue was purified by preparative chiral SFC (Column: CHIRALPAK IF, 30*250 mm, 5 μm; Mobile Phase A: CO₂, Mobile Phase B: iPrOH:ACN=1:1 (0.1% 2 M NH₃-MeOH); Gradient: 50% B) to afford Intermediate 181 (370 mg, yield: 22%) and Intermediate 182 (410 mg, yield: 23%), both as off-white solids.

Intermediate 181: OR: +44° (589 nm, 22.4° C., 5 mg in 10 mL MeOH)

Intermediate 182: OR: −42° (589 nm, 22.4° C., 5 mg in 10 mL MeOH)

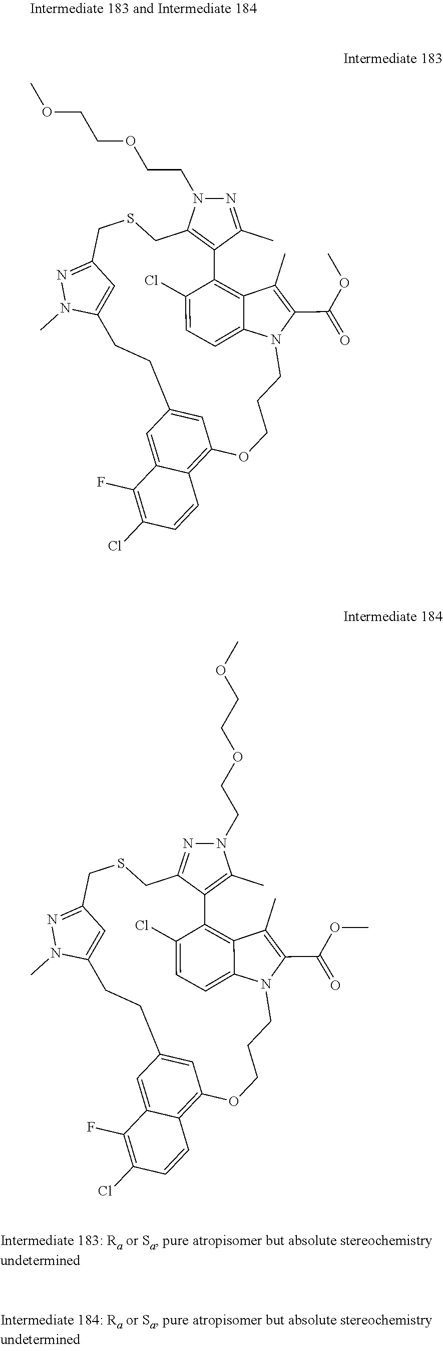

1-Bromo-2-(2-methoxyethoxy)ethane (145 mg, 0.79 mmol, 2 eq.) and Cs₂CO₃ (386 mg, 1.19 mmol, 3 eq.) were added to a solution of Intermediate 181 (280 mg, 0.40 mmol) in DMF (15 mL). The reaction mixture was stirred at 35° C. for 48 h. The reaction was quenched by adding water (20 mL). The mixture was extracted with EtOAc (3×20 mL). The combined organic layer was dried over Na₂SO₄, filtered, and evaporated. The residue was purified by reverse phase flash chromatography (40-100% 0.05% NH₄HCO₃H₂O/CH₃CN) followed by preparative chiral HPLC (Column: CHIRALPAK IC, 3*25 cm, 5 μm; Mobile Phase A: hexane (0.5% 2 M NH₃-MeOH), Mobile Phase B: EtOH; Gradient: 40% B to 40% B in 15 min) to afford Intermediate 183 (130 mg, yield: 41%) and Intermediate 184 (130 mg, yield: 41%), both as yellow oils.

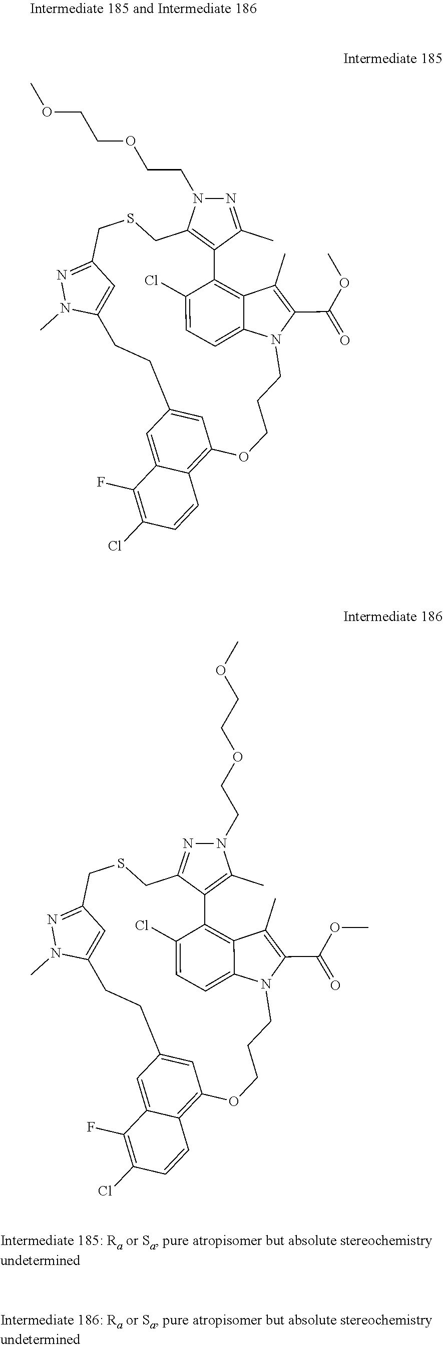

Intermediate 185 and Intermediate 186 were prepared according to an analogous procedure as for Intermediate 183 and Intermediate 184, respectively, starting from Intermediate 182 instead of Intermediate 181.

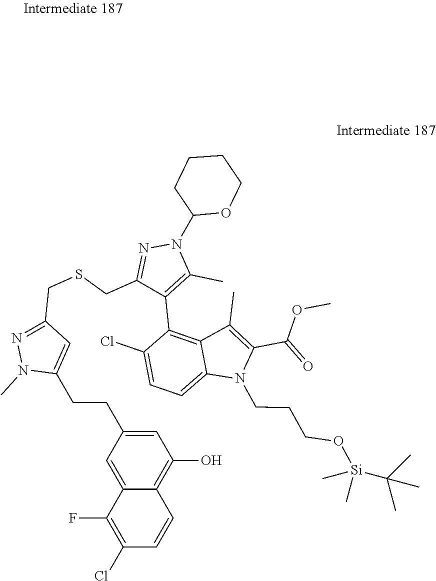 [SMILES: COC(=O)c1c(C)c2c(-c3c(CSCc4cc(CCc5cc(O)c6ccc(Cl)c(F)c6c5)n(C)n4)nn(C4CCCCO4)c3C)c(Cl)ccc2n1CCCO[Si](C)(C)C(C)(C)C]

Intermediate 187 was prepared according to an analogous procedure as for Intermediate 178, starting from Intermediate 159 instead of Intermediate 173.

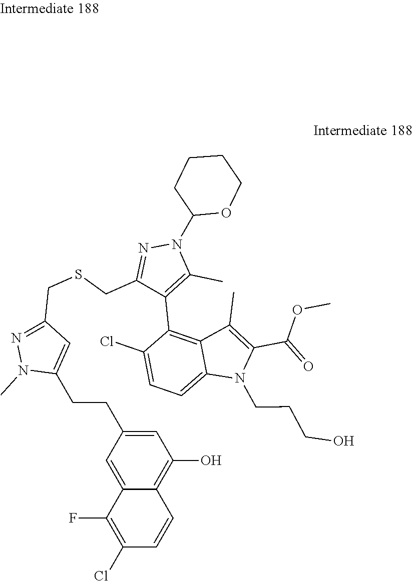 [SMILES: COC(=O)c1c(C)c2c(-c3c(CSCc4cc(CCc5cc(O)c6ccc(Cl)c(F)c6c5)n(C)n4)nn(C4CCCCO4)c3C)c(Cl)ccc2n1CCCO]

Intermediate 188 was prepared according to an analogous procedure as for Intermediate 179, starting from Intermediate 187 instead of Intermediate 178.

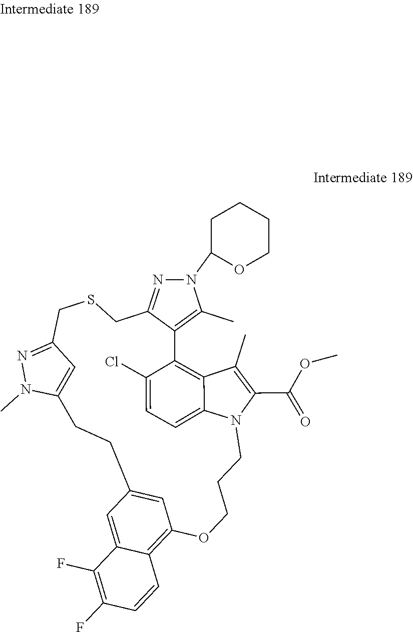

A solution of Intermediate 188 (3 g, 3.786 mmol) and di-tert-butyl azodicarboxylate (2.615 g, 11.359 mmol, 3 eq.) in toluene (40 mL) and THE (10 mL) was added dropwise over 10 min to a solution of triphenylphosphine (2.979 g, 11.359 mmol, 3 eq.) in toluene (40 mL) while stirring at 70° C. under nitrogen atmosphere. After the addition was complete, the reaction mixture was further stirred at the same temperature for 10 min. The mixture was concentrated under reduced pressure and the residue was purified by reverse-phase flash chromatography (50-99% ACN/Water-5 mmol NH₄HCO₃) to afford Intermediate 189 (1.8 g, yield: 55%) as a white solid.

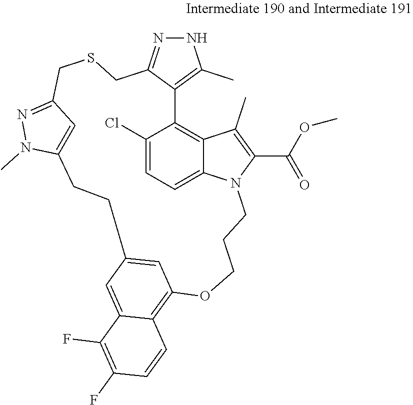

A solution of Intermediate 189 (1.7 g, 2.19 mmol) in HCl (4 M in dioxane, 50 mL) was stirred at room temperature for 2 h. The reaction mixture was concentrated under reduced pressure and the residue was purified by reverse-phase flash chromatography (50-99% ACN/Water-5 mmol NH₄HCO₃) followed by preparative chiral HPLC (Column: CHIRALPAK IG, 3*25 cm, 5 μm; Mobile Phase A: CO₂, Mobile Phase B: iPrOH (0.5% 2 M NH₃-MeOH); Gradient: 50% B) to afford Intermediate 190 (350 mg, yield: 22%) and Intermediate 191 (330 mg, yield: 21%), both as white solids.

Intermediate 190: OR: =+67.5° (589 nm, 22.5° C., 5.0 mg in 10 mL MeOH).

Intermediate 191: OR: −47.5° (589 nm, 22.5° C., 5.0 mg in 10 mL MeOH).

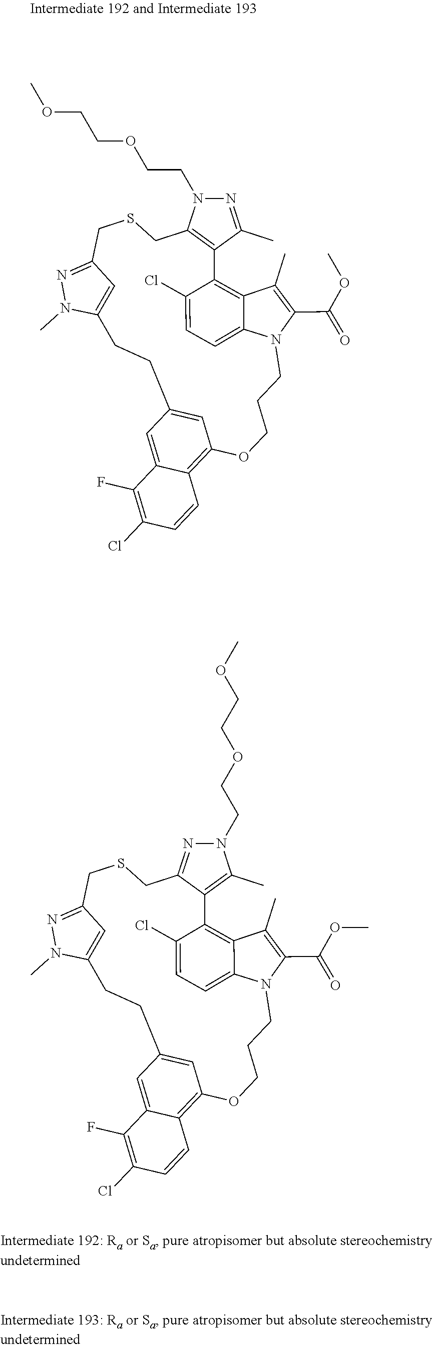

Intermediate 192 and Intermediate 193 were prepared according to an analogous procedure as for Intermediate 183 and Intermediate 184, respectively, starting from Intermediate 190 instead of Intermediate 181.

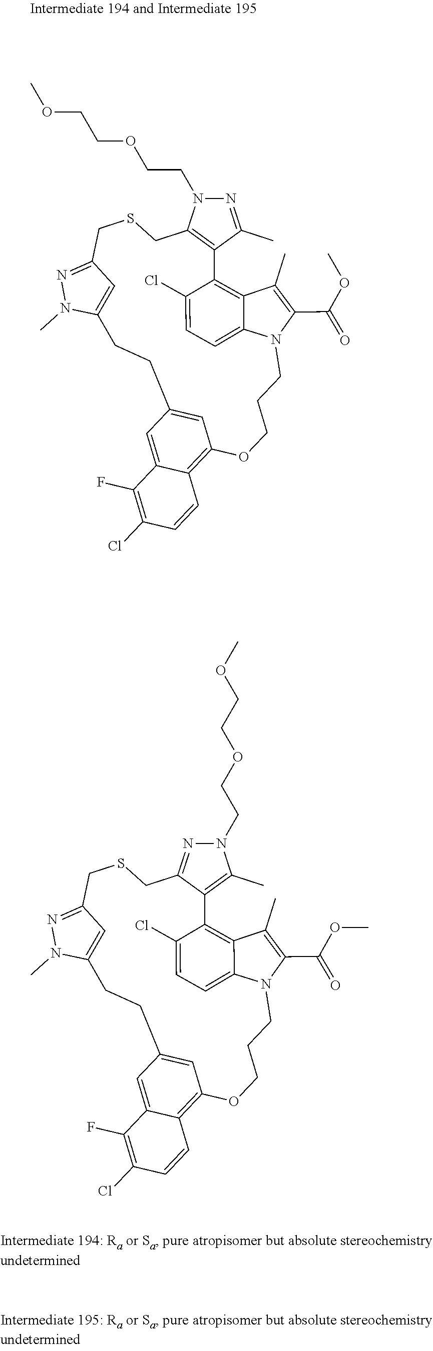

Intermediate 194 and Intermediate 195 were prepared according to an analogous procedure as for Intermediate 183 and Intermediate 184, respectively, starting from Intermediate 191 instead of Intermediate 181.

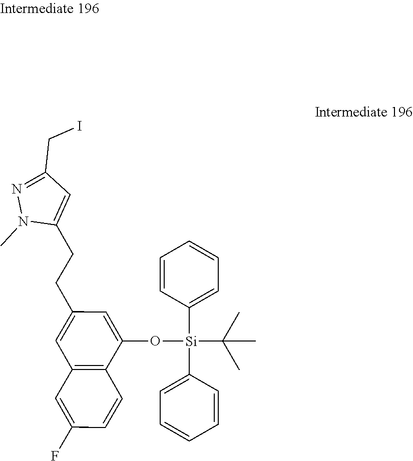 [SMILES: Cn1nc(CI)cc1CCc1cc(O[Si](c2ccccc2)(c2ccccc2)C(C)(C)C)c2ccc(F)cc2c1]

DIPEA (0.64 mL, 2 eq.) followed by methanesulfonic anhydride (0.65 g, 2 eq.) was added to a solution of Intermediate 24a (1.0 g, 1.86 mmol) in THF (45 mL), cooled to 0° C. The reaction mixture was stirred at room temperature for 0.5 h. Sodium iodide (1.39 g, 5 eq.) was then added to the mixture and it was further stirred at room temperature for 1 h. The reaction mixture was diluted with DCM (100 mL) and washed with water (20 mL). The aqueous layer was extracted with DCM/iPrOH 3:1 (2×30 mL), the combined organic layer was dried over MgSO₄, and concentrated under reduced pressure to give a dark yellow oil. This oil was purified by flash column chromatography on silica gel (SiO₂, 24 g column, 0-3% MeOH in DCM) to give Intermediate 196 (1.1 g, yield: 91%)

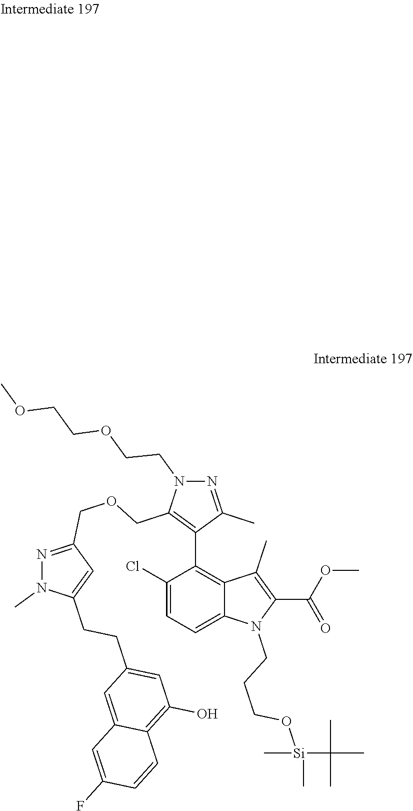 [SMILES: COCCOCCn1nc(C)c(-c2c(Cl)ccc3c2c(C)c(C(=O)OC)n3CCCO[Si](C)(C)C(C)(C)C)c1COCc1cc(CCc2cc(O)c3ccc(F)cc3c2)n(C)n1]

A solution of Intermediate 81 (540 mg, 0.888 mmol) and Intermediate 196 (691 mg, 1.065 mmol, 1.2 eq.) in THF (18 mL) was added dropwise over 20 min to a suspension of NaH (60% in mineral oil, 43 mg, 1.776 mmol, 2 eq.) in THF (18 mL) at 0° C. The reaction mixture was stirred at 0° C. for 1 h. The reaction was quenched by adding MeOH (5 mL). The solvents were evaporated and the residue was purified by preparative TLC (EtOAc) to afford Intermediate 197 (410 mg, yield: 52%) as a yellow oil.

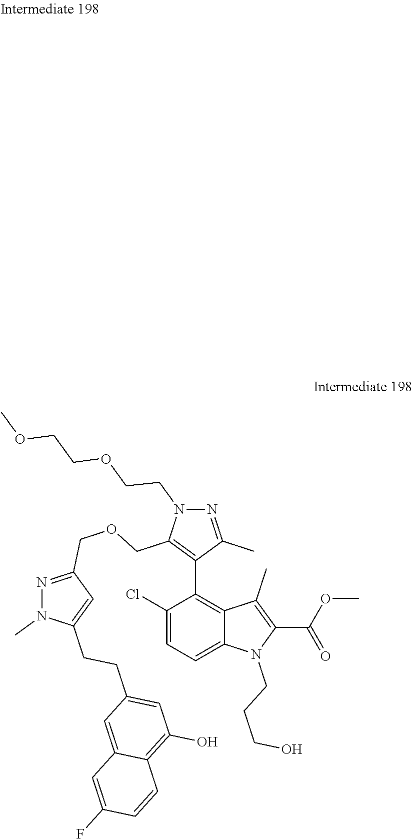

p-Toluenesulfonic acid (95 mg, 0.55 mmol, 1.2 eq.) was added to a solution of Intermediate 197 (410 mg, 0.46 mmol) in MeOH (5 mL). The reaction mixture was stirred at room temperature for 1 h. Water (5 mL) was added and the mixture was extracted with EtOAc (5 mL×3). The combined organic layer was washed with saturated aqueous NaHCO₃ (10 mL), brine (10 mL), dried over Na₂SO₄, filtered, and concentrated under reduced pressure. The residue was purified by preparative TLC (EtOAc) to afford Intermediate 198 (250 mg, yield: 70%) as a yellow oil.

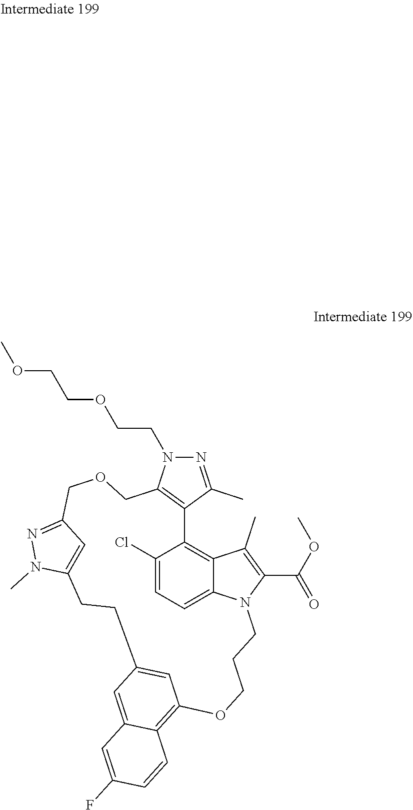 [SMILES: COCCOCCn1nc(C)c2c1COCc1cc(n(C)n1)CCc1cc(c3ccc(F)cc3c1)OCCCn1c(C(=O)OC)c(C)c3c-2c(Cl)ccc31]

Intermediate 199 was prepared according to an analogous procedure as for Intermediate 180, starting from Intermediate 198 instead of Intermediate 179.

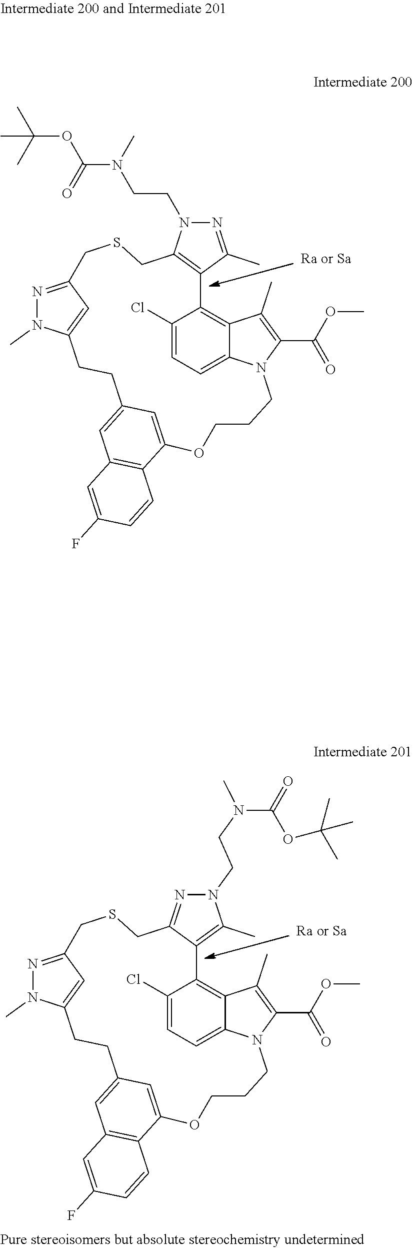

A solution of Intermediate 60 (200 mg, 0.3 mmol), tert-butyl (2-chloroethyl)(methyl)carbamate (CAS [220074-38-4], 202 mg, 1.04 mmol, 3.5 eq.), and Cs₂CO₃ (291 mg, 0.89 mmol, 3 eq.) in dry DMF (4.6 mL) was stirred at 60° C. under nitrogen atmosphere for 6.5 h. Additional tert-butyl (2-chloroethyl)(methyl)carbamate (202 mg, 1.04 mmol, 3.5 eq.) was added and the mixture was stirred at 60° C. for 16 h. Again, additional tert-butyl (2-chloroethyl)(methyl)carbamate (202 mg, 1.04 mmol, 3.5 eq.) was added and the mixture was stirred at 60° C. for 3.5 h. The solvent was removed under reduced pressure and the residue was taken up with DCM and brine. The layers were separated and the organic layer was washed with brine (×3). The combined aqueous layer was extracted with DCM (×5) and the combined organic layer was dried over MgSO₄, filtered, and concentrated under reduced pressure. The residue was purified by flash column chromatography (SiO₂, 12 g RediSep, DCM/MeOH 100/0 to 90/10) followed by preparative SFC (stationary phase: Chiralpak Daicel IG 20×250 mm, Mobile phase: CO₂, EtOH+0.4% iPrNH₂) to give Intermediate 200 (86 mg, yield: 35%) and Intermediate 201 (95 mg, yield: 38%) both as pale yellow solids.

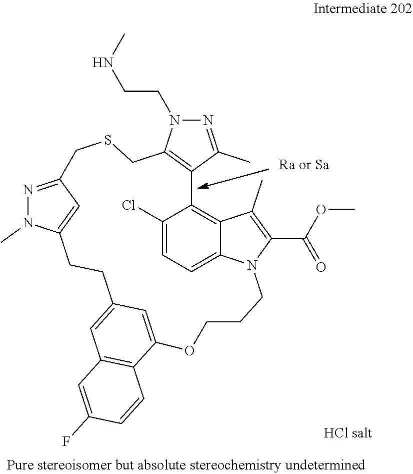

HCl (6 M in iPrOH, 2.6 mL, 15.59 mmol, 150 eq.) was added to a solution of Intermediate 200 (86 mg, 0.104 mmol) in MeOH (2 mL) under nitrogen atmosphere. The reaction mixture was stirred at room temperature for 4 h. More HCl (6 M in iPrOH, 0.52 mL, 3.12 mmol, 30 eq.) was added again and the mixture was stirred at room temperature for 1 h. The solvent was removed under reduced pressure and the solid was rinsed twice with MeOH to give Intermediate 202 (HCl salt, 82.5 mg, yield: quantitative) as a pale yellow solid.

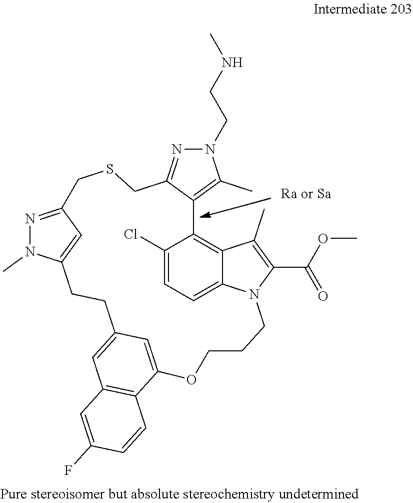

Intermediate 203 was prepared according to the same procedure as for Intermediate 202, starting from Intermediate 201 instead of Intermediate 200.

### Preparation of Compounds

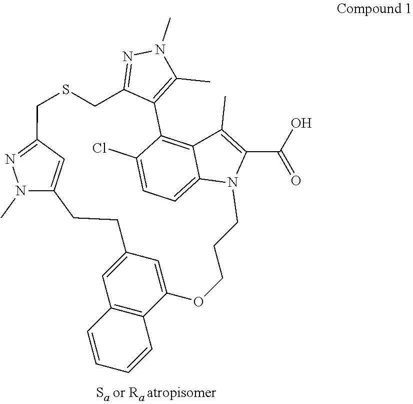 [SMILES: Cc1c(C(=O)O)n2c3ccc(Cl)c(c13)-c1c(nn(C)c1C)CSCc1cc(n(C)n1)CCc1cc(c3ccccc3c1)OCCC2]

LiOH (28 mg, 15 eq.) was added to a solution of Intermediate 17 (55 mg, 0.078 mmol) in a mixture of THE (1.25 mL), MeOH (1.25 mL) and water (0.625 mL) at room temperature. The resulting reaction mixture was stirred for 2 h at 60° C. The reaction mixture was concentrated to give a white solid. The solid was dissolved in water (5 mL) and acidified with aqueous HCl (1 M) to pH 3, a white precipitate forming upon acidification. The aqueous layer was extracted with EtOAc (20 mL) and then DCM (3×20 mL), the combined organic layer was dried over MgSO₄, filtered, and concentrated. The crude product was purified by flash column chromatography on silica gel (DCM:MeOH—1:0 to 95:5) to give a white solid that was triturated in DIPE and filtered to afford Compound 1 (32 mg, yield: 59%) as a white solid.

¹H NMR (400 MHz, DMSO-d₆) δ ppm 1.91 (s, 3H), 2.04 (s, 3H), 2.20-2.39 (m, 2H), 2.74-2.85 (m, 3H), 2.96-3.06 (m, 3H), 3.29-3.30 (m, 2H), 3.40 (s, 3H), 3.55 (q, J=8.1 Hz, 1H), 3.70-3.79 (m, 4H), 4.61 (ddd, J=14.2, 9.7, 4.0 Hz, 1H), 4.95 (s, 1H), 5.00 (dt, J=14.6, 4.8 Hz, 1H), 6.12 (d, J=1.4 Hz, 1H), 7.04 (d, J=9.0 Hz, 1H), 7.22 (s, 1H), 7.41-7.51 (m, 2H), 7.53 (d, J=9.1 Hz, 1H), 7.71-7.78 (m, 1H), 8.15-8.23 (m, 1H), 12.85-13.63 (m, 1H).

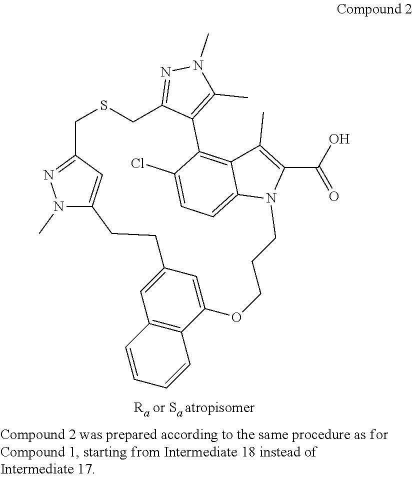 [SMILES: Cc1c(C(=O)O)n2c3ccc(Cl)c(c13)-c1c(nn(C)c1C)CSCc1cc(n(C)n1)CCc1cc(c3ccccc3c1)OCCC2]

¹H NMR (400 MHz, DMSO-d₆) δ ppm 1.91 (s, 3H), 2.04 (s, 3H), 2.21-2.38 (m, 2H), 2.74-2.86 (m, 3H), 2.96-3.06 (m, 3H), 3.29-3.30 (m, 2H), 3.40 (s, 3H), 3.55 (q, J=8.3 Hz, 1H), 3.69-3.78 (m, 4H), 4.61 (ddd, J=14.1, 9.7, 4.1 Hz, 1H), 4.95 (s, 1H), 5.00 (dt, J=14.6, 4.8 Hz, 1H), 6.12 (s, 1H), 7.04 (d, J=9.0 Hz, 1H), 7.22 (s, 1H), 7.41-7.50 (m, 2H), 7.53 (d, J=9.0 Hz, 1H), 7.70-7.79 (m, 1H), 8.19 (d, J=7.9 Hz, 1H), 12.65-13.84 (m, 1H).

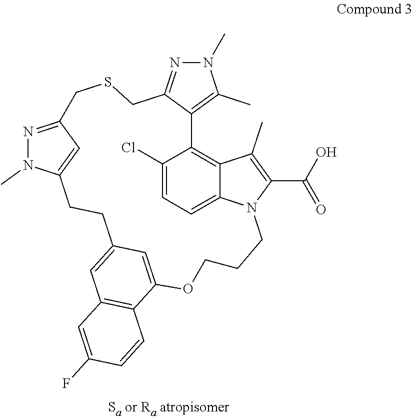 [SMILES: Cc1c(C(=O)O)n2c3ccc(Cl)c(c13)-c1c(nn(C)c1C)CSCc1cc(n(C)n1)CCc1cc(c3ccc(F)cc3c1)OCCC2]

LiOH (32 mg, 15 eq.) was added to a solution of Intermediate 27 (65 mg, 0.09 mmol) in a mixture of THE (2 mL), MeOH (2 mL), and water (1 mL). The resulting reaction mixture was stirred for 4 h at 60° C. The reaction mixture was concentrated to give a white solid. The solid was dissolved in water (5 mL) and acidified with aqueous HCl (1 M) to pH 4-5, a white precipitate forming upon acidification. The aqueous layer was extracted with DCM (3×20 mL), the combined organic layer was dried over MgSO₄, and concentrated to give a white solid. This crude product was purified by flash column chromatography on silica gel (DCM:MeOH—1:0 to 97:3). The purest fractions were combined to give a yellow solid that was triturated in Et₂O and filtered to afford Compound 3 (18 mg, yield: 28%) as a pale yellow solid. A second fraction of Compound 3 (14 mg, yield: 22%) with slightly lower purity was also isolated as a pale yellow solid.

¹H NMR (400 MHz, DMSO-d₆) δ ppm 1.91 (s, 3H), 2.04 (s, 3H), 2.19-2.39 (m, 2H), 2.75-2.86 (m, 3H), 3.00 (br d, J=13.8 Hz, 3H), 3.28-3.29 (m, 2H), 3.40 (s, 3H), 3.55 (br d, J=9.2 Hz, 1H), 3.69-3.79 (m, 4H), 4.61 (br s, 1H), 4.95 (s, 1H), 4.97-5.06 (m, 1H), 6.10 (s, 1H), 7.08 (d, J=9.0 Hz, 1H), 7.20 (s, 1H), 7.32 (td, J=8.9, 2.6 Hz, 1H), 7.47-7.56 (m, 2H), 8.24 (dd, J=9.2, 5.9 Hz, 1H), 12.88-13.64 (m, 1H).

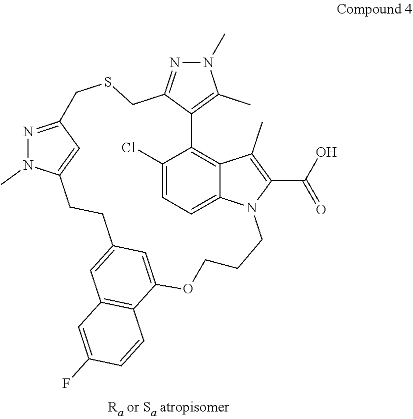 [SMILES: Cc1c(C(=O)O)n2c3ccc(Cl)c(c13)-c1c(nn(C)c1C)CSCc1cc(n(C)n1)CCc1cc(c3ccc(F)cc3c1)OCCC2]

Compound 4 was prepared according to the same procedure as for Compound 3, starting from Intermediate 28 instead of Intermediate 27.

¹H NMR (400 MHz, DMSO-d₆) δ ppm 1.91 (s, 3H), 2.04 (s, 3H), 2.20-2.38 (m, 2H), 2.76-2.86 (m, 3H), 2.96-3.06 (m, 3H), 3.28-3.29 (m, 2H), 3.40 (s, 3H), 3.55 (q, J=8.0 Hz, 1H), 3.69-3.79 (m, 4H), 4.54-4.67 (m, 1H), 4.95 (s, 1H), 4.97-5.06 (m, 1H), 6.10 (s, 1H), 7.08 (d, J=9.0 Hz, 1H), 7.20 (s, 1H), 7.32 (td, J=8.9, 2.6 Hz, 1H), 7.47-7.57 (m, 2H), 8.24 (dd, J=9.2, 5.8 Hz, 1H), 12.86-13.61 (m, 1H).

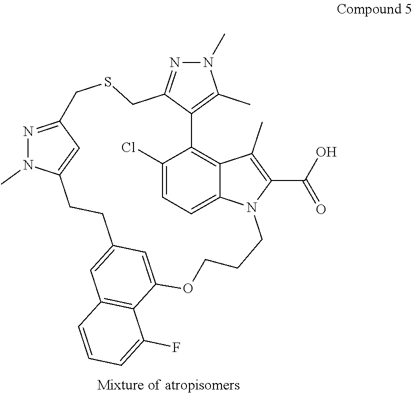 [SMILES: Cc1c(C(=O)O)n2c3ccc(Cl)c(c13)-c1c(nn(C)c1C)CSCc1cc(n(C)n1)CCc1cc(c3c(F)cccc3c1)OCCC2]

Compound 5 was prepared according to the same procedure as for Compound 3, starting from Intermediate 29 instead of Intermediate 27.

¹H NMR (400 MHz, DMSO-d₆) δ ppm 1.92 (s, 3H) 2.06 (s, 3H) 2.16-2.33 (m, 2H) 2.72-2.86 (m, 3H) 2.94-3.09 (m, 3H) 3.37 (br d, J=5.5 Hz, 10H) 3.35-3.38 (m, 4H) 3.39 (s, 5H) 3.40-3.42 (m, 2H) 3.69-3.74 (m, 1H) 3.75 (s, 4H) 4.50-4.61 (m, 1H) 4.91-4.99 (m, 1H) 5.00 (s, 1H) 6.16 (s, 1H) 7.00 (d, J=9.0 Hz, 1H) 7.18 (dd, J=13.3, 7.6 Hz, 1H) 7.33 (s, 1H) 7.43 (td, J=8.0, 4.8 Hz, 1H) 7.54 (d, J=9.1 Hz, 1H) 7.59 (d, J=8.3 Hz, 1H).

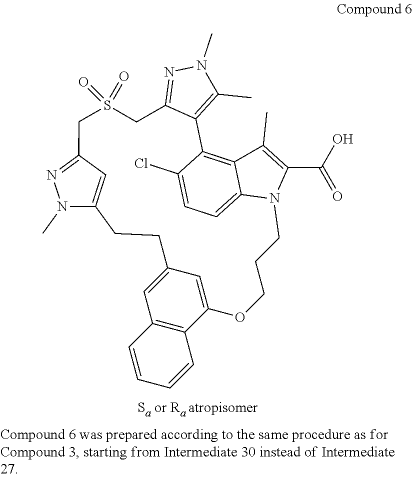 [SMILES: Cc1c(C(=O)O)n2c3ccc(Cl)c(c13)-c1c(nn(C)c1C)CS(=O)(=O)Cc1cc(n(C)n1)CCc1cc(c3ccccc3c1)OCCC2]

¹H NMR (400 MHz, DMSO-d₆) δ ppm 1.93 (s, 3H), 2.08 (s, 3H), 2.16-2.36 (m, 2H), 2.68-2.86 (m, 2H), 2.97 (s, 3H), 2.99-3.11 (m, 3H), 3.25 (br d, J=14.5 Hz, 1H), 3.49-3.57 (m, 1H), 3.66-3.72 (m, 1H), 3.77 (dd, J=14.8, 2.1 Hz, 1H), 3.82 (s, 3H), 4.64 (br s, 1H), 4.82 (d, J=14.8 Hz, 1H), 4.93 (br d, J=14.3 Hz, 1H), 5.62 (s, 2H), 7.03 (d, J=9.0 Hz, 1H), 7.35 (s, 1H), 7.44 (d, J=9.1 Hz, 1H), 7.47-7.57 (m, 2H), 7.77-7.84 (m, 1H), 8.25-8.32 (m, 1H).

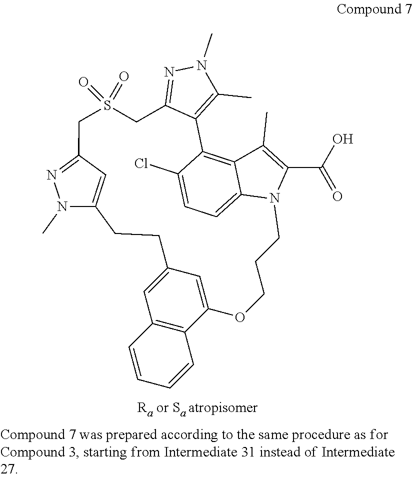 [SMILES: Cc1c(C(=O)O)n2c3ccc(Cl)c(c13)-c1c(nn(C)c1C)CS(=O)(=O)Cc1cc(n(C)n1)CCc1cc(c3ccccc3c1)OCCC2]

¹H NMR (400 MHz, DMSO-d₆) δ ppm 1.93 (s, 3H), 2.08 (s, 3H), 2.16-2.35 (m, 2H), 2.67-2.86 (m, 2H), 2.97 (s, 3H), 2.99-3.11 (m, 3H), 3.25 (br d, J=14.3 Hz, 1H), 3.53 (dt, J=9.7, 4.9 Hz, 1H), 3.65-3.73 (m, 1H), 3.74-3.81 (m, 1H), 3.83 (s, 3H), 4.58-4.69 (m, 1H), 4.82 (d, J=14.8 Hz, 1H), 4.89-4.97 (m, 1H), 5.62 (s, 2H), 7.03 (d, J=9.0 Hz, 1H), 7.35 (s, 1H), 7.44 (d, J=9.1 Hz, 1H), 7.46-7.56 (m, 2H), 7.77-7.84 (m, 1H), 8.26-8.32 (m, 1H).

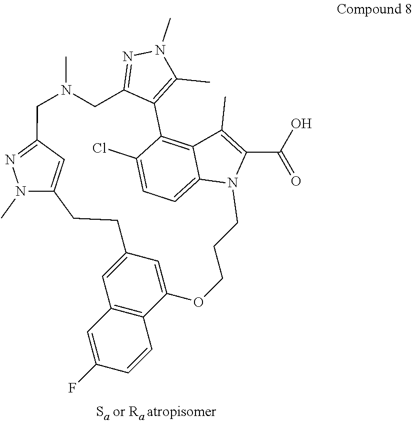 [SMILES: Cc1c(C(=O)O)n2c3ccc(Cl)c(c13)-c1c(nn(C)c1C)CN(C)Cc1cc(n(C)n1)CCc1cc(c3ccc(F)cc3c1)OCCC2]

To a solution of Intermediate 37 (40 mg, 0.055 mmol) in a mixture of THF (2 mL), MeOH (2 mL) and water (1 mL) was added LiOH (20 mg, 15 eq.). The resulting reaction mixture was stirred for 2 h at 60° C. The reaction mixture was concentrated to give a white solid. The solid was purified by preparative HPLC (Stationary phase: RP XBridge Prep C18 OBD—5 μm, 50×250 mm, Mobile phase: 0.25% NH₄HCO₃ solution in water, CH₃CN) to give a yellow solid that was triturated in Et₂O and filtered to afford Compound 8 (24 mg, yield: 61%) as a pale yellow solid.

¹H NMR (400 MHz, DMSO-d₆) δ ppm 1.75 (s, 3H), 1.88-1.93 (m, 3H), 1.98 (s, 3H), 2.24-2.42 (m, 2H), 2.80 (dd, J=28.9, 12.9 Hz, 2H), 2.87-2.98 (m, 3H), 3.06-3.12 (m, 2H), 3.62 (s, 3H), 3.72-3.82 (m, 5H), 4.03-4.13 (m, 1H), 4.50 (ddd, J=14.1, 9.5, 4.1 Hz, 1H), 4.63 (s, 1H), 5.06 (dt, J=14.5, 4.8 Hz, 1H), 6.57 (s, 1H), 7.09 (s, 1H), 7.21-7.31 (m, 2H), 7.43 (dd, J=10.5, 2.6 Hz, 1H), 7.69 (d, J=9.0 Hz, 1H), 8.09 (dd, J=9.2, 5.9 Hz, 1H).

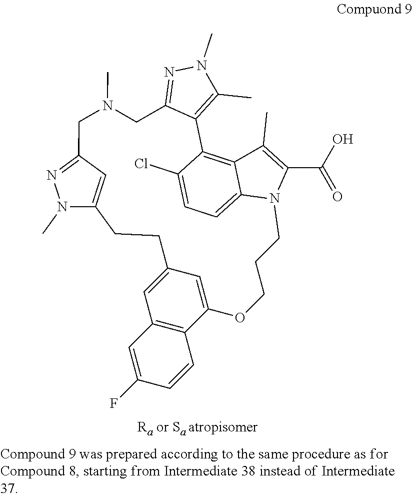 [SMILES: Cc1c(C(=O)O)n2c3ccc(Cl)c(c13)-c1c(nn(C)c1C)CN(C)Cc1cc(n(C)n1)CCc1cc(c3ccc(F)cc3c1)OCCC2]

¹H NMR (400 MHz, DMSO-d₆) δ ppm 1.75 (s, 3H), 1.90 (s, 3H), 1.98 (s, 3H), 2.24-2.41 (m, 2H), 2.79 (dd, J=28.8, 12.9 Hz, 2H), 2.86-2.98 (m, 3H), 3.04-3.13 (m, 3H), 3.62 (s, 3H), 3.73-3.82 (m, 4H), 4.08 (br d, J=8.6 Hz, 1H), 4.43-4.55 (m, 1H), 4.62 (s, 1H), 5.01-5.10 (m, 1H), 6.57 (s, 1H), 7.09 (s, 1H), 7.20-7.29 (m, 2H), 7.43 (dd, J=10.5, 2.6 Hz, 1H), 7.68 (d, J=9.0 Hz, 1H), 8.09 (dd, J=9.2, 5.9 Hz, 1H).

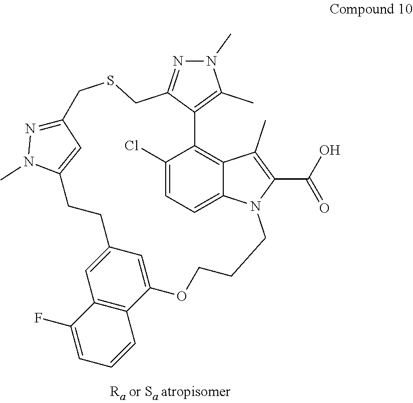 [SMILES: Cc1c(C(=O)O)n2c3ccc(Cl)c(c13)-c1c(nn(C)c1C)CSCc1cc(n(C)n1)CCc1cc(c3cccc(F)c3c1)OCCC2]

A solution of LiOH (68 mg, 15 eq.) in water (2 mL) was added to a solution of Intermediate 44 (130 mg, 0.19 mmol) in a mixture of THE (4 mL) and MeOH (4 mL). The reaction mixture was heated at 60° C. for 3 h. After cooling to room temperature, the reaction mixture was diluted with MeOH and directly injected on preparative HPLC (Stationary phase: RP XBridge Prep C18 OBD—10 μm, 30×150 mm, Mobile phase: 0.25% NH₄HCO₃ solution in water, CH₃CN) to give Compound 10 (104 mg, yield: 81%) as a white solid.

NMR: ¹H NMR (400 MHz, DMSO-d₆) δ ppm 1.91 (s, 3H), 2.04 (s, 3H), 2.21-2.37 (m, 2H), 2.77 (d, J=13.4 Hz, 1H), 2.80-2.90 (m, 2H), 2.99 (d, J=13.4 Hz, 1H), 3.01-3.09 (m, 2H), 3.25 (d, J=14.1 Hz, 1H), 3.30 (d, J=14.1 Hz, 1H), 3.41 (s, 3H), 3.51-3.60 (m, 1H), 3.72-3.80 (m, 4H), 4.56-4.65 (m, 1H), 4.95 (s, 1H), 5.01 (dt, J=14.5, 4.7 Hz, 1H), 6.21 (s, 1H), 7.07 (d, J=9.0 Hz, 1H), 7.27-7.33 (m, 1H), 7.35 (s, 1H), 7.42 (td, J=8.1, 5.5 Hz, 1H), 7.53 (d, J=9.0 Hz, 1H), 8.02 (d, J=8.4 Hz, 1H).

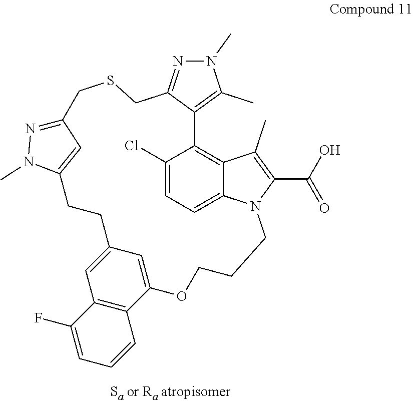 [SMILES: Cc1c(C(=O)O)n2c3ccc(Cl)c(c13)-c1c(nn(C)c1C)CSCc1cc(n(C)n1)CCc1cc(c3cccc(F)c3c1)OCCC2]

NMR: ¹H NMR (400 MHz, DMSO-d6) δ ppm 1.91 (s, 3H), 2.03 (s, 3H), 2.21-2.35 (m, 2H), 2.78 (d, J=13.6 Hz, 1H), 2.80-2.91 (m, 2H), 2.99 (d, J=13.6 Hz, 1H), 3.01-3.09 (m, 1H), 3.26 (d, J=14.1 Hz, 2H), 3.30 (d, J=14.1 Hz, 1H), 3.41 (s, 3H), 3.51-3.60 (m, 1H), 3.72-3.79 (m, 4H), 4.55-4.65 (m, 1H), 4.95 (s, 1H), 5.01 (dt, J=14.5, 4.7 Hz, 1H), 6.21 (s, 1H), 7.06 (d, J=9.0 Hz, 1H), 7.27-7.33 (m, 1H), 7.35 (s, 1H), 7.42 (td, J=8.1, 5.5 Hz, 1H), 7.52 (d, J=9.0 Hz, 1H), 8.02 (d, J=8.4 Hz, 1H).

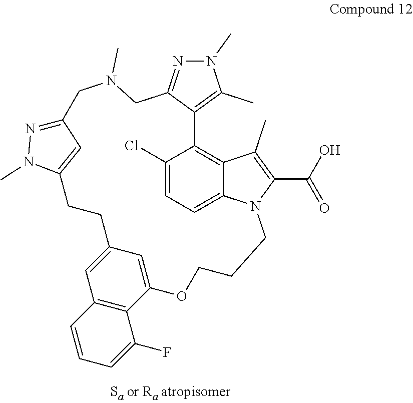 [SMILES: Cc1c(C(=O)O)n2c3ccc(Cl)c(c13)-c1c(nn(C)c1C)CN(C)Cc1cc(n(C)n1)CCc1cc(c3c(F)cccc3c1)OCCC2]

NMR: ¹H NMR (400 MHz, CDCl₃, 27° C.) δ ppm 1.93-2.12 (m, 7H) 2.16-2.33 (m, 4H) 2.59-2.80 (m, 3H) 2.93-3.19 (m, 4H) 3.35-3.45 (m, 2H) 3.49 (s, 2H) 3.55-3.89 (m, 5H) 4.41-4.61 (m, 1H) 4.99-5.26 (m, 1H) 5.30 (s, 1H) 5.79-6.00 (m, 1H) 6.78-6.93 (m, 1H) 6.99-7.13 (m, 1H) 7.14-7.24 (m, 1H) 7.36 (br s, 2H) 7.47-7.53 (m, 1H).

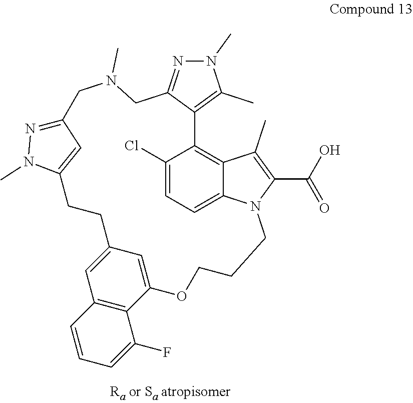 [SMILES: Cc1c(C(=O)O)n2c3ccc(Cl)c(c13)-c1c(nn(C)c1C)CN(C)Cc1cc(n(C)n1)CCc1cc(c3c(F)cccc3c1)OCCC2]

NMR: ¹H NMR (600 MHz, DMSO-d₆, 77° C.) δ ppm 1.87 (br s, 3H) 1.95 (s, 3H) 2.02 (s, 3H) 2.20-2.34 (m, 2H) 2.86-2.93 (m, 1H) 2.93-2.98 (m, 1H) 2.98-3.04 (m, 2H) 3.01-3.08 (m, 2H) 3.15-3.16 (m, 1H) 3.41-3.46 (m, 1H) 3.54 (s, 3H) 3.71-3.77 (m, 2H) 3.78 (s, 3H) 4.48-4.57 (m, 1H) 4.96 (br s, 1H) 5.01 (dt, J=14.6, 4.9 Hz, 1H) 6.48 (br s, 1H) 7.08 (dd, J=13.1, 7.5 Hz, 1H) 7.18 (d, J=8.9 Hz, 1H) 7.23 (s, 1H) 7.36 (td, J=7.9, 4.8 Hz, 1H) 7.51 (d, J=8.1 Hz, 1H) 7.62 (d, J=9.1 Hz, 1H).

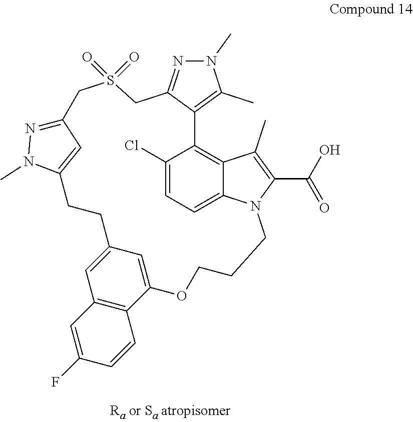 [SMILES: Cc1c(C(=O)O)n2c3ccc(Cl)c(c13)-c1c(nn(C)c1C)CS(=O)(=O)Cc1cc(n(C)n1)CCc1cc(c3ccc(F)cc3c1)OCCC2]

¹H NMR (400 MHz, DMSO-d₆) δ ppm 1.93 (s, 3H); 2.08 (s, 3H); 2.16-2.31 (m, 2H); 2.67-2.86 (m, 2H); 2.97-3.10 (m, 4H); 2.99 (s, 3H); 3.23-3.28 (m, 1H); 3.50-3.57 (m, 1H); 3.69 (br d, J=14.31 Hz, 1H); 3.76-3.81 (m, 1H); 3.83 (s, 3H); 4.64 (br t, J=10.78 Hz, 1H); 4.81 (d, J=14.75 Hz, 1H); 4.87-4.97 (m, 1H); 5.60 (s, 1H); 5.62 (s, 1H); 7.06 (d, J=9.02 Hz, 1H); 7.31-7.45 (m, 3H); 7.58 (dd, J=10.45, 2.53 Hz, 1H); 8.35 (dd, J=9.13, 5.83 Hz, 1H).

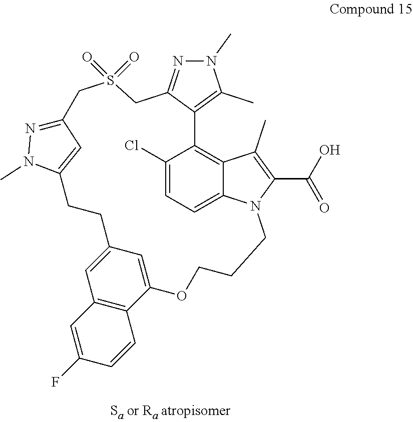 [SMILES: Cc1c(C(=O)O)n2c3ccc(Cl)c(c13)-c1c(nn(C)c1C)CS(=O)(=O)Cc1cc(n(C)n1)CCc1cc(c3ccc(F)cc3c1)OCCC2]

¹H NMR (400 MHz, DMSO-d₆) δ ppm 1.93 (s, 3H); 2.08 (s, 3H), 2.16-2.34 (m, 2H), 2.66-2.85 (m, 2H), 2.94-3.10 (m, 4H), 2.98 (s, 3H), 3.27 (br s, 3H), 3.49-3.56 (m, 1H), 3.69 (br d, J=14.32 Hz, 1H), 3.78 (br d, J=14.95 Hz, 1H), 3.83 (s, 3H), 4.64 (br t, J=10.97 Hz, 1H), 4.81 (d, J=14.63 Hz, 1H), 4.88-4.97 (m, 1H), 5.60 (s, 1H), 5.62 (s, 1H), 7.07 (d, J=8.99 Hz, 1H), 7.31-7.45 (m, 3H), 7.58 (dd, J=10.45, 2.61 Hz, 1H), 8.35 (dd, J=9.25, 5.90 Hz, 1H).

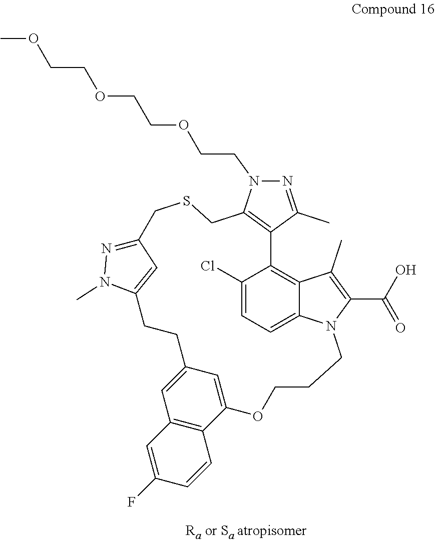 [SMILES: COCCOCCOCCn1nc(C)c2c1CSCc1cc(n(C)n1)CCc1cc(c3ccc(F)cc3c1)OCCCn1c(C(=O)O)c(C)c3c-2c(Cl)ccc31]

LiOH (19 mg, 30 eq.) was added to a solution of Intermediate 62 (21 mg, 0.026 mmol) in a mixture of THE (1 mL), MeOH (1 mL) and water (0.5 mL) at room temperature. The resulting reaction mixture was stirred overnight at 45° C. The reaction mixture was concentrated, the residue was dissolved in water (5 mL), and was acidified with aqueous HCl (1 M). The aqueous layer was extracted with CHCl₃ (3×). The combined organic layer was washed with brine, dried over MgSO₄, filtered, and evaporated to afford Compound 16 (20 mg, yield: 97%).

¹H NMR (400 MHz, CDCl₃) δ ppm 2.03 (s, 3H); 2.18 (s, 3H); 2.34 (br d, J=3.66 Hz, 2H); 2.84-2.93 (m, 5H); 3.08 (s, 3H); 3.21 (d, J=12.54 Hz, 1H); 3.34 (br t, J=5.33 Hz, 2H); 3.36 (s, 3H); 3.39 (d, J=15.57 Hz, 1H); 3.50-3.67 (m, 9H); 3.78 (d, J=15.57 Hz, 1H); 3.85-3.97 (m, 2H); 4.24-4.34 (m, 2H); 4.54 (ddd, J=14.63, 6.58, 3.66 Hz, 1H); 5.21 (ddd, J=14.76, 7.92, 3.87 Hz, 1H); 5.45 (s, 1H); 5.48 (s, 1H); 7.14-7.18 (m, 2H); 7.23-7.36 (m, 3H); 8.33 (dd, J=9.20, 5.75 Hz, 1H).

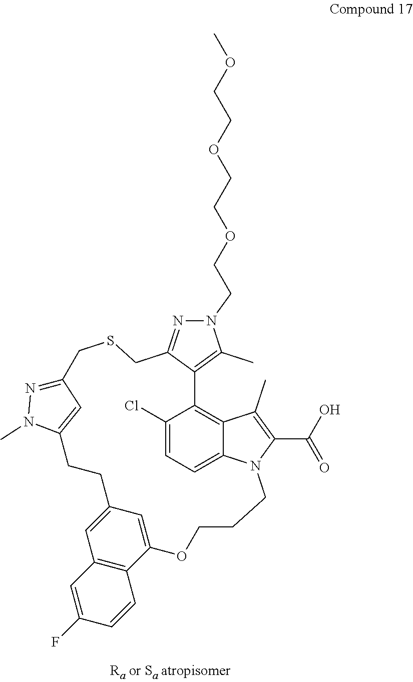 [SMILES: COCCOCCOCCn1nc2c(c1C)-c1c(Cl)ccc3c1c(C)c(C(=O)O)n3CCCOc1cc(cc3cc(F)ccc13)CCc1cc(nn1C)CSC2]

¹H NMR (400 MHz, CDCl₃) δ ppm 2.07 (s, 3H); 2.20 (s, 3H); 2.29 (br d, J=7.42 Hz, 2H); 2.78-2.97 (m, 5H); 3.08 (s, 3H); 3.16 (d, J=12.23 Hz, 1H); 3.24-3.39 (m, 6H); 3.47-3.63 (m, 8H); 3.66-3.73 (m, 1H); 3.83-3.93 (m, 2H); 4.28 (t, J=5.43 Hz, 2H); 4.46-4.60 (m, 1H); 5.15-5.27 (m, 1H); 5.46 (s, 1H); 5.49 (s, 1H); 7.13-7.20 (m, 2H); 7.22-7.37 (m, 3H); 8.33 (dd, J=9.25, 5.80 Hz, 1H).

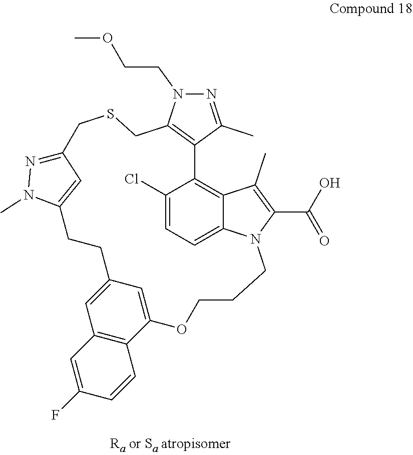 [SMILES: COCCn1nc(C)c2c1CSCc1cc(n(C)n1)CCc1cc(c3ccc(F)cc3c1)OCCCn1c(C(=O)O)c(C)c3c-2c(Cl)ccc31]

¹H NMR (400 MHz, CDCl₃) δ ppm 2.04 (s, 3H), 2.16 (s, 3H), 2.31 (br s, 2H), 2.79 (d, J=10.2 Hz, 2H), 2.93 (s, 2H), 2.95 (s, 3H), 3.18 (br d, J=4.0 Hz, 1H), 3.21-3.30 (m, 2H), 3.31 (s, 3H), 3.38-3.44 (m, 1H), 3.73-3.82 (m, 3H), 4.21-4.29 (m, 2H), 4.52 (s, 1H), 5.16-5.29 (m, 1H), 5.36 (s, 1H), 5.59 (s, 1H), 7.15 (s, 1H), 7.22-7.25 (m, 2H), 7.27-7.33 (m, 3H), 8.29 (dd, J=9.2, 5.9 Hz, 1H).

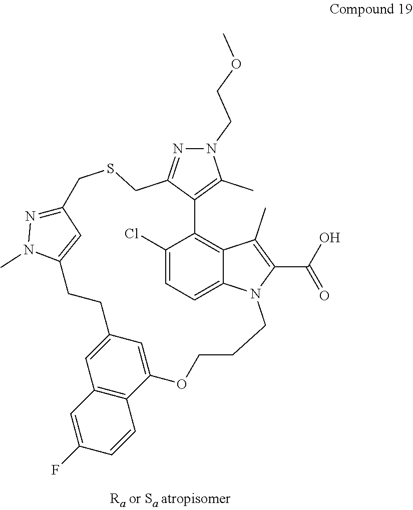 [SMILES: COCCn1nc2c(c1C)-c1c(Cl)ccc3c1c(C)c(C(=O)O)n3CCCOc1cc(cc3cc(F)ccc13)CCc1cc(nn1C)CSC2]

¹H NMR (400 MHz, CDCl₃) δ ppm 2.07 (s, 3H), 2.20 (s, 3H), 2.23-2.42 (m, 2H), 2.73-2.82 (m, 2H), 2.94 (s, 3H), 2.94-2.99 (m, 2H), 3.15-3.28 (m, 3H), 3.29 (s, 3H), 3.37-3.44 (m, 1H), 3.72-3.79 (m, 3H), 4.23-4.28 (m, 2H), 4.46-4.54 (m, 1H), 5.20-5.28 (m, 1H), 5.37 (s, 1H), 5.64 (s, 1H), 7.18 (s, 1H), 7.22-7.25 (m, 2H), 7.27-7.34 (m, 3H), 8.31 (dd, J=9.1, 5.8 Hz, 1H).

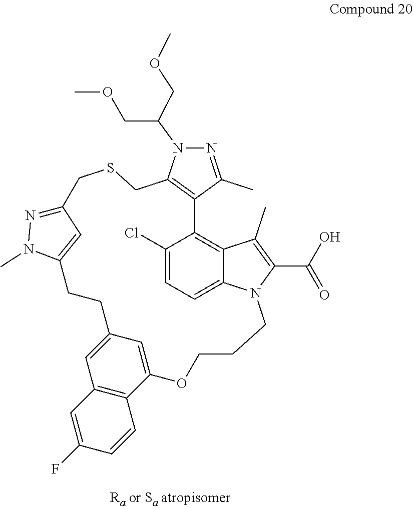 [SMILES: COCC(COC)n1nc(C)c2c1CSCc1cc(n(C)n1)CCc1cc(c3ccc(F)cc3c1)OCCCn1c(C(=O)O)c(C)c3c-2c(Cl)ccc31]

LiOH (2.5 mg, 15 eq.) was added to a solution of Intermediate 66 (5.4 mg, 0.007 mmol) in a mixture of MeOH (200 μL), THE (200 μL), and water (90 μL). The resulting reaction mixture was stirred for 4 h at 50° C. The reaction mixture was concentrated under reduced pressure to give a pale yellow solid. This solid was dissolved in water and DCM and acidified with 1 M aqueous HCl to pH 4-5, a pale yellow precipitate forming upon acidification. The aqueous layer was extracted with DCM (×4). The combined organic layer was dried over MgSO₄, filtered, and evaporated to give Compound 20 (4 mg, yield: 79%) as a pale yellow solid.

¹H NMR (400 MHz, CDCl₃) δ ppm 2.03 (s, 3H), 2.14 (s, 3H), 2.15-2.42 (m, 3H), 2.79 (br d, J=9.7 Hz, 2H), 2.92 (br s, 2H), 2.97 (br s, 3H), 3.15-3.21 (m, 1H), 3.22-3.26 (m, 2H), 3.27 (s, 3H), 3.28-3.30 (m, 1H), 3.34 (s, 3H), 3.37-3.45 (m, 1H), 3.72-3.79 (m, 2H), 3.79-3.84 (m, 1H), 3.84-3.93 (m, 2H), 4.43-4.55 (m, 2H), 5.23 (br d, J=4.5 Hz, 1H), 5.38 (br s, 1H), 5.59 (br s, 1H), 7.14 (s, 1H), 7.27-7.33 (m, 3H), 8.25-8.35 (m, 1H).

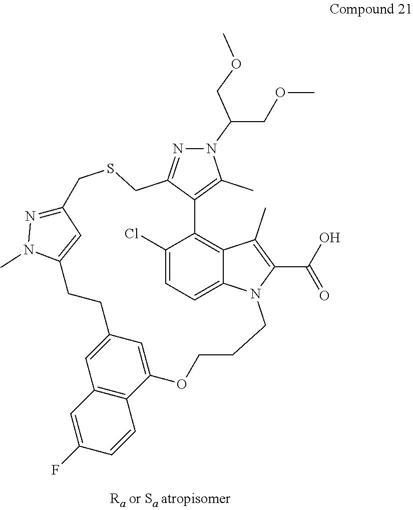 [SMILES: COCC(COC)n1nc2c(c1C)-c1c(Cl)ccc3c1c(C)c(C(=O)O)n3CCCOc1cc(cc3cc(F)ccc13)CCc1cc(nn1C)CSC2]

¹H NMR (400 MHz, CDCl₃) δ ppm 1.85-2.01 (m, 1H), 2.05 (s, 3H), 2.17-2.22 (m, 3H), 2.22-2.40 (m, 2H), 2.70-2.97 (m, 5H), 3.00 (s, 3H), 3.16 (d, J=11.8 Hz, 1H), 3.23 (br d, J=8.3 Hz, 1H), 3.29 (s, 3H), 3.32 (s, 3H), 3.34-3.43 (m, 1H), 3.70-3.91 (m, 5H), 4.45-4.56 (m, 2H), 5.18-5.27 (m, 1H), 5.40 (s, 1H), 5.59 (s, 1H), 7.18 (s, 1H), 7.21 (s, 1H), 7.25 (br s, 1H), 7.27-7.30 (m, 1H), 7.30-7.34 (m, 1H), 8.32 (dd, J=9.2, 5.7 Hz, 1H).

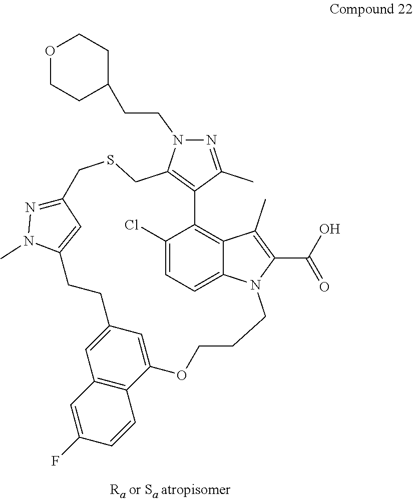 [SMILES: Cc1nn(CCC2CCOCC2)c2c1-c1c(Cl)ccc3c1c(C)c(C(=O)O)n3CCCOc1cc(cc3cc(F)ccc13)CCc1cc(nn1C)CSC2]

LiOH (18 mg, 15 eq.) was added to a solution of Intermediate 68 (39 mg, 0.05 mmol) in a mixture of MeOH (1.2 mL), THE (1.2 mL), and water (0.6 mL). The resulting reaction mixture was stirred for 4 h at 50° C. The reaction mixture was concentrated under reduced pressure to give a pale yellow solid. This solid was dissolved in water and DCM and acidified with 1 M aqueous HCl to pH 4-5, a pale yellow precipitate forming upon acidification. The aqueous layer was extracted with DCM (×4). The combined organic layer was dried over MgSO₄, filtered, and evaporated to give Compound 22 (33 mg, yield: 86%) as a pale yellow solid.

¹H NMR (400 MHz, CDCl₃) δ ppm 1.38 (br d, J=31.5 Hz, 3H), 1.60-1.67 (m, 3H), 1.85-1.95 (m, 2H), 2.04 (s, 3H), 2.17 (s, 3H), 2.32 (br d, J=8.4 Hz, 2H), 2.81 (d, J=10.3 Hz, 2H), 2.92 (s, 2H), 2.95 (s, 3H), 3.18 (br d, J=4.4 Hz, 1H), 3.23 (d, J=11.7 Hz, 1H), 3.30-3.35 (m, 2H), 3.35-3.40 (m, 2H), 3.55 (d, J=15.4 Hz, 1H), 3.89-3.97 (m, 2H), 4.07 (t, J=7.0 Hz, 2H), 4.51 (br d, J=14.7 Hz, 1H), 5.17-5.30 (m, 1H), 5.35 (s, 1H), 5.59 (s, 1H), 7.16 (s, 1H), 7.23-7.25 (m, 1H), 7.27-7.28 (m, 1H), 7.31 (s, 1H), 7.32-7.34 (m, 1H), 8.30 (dd, J=9.1, 5.8 Hz, 1H).

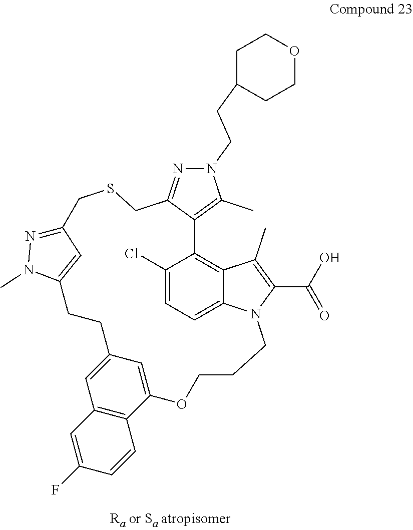 [SMILES: Cc1c(C(=O)O)n2c3ccc(Cl)c(c13)-c1c(nn(CCC3CCOCC3)c1C)CSCc1cc(n(C)n1)CCc1cc(c3ccc(F)cc3c1)OCCC2]

¹H NMR (400 MHz, CDCl₃) δ ppm 1.35 (br d, J=7.9 Hz, 1H), 1.57-1.70 (m, 4H), 1.80 (t, J=6.5 Hz, 2H), 2.05 (s, 3H), 2.20 (s, 3H), 2.25-2.37 (m, 2H), 2.77 (d, J=9.2 Hz, 2H), 2.90 (s, 3H), 2.93-3.01 (m, 3H), 3.14 (br d, J=3.7 Hz, 1H), 3.18 (d, J=11.4 Hz, 1H), 3.22 (d, J=15.0 Hz, 1H), 3.31-3.44 (m, 3H), 3.74 (s, 1H), 3.94 (br d, J=11.9 Hz, 2H), 4.14 (t, J=7.4 Hz, 2H), 4.44-4.55 (m, 1H), 5.30 (s, 1H), 5.35 (s, 1H), 5.67 (s, 1H), 7.18 (s, 1H), 7.27 (br d, J=1.3 Hz, 1H), 7.29-7.31 (m, 1H), 7.32 (s, 1H), 7.32-7.34 (m, 1H), 8.30 (dd, J=9.0, 5.9 Hz, 1H).

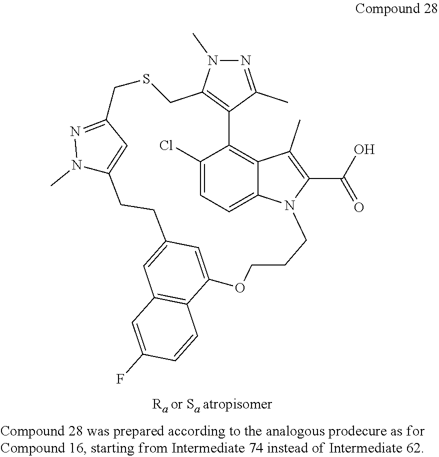 [SMILES: Cc1nn(C)c2c1-c1c(Cl)ccc3c1c(C)c(C(=O)O)n3CCCOc1cc(cc3cc(F)ccc13)CCc1cc(nn1C)CSC2]

¹H NMR (400 MHz, CDCl₃) δ ppm: 2.03 (s, 3H), 2.18 (s, 3H), 2.32 (br s, 2H), 2.77-2.85 (m, 1H), 2.87 (br s, 3H), 2.92 (br d, J=12.2 Hz, 1H), 3.04 (br s, 3H), 3.23 (d, J=12.5 Hz, 1H), 3.26-3.36 (m, 2H), 3.37-3.47 (m, 1H), 3.47-3.55 (m, 1H), 3.85 (s, 3H), 4.54 (br d, J=15.4 Hz, 1H), 5.20 (br d, J=9.0 Hz, 1H), 5.46 (br s, 2H), 7.16 (s, 2H), 7.24 (br d, J=2.5 Hz, 1H), 7.27-7.34 (m, 2H), 8.32 (dd, J=9.0, 5.7 Hz, 1H).

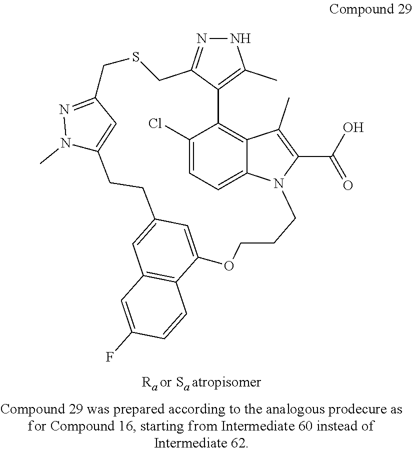 [SMILES: Cc1[nH]nc2c1-c1c(Cl)ccc3c1c(C)c(C(=O)O)n3CCCOc1cc(cc3cc(F)ccc13)CCc1cc(nn1C)CSC2]

¹H NMR (400 MHz, CDCl₃) δ ppm 2.16 (s, 2H), 2.19 (s, 3H), 2.34 (br d, J=5.3 Hz, 2H), 2.86 (s, 3H), 2.95 (d, J=12.6 Hz, 1H), 3.10 (s, 3H), 3.19 (d, J=12.5 Hz, 1H), 3.35 (br d, J=4.5 Hz, 2H), 3.41 (br d, J=14.8 Hz, 1H), 3.63 (br d, J=15.0 Hz, 1H), 4.56 (br d, J=15.4 Hz, 1H), 5.19-5.28 (m, 1H), 5.43 (s, 1H), 5.50 (s, 1H), 7.13 (s, 1H), 7.17 (d, J=8.9 Hz, 1H), 7.19-7.25 (m, 2H), 7.30 (dd, J=10.0, 2.4 Hz, 1H), 7.34 (d, J=9.1 Hz, 1H), 8.31 (dd, J=9.1, 5.7 Hz, 1H).

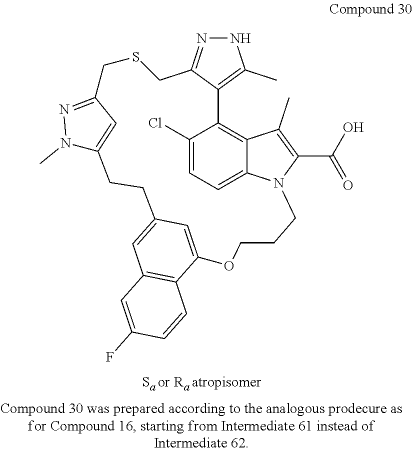 [SMILES: Cc1[nH]nc2c1-c1c(Cl)ccc3c1c(C)c(C(=O)O)n3CCCOc1cc(cc3cc(F)ccc13)CCc1cc(nn1C)CSC2]

¹H NMR (400 MHz, CDCl₃) δ ppm 2.12 (s, 2H), 2.19 (s, 3H), 2.34 (br d, J=4.6 Hz, 2H), 2.86 (br d, J=6.1 Hz, 3H), 2.97 (d, J=12.4 Hz, 1H), 3.09 (s, 3H), 3.20 (d, J=12.4 Hz, 1H), 3.28-3.36 (m, 2H), 3.36-3.40 (m, 1H), 3.58 (d, J=15.3 Hz, 1H), 4.49-4.59 (m, 1H), 5.19-5.27 (m, 1H), 5.46 (s, 1H), 5.49 (s, 1H), 7.12 (s, 1H), 7.19 (d, J=8.9 Hz, 1H), 7.21-7.25 (m, 2H), 7.28-7.32 (m, 1H), 7.32-7.35 (m, 1H), 8.29 (dd, J=9.2, 5.7 Hz, 1H).

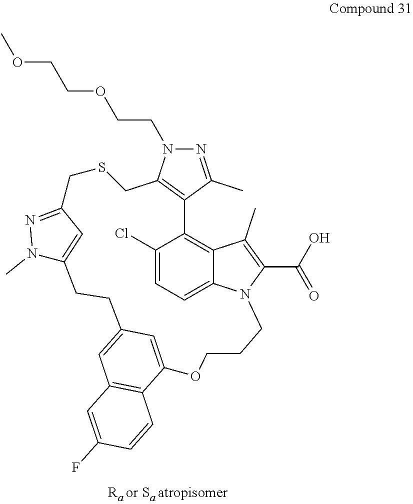 [SMILES: COCCOCCn1nc(C)c2c1CSCc1cc(n(C)n1)CCc1cc(c3ccc(F)cc3c1)OCCCn1c(C(=O)O)c(C)c3c-2c(Cl)ccc31]

LiOH (52 mg, 15 eq.) was added to a stirred solution of Intermediate 84 (112 mg, 0.144 mmol) in water (1.7 mL), THE (3.4 mL), and MeOH (3.4 mL) at room temperature. The reaction mixture was stirred at 50° C. overnight. The reaction mixture was concentrated under reduced pressure and the residue was diluted with water (15 mL) and acidified with 1 M aqueous HCl until acidic pH. This aqueous solution was extracted twice with DCM (10 mL), then with a 1:1 mixture of EtOAc:THF (10 mL). The combined organic layer was dried over MgSO₄, filtered, and evaporated. The residue was co-evaporated with DCM and tBuOMe to yield Compound 31 (109 mg, yield: 99%) as an off-white solid.

¹H NMR (400 MHz, CDCl₃) δ ppm 2.04 (s, 3H) 2.18 (s, 3H) 2.24-2.42 (m, 2H) 2.78-2.96 (m, 5H) 3.01 (s, 3H) 3.20-3.25 (m, 2H) 3.32-3.39 (m, 5H) 3.46-3.50 (m, 2H) 3.51-3.65 (m, 2H) 3.82 (d, J=15.57 Hz, 1H) 3.86-3.98 (m, 2H) 4.21-4.35 (m, 2H) 4.47-4.59 (m, 1H) 5.23 (ddd, J=14.84, 8.94, 3.61 Hz, 1H) 5.41 (s, 1H) 5.56 (s, 1H) 7.17 (s, 1H) 7.20-7.26 (m, 2H) 7.28-7.35 (m, 2H) 8.32 (dd, J=9.14, 5.80 Hz, 1H).

OR=+102.2 (c=0.21 w/v %. DMF, 20 C.

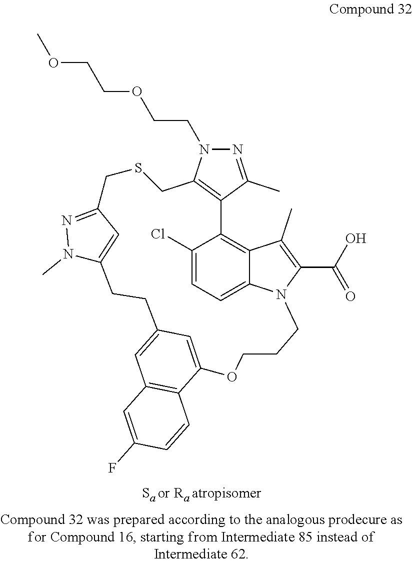 [SMILES: COCCOCCn1nc(C)c2c1CSCc1cc(n(C)n1)CCc1cc(c3ccc(F)cc3c1)OCCCn1c(C(=O)O)c(C)c3c-2c(Cl)ccc31]

¹H NMR (400 MHz, CDCl₃) δ ppm 2.03 (s, 3H) 2.19 (s, 3H) 2.25-2.41 (m, 2H) 2.81-2.95 (m, 5H) 3.10 (s, 3H) 3.22 (d, J=12.76 Hz, 1H) 3.29-3.38 (m, 5H) 3.42 (d, J=15.63 Hz, 1H) 3.46-3.56 (m, 3H) 3.57-3.65 (m, 1H) 3.76 (d, J=15.63 Hz, 1H) 3.86-3.98 (m, 2H) 4.25-4.36 (m, 2H) 4.54 (ddd, J=14.52, 6.82, 3.74 Hz, 1H) 5.21 (ddd, J=14.69, 7.65, 3.85 Hz, 1H) 5.44 (s, 1H) 5.50 (s, 1H) 7.11-7.26 (m, 2H) 7.27-7.37 (m, 2H) 8.33 (dd, J=9.24, 5.72 Hz, 1H).

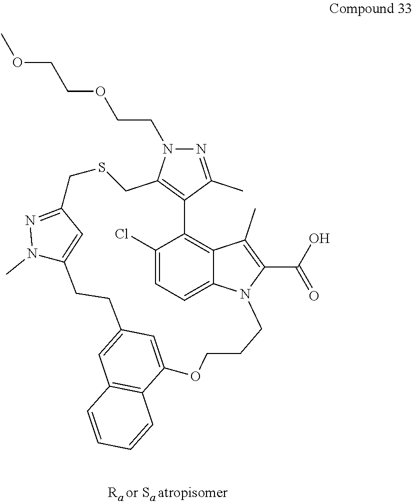 [SMILES: COCCOCCn1nc(C)c2c1CSCc1cc(n(C)n1)CCc1cc(c3ccccc3c1)OCCCn1c(C(=O)O)c(C)c3c-2c(Cl)ccc31]

LiOH (183 mg, 15 eq.) was added to a stirred solution of Intermediate 87 (389 mg, 0.514 mmol) in water (6 mL), THE (12 mL), and MeOH (12 mL) at room temperature. The reaction mixture was stirred at 50° C. for 18 h. The reaction mixture was concentrated under reduced pressure and then diluted with water (30 mL) and acidified with 1 M aqueous HCl until acidic pH. This aqueous phase was extracted twice with DCM (25 mL), then with a 1:1 mixture of EtOAc:THF (25 mL). The combined organic layer was dried over MgSO₄, filtered, and evaporated. The residue was coevaporated a couple of times with n-heptane. The obtained solid was purified by flash column chromatography (silica; MeOH in DCM 0/100 to 5/95) to yield Compound 33 (332 mg, yield: 87%) as an off-white solid.

¹H NMR (400 MHz, CDCl₃) δ ppm 2.03 (s, 3H) 2.19 (s, 3H) 2.26-2.43 (m, 2H) 2.83-2.95 (m, 5H) 3.12 (s, 3H) 3.22 (d, J=12.75 Hz, 1H) 3.27-3.37 (m, 4H) 3.43 (br d, J=15.68 Hz, 2H) 3.46-3.51 (m, 2H) 3.51-3.56 (m, 1H) 3.56-3.63 (m, 1H) 3.75 (d, J=15.57 Hz, 1H) 3.86-3.98 (m, 2H) 4.25-4.35 (m, 2H) 4.57 (ddd, J=14.79, 7.11, 3.71 Hz, 1H) 5.20 (ddd, J=14.68, 7.47, 3.76 Hz, 1H) 5.40 (s, 1H) 5.55 (s, 1H) 7.11 (d, J=8.99 Hz, 1H) 7.23 (s, 1H) 7.31 (d, J=8.99 Hz, 1H) 7.46-7.54 (m, 2H) 7.70-7.76 (m, 1H) 8.31-8.37 (m, 1H).

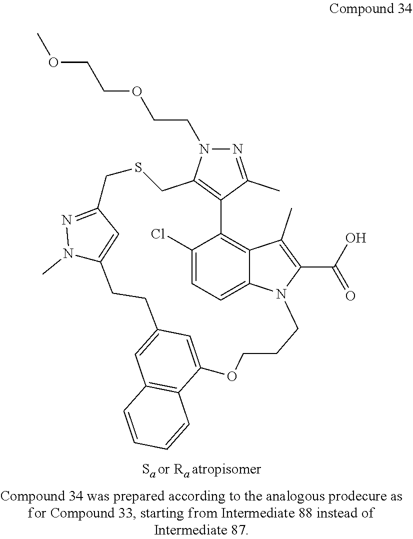 [SMILES: COCCOCCn1nc(C)c2c1CSCc1cc(n(C)n1)CCc1cc(c3ccccc3c1)OCCCn1c(C(=O)O)c(C)c3c-2c(Cl)ccc31]

¹H NMR (400 MHz, CDCl₃) δ ppm 2.03 (s, 3H) 2.19 (s, 3H) 2.25-2.44 (m, 2H) 2.83-2.94 (m, 5H) 3.09 (s, 3H) 3.22 (d, J=12.65 Hz, 1H) 3.29-3.45 (m, 6H) 3.46-3.51 (m, 2H) 3.51-3.56 (m, 1H) 3.56-3.64 (m, 1H) 3.76 (d, J=15.47 Hz, 1H) 3.86-3.98 (m, 2H) 4.24-4.36 (m, 2H) 4.57 (ddd, J=14.47, 6.95, 3.87 Hz, 1H) 5.21 (ddd, J=14.84, 7.79, 3.61 Hz, 1H) 5.43 (s, 1H) 5.53 (d, J=0.84 Hz, 1H) 7.13 (d, J=8.99 Hz, 1H) 7.23 (s, 1H) 7.31 (d, J=8.99 Hz, 1H) 7.46-7.54 (m, 2H) 7.70-7.76 (m, 1H) 8.31-8.37 (m, 1H).

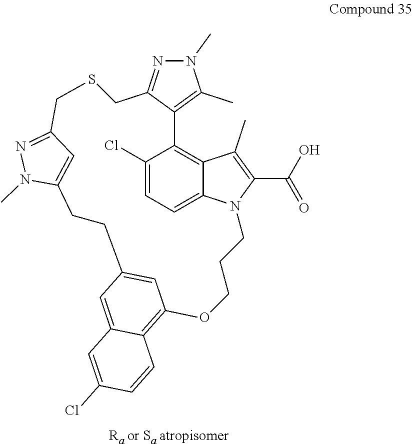 [SMILES: Cc1c(C(=O)O)n2c3ccc(Cl)c(c13)-c1c(nn(C)c1C)CSCc1cc(n(C)n1)CCc1cc(c3ccc(Cl)cc3c1)OCCC2]

LiOH (2 M in water, 4.5 mL, 15 eq.) was added to a solution of Intermediate 100 (420 mg, 0.598 mmol) in MeOH (10 mL) and THE (10 mL). The reaction mixture was stirred at 60° C. for 4 h. After cooling, the reaction mixture was concentrated under vacuum and then diluted with water (5 mL). The pH of the solution was adjusted to 1-2 with 2 M aqueous HCl. The resulting mixture was extracted with EtOAc (3×50 mL). The combined organic layer was combined, dried over Na₂SO₄, filtered, and evaporated. The 10 residue was purified by reverse-phase flash chromatography (Column: Sunfire Prep C18 OBD Column, 30*100 mm 5 um 10 nm; Mobile Phase A: Water (10 mM NH₄HCO₃), Mobile Phase B: ACN; Flow rate: 60 mL/min) to afford Compound 35 (209 mg, yield: 51%), as an off-white solid.

MP: 220° C. (Tianjin RY-2 type melting point apparatus)

OR: +32.9° (c=0.1 w/v; DMSO; 589 nm; 26.5° C.); +71.8° (c=0.1 w/v; MeOH; 589 nm; 21.6° C.)

¹H NMR (300 MHz, DMSO-d₆) δ ppm 8.19 (d, J=9.0 Hz, 1H), 7.84 (d, J=2.1 Hz, 1H), 7.48-7.41 (m, 2H), 7.19 (s, 1H), 7.01 (d, J=8.9 Hz, 1H), 6.21 (s, 1H), 5.06 (d, J=14.2 Hz, 1H), 4.94 (s, 1H), 4.56-4.51 (m, 1H), 3.75 (s, 4H), 3.60-3.51 (m, 1H), 3.43-3.28 (m, 5H), 3.17 (s, 1H), 3.07-2.92 (m, 3H), 2.87-2.73 (m, 3H), 2.29 (s, 2H), 2.00 (s, 3H), 1.91 (s, 3H).

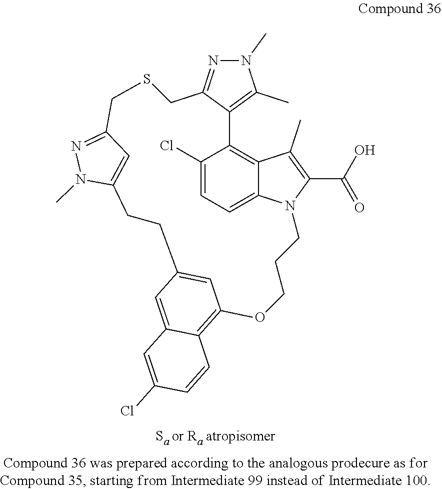 [SMILES: Cc1c(C(=O)O)n2c3ccc(Cl)c(c13)-c1c(nn(C)c1C)CSCc1cc(n(C)n1)CCc1cc(c3ccc(Cl)cc3c1)OCCC2]

MP: 211° C. (Tianjin RY-2 type melting point apparatus)

OR: −49.2° (c=0.1 w/v; DMSO; 589 nm; 27.1° C.); −76.9° (c=0.1 w/v; MeOH; 589 nm; 22.1° C.)

¹H NMR (300 MHz, DMSO-d₆) δ ppm 8.18 (d, J=9.0 Hz, 1H), 7.85 (d, J=2.2 Hz, 1H), 7.47-7.40 (m, 2H), 7.18 (s, 1H), 7.00 (d, J=8.8 Hz, 1H), 6.21 (s, 1H), 5.09 (d, J=13.8 Hz, 1H), 4.93 (s, 1H), 4.55-4.50 (m, 1H), 3.75 (s, 4H), 3.55 (d, J=7.4 Hz, 1H), 3.43 (s, 3H), 3.23 (d, J=32.7 Hz, 2H), 3.18 (s, 1H), 3.07-2.99 (m, 3H), 2.83-2.78 (m, 3H), 2.42-2.29 (m, 2H), 2.02 (s, 3H), 1.91 (s, 3H).

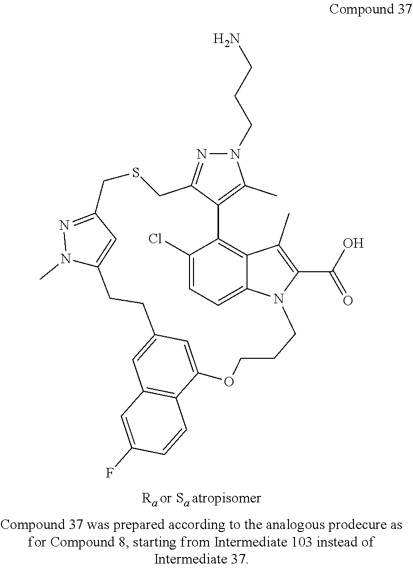 [SMILES: Cc1c(C(=O)O)n2c3ccc(Cl)c(c13)-c1c(nn(CCCN)c1C)CSCc1cc(n(C)n1)CCc1cc(c3ccc(F)cc3c1)OCCC2]

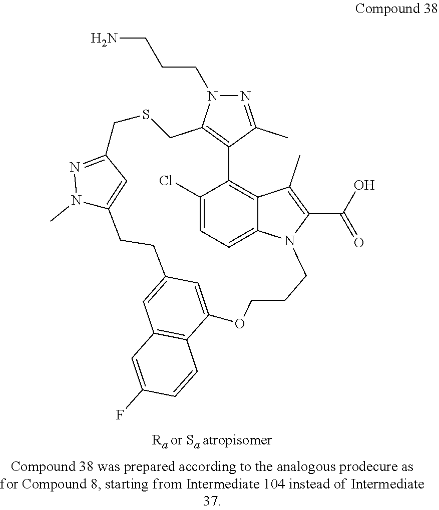 [SMILES: Cc1nn(CCCN)c2c1-c1c(Cl)ccc3c1c(C)c(C(=O)O)n3CCCOc1cc(cc3cc(F)ccc13)CCc1cc(nn1C)CSC2]

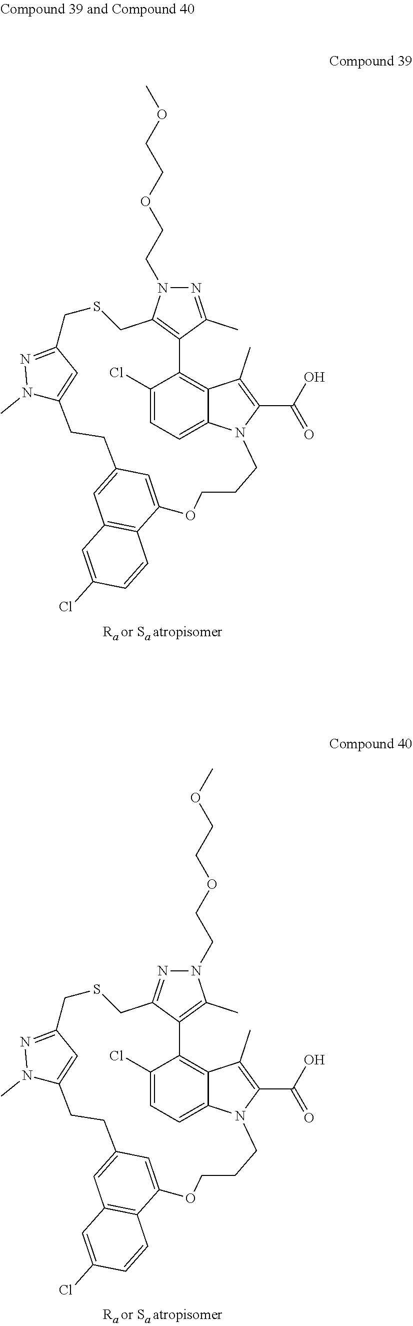 [SMILES: COCCOCCn1nc(C)c2c1CSCc1cc(n(C)n1)CCc1cc(c3ccc(Cl)cc3c1)OCCCn1c(C(=O)O)c(C)c3c-2c(Cl)ccc31 ; COCCOCCn1nc2c(c1C)-c1c(Cl)ccc3c1c(C)c(C(=O)O)n3CCCOc1cc(cc3cc(Cl)ccc13)CCc1cc(nn1C)CSC2]

LiOH (55 mg, 12 eq.) was added to the mixture of Intermediate 131 and Intermediate 132 (300 mg, 0.379 mmol) in THF (4 mL) and H₂O (4 mL) under nitrogen atmosphere. The resulting mixture was stirred at room temperature under nitrogen atmosphere for 48 h. The reaction mixture was concentrated under vacuum and then diluted with water (5 mL). The pH of the solution was adjusted to 1-2 with 3 M aqueous HCL. The resulting mixture was extracted with EtOAc (3×10 mL). The combined organic layer was dried over Na₂SO₄, filtered, and concentrated. The residue was purified by preparative chiral SFC (Column: Phenomenex Lux 5u Cellulose-3, 5×25 cm, 5 m; Mobile Phase A: CO₂, Mobile Phase B: MeOH/ACN 1/1 (0.1% 2 M NH₃-MeOH); Gradient: 40% B) to afford Compound 39 (28 mg, yield: 9%) and Compound 40 (25 mg, yield: 16%), both as a light yellow solids.

Compound 39

¹H NMR (300 MHz, CDCl₃) δ ppm 8.25 (d, J=6.0 Hz, 1H), 7.70 (s, 1H), 7.50-7.36 (m, 2H), 7.04 (s, 2H), 5.75 (s, 1H), 5.19 (d, J=9 Hz, 1H), 5.12 (s, 1H), 4.93 (s, 1H), 4.63 (s, 2H), 4.10 (s, 2H), 3.63 (s, 4H), 3.51 (s, 6H), 3.34 (s, 5H), 3.34-2.91 (m, 3H), 2.81 (s, 2H), 2.48 (s, 2H), 2.26 (s, 3H), 2.14 (s, 3H).

Compound 40

¹H NMR (300 MHz, CDCl₃) δ ppm 8.32 (d, J=9.0 Hz, 1H), 7.77 (s, 1H), 7.56-7.31 (m, 2H), 7.20 (s, 1H), 6.93 (s, 1H), 5.89 (s, 1H), 5.18 (s, 2H), 4.64 (s, 2H), 4.07 (s, 2H), 3.90-3.40 (m, 9H), 3.34 (s, 3H), 3.26-2.60 (m, 9H), 2.43 (s, 2H), 2.20 (s, 6H).

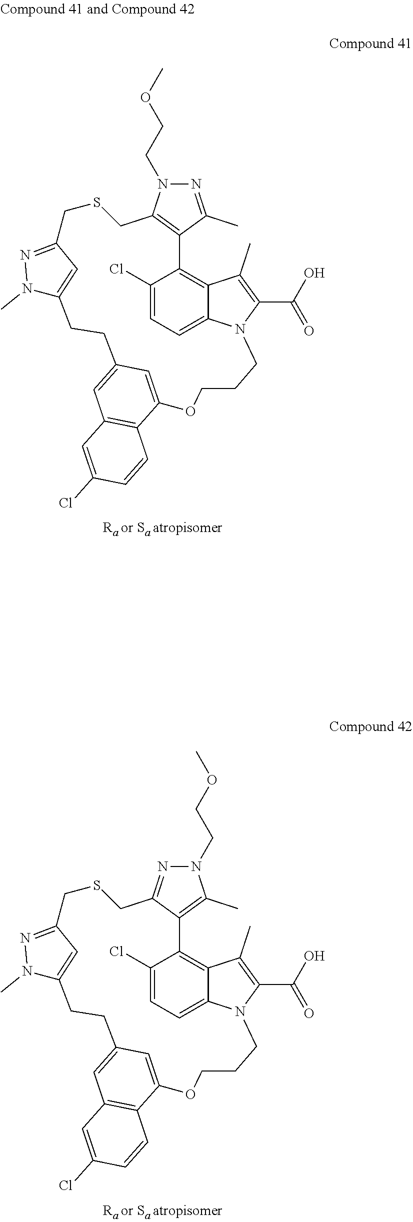 [SMILES: COCCn1nc(C)c2c1CSCc1cc(n(C)n1)CCc1cc(c3ccc(Cl)cc3c1)OCCCn1c(C(=O)O)c(C)c3c-2c(Cl)ccc31 ; COCCn1nc2c(c1C)-c1c(Cl)ccc3c1c(C)c(C(=O)O)n3CCCOc1cc(cc3cc(Cl)ccc13)CCc1cc(nn1C)CSC2]

LiOH (77 mg, 12 eq.) was added to the mixture of Intermediate 133 and Intermediate 134 (400 mg, 0.536 mmol) in THF (4 mL) and H₂O (4 mL) under nitrogen atmosphere. The resulting mixture was stirred at 40° C. under nitrogen atmosphere for 48 h. The reaction mixture was concentrated under vacuum and then diluted with water (10 mL). The pH of the solution was adjusted to 1-2 with 3 M aqueous HCl. The resulting mixture was extracted with EtOAc (3×10 mL). The combined organic layer was dried over Na₂SO₄, filtered, and concentrated. The residue was purified by preparative HPLC (Column: XSelect CSH Prep C18 OBD, 5 um, 19×150 mm; Mobile Phase A: Water (0.05% HCl), Mobile Phase B: ACN; Gradient: 63% B to 78% B in 7 min) to afford Compound 41 (89 mg, yield: 43%) and Compound 42 (89 mg, yield: 43%), both as light yellow solids.

A sample of Compound 41 (52 mg, 0.068 mmol) was dissolved in MeOH (2 mL) and NaOH (1 M in H₂O, 68 μL, 1 eq.) was added. The mixture was stirred for a few min, then volatiles were removed under reduced pressure. The residue was suspended in DIPE (2 mL) and evaporated to dryness. The residue was then triturated with DIPE, filtered, and dried under vacuum at 55° C. for 2 h to afford the sodium salt of Compound 41 (40 mg, yield: 73%) as an off-white solid.

Compound 41

¹H NMR (300 MHz, CDCl₃) δ ppm 8.15 (d, J=9.0 Hz, 1H), 7.65 (s, 1H), 7.50-7.39 (m, 2H), 7.08 (s, 1H), 6.88 (s, 1H), 5.82 (s, 1H), 5.22 (d, J=14.1 Hz, 1H), 4.90 (s, 2H), 4.64 (s, 2H), 3.97 (s, 2H), 3.85 (s, 1H), 3.54 (s, 3H), 3.52-3.43 (m, 2H), 3.37 (s, 3H), 3.33-2.89 (m, 5H), 2.85-2.63 (m, 2H), 2.63-2.31 (m, 2H), 2.22 (s, 3H), 2.09 (s, 3H).

Compound 42

¹H NMR (300 MHz, CDCl₃) δ ppm 8.35 (d, J=9.0 Hz, 1H), 7.78 (s, 1H), 7.48 (d, J=9.0 Hz, 1H), 7.34 (d, J=9.0 Hz, 1H), 7.20 (s, 1H), 6.87-6.84 (m, 1H), 5.98 (s, 1H), 5.17 (d, J=14.1 Hz, 1H), 5.04 (s, 1H), 4.78-4.47 (m, 3H), 4.11 (s, 1H), 4.02-3.58 (m, 6H), 3.35 (s, 5H), 3.07 (s, 3H), 2.83 (s, 2H), 2.64-2.31 (m, 3H), 2.24 (s, 3H), 2.18 (s, 3H).

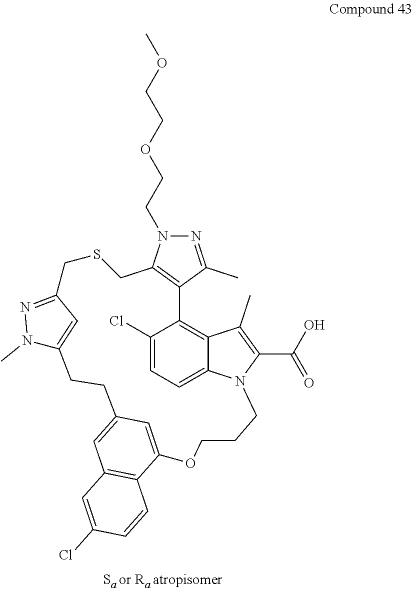 [SMILES: COCCOCCn1nc(C)c2c1CSCc1cc(n(C)n1)CCc1cc(c3ccc(Cl)cc3c1)OCCCn1c(C(=O)O)c(C)c3c-2c(Cl)ccc31]

LiOH (13 mg, 6 eq.) was added to a solution of Intermediate 135 (70 mg, 0.089 mmol) in THF (2 mL) and H₂O (2 mL) under nitrogen atmosphere. The resulting mixture was stirred under nitrogen atmosphere at room temperature for 48 h. The mixture was concentrated under vacuum and then diluted with water (5 mL). The pH of the solution was adjusted to 1-2 with 3 M HCl. The resulting mixture was extracted with EtOAc (3×10 mL). The combined organic layer was combined, dried over Na₂SO₄, filtered, and concentrated under reduced pressure. The residue was purified by preparative HPLC (Column: XBridge Prep OBD C18 Column, 19×250 mm, 5 um; Mobile Phase A: Water (0.05% HCl), Mobile Phase B: ACN; Gradient: 73% B to 83% B in 7 min) to afford Compound 43 (25 mg, yield: 37%) as a light yellow solid.

¹H NMR (300 MHz, CDCl₃) δ ppm 8.28 (d, J=9.0 Hz, 1H), 7.71 (s, 1H), 7.51-7.37 (m, 2H), 7.11 (s, 2H), 5.61 (s, 1H), 5.32 (s, 1H), 5.22 (d, J=15.0 Hz, 1H), 4.90 (s, 1H), 4.60 (s, 2H), 4.08 (s, 2H), 3.62 (s, 5H), 3.38 (s, 2H), 3.33 (s, 3H), 3.21 (s, 5H), 2.99 (s, 3H), 2.81 (s, 2H), 2.43 (s, 2H), 2.28 (s, 3H), 2.18 (s, 3H).

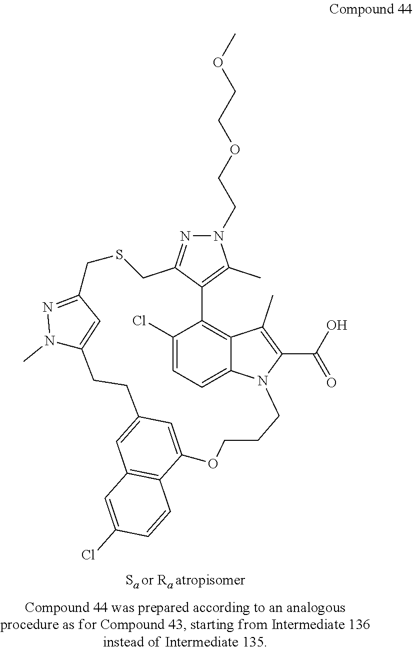 [SMILES: COCCOCCn1nc2c(c1C)-c1c(Cl)ccc3c1c(C)c(C(=O)O)n3CCCOc1cc(cc3cc(Cl)ccc13)CCc1cc(nn1C)CSC2]

1H NMR (300 MHz, CDCl₃) δ ppm 8.29 (d, J=9.0 Hz, 1H), 7.74 (s, 1H), 7.50-7.31 (m, 2H), 7.21-7.04 (m, 2H), 5.63 (s, 1H), 5.37 (s, 1H), 5.22 (d, J=9.0 Hz, 1H), 4.56 (s, 3H), 4.00 (s, 2H), 3.75 (d, J=15.0 Hz, 1H), 3.65-3.12 (m, 14H), 3.92 (s, 5H), 2.38 (s, 2H), 2.18 (d, J=12.0 Hz, 6H).

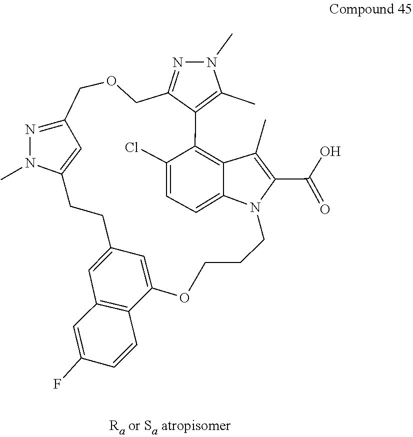 [SMILES: Cc1c(C(=O)O)n2c3ccc(Cl)c(c13)-c1c(nn(C)c1C)COCc1cc(n(C)n1)CCc1cc(c3ccc(F)cc3c1)OCCC2]

LiOH (18 mg, 6 eq.) was added to a solution of Intermediate 119 (85 mg, 0.127 mmol) in MeOH (0.5 mL), THE (3 mL), and water (3 mL). The reaction mixture was stirred at 40° C. for 16 h under a nitrogen atmosphere. After cooling, the reaction mixture was concentrated under vacuum and then diluted with water (5 mL) and diethyl ether (5 mL). The layers were separated and the aqueous layer was extracted with diethyl ether (3×10 mL). The pH of the aqueous layer was then adjusted to 3-4 with 2 M aqueous HCl. The resulting precipitate was filtered to afford Compound 45 (53 mg, yield: 63%) as an off-white solid.

¹H NMR (400 MHz, CD₃OD) δ ppm 8.24 (m, 1H), d 7.48 (m, 1H), d 7.34 (m, 1H), d 7.19 (m, 1H), d 7.10 (m, 2H), d 6.21 (s, 1H), d 5.21 (s, 2H), d 4.62 (m, 1H), d 4.16 (m, 2H), d 3.90 (m, 5H), d 3.76 (s, 1H), d 3.64 (s, 4H), d 3.06 (m, 2H), d 2.93 (m, 2H), d 2.37 (s, 2H), d 2.06 (m, 6H).

¹⁹F NMR (376 MHz, CD₃OD) 6-117.2.

OR: +5.12° (c=0.5 w/v. MeOH. 28.8° C.).

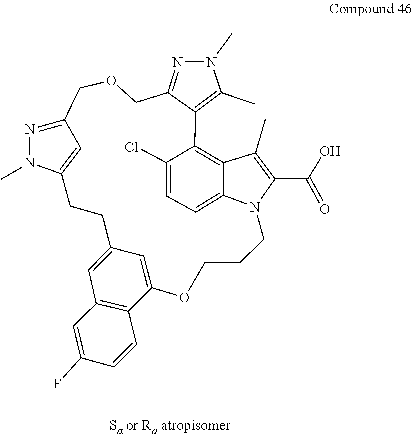 [SMILES: Cc1c(C(=O)O)n2c3ccc(Cl)c(c13)-c1c(nn(C)c1C)COCc1cc(n(C)n1)CCc1cc(c3ccc(F)cc3c1)OCCC2]

Compound 46 was prepared according to an analogous procedure as for Compound 45, starting from Intermediate 118 instead of Intermediate 119.

¹H NMR (400 MHz, CD₃OD) δ ppm 8.24 (m, 1H), d 7.48 (d, J=8.0 Hz, 1H), d 7.34 (m, 1H), d 7.19 (m, 1H), d 7.10 (m, 2H), d 6.21 (s, 1H), d 5.21 (s, 2H), d 4.61 (m, 1H), d 4.17 (m, 2H), d 3.99 (d, J=12.0 Hz, 1H), d 3.90 (d, J=12.0 Hz, 1H), d 3.85 (s, 3H), d 3.77 (m, 1H), d 3.63 (m, 1H), d 3.55 (m, 3H), d 3.06 (m, 2H), d 2.94 (m, 2H), d 2.37 (s, 2H), d 2.06 (m, 6H).

¹⁹F NMR (376 MHz, CD₃OD) 6-117.2

OR: −9.06° (c=0.5 w/v. MeOH. 28.8° C.).

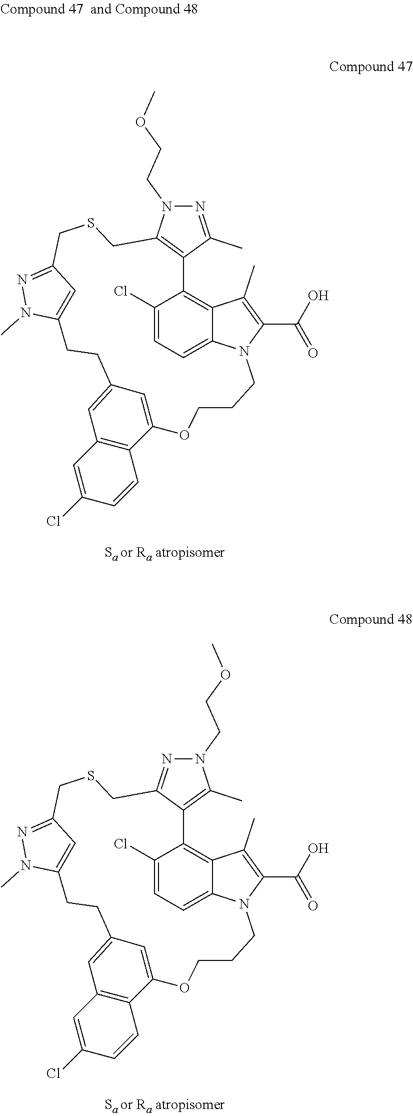 [SMILES: COCCn1nc(C)c2c1CSCc1cc(n(C)n1)CCc1cc(c3ccc(Cl)cc3c1)OCCCn1c(C(=O)O)c(C)c3c-2c(Cl)ccc31 ; COCCn1nc2c(c1C)-c1c(Cl)ccc3c1c(C)c(C(=O)O)n3CCCOc1cc(cc3cc(Cl)ccc13)CCc1cc(nn1C)CSC2]

Compound 47

Compound 47 and Compound 48 were prepared prepared according to an analogous procedure as for Compound 41 and Compound 42, starting from the mixture of Intermediate 137 and Intermediate 138 instead of the mixture of Intermediate 133 and Intermediate 134.

¹H NMR (300 MHz, CDCl₃) δ ppm 8.17 (d, J=9.0 Hz, 1H), 7.65 (s, 1H), 7.43-7.35 (m, 2H), 7.08 (d, J=8.1 Hz, 1H), 6.88 (s, 1H), 5.82 (s, 1H), 5.22 (d, J=14.1 Hz, 1H), 4.91 (s, 2H), 4.64 (s, 2H), 3.97 (s, 2H), 3.84 (s, 1H), 3.53 (s, 3H), 3.52-3.43 (m, 2H), 3.37 (s, 3H), 3.30-3.05 (m, 5H), 2.83-2.61 (m, 2H), 2.51 (s, 2H), 2.22 (s, 3H), 2.10 (s, 3H).

Compound 48

¹H NMR (300 MHz, CDCl₃) δ ppm 8.35 (d, J=9.0 Hz, 1H), 7.78 (s, 1H), 7.48 (d, J=9.0 Hz, 1H), 7.34 (d, J=9.0 Hz, 1H), 7.19 (s, 1H), 6.87 (d, J=9.0 Hz, 1H), 5.94 (s, 1H), 5.17 (d, J=14.1 Hz, 1H), 5.06 (s, 1H), 4.82-4.55 (m, 3H), 4.09 (s, 1H), 3.99-3.82 (m, 3H), 3.67 (s, 3H), 3.34 (s, 5H), 3.18-2.92 (m, 3H), 2.83 (s, 2H), 2.59 (s, 1H), 2.43 (s, 2H), 2.23 (s, 3H), 2.17 (s, 3H).

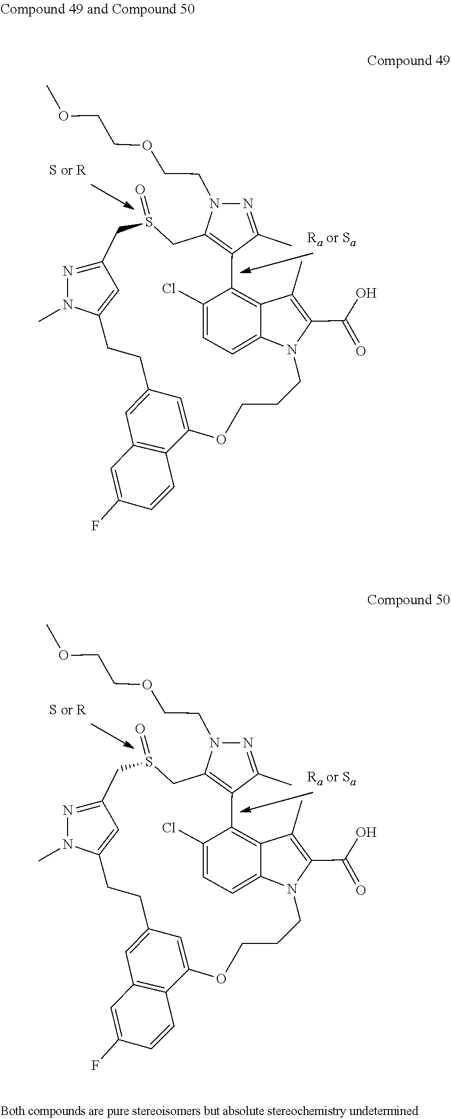 [SMILES: COCCOCCn1nc(C)c2c1C[S@](=O)Cc1cc(n(C)n1)CCc1cc(c3ccc(F)cc3c1)OCCCn1c(C(=O)O)c(C)c3c-2c(Cl)ccc31 ; COCCOCCn1nc(C)c2c1C[S@@](=O)Cc1cc(n(C)n1)CCc1cc(c3ccc(F)cc3c1)OCCCn1c(C(=O)O)c(C)c3c-2c(Cl)ccc31]

Both compounds are pure stereoisomers but absolute stereochemistry undetermined

A cooled (0° C.) solution of Compound 31 (150 mg, 0.2 mmol) in MeOH (2 mL) was added to a cold (0° C.) solution of sodium periodate (55 mg, 0.26 mmol, 1.3 eq.) in MeOH (4 mL). The reaction mixture was stirred at room temperature overnight. The solvent was evaporated and the residue was dissolved in DCM and washed with water and brine. The organic layer was dried over MgSO₄, filtered, and evaporated. The residue was purified by preparative HPLC (Stationary phase: RP XBridge Prep C18 OBD—5 μm, 50×250 mm, Mobile phase: 0.25% NH₄HCO₃ solution in water, CH₃CN) to afford Compound 49 (41 mg, yield: 27%) and Compound 50 (19 mg, yield: 13%).

Compound 49

¹H NMR (400 MHz, CDCl₃) δ ppm 2.01 (s, 3H); 2.03 (s, 1H); 2.09 (s, 3H); 2.33 (br s, 2H); 2.83 (br d, J=12.75 Hz, 2H); 2.86 (s, 3H); 2.94 (br d, J=11.29 Hz, 2H); 3.10-3.26 (m, 2H); 3.31 (s, 3H), 3.30-3.37 (m, 1H); 3.42-3.48 (m, 2H); 3.48-3.55 (m, 1H); 3.55-3.62 (m, 1H); 3.75 (d, J=13.69 Hz, 1H); 3.86-4.00 (m, 2H); 4.06 (br d, J=14.00 Hz, 1H); 4.39 (dt, J=14.47, 4.00 Hz, 1H); 4.46-4.63 (m, 2H); 4.49-4.57 (m, 1H); 5.12-5.25 (m, 1H); 5.47 (s, 1H); 5.83 (s, 1H); 7.13 (d, J=8.99 Hz, 1H); 7.20 (s, 1H); 7.23-7.29 (m, 2H); 7.30 (d, J=9.09 Hz, 1H); 7.34 (dd, J=9.98, 2.46 Hz, 1H); 8.34 (dd, J=9.20, 5.75 Hz, 1H).

Compound 50

¹H NMR (400 MHz, CDCl₃, 51° C.) δ ppm 2.00 (s, 3H); 2.25 (s, 3H); 2.32 (br s, 2H); 2.58-2.84 (m, 4H); 2.86-3.04 (m, 7H); 3.12 (br d, J=5.33 Hz, 2H); 3.31 (s, 4H); 3.39-3.60 (m, 6H); 3.86-3.97 (m, 2H); 4.12 (d, J=14.74 Hz, 1H); 4.27-4.38 (m, 1H); 4.41-4.56 (m, 3H); 5.14 (br d, J=14.63 Hz, 1H); 5.53 (s, 2H); 7.01 (d, J=8.91 Hz, 1H); 7.15-7.25 (m, 3H); 7.32 (d, J=9.88 Hz, 1H); 8.31 (dd, J=9.14, 5.80 Hz, 1H)

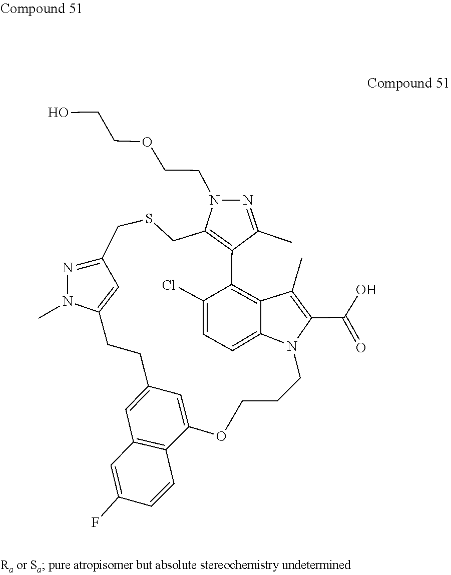 [SMILES: Cc1nn(CCOCCO)c2c1-c1c(Cl)ccc3c1c(C)c(C(=O)O)n3CCCOc1cc(cc3cc(F)ccc13)CCc1cc(nn1C)CSC2]

Trimethylsilyl iodide (CAS [16029-98-4], 1 M in DCM, 0.25 mL, 0.25 mmol, 3 eq.) was added to a slurry of Compound 31 (62 mg, 0.082 mmol) in ACN (4 mL) at 10° C. The resulting dark yellow solution was stirred at reflux for 1 h. The reaction mixture was cooled to 10° C., then treated with aqueous NaOH (1 M, 1 mL), and stirred at room temperature for 20 min. The solvents were evaporated and the residue was dissolved in water, cooled to 0° C., then treated with aqueous HCl (1M, 1 mL). The aqueous layer was extracted with CHCl₃ (3×). The combined organic layer was dried over MgSO₄, filtered, and evaporated. The residue was purified by preparative HPLC (Stationary phase: RP XBridge Prep C18 OBD—10 μm, 30×150 mm, Mobile phase: 0.25% NH₄HCO₃ solution in water, CH₃CN) to afford Compound 51 (32 mg, yield: 52%).

¹H NMR (400 MHz, CDCl₃) δ ppm 1.96 (s, 3H); 2.14 (s, 3H); 2.28-2.38 (m, 2H); 2.72-2.85 (m, 2H); 2.85-2.96 (m, 3H); 3.18 (d, J=13.38 Hz, 1H); 3.22 (s, 3H); 3.22-3.30 (m, 1H); 3.46-3.62 (m, 4H); 3.49-3.54 (m, 1H); 3.66-3.71 (m, 2H); 3.92 (br t, J=4.96 Hz, 2H); 4.19 (br s, 1H); 4.28 (br t, J=4.86 Hz, 2H); 4.43-4.52 (m, 1H); 5.04-5.16 (m, 2H); 5.07-5.09 (m, 1H); 5.19 (s, 1H); 5.61 (s, 1H); 6.98 (d, J=8.97 Hz, 1H); 7.12 (s, 1H); 7.20 (d, J=9.30 Hz, 1H); 7.21-7.25 (m, 1H); 7.32 (d, J=9.84 Hz, 1H); 8.30 (dd, J=9.14, 5.80 Hz, 1H).

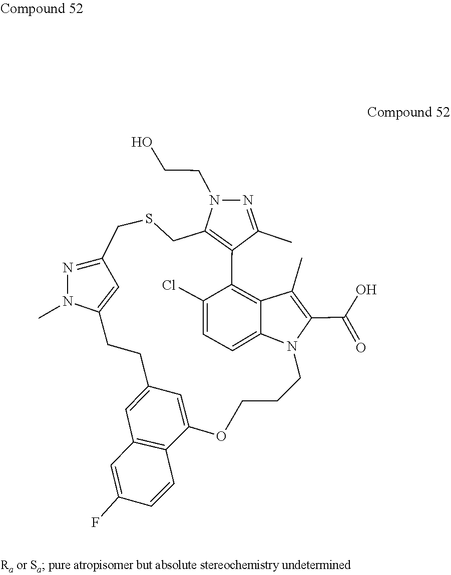 [SMILES: Cc1nn(CCO)c2c1-c1c(Cl)ccc3c1c(C)c(C(=O)O)n3CCCOc1cc(cc3cc(F)ccc13)CCc1cc(nn1C)CSC2]

A solution of lithium hydroxide (0.71 mL, 1 M in water, 0.7 mmol, 10 eq.) was added to a suspension of Intermediate 147 (50 mg, 0.07 mmol) in MeOH/THF (2 mL/2 mL) and the resulting solution was heated at 50° C. for 16 h. The solvents were evaporated and the residue was diluted with DCM (5 mL), treated with water (1 mL) and aqueous HCl (1 M) until pH=1, and the layers were separated. The aqueous layer was extracted with DCM (3×) and the combined organic layer was dried over MgSO₄, filtered, and evaporated. The residual oil was dissolved in DCM/MeOH (5 mL/5 mL) and then slowly evaporated to afford Compound 52 (45 mg, yield: 92%) as a white solid.

¹H NMR (400 MHz, CDCl₃) δ ppm 2.00-2.20 (m, 6H) 2.35 (br s, 2H) 2.80-2.97 (m, 5H) 3.11 (s, 3H) 3.22 (d, J=12.75 Hz, 1H) 3.29-3.55 (m, 4H) 3.73 (q, J=7.00 Hz, 1H) 4.02-4.26 (m, 4H) 4.48-4.65 (m, 1H) 5.14-5.27 (m, 1H) 5.45 (d, J=39.92 Hz, 2H) 7.08-7.26 (m, 3H) 7.28-7.48 (m, 3H) 8.33 (dd, J=9.14, 5.80 Hz, 1H)

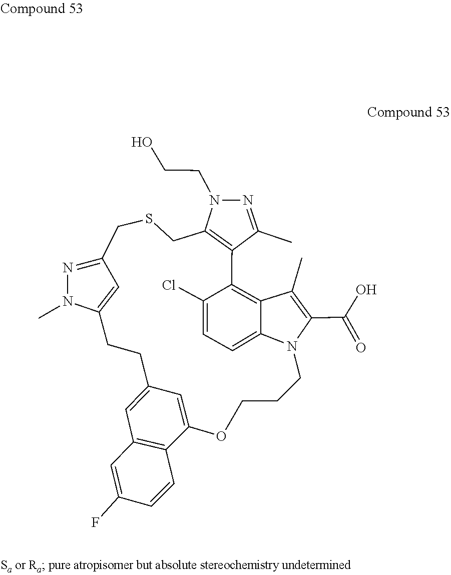 [SMILES: Cc1nn(CCO)c2c1-c1c(Cl)ccc3c1c(C)c(C(=O)O)n3CCCOc1cc(cc3cc(F)ccc13)CCc1cc(nn1C)CSC2]

Compound 53 was prepared according to an analogous procedure as for Compound 52, starting from Intermediate 148 instead of Intermediate 147.

¹H NMR (400 MHz, CDCl₃) δ ppm 2.00-2.10 (m, 4H) 2.16 (s, 3H) 2.25-2.42 (m, 3H) 2.83-2.94 (m, 5H) 3.06 (s, 3H) 3.18-3.26 (m, 1H) 3.32 (br t, J=5.38 Hz, 2H) 3.37-3.45 (m, 1H) 3.47-3.59 (m, 1H) 4.02-4.24 (m, 4H) 4.50-4.59 (m, 1H) 5.21 (ddd, J=14.79, 7.73, 4.13 Hz, 1H) 5.45 (s, 2H) 7.12-7.25 (m, 3H) 7.27-7.34 (m, 2H) 8.32 (dd, J=9.14, 5.80 Hz, 1H)

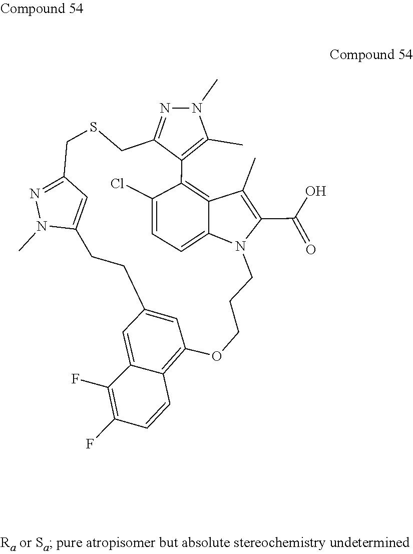 [SMILES: Cc1c(C(=O)O)n2c3ccc(Cl)c(c13)-c1c(nn(C)c1C)CSCc1cc(n(C)n1)CCc1cc(c3ccc(F)c(F)c3c1)OCCC2]

A solution of LiOH (61 mg, 2.556 mmol, 6 eq.) in water (5 mL) was added to a solution of Intermediate 162 (300 mg, 0.426 mmol) in THF (5 mL) and the mixture was stirred at 40° C. for 48 h. Most of the THF was removed under reduced pressure and the mixture was extracted with Et₂O (5 mL×3). The aqueous layer was acidified with aqueous HCl (2 M) to pH=3. The solid that appeared was collected by filtration and was triturated with DCM/petroleum ether (1 m L/10 mL). The solid was filtered to afford Compound 54 (115 mg, yield: 36%) as a white solid.

OR: +46° (589 nm, 24.7° C., 5 mg in 10 mL MeOH)

¹H NMR (300 MHz, Methanol-d4) δ (ppm) 8.06-8.09 (m, 1H), 7.01-7.45 (M, 3H), 7.00 (d, J=9.0 Hz, 1H), 6.05 (s, 1H), 5.13-5.19 (m, 1H), 4.88 (s, 1H), 4.60-4.67 (m, 1H), 3.81-3.85 (m, 4H), 3.49-3.54 (m, 4H), 3.01-3.01 (m, 5H), 2.70-2.87 (m, 3H), 2.34-2.40 (m, 2H), 2.12 (s, 3H), 1.98 (s, 3H).

¹⁹F NMR (300 MHz, Methanol-d4) δ (ppm) −144.0, −152.0

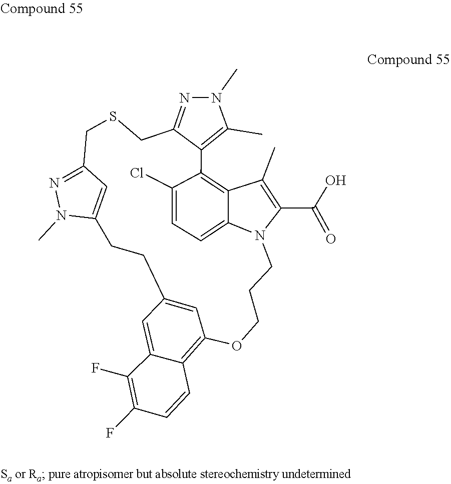 [SMILES: Cc1c(C(=O)O)n2c3ccc(Cl)c(c13)-c1c(nn(C)c1C)CSCc1cc(n(C)n1)CCc1cc(c3ccc(F)c(F)c3c1)OCCC2]

Compound 55 was prepared according to an analogous procedure as for Compound 54, starting from Intermediate 163 instead of Intermediate 162.

OR: −32° (589 nm, 24.7° C., 5 mg in 10 mL MeOH)

¹H NMR (300 MHz, Methanol-d4) δ (ppm) 8.04-8.06 (m, 1H), 7.44 (d, J=9.0 Hz, 1H), 7.29-7.36 (m, 1H), 7.27 (s, 1H), 7.00 (d, J=9.0 Hz, 1H), 6.05 (s, 1H), 5.13-5.19 (m, 1H), 4.88 (s, 1H), 4.60-4.67 (m, 1H), 3.81-3.85 (m, 4H), 3.49-3.54 (m, 4H), 3.01-3.01 (m, 5H), 2.70-2.87 (m, 3H), 2.34-2.40 (m, 2H), 2.12 (s, 3H), 1.98 (s, 3H).

¹⁹F NMR (300 MHz, Methanol-d4) δ (ppm) −144.0, −151.9

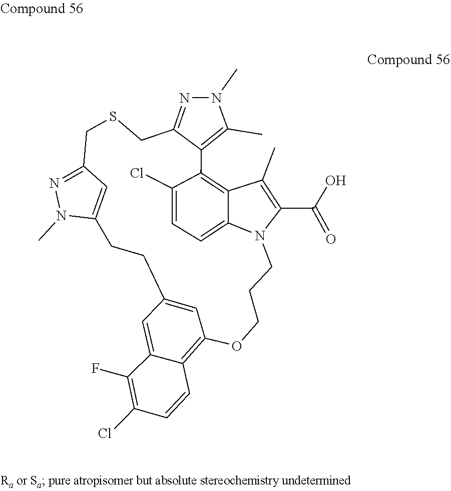 [SMILES: Cc1c(C(=O)O)n2c3ccc(Cl)c(c13)-c1c(nn(C)c1C)CSCc1cc(n(C)n1)CCc1cc(c3ccc(Cl)c(F)c3c1)OCCC2]

A solution of LiOH (50 mg, 2.08 mmol, 6 eq.) in water (5 mL) was added to a solution of Intermediate 176 (250 mg, 0.35 mmol) in THF (5 mL). The reaction mixture was stirred at 40° C. for 16 h. Most of the solvent was removed under reduced pressure. The mixture was extracted with Et₂O (5 mL×3). The aqueous layer was acidified with aqueous HCl (2 M) to pH=3. The solid that appeared was collected by filtration. The crude product was triturated with DCM/petroleum ether (1 mL/10 mL) and filtered to afford Compound 56 (115 mg, yield: 36%. as a white solid.

OR: +32° (589 nm, 22.5° C., 5 mg in 10 mL MeOH)

¹H NMR (300 MHz, CDCl₃) δ (ppm) 8.09 (d, J=9.1 Hz, 1H), 7.30-7.45 (m, 2H), 7.25-7.29 (m, 1H), 7.12 (d, J=9.3 Hz, 1H), 5.69 (s, 1H), 5.39 (s, 1H), 5.20-5.25 (m, 1H), 4.52-4.56 (m, 1H), 3.91 (s, 3H), 3.70 (d, J=14.8 Hz, 1H), 3.16-3.44 (m, 7H), 2.88-2.91 (m, 5H), 2.22-2.33 (m, 5H), 2.07 (s, 3H)

¹⁹F NMR (300 MHz, CDCl₃) δ (ppm) −124.39

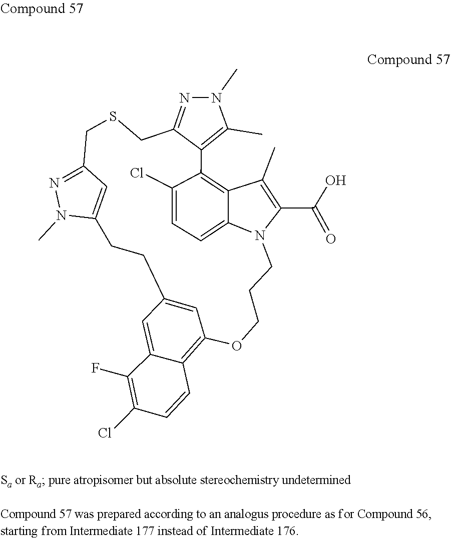 [SMILES: Cc1c(C(=O)O)n2c3ccc(Cl)c(c13)-c1c(nn(C)c1C)CSCc1cc(n(C)n1)CCc1cc(c3ccc(Cl)c(F)c3c1)OCCC2]

OR: −38° (589 nm, 22.5° C., 5 mg in 10 mL MeOH)

¹H NMR (300 MHz, CDCl₃) δ ppm 8.09 (d, J=9.0 Hz, 1H), 7.31-7.47 (m, 2H), 7.28-7.29 (s, 1H), 7.16 (d, J=8.9 Hz, 1H), 5.65 (s, 1H), 5.45 (s, 1H), 5.18-5.23 (m, 1H), 4.51-4.59 (m, 1H), 3.90 (s, 3H), 3.71 (d, J=14.8 Hz, 1H), 3.31-3.45 (m, 3H), 3.19-3.20 (m, 4H), 2.91-2.94 (m, 5H), 2.32 (s, 2H), 2.23 (s, 3H), 2.07 (s, 3H)

¹⁹F NMR (300 MHz, CDCl₃) δ ppm −124.42

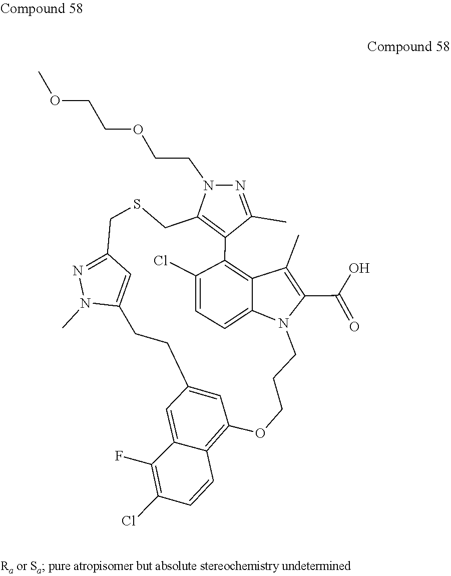 [SMILES: COCCOCCn1nc(C)c2c1CSCc1cc(n(C)n1)CCc1cc(c3ccc(Cl)c(F)c3c1)OCCCn1c(C(=O)O)c(C)c3c-2c(Cl)ccc31]

A solution of LiOH (23 mg, 0.96 mmol, 6 eq.) in water (3 mL) was added to a solution of Intermediate 183 (130 mg, 0.16 mmol) in THF (3 mL). The reaction mixture was stirred at 40° C. for 48 h. Most of the THF was removed under reduced pressure. The mixture was extracted with Et₂O (5 mL×3). The aqueous layer was acidified with aqueous HCl (2 M) to pH=3. The solid formed was collected by filtration and this crude product was triturated with DCM/petroleum ether (1 mL/10 mL) and filtered to afford Compound 58 (56 mg, yield: 43%) as a white solid.

¹H NMR (300 MHz, CDCl₃) δ ppm 8.07 (d, J=9.0 Hz, 1H), 7.44-7.47 (m, 2H), 7.28-7.31 (m, 1H), 7.19-7.21 (m, 1H), 5.55 (d, J=12.7 Hz, 2H), 5.20-5.28 (m, 1H), 4.55 (d, J=14.9 Hz, 1H), 4.33 (t, J=5.7 Hz, 2H), 3.78-4.01 (m, 3H), 3.47-3.63 (m, 5H), 3.19-3.41 (m, 6H), 3.09 (s, 3H), 2.91-2.98 (m, 5H), 2.34 (s, 2H), 2.20 (s, 3H), 2.05 (s, 3H)

¹⁹F NMR (300 MHz, CDCl₃) δ ppm −124.42

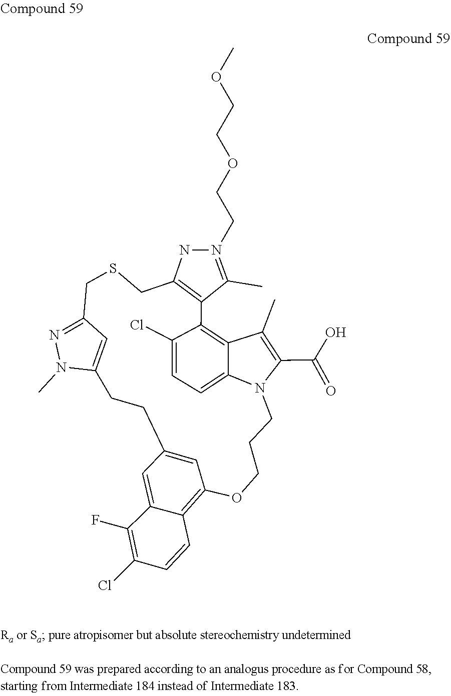

¹H NMR (300 MHz, CDCl₃) δ ppm 8.08 (d, J=9.1 Hz, 1H), 7.38-7.47 (m, 2H), 7.30 (s, 1H), 7.17 (d, J=8.9 Hz, 1H), 5.49-5.63 (m, 2H), 5.19-5.26 (m, 1H), 4.51-4.54 (m, 1H), 4.33 (t, J=5.5 Hz, 2H), 3.90-3.92 (m, 2H), 3.70-3.75 (m, 1H), 3.46-3.57 (m, 4H), 3.36-3.41 (m, 6H), 3.16-3.20 (m, 4H), 2.87-3.01 (m, 5H), 2.31 (s, 2H), 2.23 (s, 3H), 2.10 (s, 3H).

¹⁹F NMR (300 MHz, CDCl₃) δ ppm −124.42

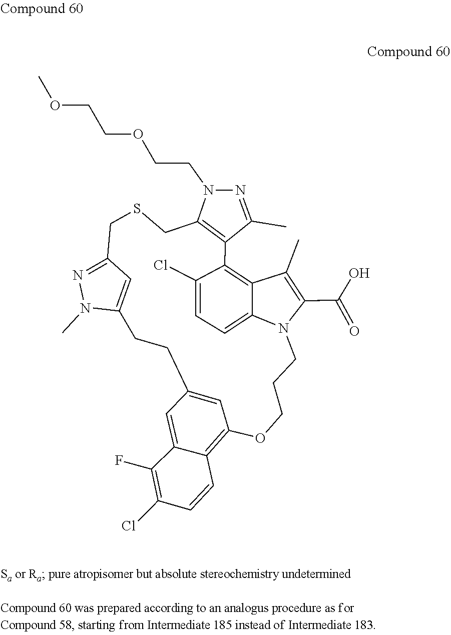 [SMILES: COCCOCCn1nc(C)c2c1CSCc1cc(n(C)n1)CCc1cc(c3ccc(Cl)c(F)c3c1)OCCCn1c(C(=O)O)c(C)c3c-2c(Cl)ccc31]

¹H NMR (300 MHz, CDCl₃) δ ppm 8.07 (d, J=9.0 Hz, 1H), 7.44-7.47 (m, 2H), 7.28-7.31 (m, 1H), 7.19-7.21 (m, 1H), 5.55 (d, J=12.7 Hz, 2H), 5.20-5.28 (m, 1H), 4.55 (d, J=14.9 Hz, 1H), 4.33 (t, J=5.7 Hz, 2H), 3.78-4.01 (m, 3H), 3.47-3.63 (m, 5H), 3.19-3.41 (m, 6H), 3.09 (s, 3H), 2.91-2.98 (m, 5H), 2.34 (s, 2H), 2.20 (s, 3H), 2.05 (s, 3H).

¹⁹F NMR (300 MHz, CDCl₃) δ ppm −124.38

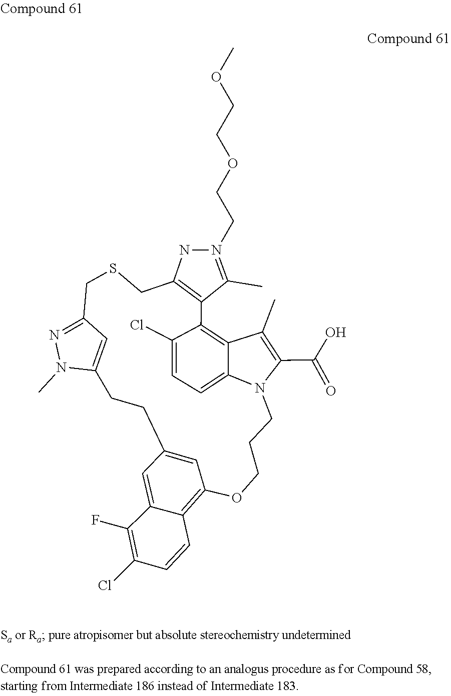

¹H NMR (300 MHz, CDCl₃) δ ppm 8.08 (d, J=9.1 Hz, 1H), 7.38-7.47 (m, 2H), 7.30 (s, 1H), 7.17 (d, J=8.9 Hz, 1H), 5.49-5.63 (m, 2H), 5.19-5.26 (m, 1H), 4.51-4.54 (m, 1H), 4.33 (t, J=5.5 Hz, 2H), 3.90-3.92 (m, 2H), 3.70-3.75 (m, 1H), 3.49-3.54 (m, 2H), 3.47-3.49 (m, 2H), 3.36-3.41 (m, 6H), 3.16-3.20 (m, 4H), 2.87-3.01 (m, 5H), 2.31 (s, 2H), 2.23 (s, 3H), 2.10 (s, 3H).

¹⁹F NMR (300 MHz, CDCl₃) δ ppm −124.42

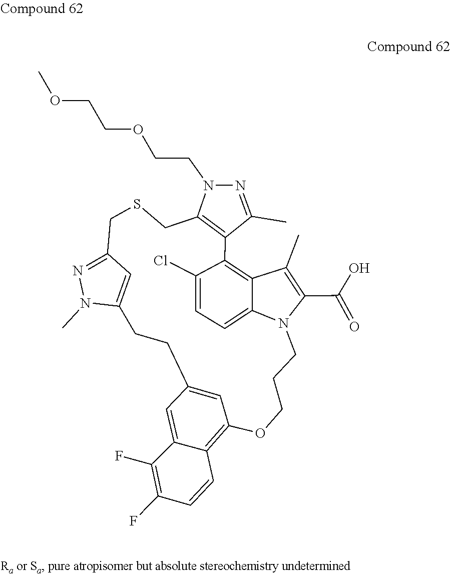 [SMILES: COCCOCCn1nc(C)c2c1CSCc1cc(n(C)n1)CCc1cc(c3ccc(F)c(F)c3c1)OCCCn1c(C(=O)O)c(C)c3c-2c(Cl)ccc31]

A solution of LiOH (18 mg, 0.76 mmol, 6 eq.) in water (4 mL) was added to a solution of Intermediate 192 (100 mg, 0.13 mmol) in THF (4 mL). The reaction mixture was stirred at 40° C. for 16 h. Most of the THF was removed under reduced pressure. The mixture was extracted with Et₂O (5 mL×3). The aqueous layer was acidified with aqueous HCl (2 M) to pH=3. The solid formed was collected by filtration and this crude product was triturated with EtOAc/petroleum ether (1 mL/10 mL) and filtered to afford Compound 62 (47 mg, yield: 47%) as an off-white solid.

¹H NMR (300 MHz, CDCl₃) δ ppm 8.10 (d, J=6 Hz, 1H), 7.48 (s, 1H), 7.35 (d, J=15 Hz, 2H), 7.23 (d, J=9 Hz, 1H), 5.57-5.49 (m, 2H), 5.26-5.18 (m, 1H), 4.54 (d, J=15 Hz, 1H), 4.33 (s, 2H), 3.95-3.83 (m, 3H), 3.68-3.42 (m, 5H), 3.36-3.17 (m, 6H), 3.05 (s, 3H), 2.92 (d, J=12 Hz, 5H), 2.34 (s, 2H), 2.20 (s, 3H), 2.08 (s, 3H)

¹⁹F NMR (282 MHz, CDCl₃) δ ppm −140.708-−140.775, −149.626-−149.693.

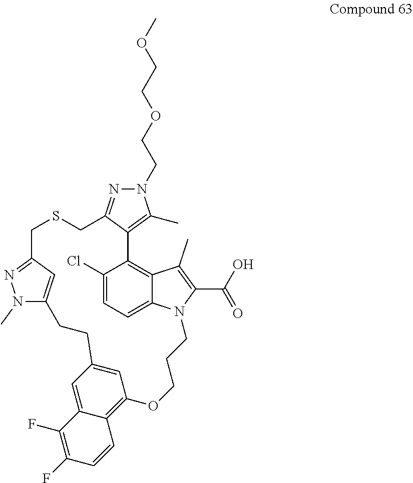 [SMILES: COCCOCCn1nc2c(c1C)-c1c(Cl)ccc3c1c(C)c(C(=O)O)n3CCCOc1cc(cc3c(F)c(F)ccc13)CCc1cc(nn1C)CSC2]

Compound 63: Rₐ or Sₐ, pure atropisomcr but absolute stereochemistry undetermined

Compound 63 was prepared according to an analogous procedure as for Compound 62, starting from Intermediate 193 instead of Intermediate 192.

¹H NMR (300 MHz, CDCl₃) δ ppm 8.11 (s, 1H), 7.49 (s, 1H), 7.33 (d, J=9 Hz, 2H), 7.21 (d, J=9 Hz, 1H), 5.55 (s, 2H), 5.24 (s, 1H), 4.57-4.37 (s, 3H), 3.93 (s, 2H), 3.77 (d, J=12 Hz, 1H), 3.57-3.49 (d, J=15, 4H), 3.36 (s, 6H), 3.15-2.93 (m, 9H), 2.34-2.12 (m, 8H) 19F NMR (282 MHz, CDCl₃) δ ppm −140.739-−140.806, −149.580-−149.648.

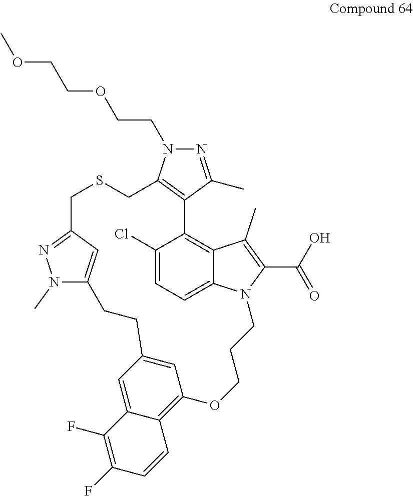 [SMILES: COCCOCCn1nc(C)c2c1CSCc1cc(n(C)n1)CCc1cc(c3ccc(F)c(F)c3c1)OCCCn1c(C(=O)O)c(C)c3c-2c(Cl)ccc31]

Compound 64: Sₐ or Rₐ, pure atropisomer but absolute stereochemistry undetermined

Compound 64 was prepared according to an analogous procedure as for Compound 62, starting from Intermediate 194 instead of Intermediate 192.

¹H NMR (300 MHz, CDCl₃) δ ppm 8.10 (d, J=9 Hz, 1H), 7.49 (s, 1H), 7.43-7.28 (m, 2H), 7.28 (d, J=6, 1H), 5.60-5.47 (d, J=12, 2H), 5.22 (m, 1H), 4.54 (d, J=15 Hz, 1H), 4.35 (s, 2H), 3.96-3.87 (m, 3H), 3.61-3.23 (m, 11H), 3.02-2.93 (m, 8H), 2.34 (s, 2H), 2.20-2.09 (d, J=18, 6H)

¹⁹F NMR (282 MHz, CDCl₃) δ ppm −140.711-−140.779, −149.618-−149.686.

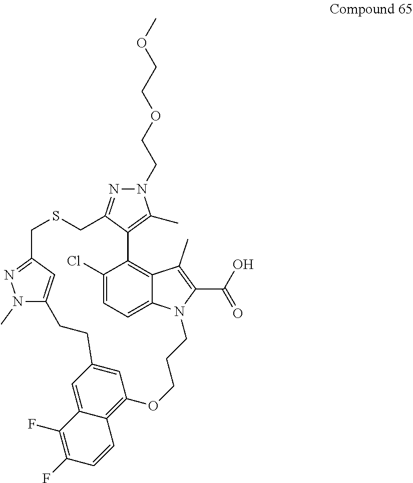 [SMILES: COCCOCCn1nc2c(c1C)-c1c(Cl)ccc3c1c(C)c(C(=O)O)n3CCCOc1cc(cc3c(F)c(F)ccc13)CCc1cc(nn1C)CSC2]

Compound 65: Sₐ or Rₐ, pure atropisomer but absolute stereochemistry undetermined

Compound 65 was prepared according to an analogous procedure as for Compound 62, starting from Intermediate 195 instead of Intermediate 192.

¹H NMR (300 MHz, CDCl₃) δ ppm 8.10 (m, 1H), 7.49 (s, 1H), 7.33 (d, J=9 Hz, 2H), 7.24 (d, J=9 Hz, 1H), 5.61-5.50 (d, J=15, 2H), 5.25 (m, 1H), 4.53-4.35 (m, 3H), 3.91-3.78 (m, 3H), 3.56-3.49 (m, 4H), 3.36-3.20 (m, 6H), 3.19-2.88 (m, 9H), 2.32 (s, 2H), 2.22 (s, 3H), 2.12 (s, 3H)

¹⁹F NMR (282 MHz, CDCl₃) δ ppm −140.797-−140.863, −149.672-−149.739.

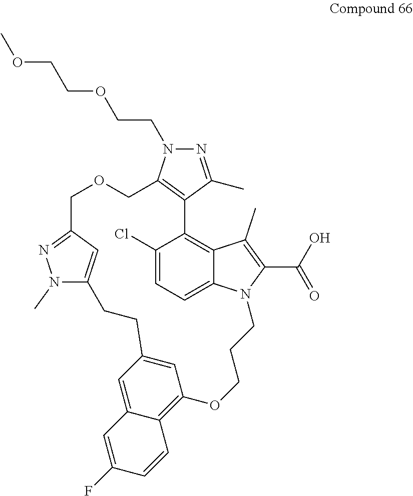 [SMILES: COCCOCCn1nc(C)c2c1COCc1cc(n(C)n1)CCc1cc(c3ccc(F)cc3c1)OCCCn1c(C(=O)O)c(C)c3c-2c(Cl)ccc31]

Compound 66: mixture of atropisomers

A solution of LiOH (4 mg, 0.13 mmol, 10 eq.) in water (0.5 mL) was added to a solution of Intermediate 199 (10 mg, 0.013 mmol) in THF (0.5 mL). The reaction mixture was stirred at 40° C. for 3 days. Most of the THF was removed under reduced pressure. The aqueous layer was acidified with aqueous HCl (2 M) to pH=3. The solid that appeared was collected by filtration to afford Compound 66 (3 mg, yield: 29%) as a white solid.

¹H NMR (300 MHz, Methanol-d4) δ ppm 8.17 (m, 1H), 7.58 (d, J=6 Hz, 1H), 7.28-7.10 (m, 3H), 6.82 (s, 1H), 6.27 (s, 1H), 5.22 (m, 1H), 4.67 (m, 1H), 4.35 (m, 1H), 4.27 (m, 1H), 4.05 (m, 3H), 3.99 (m, 3H), 3.77 (m, 1H), 3.69 (m, 4H), 3.65 (s, 2H), 3.66-3.55 (m, 2H), 3.35 (s, 3H), 3.14-2.94 (m, 2H), 2.86 (d, J=9 Hz, 2H), 2.44 (s, 2H), 2.04 (m, 1H), 1.96 (d, J=6 Hz, 6H)

¹⁹F NMR (282 MHz, Methanol-d4) δ ppm −117.23.

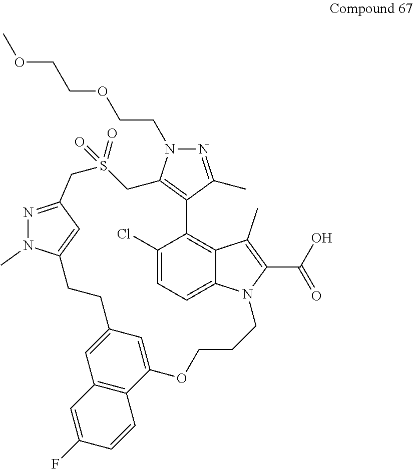 [SMILES: COCCOCCn1nc(C)c2c1CS(=O)(=O)Cc1cc(n(C)n1)CCc1cc(c3ccc(F)cc3c1)OCCCn1c(C(=O)O)c(C)c3c-2c(Cl)ccc31]

Compound 67: Rₐ or Sₐ, pure atropisomer but absolute stereochemistry undetermined

mCPBA (31 mg, 0.177 mmol, 2.2 eq.) was added in one portion to a solution of Compound 31 (61 mg, 0.080 mmol) in DCM (10 mL) at room temperature. The reaction mixture was stirred for 5 h at room temperature. Water was added to the reaction mixture and the layers were separated. The combined organic layer was dried by filtration on Extrelut NT3, and evaporated. The residue was purified by column 10 chromatography (Biotage Sfar 10 g; eluent: DCM/MeOH 100:0->90:10) to give Compound 67 (35 mg, yield: 55%) as a white solid.

¹H NMR (400 MHz, CDCl₃) δ ppm 2.01 (s, 3H) 2.25 (s, 3H) 2.35 (br s, 2H) 2.67-2.77 (m, 5H) 2.86-3.06 (m, 3H) 3.30 (s, 3H) 3.41-3.51 (m, 5H) 3.56 (t, J=4.8 Hz, 1H) 3.57-3.64 (m, 1H) 3.88-3.96 (m, 2H) 4.35-4.46 (m, 2H) 4.55-4.68 (m, 2H) 5.01-5.16 (m, 2H) 5.34 (s, 1H) 5.89 (s, 1H) 7.12 (d, J=9.0 Hz, 1H) 7.22 (s, 1H) 7.27-7.33 (m, 2H) 7.36 (dd, J=9.9, 2.4 Hz, 1H) 8.37 (dd, J=9.1, 5.6 Hz, 1H)

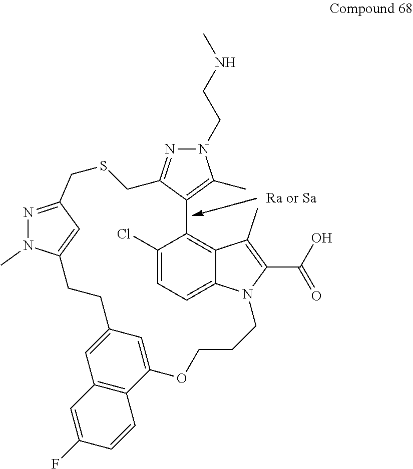 [SMILES: CNCCn1nc2c(c1C)-c1c(Cl)ccc3c1c(C)c(C(=O)O)n3CCCOc1cc(cc3cc(F)ccc13)CCc1cc(nn1C)CSC2]

Pure stereoisomer but absolute stereochemistry undetermined

LiOH (13 mg, 0.54 mmol, 20 eq.) was added to a solution of Intermediate 203 (20.6 mg, 0.027 mmol) in a mixture of MeOH (0.7 mL), THE (0.7 mL), and water (0.4 mL). The reaction mixture was stirred for 4 h at 50° C. The solvents were evaporated and the residue was purified by preparative HPLC (stationary phase: RP XBridge Prep C18 OBD—5 μm, 50×250 mm, Mobile phase: 0.25% NH₄HCO₃ solution in water, CH₃CN) to give Compound 68 (14 mg, yield: 73%) as a pale yellow solid.

¹H NMR (400 MHz, DMSO-d6) δ ppm 1.86 (br s, 3H), 1.95 (s, 3H), 2.23-2.31 (m, 2H), 2.42-2.46 (m, 3H), 2.75-2.93 (m, 4H), 3.03 (br d, J=13.7 Hz, 6H), 3.45 (s, 3H), 3.54 (br d, J=8.8 Hz, 2H), 3.74 (br s, 1H), 4.17-4.49 (m, 3H), 4.99 (s, 1H), 5.10 (br s, 1H), 6.20 (s, 1H), 6.93 (d, J=8.6 Hz, 1H), 7.20 (s, 1H), 7.31 (td, J=8.9, 2.8 Hz, 1H), 7.39 (d, J=8.9 Hz, 1H), 7.51 (dd, J=10.5, 2.6 Hz, 1H), 8.22 (dd, J=9.2, 6.1 Hz, 1H).

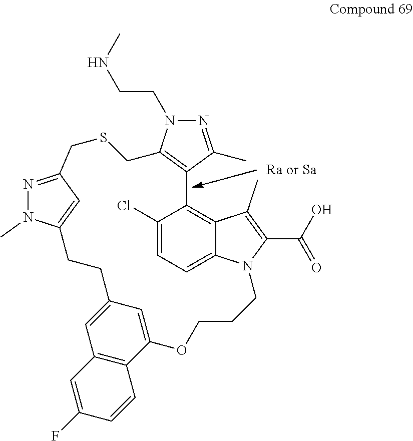 [SMILES: CNCCn1nc(C)c2c1CSCc1cc(n(C)n1)CCc1cc(c3ccc(F)cc3c1)OCCCn1c(C(=O)O)c(C)c3c-2c(Cl)ccc31]

Pure stereoisomer but absolute stereochemistry undetermined

Compound 69 was prepared according to the same procedure as for Compound 68, starting from Intermediate 202 instead of Intermediate 203.

¹H NMR (400 MHz, DMSO-d6) δ ppm 1.82 (s, 3H), 1.86-1.93 (m, 3H), 2.28 (br s, 2H), 2.43-2.47 (m, 3H), 2.69-2.86 (m, 3H), 2.89 (d, J=13.9 Hz, 1H), 2.96-3.02 (m, 2H), 3.03-3.13 (m, 4H), 3.44-3.47 (m, 3H), 3.49-3.54 (m, 2H), 3.76-3.83 (m, 1H), 4.13-4.29 (m, 2H), 4.41-4.53 (m, 1H), 4.87 (s, 1H), 5.06 (br d, J=14.6 Hz, 1H), 6.13 (s, 1H), 6.83 (d, J=8.8 Hz, 1H), 7.16 (s, 1H), 7.29-7.37 (m, 2H), 7.50 (dd, J=10.4, 2.6 Hz, 1H), 8.29 (dd, J=9.1, 5.8 Hz, 1H).

### LCMS Results (RT Means Retention Time)

| Compound number | LCMS results |
|---|---|
| 1 | confirms the MW (RT: 1.75, [M + H] + 654, LCMS Method 4) |
| 2 | confirms the MW (RT: 1.75, [M + H] + 654, LCMS Method 4) |
| 3 | confirms the MW (RT: 1.82, [M + H] + 672, LCMS Method 2) |
| 4 | confirms the MW (RT: 1.82, [M + H] + 672, LCMS Method 2) |
| 5 | confirms the MW (RT: 0.90, [M + H] + 672, LCMS Method 3) |
| 6 | confirms the MW (RT: 1.73, [M + H] + 686, LCMS Method 4) |
| 7 | confirms the MW (RT: 1.73, [M + H] + 686, LCMS Method 4) |
| 8 | confirms the MW (RT: 1.70, [M + H] + 669, LCMS Method 1) |
| 9 | confirms the MW (RT: 1.70, [M + H] + 669, LCMS Method 1) |
| 10 | confirms the MW (RT: 0.93, [M + H] + 672, LCMS Method 3) |
| 11 | confirms the MW (RT: 0.93, [M + H] + 672, LCMS Method 3) |
| 12 | confirms the MW (RT: 1.69, [M + H] + 669, LCMS Method 5) |
| 13 | confirms the MW (RT: 1.70, [M + H] + 629, LCMS Method 1) |
| 14 | confirms the MW (RT: 1.88, [M + H] + 704, LCMS Method: 2) |
| 15 | confirms the MW (RT: 1.87, [M + H] + 704, LCMS Method: 2) |
| 16 | confirms the MW (RT: 1.03, [M + H] + 804, LCMS Method 6) |
| 17 | confirms the MW (RT: 0.98, [M + H] + 804, LCMS Method 6) |
| 18 | confirms the MW (RT: 0.93, [M + H] + 716, LCMS Method 3) |
| 19 | confirms the MW (RT: 0.93, [M + H] + 716, LCMS Method 3) |
| 20 | confirms the MW (RT: 0.95, [M + H] + 760, LCMS Method 3) |
| 21 | confirms the MW (RT: 0.96, [M + H] + 760, LCMS Method 3) |
| 22 | confirms the MW (RT: 0.97, [M + H] + 770, LCMS Method 3) |
| 23 | confirms the MW (RT: 0.97, [M + H] + 770, LCMS Method 3) |
| 28 | confirms the MW (RT: 0.92, [M + H] + 672, LCMS Method 3) |
| 29 | confirms the MW (RT: 0.88, [M + H] + 658, LCMS Method 3) |
| 30 | confirms the MW (RT: 0.88, [M + H] + 658, LCMS Method 3) |
| 31 | confirms the MW (RT: 0.93, [M + H] + 760, LCMS Method 3) |
| 32 | confirms the MW (RT: 0.93, [M + H] + 760, LCMS Method 3) |
| 33 | confirms the MW (RT: 1.75, [M + H] + 742, LCMS Method 8) |
| 34 | confirms the MW (RT: 1.75, [M + H] + 742, LCMS Method 8) |
| 35 | confirms the MW (RT: 1.35, [M + H] + 688, LCMS Method 9) |
| 36 | confirms the MW (RT: 1.37, [M + H] + 688, LCMS Method 9) |
| 37 | confirms the MW (RT: 0.96, [M + H] + 715, LCMS Method 7) |
| 38 | confirms the MW (RT: 0.96, [M + H] + 715, LCMS Method 7) |
| 39 | confirms the MW (RT: 1.56, [M + H] + 776, LCMS Method 10 |
| 40 | confirms the MW (RT: 2.90, [M + H] + 776, LCMS Method 11 |
| 41 | confirms the MW (RT: 2.96, [M + H] + 732, LCMS Method 12 |
| 42 | confirms the MW (RT: 3.13, [M + H] + 732, LCMS Method 12 |
| 43 | confirms the MW (RT: 2.84, [M + H] + 776, LCMS Method 11 |
| 44 | confirms the MW (RT: 1.59, [M + H] + 776, LCMS Method 10 |
| 45 | confirms the MW (RT: 1.62, [M + H] + 656, LCMS Method 13 |
| 46 | confirms the MW (RT: 1.62, [M + H] + 656, LCMS Method 13 |
| 47 | confirms the MW (RT: 2.79, [M + H] + 732, LCMS Method 11 |
| 48 | confirms the MW (RT: 2.93, [M + H] + 732, LCMS Method 11 |
| 49 | confirms the MW (RT: 1.70, [M + H] + 776, LCMS Method 4 |
| 50 | confirms the MW (RT: 1.71, [M + H] + 776, LCMS Method 4 |
| 51 | confirms the MW (RT: 1.68, [M + H] + 746, LCMS Method 4 |
| 52 | confirms the MW (RT: 2.34, [M + H] + 702, LCMS Method 14 |
| 53 | confirms the MW (RT: 2.33, [M + H] + 702, LCMS Method 14 |
| 54 | confirms the MW (RT: 2.74, [M + H] + 690, LCMS Method 15 |
| 55 | confirms the MW (RT: 1.51, [M + H] + 690, LCMS Method 10 |
| 56 | confirms the MW (RT: 2.92, [M + H] + 706, LCMS Method 16 |
| 57 | confirms the MW (RT: 1.59, [M + H] + 706, LCMS Method 10 |
| 58 | confirms the MW (RT: 2.64, [M + H] + 706, LCMS Method 17 |
| 59 | confirms the MW (RT: 2.97, [M + H] + 794, LCMS Method 11 |
| 60 | confirms the MW (RT: 2.89, [M + H] + 794, LCMS Method 11 |
| 61 | confirms the MW (RT: 1.61, [M + H] + 794, LCMS Method 10 |
| 62 | confirms the MW (RT: 1.51, [M + H] + 778, LCMS Method 10 |
| 63 | confirms the MW (RT: 1.58, [M + H] + 778, LCMS Method 10 |
| 64 | confirms the MW (RT: 1.51, [M + H] + 778, LCMS Method 10 |
| 65 | confirms the MW (RT: 1.53, [M + H] + 778, LCMS Method 10 |
| 66 | confirms the MW (RT: 1.53, [M + H] + 778, LCMS Method 18 |
| 67 | confirms the MW (RT: 0.99, [M + H] + 792, LCMS Method 3 |
| 68 | confirms the MW (RT: 1.69, [M + H] + 715, LCMS Method 19) |
| 69 | confirms the MW (RT: 0.90, [M + H] + 715, LCMS Method 3) |

TABLE
Analytical SFC data

| Compound | SFC | | |
|---|---|---|---|
| No. | Method | Rt | [M + H]⁺ |
| 1 | 1 | 4.32 | 654 |
| 2 | 1 | 3.93 | 654 |
| 3 | 2 | 4.48 | 672 |
| 4 | 2 | 4.87 | 672 |
| 6 | 3 | 7.94 | 686 |
| 7 | 3 | 7.26 | 686 |
| 8 | 4 | 6.59 | 669 |
| 9 | 4 | 7.26 | 669 |
| 10 | 5 | 4.35 | 672 |
| 11 | 6 | 3.57 | 672 |
| 14 | 7 | 7.88 | 704 |
| 15 | 7 | 6.94 | 704 |
| 18 | 6 | 3.49 | 716 |
| 19 | 2 | 4.47 | 716 |
| 20 | 9 | 3.94 | 760 |
| 21 | 9 | 3.96 | 760 |
| 22 | 10 | 4.39 | 770 |
| 23 | 10 | 4.90 | 770 |
| 28 | 5 | 4.07 | 672 |
| 29 | 6 | 3.63 | 658 |
| 30 | 6 | 4.06 | 658 |
| 31 | 10 | 3.99 | 760 |
| 32 | 10 | 4.46 | 760 |
| 35 | 12 | 1.45 | 688 |
| 36 | 12 | 1.36 | 688 |
| 39 | 13 | 1.70 | 776 |
| 40 | 13 | 1.73 | 776 |
| 41 | 13 | 1.73 | 732 |
| 42 | 13 | 1.76 | 732 |
| 43 | 14 | 1.97 | 776 |
| 44 | 14 | 2.00 | 776 |
| 45 | 15 | 1.93 | 656 |
| 46 | 16 | 2.11 | 656 |
| 47 | 13 | 1.82 | 732 |
| 48 | 13 | 1.78 | 732 |
| 52 | 17 | 4.94 | 702 |
| 53 | 17 | 4.44 | 702 |
| 54 | 15 | 1.91 | 690 |
| 55 | 18 | 1.84 | 690 |
| 56 | 18 | 2.08 | 706 |
| 57 | 15 | 2.00 | 706 |
| 58 | 14 | 1.80 | 794 |
| 59 | 20 | 4.15 | 794 |
| 60 | 14 | 1.86 | 794 |
| 61 | 20 | 5.51 | 794 |
| 62 | 19 | 0.93 | 778 |
| 63 | 21 | 1.61 | 778 |
| 64 | 19 | 1.29 | 778 |
| 65 | 21 | 1.61 | 778 |
| 68 | 7 | 6.51 | 715 |
| 69 | 7 | 6.50 | 715 |

Rₜ means retention time (in minutes),
[M + H]⁺ means the protonated mass of the compound,
method refers to the method used for (SFC)MS analysis of enantiomerically pure compounds.
No. means number.

### Analytical Analysis

The High Performance Liquid Chromatography (HPLC) measurement was performed using a LC pump, a diode-array (DAD) or a U.V detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (R₄) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

LCMS Method Codes (Flow expressed in mL/min; column temperature (T) in ° C.; Run time in minutes)

LC-MS methods:

| Method | | | | | Flow | Run |
|---|---|---|---|---|---|---|
| Code | Instrument | column | mobile phase | gradient | Col T | time |
| 1 | Waters: Acquity ® | Waters: BEH | A: 10 mM | From 100% A to 5% | 0.6 | 3.5 |
| | UPLC ® - DAD and | (1.8 μm, | CH₃COONH₄ in 95% | A in 2.1 min, to 0% | 55 | |
| | SQD | 2.1*100 mm) | H₂O + 5% CH₃CN | A in 0.9 min, to 5% | | |
| | | | B: CH₃CN | A in 0.5 min | | |
| 2 | Waters: Acquity ® | Waters: BEH | A: 10 mM | From 100% A to 5% | 0.7 | 3.5 |
| | UPLC ® - DAD and | (1.8 μm, | CH₃COONH₄ in 95% | A in 2.1 min, to 0% | 55 | |
| | SQD | 2.1*100 mm) | H₂O + 5% CH₃CN | A in 0.9 min, to 5% | | |
| | | | B: CH₃CN | A in 0.5 min | | |
| 3 | Waters: Acquity ® | Waters: BEH C18 | A: 10 mM | From 95% A to 5% | 0.8 | 2 |
| | UPLC ® - DAD and | (1.7 μm, | CH₃COONH₄ in 95% | A in 1.3 min, held | 55 | |
| | SQD | 2.1*50 mm) | H₂O + 5% CH₃CN | for 0.7 min. | | |
| | | | B: CH₃CN | | | |
| 4 | Waters: Acquity ® | Waters: BEH | A: 10 mM | From 100% A to 5% | 0.7 | 3.5 |
| | UPLC ® - DAD and | (1.8 μm, | CH₃COONH₄ in 95% | A in 2.10 min, to 0% | 55 | |
| | SQD | 2.1*100 mm) | H₂O + 5% CH₃CN | A in 0.9 min, to 5% | | |
| | | | B: CH₃CN | A in 0.5 min | | |
| 5 | Waters: Acquity ® | Waters: BEH | A: 0.1% | From 100% A to 5% | 0.6 | 4.5 |
| | UPLC ® - DAD and | (1.8 μm, | NH₄HCO₃ in 95% | A in 2.10 min, to 0% | 55 | |
| | SQD | 2.1*100 mm) | H₂O + 5% CH₃CN | A in 0.9 min, to 5% | | |
| | | | B: CH₃CN | A in 0.5 min | | |
| 6 | Waters: Acquity ® | Waters: BEH | A: 0.1% | From 100% A to 5% | 0.8 | 2.0 |
| | UPLC ® - DAD and | (1.8 μm, | NH₄HCO₃ in 95% | A in 1.3 min, hold | 55 | |
| | SQD | 2.1*50 mm) | H₂O + 5% CH₃CN | 0.7 min | | |
| | | | B: CH₃CN | | | |
| 7 | Waters: Acquity ® | Waters: BEH | A: 0.1% | From 100% A to 5% | 0.8 | 2.0 |
| | UPLC ® - DAD and | (1.8 μm, | NH₄HCO₃ in 95% | A in 1.3 min, hold | 55 | |
| | SQD2 | 2.1*50 mm) | H₂O + 5% CH₃CN | 0.7 min | | |
| | | | B: CH₃CN | | | |
| 8 | Waters: Acquity ® | Waters: BEH | A: 0.1% | From 100% A to 5% | 0.6 | 3.5 |
| | UPLC ® - DAD and | (1.8 μm, | NH₄HCO₃ in 95% | A in 2.10 min, to 0% | 55 | |
| | SQD2 | 2.1*100 mm) | H₂O + 5% CH₃CN | A in 1.4 min | | |
| | | | B: CH₃CN | | | |
| 9 | Shimadzu | Kinetex EVO | A: Water - 5 mM | 10% A to 95% A in | 1.2 | 2.85 |
| | LCMS-2020 | C18 100 A, | NH₄HCO₃ | 2.0 min, hold 0.7 min | 40 | |
| | | 3.0 * 50 mm, | B: CH₃CN | at 95% A, 95% A to 10% | | |
| | | 2.6 um | | A in 0.05 min, hold | | |
| | | | | 0.1 min at 10% A | | |
| 10 | Shimadzu | HALO 90 A C18, | A: Water/0.05% TFA | 5% A to 100% A in | 1.5 | 3 |
| | LCMS-2020 | 3.0 × 30 mm, | B: ACN/0.05% TFA | 2 min, hold 0.7 min | 40 | |
| | | 2.0 um | | at 100% A | | |
| 11 | Shimadzu | HALO 90 A C18, | A: Water/0.05% TFA | 5% A to 70% A in | 1.5 | 4 |
| | LCMS-2020 | 3.0 × 30 mm, | B: ACN/0.05% TFA | 3 min, 70% A to 95% | 40 | |
| | | 2.0 um | | A in 0.3 min, hold | | |
| | | | | 0.45 min at 95% A | | |
| 12 | Shimadzu | HALO 90 A C18, | A: Water/0.05% TFA | 5% A to 65% A in | 1.5 | 4 |
| | LCMS-2020 | 3.0 × 30 mm, | B: ACN/0.05% TFA | 3 min, 65% A to 95% | 40 | |
| | | 2.0 um | | A in 0.3 min, hold | | |
| | | | | 0.45 min at 95% A | | |
| 13 | Shimadzu | Ascentis | A: Water/0.05% TFA | 5% A to 100% A in | 1.5 | 3 |
| | LCMS-2020 | Express C18, | B: ACN/0.05% TFA | 2 min, hold 0.70 min | 40 | |
| | | 3.0 × 50 mm, | | at 100% A | | |
| | | 2.7 um | | | | |
| 14 | Waters: Acquity ® | Waters: BEH | A: 10 mM NH₄HCO₃ in | From 100% A to 5% A in | 0.6 | 3.5 |
| | UPLC ® - DAD and | 1.8 μm, | 95% H₂O + 5% CH₃CN | 2.10 min, to 0% A in | 55 | |
| | SQD | 2.1 × 100 mm) | B: MeOH | 0.9 min, to 5% A in | | |
| | | | | 0.5 min | | |
| 15 | Shimadzu | HALO 90 A C18, | A: Water/6.5 mM | 10% to 50% in 3.0 min, | 1.2 | 4 |
| | LCMS-2020 | 3.0 × 30 mm, | NH₄HCO₃ + NH₄OH | 50% to 95% in 0.3 min, | 40 | |
| | | 2.0 um | (pH = 10) | hold 0.45 min at 95% | | |
| | | | B: ACN | | | |
| 16 | Shimadzu | HALO 90 A C18, | A: Water/0.05% TFA | 5% A to 70% A in | 1.5 | 4 |
| | LCMS-2020 | 3.0 × 30 mm, | B: ACN/0.05% TFA | 3 min, 70% A to 95% | 40 | |
| | | 2.0 um | | A in 0.3 min, hold | | |
| | | | | 0.45 min at 100% A | | |
| 17 | Shimadzu | HALO 90 A C18, | A: Water/0.05% TFA | 30% A to 70% A in | 1.5 | 4 |
| | LCMS-2020 | 3.0 × 30 mm, | B: ACN/0.05% TFA | 3 min, 70% A to 100% | 40 | |
| | | 2.0 um | | A in 0.3 min, hold | | |
| | | | | 0.45 min at 100% A | | |
| 18 | Shimadzu | HALO 90 A C18, | A: Water/0.05% TFA | 30% A to 70% A in | 1.5 | 3 |
| | LCMS-2020 | 3.0 × 30 mm, | B: ACN/0.05% TFA | 1.7 min, 70% A to 95% | 40 | |
| | | 2.0 um | | A in 0.6 min, hold | | |
| | | | | 0.5 min at 95% A | | |
| 19 | Waters: Acquity ® | Waters: BEH | A: 10 mM CH₃COONH₄ | From 100% A to 5% A | 0.6 | 3.5 |
| | UPLC ® - DAD and | (1.8 μm, | in 95% H₂O + 5% | in 2.10 min, to 0% | 55 | |
| | SQD | 2.1 * 100 mm) | CH₃CN | A in 0.9 min, to 5% | | |
| | | | B: CH₃CN | A in 0.5 min | | |

### SFC-MS Methods:

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Analytical SFC-MS Methods (Flow expressed in mL/min; column temperature (Col T) in ° C.; Run time in minutes, Backpressure (BPR) in bars. “iPrNH₂” means isopropylamine, “iPrOH” means 2-propanol, “EtOH” means ethanol, “min” mean minutes, “DEA” means diethylamine.

### SFC Methods:

| SFC | | | | Flow | Run time |
|---|---|---|---|---|---|
| Method | Column | mobile phase | gradient | Col T | BPR |
| 1 | Daicel | A:CO₂ | 5% B hold 6 | 2.5 | 9.5 |
| | Chiralpak ® AS3 | B: EtOH + 0.2% | min, to 50% in 1 | 40 | 130 |
| | column (3.0 pm, | iPrNH₂ | min hold 2.5 min | | |
| | 150 × 4.6 mm) | | | | |
| 2 | Daicel | A:CO₂ | 5% B hold 6 | 2.5 | 9.5 |
| | Chiralpak ® | B: EtOH + 0.2% | min, to 50% in | 40 | 130 |
| | AD3 column | iPrNH₂ | 1 min hold | | |
| | (3.0 pm, 150 × | | 2.5 min | | |
| | 4.6 mm) | | | | |
| 3 | Daicel | A:CO₂ | 5% B hold 6 | 2.5 | 9.5 |
| | Chiralpak ® ID3 | B: EtOH + 0.2% | min, to 50% in | 40 | 130 |
| | column (3.0 | iPrNH₂ | 1 min hold 2.5 | | |
| | pm, 150 × 4.6 mm) | | min | | |
| 4 | Daicel | A:CO₂ | 5% B hold 6 | 2.5 | 9.5 |
| | Chiralpak ® IG3 | B: iPrOH + 0.2% | min, to 50% in | 40 | 130 |
| | column (3.0 | iPrNH₂ | 1 min hold 2.5 min | | |
| | μm, 150 × 4.6 mm) | | | | |
| 5 | Daicel | A:CO₂ | 10%-50% B | 2.5 | 9.5 |
| | Chiralpak ® | B: EtOH + 0.2% | in 6 min, hold | 40 | 130 |
| | AD3 column | iPrNH₂ | 3.5 min | | |
| | (3.0 μm, 150 × | | | | |
| | 4.6 mm) | | | | |
| 6 | Daicel | A:CO₂ | 10%-50% B | 2.5 | 9.5 |
| | Chiralpak ® OJ3 | B: EtOH + 0.2% | in 6 min, hold | 40 | 130 |
| | column (3.0 | iPrNH₂ | 3.5 min | | |
| | μm, 150 × 4.6 mm) | | | | |
| 7 | Daicel | A:CO₂ | 10%-50% B in 6 | 2.5 | 9.5 |
| | Chiralpak ® IG3 | B: EtOH+0.2% | min, hold 3.5 | 40 | 130 |
| | column (3.0 | iPrNH₂ | min | | |
| | μm, 150 × 4.6 mm) | | | | |
| 9 | Daicel | A:CO₂ | 10%-50% B | 2.5 | 9.5 |
| | Chiralpak ® AS3 | B: EtOH + 0.2% | in 6 min, hold | 40 | 130 |
| | column (3.0 | iPrNH₂ | 3.5 min | | |
| | μm, 150 × 4.6 mm) | | | | |
| 10 | Daicel | A:CO₂ | 10%-50% B | 2.5 | 9.5 |
| | Chiralpak ® | B: EtOH + 0.2% | in 6 min, hold | 40 | 130 |
| | AD3 column | iPrNH₂ | 3.5 min | | |
| | (3.0 μm, 150 × | | | | |
| | 4.6 mm) | | | | |
| 11 | Daicel | A:CO₂ | 10%-50% B | 2.5 | 9.5 |
| | Chiralpak ® IC3 | B: EtOH + 0.2% | in 6 min, hold | 40 | 130 |
| | column (3.0 | iPrNH₂ | 3.5 min | | |
| | μm, 150 × 4.6 mm) | | | | |
| 12 | CHIRALPAK | A:CO₂ | 10% to 50% in | 2 | 3 |
| | AS, 3.0 × 50 | B: MeOH + 0.1% | 2 min, hold 1 | 35 | 100 |
| | mm, 3 um | EtNH | min at 50% | | |
| 13 | CHIRALPAK | A: CO₂ | 10% to 50% in | 2 | 3 |
| | OJ-3, 4.6 × 50 | B: MeOH (0.1% | 2.0 min, hold 1 | 35 | 103 |
| | mm, 3 um | DEA) | min at 50% | | |
| 14 | CHIRALPAK | A: CO₂ | 10% to 50% in | 2 | 3 |
| | IA-3 3.0 × 50 | B: MeOH (1% 2 | 2.0 min, hold | 35 | 100 |
| | mm 3 um | M NH₃ in MeOH) | 1.0 min at 50% | | |
| 15 | CHIRALPAK | A: CO₂ | 10% to 50% in | 4 | 3 |
| | IB N-3, 4.6 × | B: MeOH (0.1% | 2.0 min, hold | 35 | 103 |
| | 100 mm, 3 um | DEA) | 1.0 min at 50% | | |
| 16 | CHIRALPAK | A: CO₂ | 10% to 50% in | 2 | 3 |
| | OD, 3.0 × 100 | B: MeOH (0.1% | 2.0 min, hold | 35 | 103 |
| | mm, 3 um | DEA) | 1.0 min at 50% | | |
| 17 | Daicel | A:CO₂ | 10%-50% B | 2.5 | 9.5 |
| | Chiralpak ® IH3 | B: EtOH + 0.2% | in 6 min, hold | 40 | 130 |
| | column (3.0 μm, | iPrNH | 3.5 min | | |
| | 150 × 4.6 mm) | | | | |
| 18 | (S,S) Whelk-01, | A:CO₂ | 10%-50% B | 4 | 3 |
| | 4.6 * 100 mm, | B: MeOH+ 0.1% | in 2 min, hold 1 | 35 | 103 |
| | 5 μm | Et₂NH | min at 50% | | |
| 19 | CHIRALPAK | MeOH (1% 2 M | 30% to 30% in | 4 | 3 |
| | IG-3 4.6 * 50 | NH₃ in MeOH) | 3.0 min | 35 | 100 |
| | mm 3 μm | | | | |
| 20 | CHIRALPAK | MeOH:ACN: | 30% to 30% in | 2 | 7 |
| | IC 3.0 × 100 | DCM = 1:1:1 | 7.0 min | 35 | 103 |
| | mm, 3 μm | (0.1% DEA) | | | |
| 21 | Lux 3u | MeOH (1% 2 M | 10% to 50% B | 4 | 3 |
| | Cellulose-3 | NH₃ in MeOH) | in 2.0 min, hold | 35 | 100 |
| | 4.6 * 100 mm | | 1.0 min at 50% | | |
| | 3 μm | | | | |

### NMR

¹H NMR and ¹⁹F NMR spectra were recorded on Bruker Avance III 400 MHz and Avance NEO 400 MHz spectrometers. CDCl₃ was used as solvent, unless otherwise mentioned. The chemical shifts are expressed in ppm relative to tetramethylsilane.

### Pharmacological Analysis

### Biological Example 1

Terbium labeled Myeloid Cell Leukemia 1 (Mcl-1) homogeneous time-resolved fluorescence (HTRF) binding assay utilizing the BIM BH3 peptide (H₂N—(C/Cy5Mal) WIAQELRRIGDEFN-OH) as the binding partner for Mcl-1.

Apoptosis, or programmed cell death, ensures normal tissue homeostasis, and its dysregulation can lead to several human pathologies, including cancer. Whilst the extrinsic apoptosis pathway is initiated through the activation of cell-surface receptors, the intrinsic apoptosis pathway occurs at the mitochondrial outer membrane and is governed by the binding interactions between pro- and anti-apoptotic Bcl-2 family proteins, including Mcl-1. In many cancers, the anti-apoptotic Bcl-2 protein(s), such as the Mcl-1, are upregulated, and in this way the cancer cells can evade apoptosis. Thus, inhibition of the Bcl-2 protein(s), such as Mcl-1, may lead to apoptosis in cancer cells, providing a method for the treatment of said cancers.

This assay evaluated inhibition of the BH3 domain: Mcl-1 interaction by measuring the displacement of Cy5-labeled BIM BH3 peptide (H₂N—(C/Cy5Mal) WIAQELRRIGDEFN-OH) in the HTRF assay format.

### Assay Procedure

The following assay and stock buffers were prepared for use in the assay: (a) Stock buffer: 10 mM Tris-HCl, pH=7.5+150 mM NaCl, filtered, sterilized, and stored at 4° C.; and (b) 1× assay buffer, where the following ingredients were added fresh to stock buffer: 2 mM dithiothreitol (DTT), 0.0025% Tween-20, 0.1 mg/mL bovine serum albumin (BSA). The 1× Tb-Mcl-1+Cy5 Bim peptide solution was prepared by diluting the protein stock solution using the 1× assay buffer (b) to 25 pM Tb-Mcl-1 and 8 nM Cy5 Bim peptide.

Using the Acoustic ECHO, 100 nL of 100× test compound(s) were dispensed into individual wells of a white 384-well Perkin Elmer Proxiplate, for a final compound concentration of Ix and final DMSO concentration of 1%. Inhibitor control and neutral control (NC, 100 nL of 100% DMSO) were stamped into columns 23 and 24 of assay plate, respectively. Into each well of the plate was then dispensed 10 μL of the 1× Tb-Mcl-1+Cy5 Bim peptide solution. The plate was centrifuged with a cover plate at 1000 rpm for 1 minute, then incubated for 60 minutes at room temperature with plates covered.

The TR-FRET signal was read on an BMG PHERAStar FSX MicroPlate Reader at room temperature using the HTRF optic module (HTRF: excitation: 337 nm, light source: laser, emission A: 665 nm, emission B: 620 nm, integration start: 60 μs, integration time: 400 μs).

### Data Analysis

The BMG PHERAStar FSX MicroPlate Reader was used to measure fluorescence intensity at two emission wavelengths—665 nm and 620 nm—and report relative fluorescence units (RFU) for both emissions, as well as a ratio of the emissions (665 nm/620 nm)*10,000. The RFU values were normalized to percent inhibition as follows:

% inhibition=(((NC−IC)−(compound−IC))/(NC−IC))*100

where IC (inhibitor control, low signal)=mean signal of 1× Tb-MCl-1+Cy5 Bim peptide+inhibitor control or 100% inhibition of Mcl-1; NC (neutral control, high signal)=mean signal 1× Tb-MCl-1+Cy5 Bim peptide with DMSO only or 0% inhibition

An 11-point dose response curve was generated to determine IC₅₀ values (using GenData) based on the following equation:

Y=Bottom+(Top−Bottom)/(1+10\hat{ }((log IC₅₀−X)*HillSlope))

where Y=% inhibition in the presence of X inhibitor concentration; Top=100% inhibition derived from the IC (mean signal of Mcl-1+inhibitor control); Bottom=0% inhibition derived from the NC (mean signal of Mcl-1+DMSO); Hillslope=Hill coefficient; and IC₅₀=concentration of compound with 50% inhibition in relation to top/neutral control (NC).

Kᵢ=IC₅₀/(1+[L]/Kd)

In this assay [L]=8 nM and Kd=10 nM

Representative compounds of the present invention were tested according to the procedure as described above, with results as listed in the Table below (n.d. means not determined).

| Compound | TB-MCL1 Kᵢ (nM) |
|---|---|
| 1 | NT |
| 2 | 0.483 |
| 3 | 0.023 |
| 4 | 0.713 |
| 5 | 0.411 |
| 6 | 0.059 |
| 7 | 1.71 |
| 8 | 0.027 |
| 9 | 0.509 |
| 10 | 0.042 |
| 11 | 7.48 |
| 12 | 0.141 |
| 13 | 15.34 |
| 14 | 0.021 |
| 15 | 28.02 |
| 16 | 0.024 |
| 17 | 0.018 |
| 18 | 0.025 |
| 19 | 0.035 |
| 20 | 0.026 |
| 21 | 0.066 |
| 22 | 0.029 |
| 23 | 0.016 |
| 28 | 0.028 |
| 29 | 0.033 |
| 30 | 6.95 |
| 31 | 0.015 |
| 32 | 1.51 |
| 33 | 0.027 |
| 34 | 2.36 |
| 35 | 0.026 |
| 36 | 3.07 |
| 38 | 0.019 |
| 39 | 0.032 |
| 40 | 0.053 |
| 41 | 0.66 |
| 42 | 0.030 |
| 43 | 2.50 |
| 44 | 3.45 |
| 45 | 1.17 |
| 46 | 0.018 |
| 47 | 2.95 |
| 48 | 4.35 |
| 49 | 0.010 |
| 50 | 0.013 |
| 51 | 0.013 |
| 52 | 0.013 |
| 53 | 0.306 |
| 54 | 0.020 |
| 55 | 3.96 |
| 56 | 0.038 |
| 57 | 2.97 |
| 58 | 0.081 |
| 59 | 0.104 |
| 60 | 4.73 |
| 61 | 114.90 |
| 62 | 0.027 |
| 63 | 0.026 |
| 64 | 2.33 |
| 65 | 2.98 |
| 66 | 0.015 |
| 67 | 0.017 |
| 68 | 0.008 |
| 69 | 0.014 |

### Biological Example 2

MCL-1 is a regulator of apoptosis and is highly over-expressed in tumor cells that escape cell death. The assay evaluates the cellular potency of small-molecule compounds targeting regulators of the apoptosis pathway, primarily MCL-1, Bfl-1, Bcl-2, and other proteins of the Bcl-2 family. Protein-protein inhibitors disrupting the interaction of anti-apoptotic regulators with BH3-domain proteins initiate apoptosis.

The Caspase-Glo® 3/7 Assay is a luminescent assay that measures caspase-3 and −7 activities in purified enzyme preparations or cultures of adherent or suspension cells. The assay provides a proluminescent caspase-3/7 substrate, which contains the tetrapeptide sequence DEVD. This substrate is cleaved to release aminoluciferin, a substrate of luciferase used in the production of light. Addition of the single Caspase-Glo® 3/7 Reagent in an “add-mix-measure” format results in cell lysis, followed by caspase cleavage of the substrate and generation of a “glow-type” luminescent signal.

This assay uses the MOLP-8 human multiple myeloma cell line, which is sensitive to MCL-1 inhibition.

### Materials:

Perkin Elmer Envision
Multidrop 384 and small volume dispensing cassettes
Centrifuge
Countess automated cell counter
Countess counting chamber slides
Assay plate: ProxiPlate-384 Plus, White 384-shallow well Microplate
Sealing tape: Topseal A plus
T175 culture flask

| Product | Units | | Storage |
|---|---|---|---|
| RPMI1640 (no L-Glutamine, no | 500 | mL | 4° C. |
| phenol red) | | | |
| Foetal Bovine Serum (FBS) (Heat | 500 | mL | 4° C. |
| inactivated) | | | |
| L-Glutamine (200 mM) | 100 | ml | −20° C. |
| Gentamicin (50 mg/mL) | 100 | mL | 4° C. |
| Caspase 3/7 Detection kit | 100 | mL | −20° C. |

10 × 10 mL

### Cell Culture Media:

| MOLP8 | | |
|---|---|---|
| RPMI-1640 medium | 500 | mL |
| 20% FBS (heat inactivated) | 120 | mL |
| 2 mM L-Glutamine | 6.2 | mL |
| 50 μg/mL Gentamicin | 620 | μL |
| Assay media | | |
| RPMI-1640 medium | 500 | mL |
| 10% FBS (Heat inactivated) | 57 | mL |
| 2 mM L-Glutamine | 5.7 | mL |
| 50 μg/mL Gentamicin | 570 | μL |

### Cell Culture:

Cell cultures were maintained between 0.2 and 2.0×10⁶ cells/mL. The cells were harvested by collection in 50 mL conical tubes. The cells were then pelleted at 500 g for 5 mins before removing supernatant and resuspension in fresh pre-warmed culture medium. The cells were counted and diluted as needed.

### Caspase-Glo Reagent:

The assay reagent was prepared by transferring the buffer solution to the substrate vial and mixing. The solution may be stored for up to 1 week at 4° C. with negligible loss of signal.

### Assay Procedure:

Compounds were delivered in assay-ready plates (Proxiplate) and stored at −20° C.

Assays always include 1 reference compound plate containing reference compounds. The plates were spotted with 40 nL of compounds (0.5% DMSO final in cells; serial dilution; 30 pM highest conc. 1/3 dilution, 10 doses, duplicates). The compounds were used at room temperature and 4 μL of pre-warmed media was added to all wells except column 2 and 23. The negative control was prepared by adding 1% DMSO in media. The positive control was prepared by adding the appropriate positive control compound in final concentration of 60 pM in media. The plate was prepared by adding 4 μL negative control to column 23, 4 μL positive control to column 2 and 4 μL cell suspension to all wells in the plate. The plate with cells was then incubated at 37° C. for 2 hours. The assay signal reagent is the Caspase-Glo solution described above, and 8 μL was added to all wells. The plates were then sealed and measured after 30 minutes.

The activity of a test compound was calculated as percent change in apoptosis induction as follows:

$$\begin{matrix}LC = median of the Low Control values \\ = Central Reference in Screener \\ = DMSO \\ = 0 \%\end{matrix}$$

$$\begin{matrix}HC = Median of the High control values \\ = Scale Reference in Screener \\ = 30 µM of positive control \\ = 100 \% apoptosis induction\end{matrix}$$

$$\% Effect ( A {C}_{50} ) = 100 - ( ( sample - LC ) / ( HC - LC ) ) ⋆ 100$$

$$\% Control = ( sample / HC ) ⋆ 100$$

$$\% Control min = ( ( sample - LC ) / ( HC - LC ) ) ⋆ 100$$

TABLE
Measured AC₅₀ for Representative Compounds
of Formula (I). Averaged values are reported
over all runs on all batches of a particular compound.

| | MOLP8 Caspase-Glo |
|---|---|
| Compound | AC50 (nM) LD value |
| 1 | NT |
| 2 | 1265.0 |
| 3 | 21.0 |
| 4 | 247.5 |
| 5 | 1287.9 |
| 6 | 930.7 |
| 7 | 11888.0 |
| 8 | 68.8 |
| 9 | 1169.5 |
| 10 | 37.6 |
| 11 | 8928.9 |
| 12 | 3416.6 |
| 13 | 24998.0 |
| 14 | 343.9 |
| 15 | >30000 |
| 16 | 19.7 |
| 17 | 46.8 |
| 18 | 17.0 |
| 19 | 20.7 |
| 20 | 8.8 |
| 21 | 49.5 |
| 22 | 20.1 |
| 23 | 30.6 |
| 28 | 16.7 |
| 29 | 126.4 |
| 30 | 20578 |
| 31 | 11.5 |
| 32 | 743.4 |
| 33 | 26.1 |
| 34 | 2087.8 |
| 35 | 54.1 |
| 36 | 2900 |
| 38 | 539.9 |
| 39 | 28.5 |
| 40 | 57.6 |
| 41 | 568.5 |
| 42 | 59.8 |
| 43 | 2007.5 |
| 44 | 4263.8 |
| 45 | 1597 |
| 46 | 14.6 |
| 47 | 2714.6 |
| 48 | 3693.8 |
| 49 | 141.2 |
| 50 | 83.6 |
| 51 | 87.3 |
| 52 | 39.4 |
| 53 | 1249.7 |
| 54 | 10.7 |
| 55 | 1642.9 |
| 56 | 43.6 |
| 57 | 4812.8 |
| 58 | 29.7 |
| 59 | 82.4 |
| 60 | 2152.3 |
| 61 | >30000 |
| 62 | 11.3 |
| 63 | 22.0 |
| 64 | 2102.3 |
| 65 | 1597.7 |
| 66 | 58.2 |
| 67 | 104.1 |
| 68 | 439.8 |
| 69 | 401.9 |

1. A compound of Formula (I) 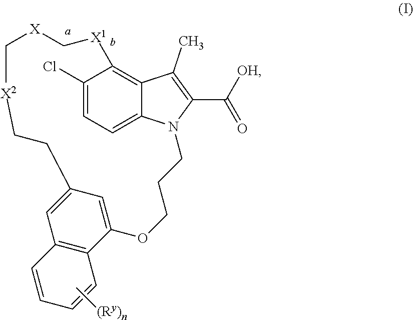
or a tautomer or a stereoisomeric form thereof, wherein
X¹ represents 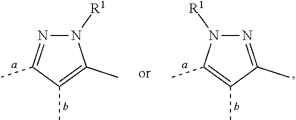
wherein ‘a’ and ‘b’ indicate how variable X¹ is attached to the remainder of the molecule;
R¹ and R² each independently represent hydrogen; methyl; or C₂₋₆alkyl optionally substituted with one or two substituents each independently selected from the group consisting of Het¹, —OR³, and —NR^{4a}R^{4b};
Het¹ represents morpholinyl or tetrahydropyranyl;
R³ represents hydrogen, C₁₋₄alkyl, —C₂₋₄alkyl-O—C₁₋₄alkyl, —C₂₋₄alkyl-OH, or —C₂₋₄alkyl-O—C₂₋₄alkyl-O—C₁₋₄alkyl;
R^{4a} and R^{4b} are each independently selected from the group consisting of hydrogen and C₁₋₄alkyl;
X² represents 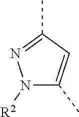
which can be attached to the remainder of the molecule in both directions;
X represents —O—, —S—, —S(═O)—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
R^{y} represents halo;
n represents 0, 1 or 2;
or a pharmaceutically acceptable salt, or a solvate thereof.

2. The compound according to claim 1, wherein
R³ represents hydrogen, C₁₋₄alkyl, —C₂₋₄alkyl-O—C₁₋₄alkyl, or
C₂₋₄alkyl-O—C₂₋₄alkyl-O—C₁₋₄alkyl;
X represents —O—, —S—, —S(═O)₂—, or —N(R^{x})—; and
n represents 0 or 1.

3. The compound according to claim 1, wherein
R¹ and R² each independently represent hydrogen; methyl; or C₂₋₆alkyl optionally substituted with one substituent selected from the group consisting of Het¹, —OR³, and —NR^{4a}R^{4b};
R³ represents hydrogen, C₁₋₄alkyl, or —C₂₋₄alkyl-O—C₁₋₄alkyl.

4. The compound according to claim 1, wherein
R^{x} represents methyl.

5. The compound according to claim 1, wherein
X¹ represents 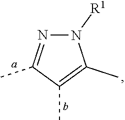
wherein ‘a’ and ‘b’ indicate how variable X¹ is attached to the remainder of the molecule;
R¹ and R² represent methyl;
X² represents 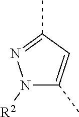
which can be attached to the remainder of the molecule in both directions;
X represents —S—, —S(═O)₂—, or —N(R^{x})—;
R^{x} represents methyl.

6. The compound according to claim 1, wherein
X¹ represents 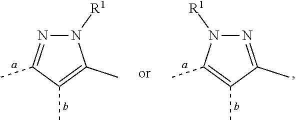
wherein ‘a’ and ‘b’ indicate how variable X¹ is attached to the remainder of the molecule;
R¹ and R² each independently represent methyl; or C₂₋₆alkyl optionally substituted with one or two substituents each independently selected from the group consisting of Het¹, —OR³, and —NR^{4a}R^{4b};
Het¹ represents tetrahydropyranyl;
R³ represents C₁₋₄alkyl, —C₂₋₄alkyl-O—C₁₋₄alkyl, or —C₂₋₄alkyl-O—C₂₋₄alkyl-O—C₁₋₄alkyl;
R^{4a} and R^{4b} represent hydrogen;
X² represents 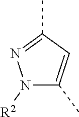
which can be attached to the remainder of the molecule in both directions;
X represents —S—, —S(═O)₂—, or —N(R^{x})—;
R^{x} represents methyl;
R^{y} represents halo;
n represents 0 or 1.

7. The compound according to claim 1,
wherein
X represents —S—.

8. The compound according to claim 1, wherein
R^{y} represents fluoro.

9. The compound according to claim 1, wherein
X¹ represents

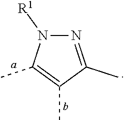

10. A pharmaceutical composition comprising a compound as claimed in claim 1 and a pharmaceutically acceptable carrier or diluent.

11. A process for preparing a pharmaceutical composition as defined in claim 10 comprising mixing a pharmaceutically acceptable carrier with a therapeutically effective amount of a compound according to claim 1.

12. A compound as claimed in claim 1 for use as a medicament.

13. A compound as claimed in claim 1 for use in the prevention or treatment of cancer.

14. The compound for use according to claim 13, wherein cancer is selected from prostate, lung, pancreatic, breast, ovarian, cervical, melanoma, B-cell chronic lymphocytic leukemia (CLL), acute myeloid leukemia (AML), and acute lymphoblastic leukemia (ALL).

15. A method of treating or preventing cancer, comprising administering to a subject in need thereof, a therapeutically effective amount of a compound as claimed in claim 1.
